(19) United States
(12) Patent Application Publication
(10) Pub. No.: US 2022/0274954 A1
(43) Pub. Date: Sep. 1, 2022

(54) SUBSTITUTED N-PHENYL-N-AMINOUARCILS AND SALTS THEREOF AND USE THEREOF AS HERBICIDAL AGENTS

(71) Applicant: Bayer Aktiengesellschaft, Leverkusen (DE)

(72) Inventors: Jens FRACKENPOHL, Frankfurt (DE); Ines HEINEMANN, Hofheim (DE); Lothar WILLMS, Hillscheid (DE); Harald JAKOBI, Frankfurt (DE); Hendrik HELMKE, Liederbach (DE); Christopher Hugh ROSINGER, Hofheim (DE); Elisabeth ASMUS, Hoesbach (DE)

(21) Appl. No.: 17/628,830

(22) PCT Filed: Jul. 20, 2020
(86) PCT No.: PCT/EP2020/070464

§ 371 Date: Jan. 20, 2022

(30) Foreign Application Priority Data
Jul. 22, 2019 (EP) 19187525.1

(51) Int. Cl. C07D 401/12; A01N 43/54; A01N 43/60; A01P 13/02

(52) U.S. Cl. CPC C07D 401/12; A01N 43/54; A01N 43/60; A01P 13/02

(57) ABSTRACT

The present invention relates to substituted N-phenyl-N-aminouracils of the general formula (I) or salts thereof

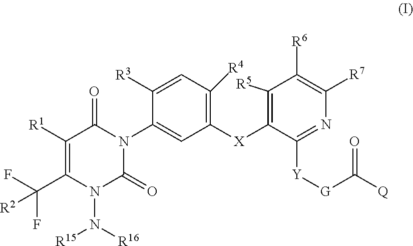

wherein the radicals in the general formula (I) correspond to the definitions given in the description,
and to their use as herbicides, in particular for controlling broad-leaved weeds and/or weed grasses in crops of useful plants and/or as plant growth regulators for influencing the growth of crops of useful plants.

The invention relates to the technical field of crop protection agents, in particular that of herbicides for the selective control of broad-leaved weeds and weed grasses in crops of useful plants.

Specifically, the present invention relates to substituted N-phenyl-N-aminouracils and to their salts, to processes for their preparation and to their use as herbicides, in particular for controlling broad-leaved weeds and/or weed grasses in crops of useful plants and/or as plant growth regulators for influencing the growth of crops of useful plants.

In their application, crop protection agents known to date for the selective control of harmful plants in crops of useful plants or active compounds for controlling unwanted vegetation sometimes have disadvantages, be it (a) that they have no or else insufficient herbicidal activity against particular harmful plants, (b) that the spectrum of harmful plants which can be controlled with an active compound is not wide enough, (c) that their selectivity in crops of useful plants is too low and/or (d) that they have a toxicologically unfavorable profile. Furthermore, some active compounds which can be used as plant growth regulators for a number of useful plants cause unwanted reduced harvest yields in other useful plants or are not compatible with the crop plant, or only within a narrow application rate range. Some of the known active compounds cannot be produced economically on an industrial scale owing to precursors and reagents which are difficult to obtain, or they have only insufficient chemical stabilities. In the case of other active compounds, the activity is too highly dependent on environmental conditions, such as weather and soil conditions.

The herbicidal activity of these known compounds, in particular at low application rates, and/or their compatibility with crop plants remain in need of improvement.

From various publications, it is known that certain substituted N-attached aryluracils can be employed as herbicidally active compounds (cf. EP408382, EP473551, EP648749, U.S. Pat. Nos. 4,943,309, 5,084,084, 5,127,935, WO91/00278, WO95/29168, WO95/30661, WO96/35679, WO97/01541, WO98/25909, WO2001/39597). However, the known aryluracils have a number of activity gaps, in particular with regard to monocotyledonous weeds. A number of herbicidal active compound combinations based on N-attached aryluracils are also known (cf. DE4437197, EP714602, WO96/07323, WO96/08151, JP11189506). However, the properties of these active compound combinations are not entirely satisfactory.

Furthermore, it is known that certain N-aryluracils having lactic acid groups, optionally with further substitution, can likewise be employed as herbicidally active compounds (cf. JP2000/302764, JP2001/172265, U.S. Pat. No. 6,403,534, EP408382A1). In addition, it is known that N-aryluracils having specific thiolactic acid groups, optionally with further substitution, are likewise herbicidally active (cf. WO2010/038953, KR2011110420). Selected substituted tetrahydrofuryl esters of N-aryluracilen having thiolactic acid groups, optionally with further substitution, are described in JP09188676.

Also known are substituted uracils having an N-attached and further substituted diaryl ether group or a corresponding heteroaryl aryl ether radical (cf. U.S. Pat. Nos. 6,333,296, 6,121,201, WO2001/85907, EP1122244A1, EP1397958A1, EP1422227A1, WO 2002/098227, WO 2018/019842). Furthermore, highly substituted N-aryluracils having a carbonylalkyloxy group with specific substitution have been described (cf. WO2011/137088). Substituted 3-phenyl-5-alkyl-6-(trifluoromethyl)pyrimidine-2,4(1H,3H)-diones (cf. WO2019/101551) and related substituted 3-(pyridin-2-yl)-5-alkyl-6-(trifluoromethyl)pyrimidine-2,4(1H,3H)-diones (cf. WO2019/101513) are likewise known.

Surprisingly, it has now been found that certain substituted N-phenyl-N-aminouracils or salts thereof are very suitable as herbicides and can be employed particularly advantageously as active compounds for controlling monocotyledonous and dicotyledonous weeds in crop plant cultures.

Accordingly, the present invention provides substituted N-phenyl-N-aminouracils of the general formula (I) or salts thereof

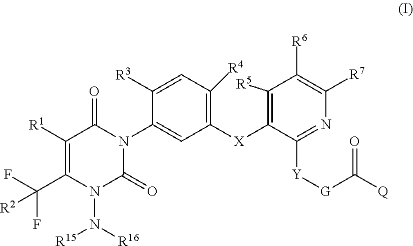

in which
R¹ represents hydrogen, (C₁-C₈)-haloalkyl,
R² represents hydrogen, fluorine, chlorine, bromine, trifluoromethyl, (C₁-C₈)-alkoxy,
R³ represents hydrogen, halogen, (C₁-C₈)-alkoxy,
R⁴ represents halogen, cyano, NO₂, C(O)NH₂, C(S)NH₂, (C₁-C₈)-haloalkyl, (C₂-C₈)-alkynyl,
R⁵, R⁶ and R⁷ independently of one another represent hydrogen, halogen, cyano, (C₁-C₈)-alkyl, (C₁-C₈-haloalkyl, (C₁-C₈-alkoxy, (C₁-C₈-haloalkoxy,
G represents straight-chain or branched (C₁-C₈-alkylene,
Q represents hydroxy or a radical of the formula below

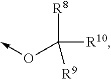

R⁸ represents hydrogen, (C₁-C₈)-alkyl, (C₁-C₈)-haloalkyl, aryl, aryl-(C₁-C₈-alkyl, heteroaryl, (C₂-C₈)-alkynyl, (C₂-C₈)-alkenyl, C(O)R¹³, C(O)OR¹³, (C₁-C₈-alkoxy-(C₁-C₈-alkyl,
R⁹ represents hydrogen or (C₁-C₈)-alkyl,
R¹⁰ represents hydrogen, halogen, cyano, NO₂, (C₁-C₈)-alkyl, (C₁-C₈)-haloalkyl, (C₃-C₈)-cycloalkyl, (C₃-C₈)-cycloalkyl-(C₁-C₈)-alkyl, (C₃-C₈)-halocycloalkyl, (C₃-C₈)-halocycloalkyl-(C₁-C₈)-alkyl, (C₂-C₈)-alkenyl, (C₂-C₈)-alkynyl, aryl, aryl-(C₁-C₈-alkyl, heteroaryl, heteroaryl-(C₁-C₈-alkyl, heterocyclyl, heterocyclyl-(C₁-C₈)-alkyl, R¹¹R¹²N—(C₁-C₈)-alkyl, R¹³O—(C₁-C₈-alkyl, cyano-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈-alkyl, heteroarylcarbonyloxy-(C₁-C₈-alkyl, heterocyclylcarbonyloxy-(C₁-C₈-alkyl, OR¹³, NR¹¹R¹², SR¹⁴, S(O)R¹⁴, SO₂R¹⁴, R¹⁴, S—(C₁-C₈)-alkyl, R¹⁴(O)S—(C₁-C₈)-alkyl, R¹⁴O₂S—(C₁-C₈)-alkyl, tris-[(C₁-C₈)-alkyl]silyl-(C₁-C₈)-alkyl, bis-[(C₁-C₈)-alkylyaryl]silyl(C₁-C₈)-alkyl, [(C₁-C₈)-alkyl]-bis-(aryl)silyl-(C₁-C₈)-alkyl, tris-[(C₁-C₈)-alkyl]silyl, bis-hydroxyboryl-(C₁-C₈)-alkyl, bis-[(C₁-C₈)-alkoxy]boryl-(C₁-C₈)-alkyl, tetramethyl-1,3,2-dioxaborolan-2-yl, tetramethyl-1,3,2-dioxaborolan-2-yl-(C₁-C₈)-alkyl, nitro-(C₁-C₈-alkyl, C(O)OR¹³, C(O)R¹³, C(O)NR¹¹, R¹², R¹³O(O)C—(C₁-C₈)-alkyl, R¹¹R¹²N(O)C—(C₁-C₈)-alkyl, bis-(C₁-C₈-alkoxy-(C₁-C₈-alkyl, or
R⁸ and R¹⁰ together with the carbon atom to which they are attached form a fully saturated or partially saturated 3- to 10-membered monocyclic or bicyclic ring optionally interrupted by heteroatoms and optionally having further substitution,
R¹¹ and R¹² are identical or different and independently of one another represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈-alkoxy-(C₁-C₈-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₅)-cycloalkyl-(C₁-C₈)-alkyl, (C₄-C₁₀)-cycloalkenyl-(C₁-C₈)-alkyl, COR¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, heterocyclyl-(C₁-C₈)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution,
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₅)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, (C₁-C₈)-alkoxycarbonyl,
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkyl-amino, aryl-(C₁-C₈)-amino, aryl-(C₁-C₆)-alkyl-amino, aryl-[(C₁-C₈)-alkyl]amino, (C₃-C₈)-cycloalkyl-amino, (C₃-C₈)-cycloalkyl-[(C₁-C₈)-alkyl]amino; N-azetidinyl, N-pyrrolidinyl, N-piperidinyl, N-morpholinyl,
R¹⁵ and R¹⁶ independently of one another represent hydrogen, (C₁-C₈)-alkyl, (C₂-C₈)-alkenyl, C(O)R¹³, C(O)OR¹³, C(O)NR¹¹R¹², SO₂R¹⁴, or
R¹⁵ and R¹⁶ together with the nitrogen atom to which they are attached form an imino group which is optionally substituted further by hydrogen, (C₁-C₈)-alkyl, aryl-(C₁-C₈)-alkyl, (C₃-C₈)-cycloalkyl, aryl, heteroaryl, heterocyclyl, (C₁-C₈)-alkoxycarbonyl-(C₁-C₈)-alkyl, aryl-(C₁-C₈)-alkoxycarbonyl-(C₁-C₈)-alkyl and
X and Y independently of one another represent O (oxygen) or S (sulfur).

The invention preferably provides compounds of the general formula (I) in which
R¹ represents hydrogen,
R² represents hydrogen, fluorine, chlorine, bromine, trifluoromethyl, (C₁-C₇)-alkoxy,
R³ represents hydrogen, halogen, (C₁-C₇)-alkoxy,
R⁴ represents halogen, cyano, NO₂, C(O)NH₂, C(S)NH₂, (C₁-C₇)-haloalkyl, (C₂-C₇)-alkynyl,
R⁵, R⁶ and R⁷ independently of one another represent hydrogen, halogen, cyano, (C₁-C₇)-alkyl, (C₁-C₇)-haloalkyl, (C₁-C₇)-alkoxy, (C₁-C₇)-haloalkoxy,
G represents straight-chain or branched (C₁-C₇)-alkylene,
Q represents hydroxy or a radical of the formula below

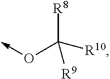

R⁸ represents hydrogen, (C₁-C₇)-alkyl, (C₁-C₇)-haloalkyl, aryl, aryl-(C₁-C₇)-alkyl, heteroaryl, (C₂-C₇)-alkynyl, (C₂-C₇)-alkenyl, C(O)R¹³, C(O)OR¹³, (C₁-C₇)-alkoxy-(C₁-C₇)-alkyl,
R⁹ represents hydrogen or (C₁-C₆)-alkyl,
R¹⁰ represents hydrogen, halogen, cyano, NO₂, (C₁-C₇)-alkyl, (C₁-C₇)-haloalkyl, (C₃-C₇)-cycloalkyl, (C₃-C₇)-cycloalkyl-(C₁-C₇)-alkyl, (C₃-C₇)-halocycloalkyl, (C₃-C₇)-halocycloalkyl-(C₁-C₇)-alkyl, (C₂-C₇)-alkenyl, (C₂-C₇)-alkynyl, aryl, aryl-(C₁-C₇)-alkyl, heteroaryl, heteroaryl-(C₁-C₇)-alkyl, heterocyclyl, heterocyclyl-(C₁-C₇)-alkyl, R¹¹R¹²N—(C₁-C₇)-alkyl, R¹³O—(C₁-C₇)-alkyl, cyano-(C₁-C₇)-alkyl, (C₁-C₇)-alkylcarbonyloxy-(C₁-C₇)-alkyl, (C₃-C₇)-cycloalkylcarbonyloxy-(C₁-C₇)-alkyl, arylcarbonyloxy-(C₁-C₇)-alkyl, heteroarylcarbonyloxy-(C₁-C₇)-alkyl, heterocyclylcarbonyloxy-(C₁-C₇)-alkyl, OR¹³, NR¹¹R¹², SR¹⁴, S(O)R¹⁴, SO₂R¹⁴, R¹⁴S—(C₁-C₇)-alkyl, R¹⁴(O)S—(C₁-C₇)-alkyl, R¹⁴O₂S—(C₁-C₇)-alkyl, tris-[(C₁-C₇)-alkyl]silyl-(C₁-C₇)-alkyl, bis-[(C₁-C₇)-alkyl](aryl)silyl(C₁-C₇)-alkyl, [(C₁-C₇)-alkyl]-bis-(aryl)silyl-(C₁-C₇)-alkyl, tris-[(C₁-C₇)-alkyl]silyl, bis-hydroxyboryl-(C₁-C₇)-alkyl, bis-[(C₁-C₇)-alkoxy]boryl-(C₁-C₇)-alkyl, tetramethyl-1,3,2-dioxaborolan-2-yl, tetramethyl-1,3,2-dioxaborolan-2-yl-(C₁-C₇)-alkyl, nitro-(C₁-C₇)-alkyl, C(O)OR¹³, C(O)R¹³, C(O)NR¹¹R¹², R¹³O(O)C—(C₁-C₇)-alkyl, R¹¹R¹²N(O)C—(C₁-C₇)-alkyl, bis-(C₁-C₇)-alkoxy-(C₁-C₇)-alkyl,
R¹¹ and R¹² are identical or different and independently of one another represent hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₃-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-haloalkoxy-(C₁-C₇)-alkyl, (C₁-C₇)-alkylthio-(C₁-C₇)-alkyl, (C₁-C₇)-haloalkylthio-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, (C₃-C₇)-cycloalkyl-(C₁-C₇)-alkyl, (C₄-C₁₀)-cycloalkenyl-(C₁-C₇)-alkyl, COR¹³, SO₂R¹⁴, heterocyclyl, (C₁-C₇)-alkoxycarbonyl, bis-[(C₁-C₇)-alkyl]aminocarbonyl-(C₁-C₇)-alkyl, (C₁-C₇)-alkyl-aminocarbonyl-(C₁-C₇)-alkyl, aryl-(C₁-C₇)-alkyl-aminocarbonyl-(C₁-C₇)-alkyl, aryl-(C₁-C₇)-alkoxycarbonyl, heteroaryl-(C₁-C₇)-alkoxycarbonyl, (C₂-C₇)-alkenyloxycarbonyl, (C₂-C₇)-alkynyloxycarbonyl, heterocyclyl-(C₁-C₇)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution,
R¹³ represents hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₃-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-haloalkoxy-(C₁-C₇)-alkyl, (C₁-C₇)-alkoxy-(C₁-C₇)-haloalkyl, (C₁-C₇)-alkoxy-(C₁-C₇)-alkoxy-(C₁-C₇)-alkyl, (C₁-C₇)-alkoxy-(C₁-C₇)-alkoxy-(C₁-C₇)-alkoxy-(C₁-C₇)-alkyl, (C₁-C₇)-alkoxy-(C₁-C₇)-alkoxy-(C₁-C₇)-alkoxy-(C₁-C₇)-alkoxy-(C₁-C₇)-alkyl, aryl, aryl-(C₁-C₇)-alkyl, aryl-(C₁-C₇)-alkoxy-(C₁-C₇)-alkyl, heteroaryl, heteroaryl-(C₁-C₇)-alkyl, (C₃-C₇)-cycloalkyl-(C₁-C₇)-alkyl, (C₄-C₁₀)-cycloalkenyl-(C₁-C₇)-alkyl, bis-[(C₁-C₇)-alkyl]aminocarbonyl-(C₁-C₇)-alkyl, (C₁-C₇)-alkyl-aminocarbonyl-(C₁-C₇)-alkyl, aryl-(C₁-C₇)-alkyl-aminocarbonyl-(C₁-C₇)-alkyl, bis-[(C₁-C₇)-alkyl]amino-(C₂-C₅)-alkyl, (C₁-C₇)-alkyl-amino-(C₂-C₅)-alkyl, aryl-(C₁-C₇)-alkyl-amino-(C₂-05)-alkyl, R¹⁴S—(C₁-C₇)-alkyl, R¹⁴(O)S—(C₁-C₇)-alkyl, R¹⁴O₂S—(C₁-C₇)-alkyl, hydroxycarbonyl-(C₁-C₇)-alkyl, heterocyclyl, heterocyclyl-(C₁-C₇)-alkyl, tris-[(C₁-C₇)-alkyl]silyl-(C₁-C₇)-alkyl, bis-[(C₁-C₇)-alkyl](aryl)silyl(C₁-C₇)-alkyl, [(C₁-C₇)-alkyl]-bis-(aryl)silyl-(C₁-C₇)-alkyl, (C₁-C₇)-alkylcarbonyloxy-(C₁-C₇)-alkyl, (C₃-C₇)-cycloalkylcarbonyloxy-(C₁-C₇)-alkyl, arylcarbonyloxy-(C₁-C₇)-alkyl, heteroarylcarbonyloxy-(C₁-C₇)-alkyl, heterocyclylcarbonyloxy-(C₁-C₇)-alkyl, aryloxy-(C₁-C₇)-alkyl, heteroaryloxy-(C₁-C₇)-alkyl, (C₁-C₇)-alkoxycarbonyl,
R¹⁴ represents hydrogen, (C₁-C₇)-alkyl, (C₂-C₇)-alkenyl, (C₂-C₇)-alkynyl, (C₁-C₇)-cyanoalkyl, (C₁-C₁₀)-haloalkyl, (C₂-C₇)-haloalkenyl, (C₃-C₇)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₇)-alkoxy-(C₁-C₇)-alkyl, (C₁-C₇)-alkoxy-(C₁-C₇)-haloalkyl, aryl, aryl-(C₁-C₇)-alkyl, heteroaryl, heteroaryl-(C₁-C₇)-alkyl, heterocyclyl-(C₁-C₇)-alkyl, (C₃-C₇)-cycloalkyl-(C₁-C₇)-alkyl, (C₄-C₁₀)-cycloalkenyl-(C₁-C₇)-alkyl, bis-[(C₁-C₇)-alkyl]amino, (C₁-C₇)-alkyl-amino, aryl-(C₁-C₇)-amino, aryl-(C₁-C₄)-alkyl-amino, aryl-[(C₁-C₇)-alkyl]amino; (C₃-C₇)-cycloalkyl-amino, (C₃-C₇)-cycloalkyl-[(C₁-C₇)-alkyl]amino; N-azetidinyl, N-pyrrolidinyl, N-piperidinyl, N-morpholinyl,
R¹⁵ and R¹⁶ independently of one another represent hydrogen, (C₁-C₇)-alkyl, (C₂-C₇)-alkenyl, C(O)R¹³, C(O)OR¹³, C(O)NR¹¹R¹², SO₂R¹⁴, or
R¹⁵ and R¹⁶ together with the nitrogen atom to which they are attached form an imino group which is optionally substituted further by hydrogen, (C₁-C₇)-alkyl, aryl-(C₁-C₇)-alkyl, (C₃-C₇)-cycloalkyl, aryl, heteroaryl, heterocyclyl, (C₁-C₇)-alkoxycarbonyl-(C₁-C₇)-alkyl, aryl-(C₁-C₇)-alkoxycarbonyl-(C₁-C₇)-alkyl and
X and Y independently of one another represent O (oxygen) or S (sulfur).

The invention particularly preferably provides compounds of the general formula (I), in which
R¹ represents hydrogen,
R² represents hydrogen, fluorine, chlorine, bromine, trifluoromethyl, (C₁-C₆)-alkoxy,
R³ represents hydrogen, halogen, (C₁-C₆)-alkoxy,
R⁴ represents halogen, cyano, NO₂, C(O)NH₂, C(S)NH₂, (C₁-C₆)-haloalkyl, (C₂-C₆)-alkynyl,
R⁵, R⁶ and R⁷ independently of one another represent hydrogen, halogen, cyano, (C₁-C₆)-alkyl, (C₁-C₆)-haloalkyl, (C₁-C₆)-alkoxy, (C₁-C₆)-haloalkoxy,
G represents straight-chain or branched (C₁-C₆)-alkylene,
Q represents hydroxy or a radical of the formula below

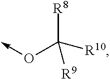

R⁸ represents hydrogen, (C₁-C₆)-alkyl, (C₁-C₆)-haloalkyl, aryl, aryl-(C₁-C₆)-alkyl, heteroaryl, (C₂-C₆)-alkynyl, (C₂-C₆)-alkenyl, C(O)R¹³, C(O)OR¹³, (C₁-C₆)-alkoxy-(C₁-C₆)-alkyl,
R⁹ represents hydrogen or (C₁-C₄)-alkyl,
R¹⁰ represents hydrogen, halogen, cyano, nitro, (C₁-C₆)-alkyl, (C₁-C₆)-haloalkyl, (C₃-C₆)-cycloalkyl, (C₃-C₆)-cycloalkyl-(C₁-C₆)-alkyl, (C₃-C₆)-halocycloalkyl, (C₃-C₆)-halocycloalkyl-(C₁-C₆)-alkyl, (C₂-C₆)-alkenyl, (C₂-C₆)-alkynyl, aryl, aryl-(C₁-C₆)-alkyl, heteroaryl, heteroaryl-(C₁-C₆)-alkyl, heterocyclyl, heterocyclyl-(C₁-C₆)-alkyl, R¹¹R¹²N—(C₁-C₆)-alkyl, R¹³O—(C₁-C₆)-alkyl, cyano-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, OR¹³, NR¹¹R¹², SR¹⁴, S(O)R¹⁴, SO₂R¹⁴, R¹⁴S—(C₁-C₆)-alkyl, R¹⁴(O)S—(C₁-C₆)-alkyl, R¹⁴O₂S—(C₁-C₆)-alkyl, tris-[(C₁-C₆)-alkyl]silyl-(C₁-C₆)-alkyl, bis-[(C₁-C₆)-alkyl](aryl)silyl(C₁-C₆)-alkyl, [(C₁-C₆)-alkyl]-bis-(aryl)silyl-(C₁-C₆)-alkyl, tris-[(C₁-C₆)-alkyl]silyl, bis-hydroxyboryl-(C₁-C₆)-alkyl, bis-[(C₁-C₆)-alkoxy]boryl-(C₁-C₆)-alkyl, tetramethyl-1,3,2-dioxaborolan-2-yl, tetramethyl-1,3,2-dioxaborolan-2-yl-(C₁-C₆)-alkyl, nitro-(C₁-C₆)-alkyl, C(O)OR¹³, C(O)R¹³, C(O)NR¹¹R¹², R¹³O(O)C—(C₁-C₆)-alkyl, R¹¹R¹²N(O)C—(C₁-C₆)-alkyl, bis-(C₁-C₆)-alkoxy-(C₁-C₆)-alkyl, or
R⁸ and R¹⁰ together with the carbon atom to which they are attached form a fully saturated or partially saturated 3- to 10-membered monocyclic or bicyclic ring optionally interrupted by heteroatoms and optionally having further substitution,
R¹¹ and R¹² are identical or different and independently of one another represent hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, COR¹³, SO₂R¹⁴, heterocyclyl, (C₁-C₆)-alkoxycarbonyl, bis-[(C₁-C₆)-alkyl]aminocarbonyl-(C₁-C₆)-alkyl, (C₁-C₆)-alkyl-aminocarbonyl-(C₁-C₆)-alkyl, aryl-(C₁-C₆)-alkyl-aminocarbonyl-(C₁-C₆)-alkyl, aryl-(C₁-C₆)-alkoxycarbonyl, heteroaryl-(C₁-C₆)-alkoxycarbonyl, (C₂-C₆)-alkenyloxycarbonyl, (C₂-C₆)-alkynyloxycarbonyl, heterocyclyl-(C₁-C₆)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution,
R¹³ represents hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkoxy-(C₁-C₆)-haloalkyl, (C₁-C₆)-alkoxy-(C₁-C₆)-alkoxy-(C₁-C₆)-alkyl, (C₁-C₆)-alkoxy-(C₁-C₆)-alkoxy-(C₁-C₆)-alkoxy-(C₁-C₆)-alkyl, (C₁-C₆)-alkoxy-(C₁-C₆)-alkoxy-(C₁-C₆)-alkoxy-(C₁-C₆)-alkoxy-(C₁-C₆)-alkyl, aryl, aryl-(C₁-C₆)-alkyl, aryl-(C₁-C₆)-alkoxy-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, bis-[(C₁-C₆)-alkyl]aminocarbonyl-(C₁-C₆)-alkyl, (C₁-C₆)-alkyl-aminocarbonyl-(C₁-C₆)-alkyl, aryl-(C₁-C₆)-alkyl-aminocarbonyl-(C₁-C₆)-alkyl, bis-[(C₁-C₆)-alkyl]amino-(C₂-C₄)-alkyl, (C₁-C₆)-alkyl-amino-(C₂-C₄)-alkyl, aryl-(C₁-C₆)-alkyl-amino-(C₂-C₄)-alkyl, R¹⁴S—(C₁-C₆)-alkyl, R¹⁴(O)S—(C₁-C₆)-alkyl, R¹⁴O₂S—(C₁-C₆)-alkyl, hydroxycarbonyl-(C₁-C₆)-alkyl, heterocyclyl, heterocyclyl-(C₁-C₆)-alkyl, tris-[(C₁-C₆)-alkyl]silyl-(C₁-C₆)-alkyl, bis-[(C₁-C₆)-alkyl](aryl)silyl(C₁-C₆)-alkyl, [(C₁-C₆)-alkyl]-bis-(aryl)silyl-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, aryloxy-(C₁-C₆)-alkyl, heteroaryloxy-(C₁-C₆)-alkyl, (C₁-C₆)-alkoxycarbonyl,
R¹⁴ represents hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, heterocyclyl-(C₁-C₆)-alkyl, (C₃-C₆)-cycloalkyl-(C₁-C₆)-alkyl, (C₄-C₁₀)-cycloalkenyl-(C₁-C₆)-alkyl, bis-[(C₁-C₆)-alkyl]amino, (C₁-C₆)-alkyl-amino, aryl-(C₁-C₆)-amino, aryl-(C₁-C₂)-alkyl-amino, aryl-[(C₁-C₆)-alkyl]amino; (C₃-C₆)-cycloalkyl-amino, (C₃-C₆)-cycloalkyl-[(C₁-C₆)-alkyl]amino; N-azetidinyl, N-pyrrolidinyl, N-piperidinyl, N-morpholinyl,
R¹⁵ and R¹⁶ independently of one another represent hydrogen, (C₁-C₆)-alkyl, (C₂-C₆)-alkenyl, C(O)R¹³, C(O)OR¹³, C(O)NR¹¹R¹², SO₂R¹⁴, or
R¹⁵ and R¹⁶ together with the nitrogen atom to which they are attached form an imino group which is optionally substituted further by hydrogen, (C₁-C₆)-alkyl, aryl-(C₁-C₆)-alkyl, (C₃-C₆)-cycloalkyl, aryl, heteroaryl, heterocyclyl, (C₁-C₆)-alkoxycarbonyl-(C₁-C₆)-alkyl, aryl-(C₁-C₆)-alkoxycarbonyl-(C₁-C₆)-alkyl and
X and Y independently of one another represent O (oxygen) or S (sulfur).

The invention very particularly preferably provides compounds of the general formula (I) in which
R¹ represents hydrogen,
R² represents hydrogen, fluorine, chlorine, bromine, trifluoromethyl, methoxy, ethoxy, prop-1-yloxy, but-1-yloxy,
R³ represents hydrogen, fluorine, chlorine, bromine, methoxy, ethoxy, prop-1-yloxy, prop-2-yloxy, but-1-yloxy, but-2-yloxy, 2-methylprop-1-yloxy, 1,1-dimethyleth-1-yloxy,
R⁴ represents fluorine, chlorine, bromine, cyano, NO₂, C(O)NH₂, C(S)NH₂, trifluoromethyl, difluoromethyl, pentafluoroethyl, ethynyl, propyn-1-yl, 1-butyn-1-yl, pentyn-1-yl, hexyn-1-yl,
R⁵, R⁶ and R⁷ independently of one another represent hydrogen, fluorine, chlorine, bromine, iodine, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, difluoromethyl, pentafluoroethyl, 2,2-difluoroethyl, 2,2,2-trifluoroethyl, methoxy, ethoxy, prop-1-yloxy, prop-2-yloxy, but-1-yloxy, but-2-yloxy, 2-methylprop-1-yloxy, 1,1-dimethyleth-1-yloxy, difluoromethoxy, trifluoromethoxy, pentafluoroethoxy, 2,2-difluoroethoxy, 2,2,2-trifluoroethoxy,
G represents methylene, (methyl)methylene, (ethyl)methylene, (prop-1-yl)methylene, (prop-2-yl)methylene, (but-1-yl)methylene, (but-2-yl)methylene, (pent-1-yl)methylene, (pent-2-yl)methylene, (pent-3-yl)methylene, (dimethyl)methylene, (diethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, 1,1,2-trimethylethyl-1-ene, 1,2,2-trimethylethyl-1-ene, 1,1,2,2-tetramethylethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene, 1-methylpentyl-1-ene, 2-methylpentyl-1-ene, 3-methylpentyl-1-ene, 4-methylpentyl-1-ene, 1,1-dimethylbutyl-1-ene, 1,2-dimethylbutyl-1-ene, 1,3-dimethylbutyl-1-ene, 2,2-dimethylbutyl-1-ene, 2,3-dimethylbutyl-1-ene, 3,3-dimethylbutyl-1-ene, 1-ethylbutyl-1-ene, 2-ethylbutyl-1-ene, 1,1,2-trimethylpropyl-1-ene, 1,2,2-trimethylpropyl-1-ene, 1-ethyl-1-methylpropyl-1-ene, 1-ethyl-2-methylpropyl-1-ene,
R¹⁵ and R¹⁶ independently of one another represent hydrogen, methyl, ethyl, methylcarbonyl, ethylcarbonyl, methoxycarbonyl, ethoxycarbonyl, tert-butyloxycarbonyl,
X and Y independently of one another represent O (oxygen) or S (sulfur)
   and Q represents one of the moieties Q-1 to Q-486 specifically mentioned below:

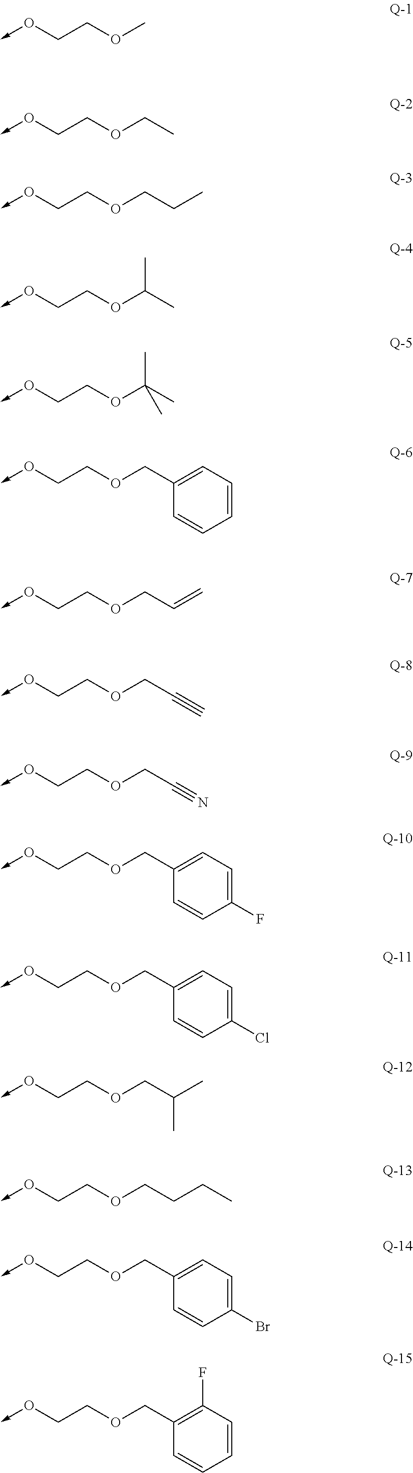

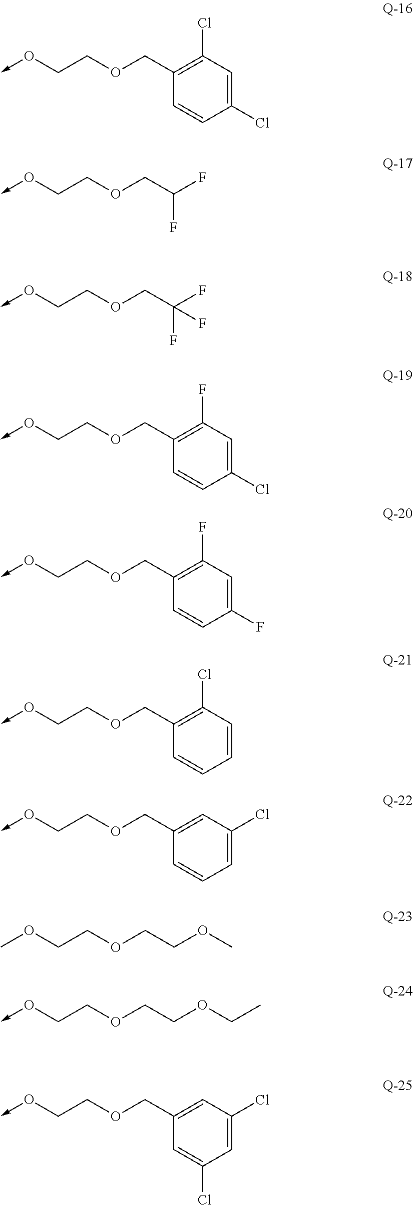

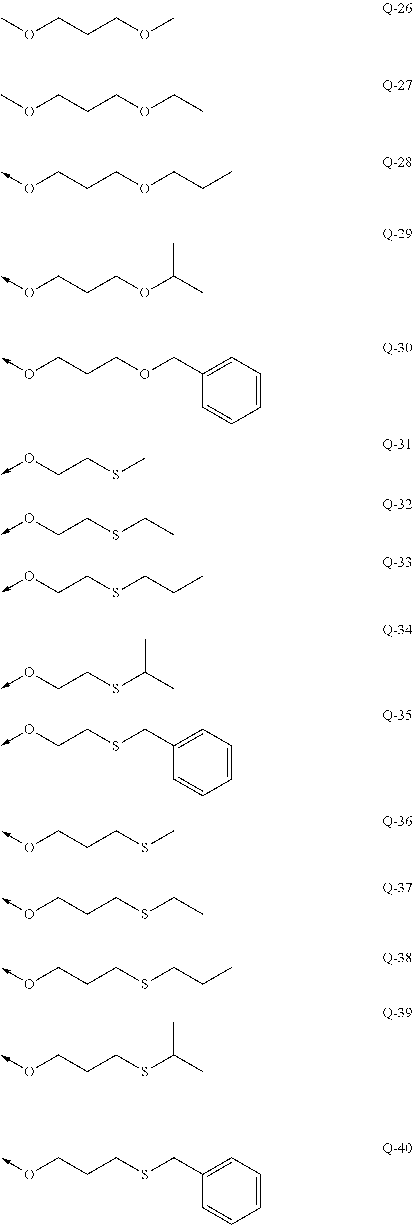

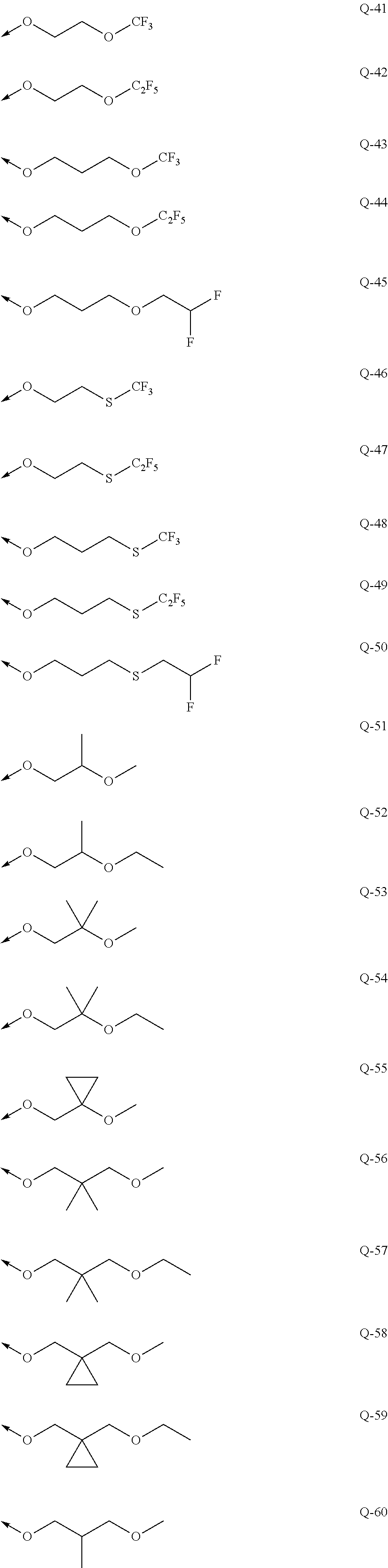

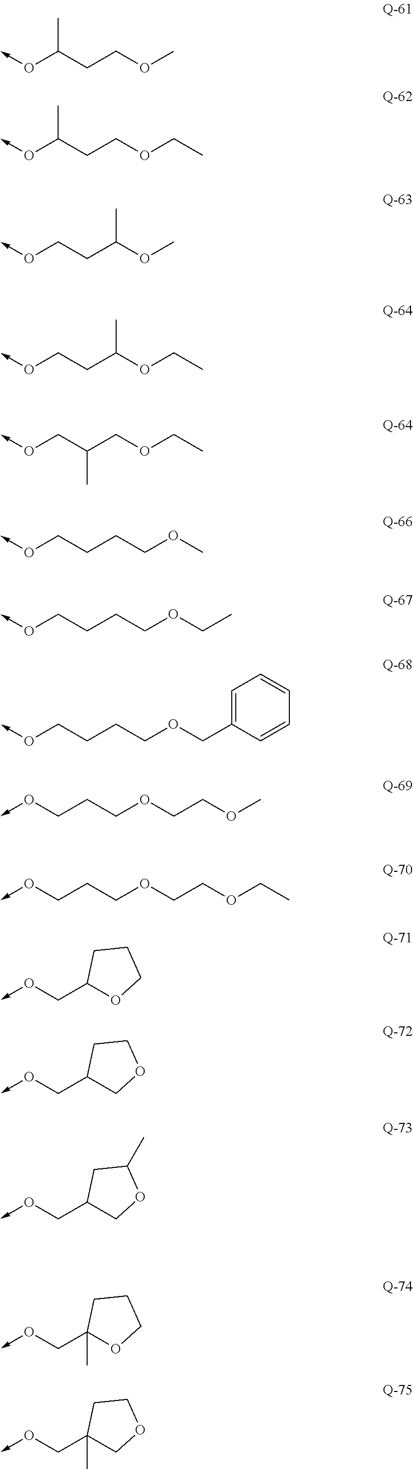

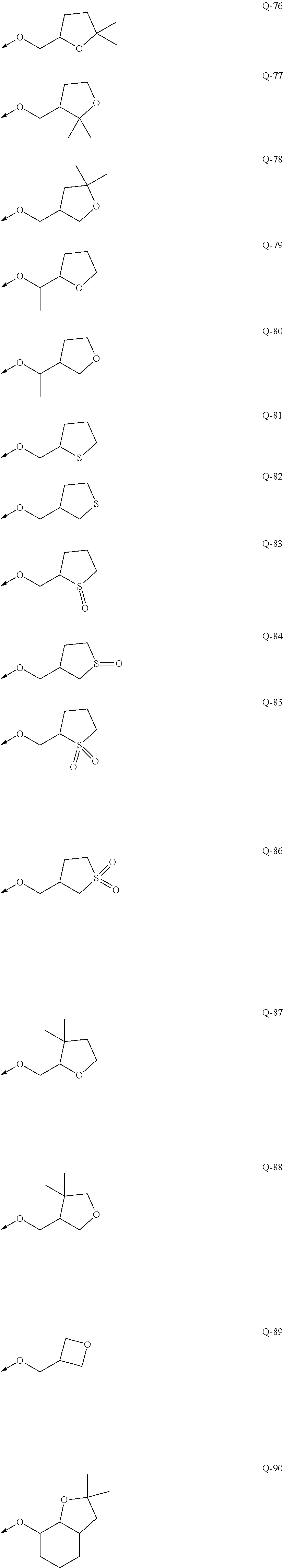

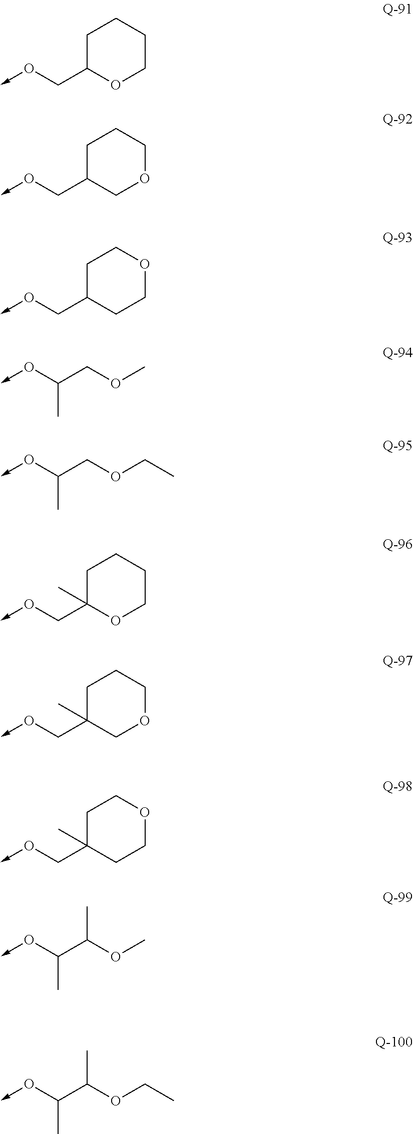

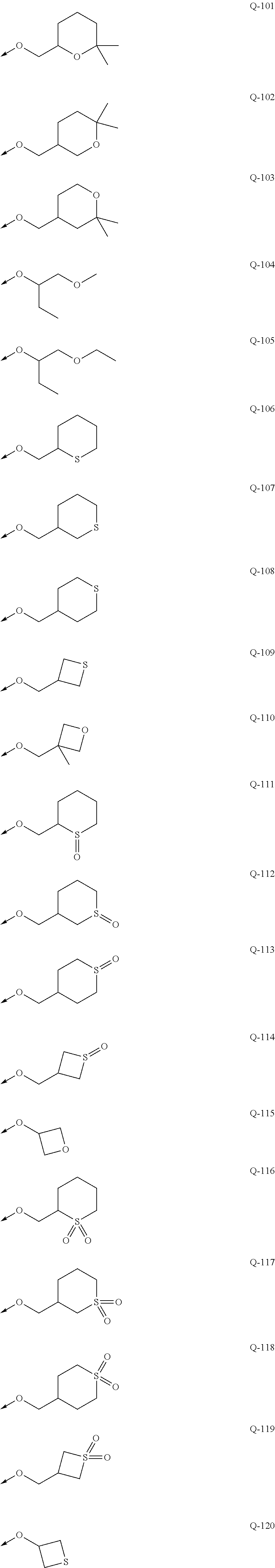

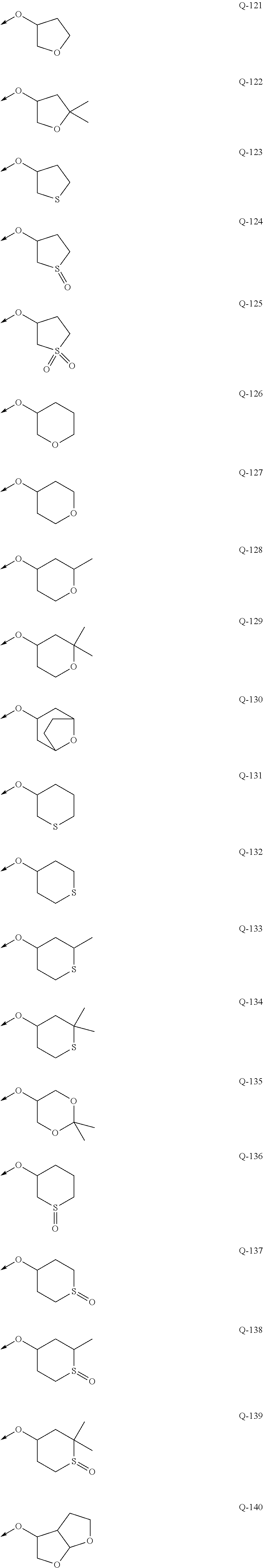

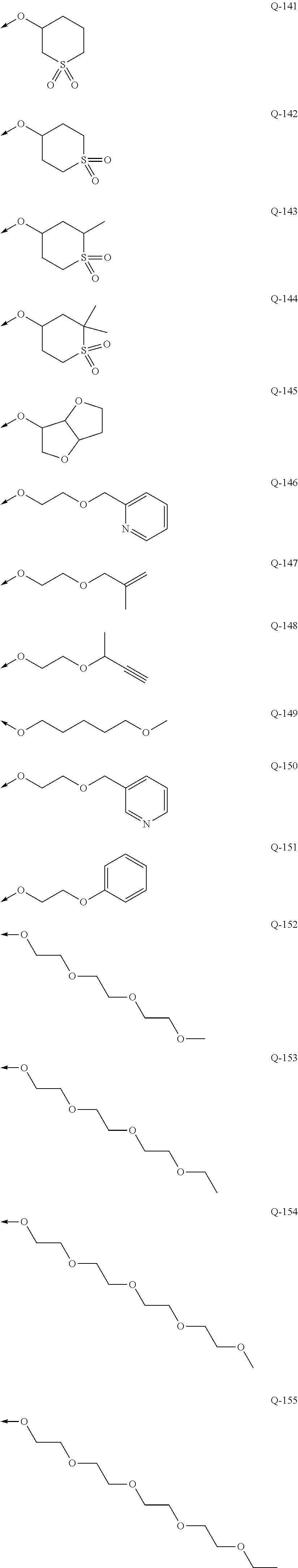

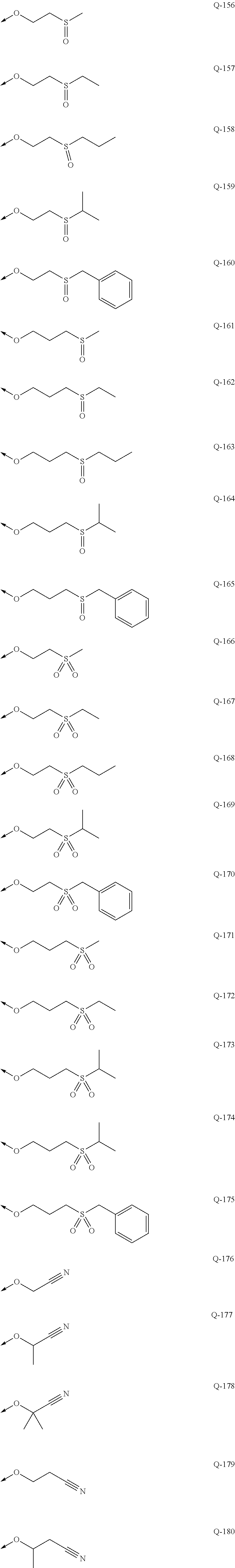

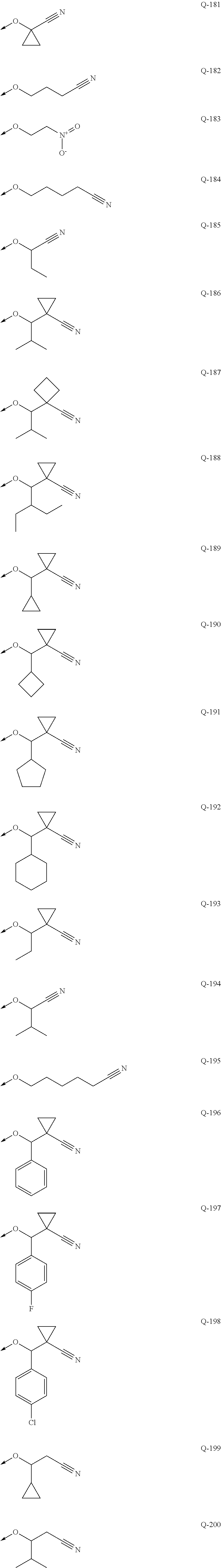

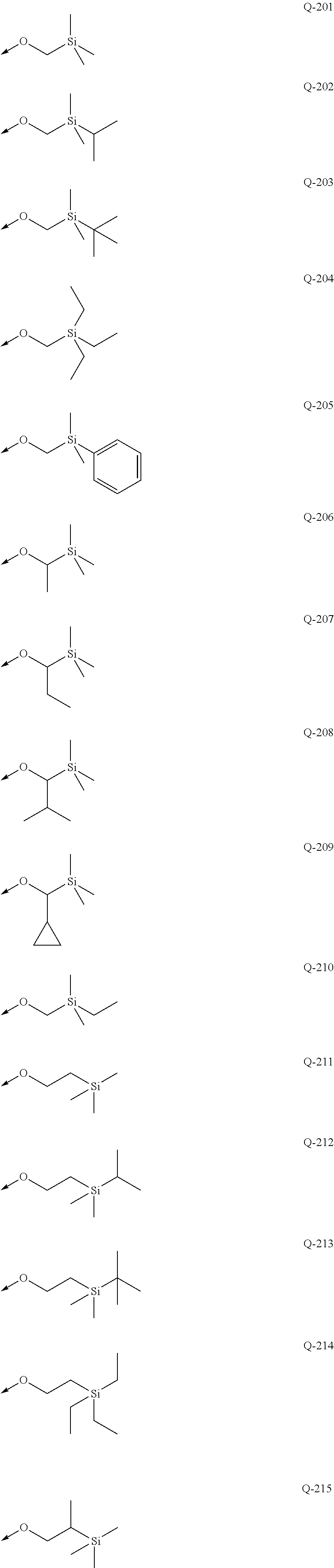

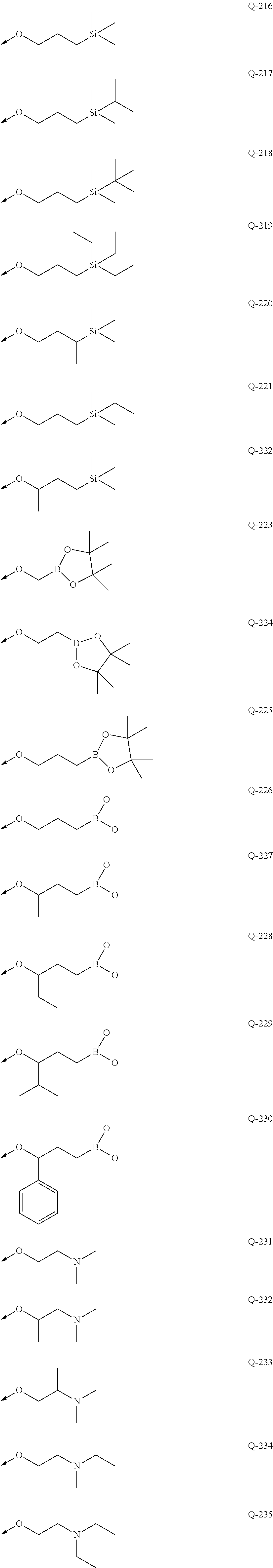

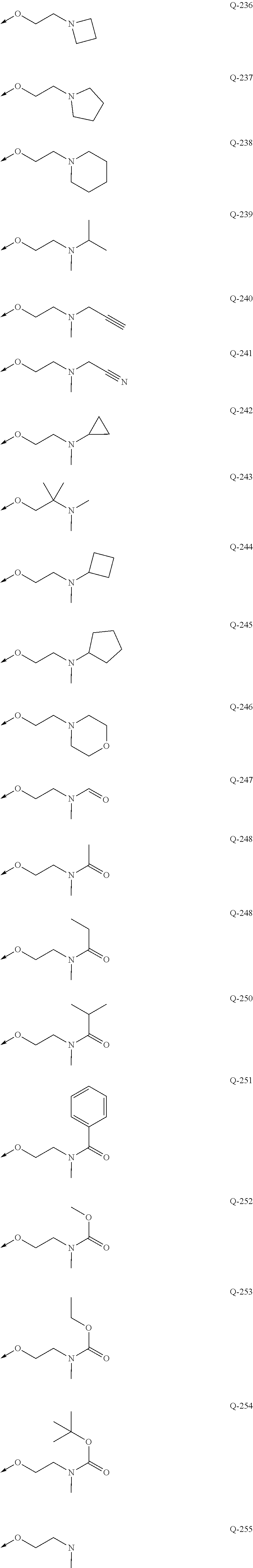

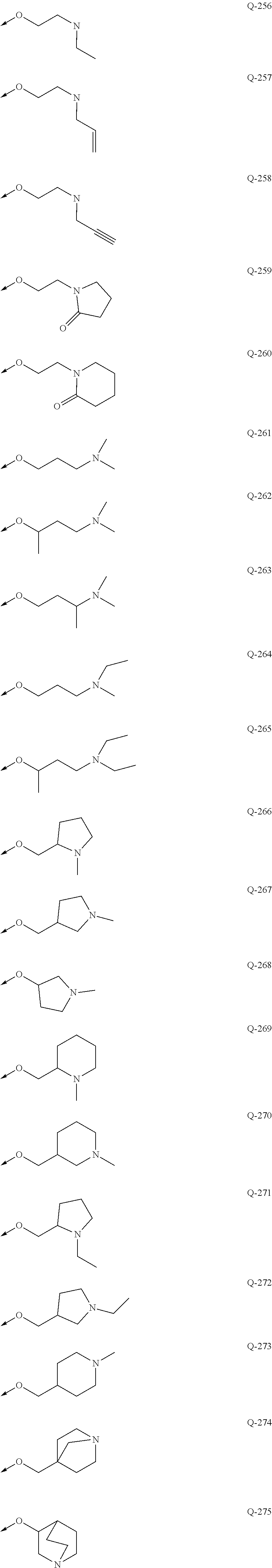

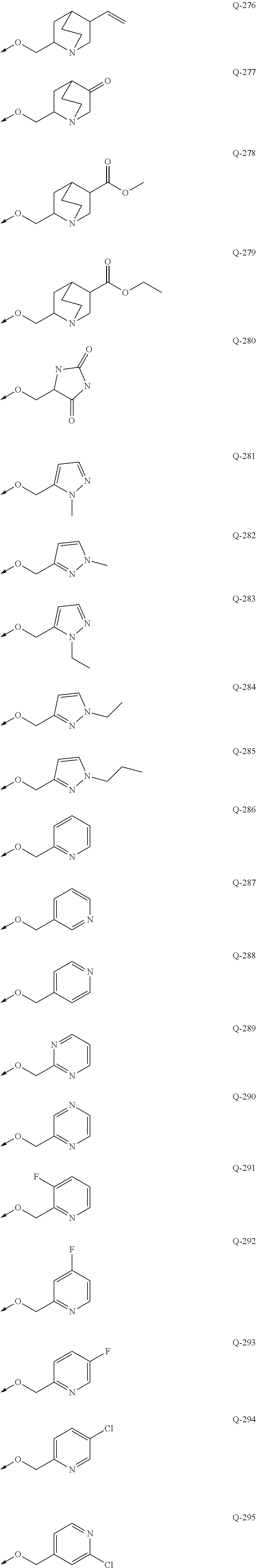

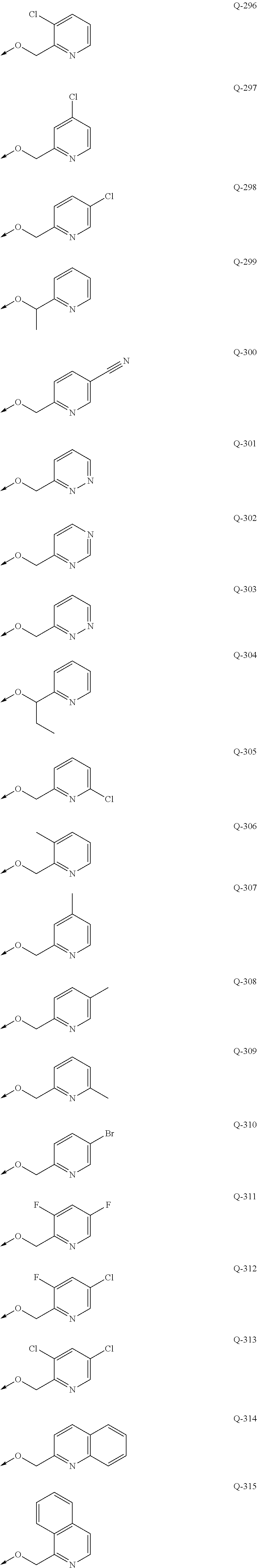

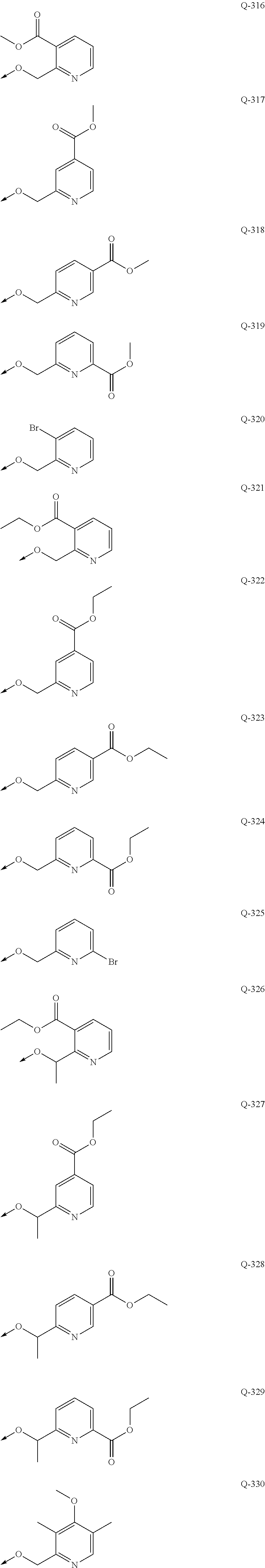

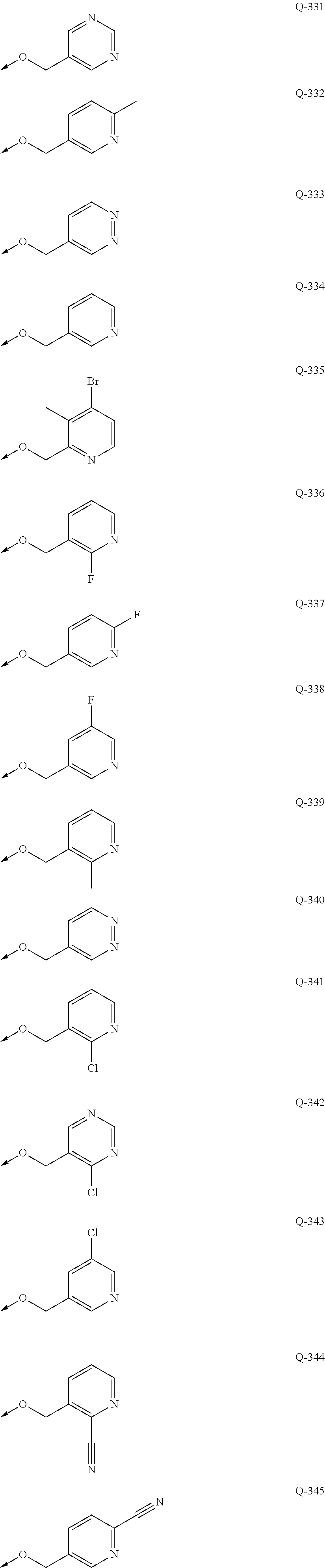

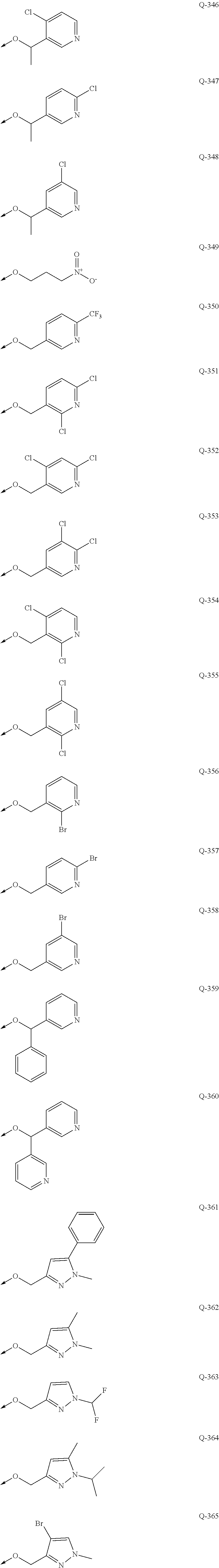

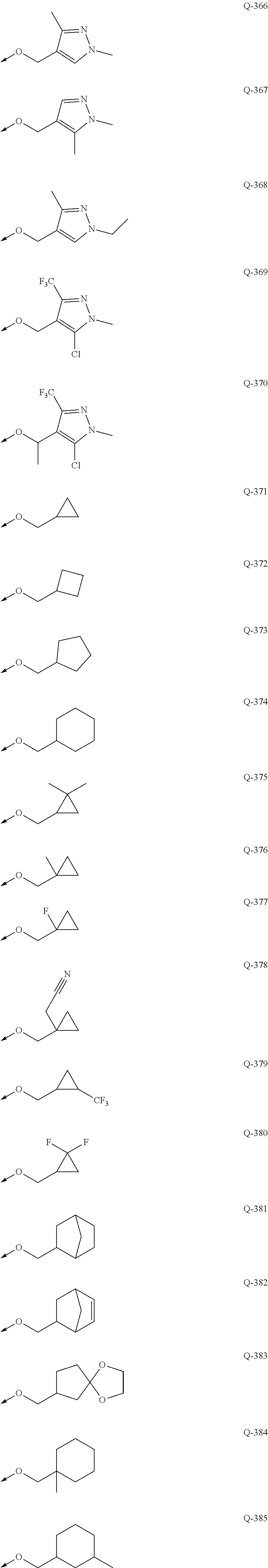

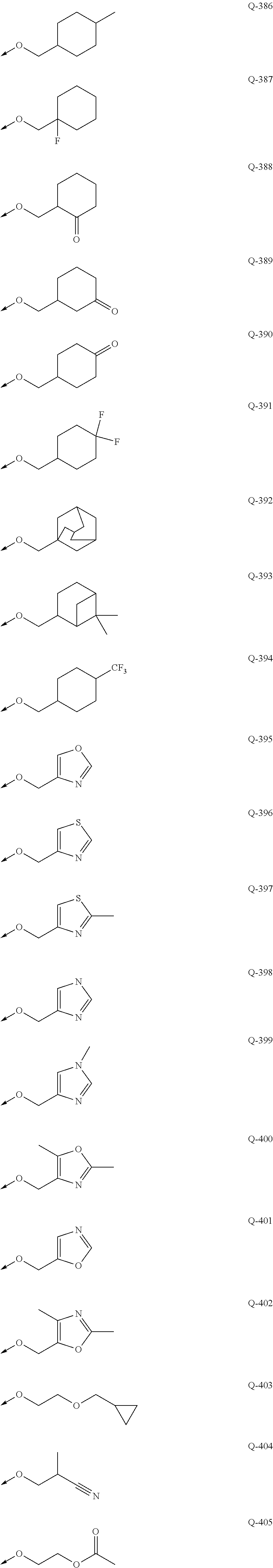

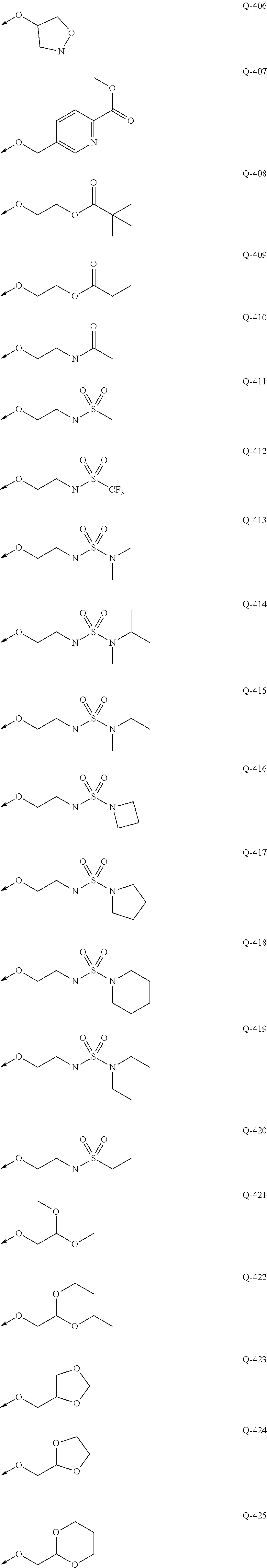

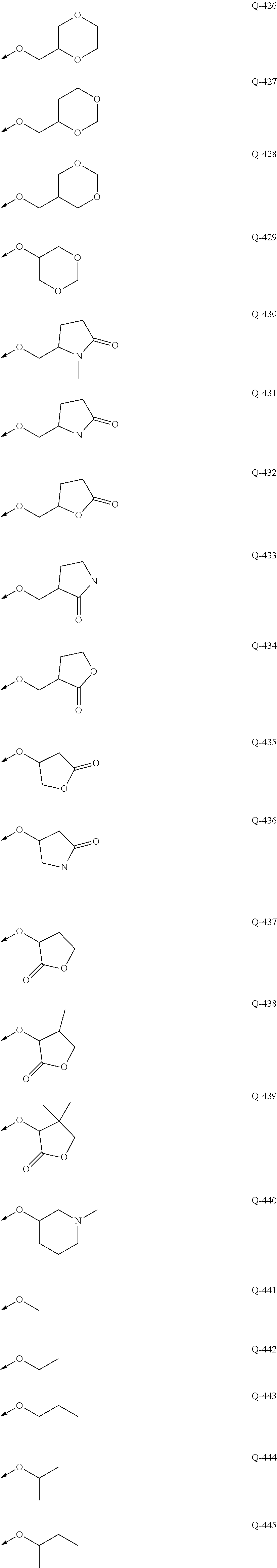

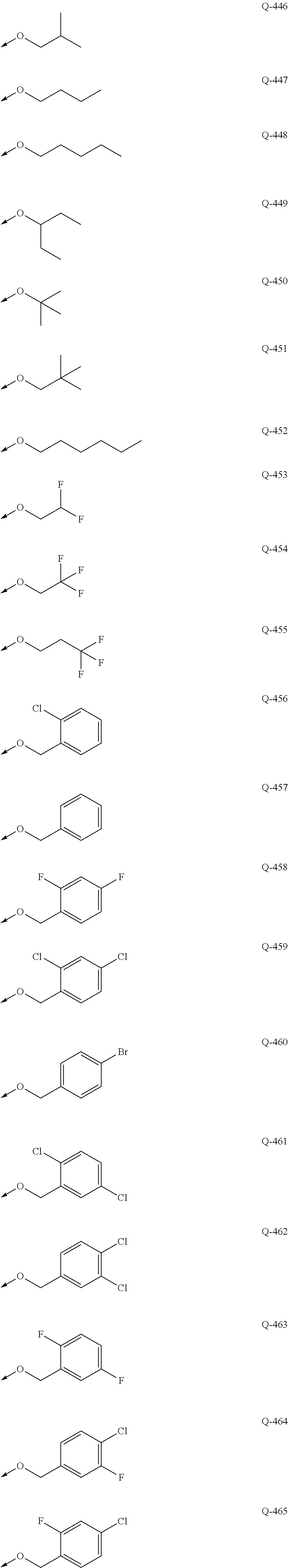

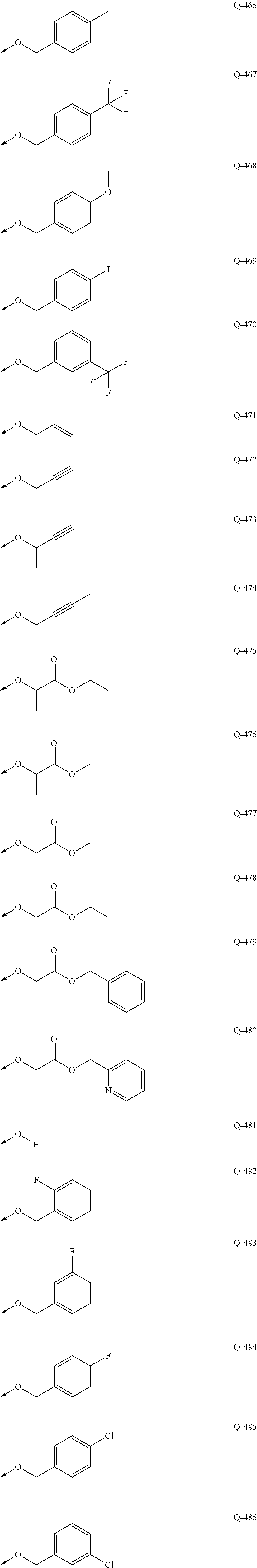

The invention especially preferably provides compounds of the general formula (I) in which
R¹ represents hydrogen,
R² represents fluorine,
R³ represents hydrogen, fluorine, chlorine, bromine, methoxy,
R⁴ represents fluorine, chlorine, bromine, cyano, NO₂, C(O)NH₂, C(S)NH₂, trifluoromethyl, ethynyl, propyn-1-yl,
R⁵, R⁶, R⁷ independently of one another represent hydrogen, fluorine, chlorine, bromine, iodine, cyano, methyl, ethyl, trifluoromethyl, difluoromethyl, methoxy, ethoxy, difluoromethoxy, trifluoromethoxy,
G represents methylene, (methyl)methylene, (ethyl)methylene, (dimethyl)methylene, Ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene,
R¹⁵ and R¹⁶ independently of one another represent hydrogen, methyl,
X and Y independently of one another represent O (oxygen) or S (sulfur) and
Q represents one of the moieties Q-1 to Q-486 specifically mentioned above,

The invention very especially preferably provides compounds of the general formula (I) in which
R¹ represents hydrogen,
R² represents fluorine,
R³ represents fluorine,
R⁴ represents chlorine, bromine, cyano, NO₂, C(O)NH₂, C(S)NH₂,
R⁵, R⁶ and R⁷ independently of one another represent hydrogen, fluorine, chlorine, bromine, cyano, methyl, trifluoromethyl, methoxy, trifluoromethoxy,
G represents methylene, (methyl)methylene, (ethyl)methylene, (dimethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, n-pentylene, n-hexylene,
R¹⁵ and R¹⁶ represent hydrogen,
X and Y independently of one another represent O (oxygen) or S (sulfur) and
Q represents one of the moieties Q-1 to Q-486 specifically mentioned above,

The invention particularly especially preferably provides compounds of the general formula (I) in which
R¹ represents hydrogen,
R² represents fluorine,
R³ represents fluorine,
R⁴ represents chlorine, bromine, cyano, NO₂, C(O)NH₂, C(S)NH₂,
R⁵, R⁶ and R⁷ independently of one another represent hydrogen, fluorine, chlorine, bromine, cyano, methyl, trifluoromethyl, methoxy, trifluoromethoxy,
G represents methylene, (methyl)methylene, (ethyl)methylene, (dimethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, n-pentylene, n-hexylene,
R¹⁵ and R¹⁶ represent hydrogen,
X and Y independently of one another represent O (oxygen) or S (sulfur) and
Q represents one of the moieties Q-1 to Q-486 specifically mentioned above,

The invention very particularly especially preferably provides compounds of the general formula (I) in which
R¹ represents hydrogen,
R² represents fluorine,
R³ represents fluorine,
R⁴ represents chlorine, bromine, cyano, NO₂,
R⁵, R⁶ and R⁷ independently of one another represent hydrogen, fluorine, chlorine, bromine, methyl,
G represents methylene, (methyl)methylene, (ethyl)methylene, (dimethyl)methylene, ethylene, n-propylene, n-butylene, n-pentylene, n-hexylene,
R¹⁵ and R¹⁶ represent hydrogen,
X and Y independently of one another represent O (oxygen) or S (sulfur) and
Q represents one of the moieties Q-1 to Q-486 specifically mentioned above,

The invention very especially preferably provides compounds of the general formula (I) in which
R¹ represents hydrogen,
R² represents fluorine,
R³ represents fluorine,
R⁴ represents chlorine, NO₂,
R⁵ represents hydrogen,
R⁶ represents hydrogen, fluorine,
R⁷ represents hydrogen,
G represents methylene, (methyl)methylene, ethylene, n-propylene,
R¹⁵ and R¹⁶ represent hydrogen,
X represents O (oxygen) or S (sulfur),
Y represents O (oxygen) and
Q represents one of the moieties Q-1, Q-2, Q-23, Q-115, Q-176, Q-286, Q-441, Q-442, Q-447, Q-448, Q-457, Q-471, Q-481 specifically mentioned above.

The definitions of radicals listed above in general terms or within areas of preference apply both to the end products of the formula (I) and to the starting materials or intermediates required for preparation. These radical definitions can be combined with one another as desired, i.e. including combinations between the given preferred ranges.

Primarily for reasons of higher herbicidal activity, better selectivity and/or better producibility, compounds of the abovementioned formula (I) according to the invention or their salts or their use according to the invention are of particular interest in which individual radicals have one of the preferred meanings already specified or specified below, or in particular those in which one or more of the preferred meanings already specified or specified below occur in combination.

If the compounds can form, through a hydrogen shift, tautomers whose structure is not formally covered by the formula (I), these tautomers are nevertheless covered by the definition of the inventive compounds of the formula (I), unless a particular tautomer is under consideration. For example, many carbonyl compounds may be present both in the keto form and in the enol form, both forms being encompassed by the definition of the compounds of the formula (I).

Depending on the nature of the substituents and the manner in which they are attached, the compounds of the general formula (I) may be present as stereoisomers. The formula (I) embraces all possible stereoisomers defined by the specific three-dimensional form thereof, such as enantiomers, diastereomers, Z and E isomers. If, for example, one or more alkenyl groups are present, diastereomers (Z and E isomers) may occur. If, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or else on the preparative scale to produce test specimens for biological testing. It is likewise possible to selectively prepare stereoisomers by using stereoselective reactions with use of optically active starting materials and/or auxiliaries. The invention thus also relates to all stereoisomers which are embraced by the general formula (I) but are not shown in their specific stereomeric form, and to mixtures thereof.

If the compounds are obtained as solids, the purification can also be carried out by recrystallization or digestion. If individual compounds of the general formula (I) cannot be obtained in a satisfactory manner by the routes described below, they can be prepared by derivatization of other compounds of the general formula (I).

Suitable isolation methods, purification methods and methods for separating stereoisomers of compounds of the formula (I) are methods generally known to the person skilled in the art from analogous cases, for example by physical processes such as crystallization, chromatographic methods, in particular column chromatography and HPLC (high pressure liquid chromatography), distillation, optionally under reduced pressure, extraction and other methods, any mixtures that remain can generally be separated by chromatographic separation, for example on chiral solid phases. Suitable for preparative amounts or on an industrial scale are processes such as crystallization, for example of diastereomeric salts which can be obtained from the diastereomer mixtures using optically active acids and, if appropriate, provided that acidic groups are present, using optically active bases.

With regard to the compounds of the general formula (I) according to the invention, the terms used above and further below will be elucidated. These are familiar to the person skilled in the art and especially have the definitions elucidated hereinafter:

Unless defined differently, names of chemical groups are generally to be understood such that attachment to the skeleton or the remainder of the molecule is via the structural element of the relevant chemical group mentioned last, i.e. for example in the case of (C₂-C₈)-alkenyloxy via the oxygen atom and in the case of heterocyclyl-(C₁-C₈)-alkyl or R¹⁷O(O)C—(C₁-C₈)-alkyl in each case via the carbon atom of the alkyl group.

According to the invention, “alkylsulfonyl”—alone or as part of a chemical group—refers to straight-chain or branched alkylsulfonyl, preferably having 1 to 8 or 1 to 6 carbon atoms, for example (but not limited to) (C₁-C₆)-alkylsulfonyl such as methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl.

According to the invention, “heteroarylsulfonyl” refers to optionally substituted pyridylsulfonyl, pyrimidinylsulfonyl, pyrazinylsulfonyl or optionally substituted polycyclic heteroarylsulfonyl, here in particular optionally substituted quinolinylsulfonyl, for example substituted by fluorine, chlorine, bromine, iodine, cyano, nitro, alkyl, haloalkyl, haloalkoxy, amino, alkylamino, alkylcarbonylamino, dialkylamino or alkoxy groups.

According to the invention, “alkylthio”—alone or as part of a chemical group—refers to straight-chain or branched S-alkyl, preferably having 1 to 8 or 1 to 6 carbon atoms, such as (C₁-C₁₀)-, (C₁-C₆)- or (C₁-C₄)-alkylthio, for example (but not limited to) (C₁-C₆)-alkylthio such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio.

According to the invention, “alkenylthio” denotes an alkenyl radical bonded via a sulfur atom, alkynylthio denotes an alkynyl radical bonded via a sulfur atom, cycloalkylthio denotes a cycloalkyl radical bonded via a sulfur atom, and cycloalkenylthio denotes a cycloalkenyl radical bonded via a sulfur atom.

According to the invention, “alkylsulfinyl (alkyl-S(═O)—)”, unless defined differently elsewhere, denotes alkyl radicals which are bonded to the skeleton via —S(═O)—, such as (C₁-C₁₀)-, (C₁-C₆)- or (C₁-C₄)-alkylsulfinyl, for example (but not limited to) (C₁-C₆)-alkylsulfinyl such as methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl.

Analogously, “alkenylsulfinyl” and “alkynylsulfinyl” are defined in accordance with the invention respectively as alkenyl and alkynyl radicals bonded to the skeleton via —S(═O)—, such as (C₂-C₁₀)-, (C₂-C₆)- or (C₂-C₄)-alkenylsulfinyl or (C₃-C₁₀)-, (C₃-C₆)- or (C₃-C₄)-alkynylsulfinyl.

Analogously, “alkenylsulfonyl” and “alkynylsulfonyl” are defined in accordance with the invention respectively as alkenyl and alkynyl radicals bonded to the skeleton via —S(═O)₂—, such as (C₂-C₁₀)-, (C₂-C₆)- or (C₂-C₄)-alkenylsulfonyl or (C₃-C₁₀)-, (C₃-C₆)- or (C₃-C₄)-alkynylsulfonyl.

“Alkoxy” denotes an alkyl radical attached via an oxygen atom, for example (but not limited to) (C₁-C₆)-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. Alkenyloxy denotes an alkenyl radical attached via an oxygen atom, and alkynyloxy denotes an alkynyl radical attached via an oxygen atom, such as (C₂-C₁₀)-, (C₂-C₆)- or (C₂-C₄)-alkenoxy and (C₃-C₁₀)-, (C₃-C₆)- or (C₃-C₄)-alkynoxy.

“Cycloalkyloxy” denotes a cycloalkyl radical bonded via an oxygen atom and cycloalkenyloxy denotes a cycloalkenyl radical bonded via an oxygen atom.

According to the invention, “alkylcarbonyl” (alkyl-C(═O)-), unless defined differently elsewhere, represents alkyl radicals bonded to the skeleton via —C(═O)—, such as (C₁-C₁₀)-, (C₁-C₆)- or (C₁-C₄)-alkylcarbonyl. Here, the number of the carbon atoms refers to the alkyl radical in the alkylcarbonyl group.

Analogously, “alkenylcarbonyl” and “alkynylcarbonyl”, unless defined differently elsewhere, in accordance with the invention, respectively represent alkenyl and alkynyl radicals bonded to the skeleton via —C(═O)—, such as (C₂-C₁₀)-, (C₂-C₆)- or (C₂-C₄)-alkenylcarbonyl and (C₂-C₁₀)-, (C₂-C₆)- or (C₂-C₄)-alkynylcarbonyl. Here, the number of the carbon atoms refers to the alkenyl or alkynyl radical in the alkenylcarbonyl or alkynylcarbonyl group.

“Alkoxycarbonyl (alkyl-O—C(═O)—)”, unless defined differently elsewhere: alkyl radicals bonded to the skeleton via —O—C(═O)—, such as (C₁-C₁₀)-, (C₁-C₆)- or (C₁-C₄)-alkoxycarbonyl. Here, the number of the carbon atoms refers to the alkyl radical in the alkoxycarbonyl group. Analogously, “alkenyloxycarbonyl” and “alkynyloxycarbonyl”, unless defined differently elsewhere, in accordance with the invention, respectively represent alkenyl and alkynyl radicals bonded to the skeleton via —O—C(═O)—, such as (C₂-C₁₀)-, (C₂-C₆)- or (C₂-C₄)-alkenyloxycarbonyl and (C₃-C₁₀)-, (C₃-C₆)- or (C₃-C₄)-alkynyloxycarbonyl. Here, the number of the carbon atoms refers to the alkenyl or alkynyl radical in the alkenyloxycarbonyl or alkynyloxycarbonyl group.

According to the invention, the term “alkylcarbonyloxy” (alkyl-C(═O)—O—), unless defined differently elsewhere, represents alkyl radicals bonded to the skeleton via the oxygen of a carbonyloxy group (—C(═O)—O—), such as (C₁-C₁₀)-, (C₁-C₆)- or (C₁-C₄)-alkylcarbonyloxy. Here, the number of the carbon atoms refers to the alkyl radical in the alkylcarbonyloxy group.

Analogously, “alkenylcarbonyloxy” and “alkynylcarbonyloxy” are defined in accordance with the invention respectively as alkenyl and alkynyl radicals bonded to the skeleton via the oxygen of (—C(═O)—O—), such as (C₂-C₁₀)-, (C₂-C₆)- or (C₂-C₄)-alkenylcarbonyloxy or (C₂-C₁₀)-, (C₂-C₆)- or (C₂-C₄)-alkynylcarbonyloxy. Here, the number of the carbon atoms refers to the alkenyl or alkynyl radical in the alkenyl- or alkynylcarbonyloxy group respectively.

In short forms such as C(O)R¹³, C(O)OR¹³, OC(O)NR¹¹R¹² or C(O)NR¹¹R¹², the short form 0 shown in brackets represents an oxygen atom attached to the adjacent carbon atom via a double bond.

In short forms such as OC(S)OR¹³, OC(S)SR¹⁴, OC(S)NR¹¹R¹², the short form S shown in brackets represents a sulfur atom attached to the adjacent carbon atom via a double bond.

The term “aryl” denotes an optionally substituted mono-, bi- or polycyclic aromatic system having preferably 6 to 14, especially 6 to 10, ring carbon atoms, for example phenyl, naphthyl, anthryl, phenanthrenyl and the like, preferably phenyl.

The term “optionally substituted aryl” also includes polycyclic systems, such as tetrahydronaphthyl, indenyl, indanyl, fluorenyl, biphenylyl, where the bonding site is on the aromatic system. In systematic terms, “aryl” is generally also encompassed by the term “optionally substituted phenyl”. Preferred aryl substituents here are, for example, hydrogen, halogen, alkyl, cycloalkyl, cycloalkylalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, bisalkylaminoalkoxy, tris[alkyl]silyl, bis[alkyl]arylsilyl, bis[alkyl]alkylsilyl, tris[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bis-alkylamino, alkylcarbonylamino, cycloalkylcarbonylamino, arylcarbonylamino, alkoxycarbonylamino, alkoxycarbonylalkylamino, arylalkoxycarbonylalkylamino, hydroxycarbonyl, alkoxycarbonyl, aminocarbonyl, alkylaminocarbonyl, cycloalkylaminocarbonyl, bisalkylaminocarbonyl, heteroarylalkoxy, arylalkoxy.

A heterocyclic radical (heterocyclyl) contains at least one heterocyclic ring (=carbocyclic ring in which at least one carbon atom has been replaced by a heteroatom, preferably by a heteroatom from the group of N, O, S, P) which is saturated, unsaturated, partially saturated or heteroaromatic and may be unsubstituted or substituted, in which case the bonding site is localized on a ring atom. If the heterocyclyl radical or the heterocyclic ring is optionally substituted, it may be fused to other carbocyclic or heterocyclic rings. In the case of optionally substituted heterocyclyl, polycyclic systems are also included, for example 8-azabicyclo[3.2.1]octanyl, 8-azabicyclo[2.2.2]octanyl or 1-azabicyclo[2.2.1]heptyl. Optionally substituted heterocyclyl also includes spirocyclic systems, such as, for example, 1-oxa-5-azaspiro[2.3]hexyl. Unless defined differently, the heterocyclic ring preferably contains 3 to 9 ring atoms, especially 3 to 6 ring atoms, and one or more, preferably 1 to 4, especially 1, 2 or 3, heteroatoms in the heterocyclic ring, preferably from the group of N, O and S, but no two oxygen atoms should be directly adjacent, for example with one heteroatom from the group of N, O and S: 1- or 2- or 3-pyrrolidinyl, 3,4-dihydro-2H-pyrrol-2- or -3-yl, 2,3-dihydro-1H-pyrrol-1- or -2- or -3- or -4- or -5-yl; 2,5-dihydro-1H-pyrrol-1- or -2- or -3-yl, 1- or 2- or 3- or 4-piperidinyl; 2,3,4,5-tetrahydropyridin-2- or -3- or -4- or -5-yl or -6-yl; 1,2,3,6-tetrahydropyridin-1- or -2- or -3- or -4- or -5- or -6-yl; 1,2,3,4-tetrahydropyridin-1- or -2- or -3- or -4- or -5- or -6-yl; 1,4-dihydropyridin-1- or -2- or -3- or -4-yl; 2,3-dihydropyridin-2- or -3- or -4- or -5- or -6-yl; 2,5-dihydropyridin-2- or -3- or -4- or -5- or -6-yl, 1- or 2- or 3- or 4-azepanyl; 2,3,4,5-tetrahydro-1H-azepin-1- or -2- or -3- or -4- or -5- or -6- or -7-yl; 2,3,4,7-tetrahydro-1H-azepin-1- or -2- or -3- or -4- or -5- or -6- or -7-yl; 2,3,6,7-tetrahydro-1H-azepin-1- or -2- or -3- or -4-yl; 3,4,5,6-tetrahydro-2H-azepin-2- or -3- or -4- or -5- or -6- or -7-yl; 4,5-dihydro-1H-azepin-1- or -2- or -3- or -4-yl; 2,5-dihydro-1H-azepin-1- or -2- or -3- or -4- or -5- or -6- or -7-yl; 2,7-dihydro-1H-azepin-1- or -2- or -3- or -4-yl; 2,3-dihydro-1H-azepin-1- or -2- or -3- or -4- or -5- or -6- or- 7-yl; 3,4-dihydro-2H-azepin-2- or -3- or -4- or -5- or -6- or -7-yl; 3,6-dihydro-2H-azepin-2- or -3- or -4- or -5- or -6- or -7-yl; 5,6-dihydro-2H-azepin-2- or -3- or -4- or -5- or -6- or -7-yl; 4,5-dihydro-3H-azepin-2- or -3- or -4- or -5- or -6- or -7-yl; 1H-azepin-1- or -2- or -3- or -4- or -5- or -6- or -7-yl; 2H-azepin-2- or -3- or -4- or -5- or -6- or -7-yl; 3H-azepin-2- or -3- or -4- or -5- or -6- or -7-yl; 4H-azepin-2- or -3- or -4- or -5- or -6- or -7-yl, 2- or 3-oxolanyl (=2- or 3-tetrahydrofuranyl); 2,3-dihydrofuran-2- or -3- or -4- or -5-yl; 2,5-dihydrofuran-2- or -3-yl, 2- or 3- or 4-oxanyl (=2- or 3- or 4-tetrahydropyranyl); 3,4-dihydro-2H-pyran-2- or -3- or -4- or -5- or -6-yl; 3,6-dihydro-2H-pyran-2- or -3- or -4- or -5- or -6-yl; 2H-pyran-2- or -3- or -4- or -5- or -6-yl; 4H-pyran-2- or -3- or -4-yl, 2- or 3- or 4-oxepanyl; 2,3,4,5-tetrahydrooxepin-2- or -3- or -4- or -5- or -6- or -7-yl; 2,3,4,7-tetrahydrooxepin-2- or -3- or -4- or -5- or -6- or -7-yl; 2,3,6,7-tetrahydrooxepin-2- or -3- or -4-yl; 2,3-dihydrooxepin-2- or -3- or- 4- or -5- or -6- or -7-yl; 4,5-dihydrooxepin-2- or -3- or -4-yl; 2,5-dihydrooxepin-2- or -3- or -4- or -5- or -6- or -7-yl; oxepin-2- or -3- or -4- or -5- or -6- or -7-yl; 2- or 3-tetrahydrothiophenyl; 2,3-dihydrothiophen-2- or -3- or -4- or -5-yl; 2,5-dihydrothiophen-2- or -3-yl; tetrahydro-2H-thiopyran-2- or- 3- or -4-yl; 3,4-dihydro-2H-thiopyran-2- or -3- or -4- or -5- or -6-yl; 3,6-dihydro-2H-thiopyran-2- or -3- or -4- or -5- or -6-yl; 2H-thiopyran-2- or -3- or -4- or -5- or -6-yl; 4H-thiopyran-2- or -3- or -4-yl. Preferred 3-membered and 4-membered heterocycles are, for example, 1- or 2-aziridinyl, oxiranyl, thiiranyl, 1- or 2- or 3-azetidinyl, 2- or 3-oxetanyl, 2- or 3-thietanyl, 1,3-dioxetan-2-yl. Further examples of “heterocyclyl” are a partially or fully hydrogenated heterocyclic radical having two heteroatoms from the group consisting of N, O and S, such as, for example, 1- or 2- or 3- or 4-pyrazolidinyl; 4,5-dihydro-3H-pyrazol-3- or -4- or -5-yl; 4,5-dihydro-1H-pyrazol-1- or -3- or -4- or -5-yl; 2,3-dihydro-1H-pyrazol-1- or -2- or -3- or -4- or -5-yl; 1- or 2- or 3- or 4-imidazolidinyl; 2,3-dihydro-1H-imidazol-1- or -2- or -3- or -4-yl; 2,5-dihydro-1H-imidazol-1- or -2- or -4- or -5-yl; 4,5-dihydro-1H-imidazol-1- or -2- or -4- or -5-yl; hexahydropyridazin-1- or -2- or -3- or -4-yl; 1,2,3,4-tetrahydropyridazin-1- or -2- or -3- or -4- or -5- or -6-yl; 1,2,3,6-tetrahydropyridazin-1- or -2- or -3- or -4- or -5- or -6-yl; 1,4,5,6-tetrahydropyridazin-1- or -3- or -4- or -5- or -6-yl; 3,4,5,6-tetrahydropyridazin-3- or -4- or -5-yl; 4,5-dihydropyridazin-3- or -4-yl; 3,4-dihydropyridazin-3- or -4- or -5- or -6-yl; 3,6-dihydropyridazin-3- or -4-yl; 1,6-dihydropyriazin-1- or -3- or -4- or -5- or -6-yl; hexahydropyrimidin-1- or -2- or -3- or -4-yl; 1,4,5,6-tetrahydropyrimidin-1- or -2- or -4- or -5- or -6-yl; 1,2,5,6-tetrahydropyrimidin-1- or -2- or -4- or- 5- or -6-yl; 1,2,3,4-tetrahydropyrimidin-1- or -2- or -3- or -4- or -5- or -6-yl; 1,6-dihydropyrimidin-1- or- 2- or -4- or -5- or -6-yl; 1,2-dihydropyrimidin-1- or -2- or -4- or -5- or -6-yl; 2,5-dihydropyrimidin-2- or- 4- or -5-yl; 4,5-dihydropyrimidin-4- or -5- or -6-yl; 1,4-dihydropyrimidin-1- or -2- or -4- or -5- or -6-yl; 1- or 2- or 3-piperazinyl; 1,2,3,6-tetrahydropyrazin-1- or -2- or -3- or -5- or -6-yl; 1,2,3,4-tetrahydropyrazin-1- or -2- or -3- or -4- or -5- or -6-yl; 1,2-dihydropyrazin-1- or -2- or -3- or -5- or -6-yl; 1,4-dihydropyrazin-1- or -2- or -3-yl; 2,3-dihydropyrazin-2- or -3- or -5- or -6-yl; 2,5-dihydropyrazin-2- or -3-yl; 1,3-dioxolan-2- or -4- or -5-yl; 1,3-dioxol-2- or -4-yl; 1,3-dioxan-2- or -4- or -5-yl; 4H-1,3-dioxin-2- or -4- or -5- or -6-yl; 1,4-dioxan-2- or -3- or -5- or -6-yl; 2,3-dihydro-1,4-dioxin-2- or -3- or -5- or -6-yl; 1,4-dioxin-2- or -3-yl; 1,2-dithiolan-3- or -4-yl; 3H-1,2-dithiol-3- or -4- or -5-yl; 1,3-dithiolan-2- or -4-yl; 1,3-dithiol-2- or -4-yl; 1,2-dithian-3- or -4-yl; 3,4-dihydro-1,2-dithiin-3- or -4- or -5- or -6-yl; 3,6-dihydro-1,2-dithiin-3- or -4-yl; 1,2-dithiin-3- or -4-yl; 1,3-dithian-2- or -4- or -5-yl; 4H-1,3-dithiin-2- or -4- or -5- or -6-yl; isoxazolidin-2- or -3- or -4- or -5-yl; 2,3-dihydroisoxazol-2- or -3- or- 4- or -5-yl; 2,5-dihydroisoxazol-2- or -3- or -4- or -5-yl; 4,5-dihydroisoxazol-3- or -4- or -5-yl; 1,3-oxazolidin-2- or -3- or -4- or -5-yl; 2,3-dihydro-1,3-oxazol-2- or -3- or -4- or -5-yl; 2,5-dihydro-1,3-oxazol-2- or -4- or -5-yl; 4,5-dihydro-1,3-oxazol-2- or -4- or -5-yl; 1,2-oxazinan-2- or -3- or -4- or -5- or- 6-yl; 3,4-dihydro-2H-1,2-oxazin-2- or -3- or -4- or -5- or -6-yl; 3,6-dihydro-2H-1,2-oxazin-2- or -3- or -4- or -5- or -6-yl; 5,6-dihydro-2H-1,2-oxazin-2- or -3- or -4- or -5- or -6-yl; 5,6-dihydro-4H-1,2-oxazin-3- or -4- or -5- or -6-yl; 2H-1,2-oxazin-2- or -3- or -4- or -5- or -6-yl; 6H-1,2-oxazin-3- or -4- or -5- or -6-yl; 4H-1,2-oxazin-3- or -4- or -5- or -6-yl; 1,3-oxazinan-2- or -3- or -4- or -5- or -6-yl; 3,4-dihydro-2H-1,3-oxazin-2- or -3- or -4- or -5- or -6-yl; 3,6-dihydro-2H-1,3-oxazin-2- or -3- or -4- or -5- or -6-yl; 5,6-dihydro-2H-1,3-oxazin-2- or -4- or -5- or -6-yl; 5,6-dihydro-4H-1,3-oxazin-2- or -4- or -5- or -6-yl; 2H-1,3-oxazin-2- or -4- or -5- or -6-yl; 6H-1,3-oxazin-2- or -4- or -5- or -6-yl; 4H-1,3-oxazin-2- or -4- or -5- or -6-yl; morpholin-2- or -3- or -4-yl; 3,4-dihydro-2H-1,4-oxazin-2- or -3- or -4- or -5- or -6-yl; 3,6-dihydro-2H-1,4-oxazin-2- or -3- or -5- or -6-yl; 2H-1,4-oxazin-2- or -3- or -5- or -6-yl; 4H-1,4-oxazin-2- or -3-yl; 1,2-oxazepan-2- or -3- or -4- or -5- or -6- or -7-yl; 2,3,4,5-tetrahydro-1,2-oxazepin-2- or -3- or -4- or -5- or -6- or -7-yl; 2,3,4,7-tetrahydro-1,2-oxazepin-2- or -3- or -4- or -5- or -6- or -7-yl; 2,3,6,7-tetrahydro-1,2-oxazepin-2- or -3- or -4- or -5- or -6- or -7-yl; 2,5,6,7-tetrahydro-1,2-oxazepin-2- or -3- or -4- or -5- or -6- or -7-yl; 4,5,6,7-tetrahydro-1,2-oxazepin-3- or -4- or -5- or -6- or -7-yl; 2,3-dihydro-1,2-oxazepin-2- or -3- or -4- or -5- or -6- or -7-yl; 2,5-dihydro-1,2-oxazepin-2- or -3- or -4- or -5- or -6- or -7-yl; 2,7-dihydro-1,2-oxazepin-2- or -3- or -4- or -5- or -6- or -7-yl; 4,5-dihydro-1,2-oxazepin-3- or -4- or -5- or -6- or -7-yl; 4,7-dihydro-1,2-oxazepin-3- or -4- or -5- or -6- or -7-yl; 6,7-dihydro-1,2-oxazepin-3- or -4- or -5- or -6- or -7-yl; 1,2-oxazepin-3- or -4- or -5- or -6- or -7-yl; 1,3-oxazepan-2- or -3- or -4- or -5- or -6- or -7-yl; 2,3,4,5-tetrahydro-1,3-oxazepin-2- or -3- or -4- or -5- or -6- or -7-yl; 2,3,4,7-tetrahydro-1,3-oxazepin-2- or -3- or -4- or -5- or -6- or -7-yl; 2,3,6,7-tetrahydro-1,3-oxazepin-2- or -3- or -4- or -5- or -6- or -7-yl; 2,5,6,7-tetrahydro-1,3-oxazepin-2- or -4- or -5- or -6- or -7-yl; 4,5,6,7-tetrahydro-1,3-oxazepin-2- or -4- or -5- or -6- or -7-yl; 2,3-dihydro-1,3-oxazepin-2- or -3- or -4- or -5- or -6- or -7-yl; 2,5-dihydro-1,3-oxazepin-2- or -4- or -5- or -6- or -7-yl; 2,7-dihydro-1,3-oxazepin-2- or -4- or -5- or -6- or -7-yl; 4,5-dihydro-1,3-oxazepin-2- or -4- or -5- or -6- or -7-yl; 4,7-dihydro-1,3-oxazepin-2- or -4- or -5- or -6- or -7-yl; 6,7-dihydro-1,3-oxazepin-2- or -4- or -5- or -6- or -7-yl; 1,3-oxazepin-2- or -4- or -5- or -6- or -7-yl; 1,4-oxazepan-2- or -3- or -5- or -6- or -7-yl; 2,3,4,5-tetrahydro-1,4-oxazepin-2- or -3- or -4- or -5- or -6- or -7-yl; 2,3,4,7-tetrahydro-1,4-oxazepin-2- or -3- or -4- or -5- or -6- or -7-yl; 2,3,6,7-tetrahydro-1,4-oxazepin-2- or -3- or -5- or -6- or -7-yl; 2,5,6,7-tetrahydro-1,4-oxazepin-2- or -3- or -5- or -6- or -7-yl; 4,5,6,7-tetrahydro-1,4-oxazepin-2- or -3- or -4- or -5- or -6- or -7-yl; 2,3-dihydro-1,4-oxazepin-2- or -3- or -5- or -6- or -7-yl; 2,5-dihydro-1,4-oxazepin-2- or -3- or -5- or -6- or -7-yl; 2,7-dihydro-1,4-oxazepin-2- or -3- or -5- or -6- or -7-yl; 4,5-dihydro-1,4-oxazepin-2- or -3- or -4- or -5- or -6- or -7-yl; 4,7-dihydro-1,4-oxazepin-2- or -3- or -4- or -5- or -6- or -7-yl; 6,7-dihydro-1,4-oxazepin-2- or -3- or -5- or -6- or -7-yl; 1,4-oxazepin-2- or -3- or -5- or -6- or -7-yl; isothiazolidin-2- or -3- or -4- or -5-yl; 2,3-dihydroisothiazol-2- or -3- or -4- or -5-yl; 2,5-dihydroisothiazol-2- or -3- or -4- or -5-yl; 4,5-dihydroisothiazol-3- or -4- or -5-yl; 1,3-thiazolidin-2- or -3- or -4- or -5-yl; 2,3-dihydro-1,3-thiazol-2- or -3- or -4- or -5-yl; 2,5-dihydro-1,3-thiazol-2- or -4- or -5-yl; 4,5-dihydro-1,3-thiazol-2- or -4- or -5-yl; 1,3-thiazinan-2- or -3- or -4- or -5- or -6-yl; 3,4-dihydro-2H-1,3-thiazin-2- or -3- or -4- or -5- or -6-yl; 3,6-dihydro-2H-1,3-thiazin-2- or -3- or -4- or -5- or -6-yl; 5,6-dihydro-2H-1,3-thiazin-2- or -4- or -5- or -6-yl; 5,6-dihydro-4H-1,3-thiazin-2- or -4- or -5- or -6-yl; 2H-1,3-thiazin-2- or -4- or -5- or -6-yl; 6H-1,3-thiazin-2- or -4- or -5- or -6-yl; 4H-1,3-thiazin-2- or -4- or -5- or -6-yl. Further examples of “heterocyclyl” are a partially or fully hydrogenated heterocyclic radical having 3 heteroatoms from the group of N, O and S, for example 1,4,2-dioxazolidin-2- or -3- or -5-yl; 1,4,2-dioxazol-3- or -5-yl; 1,4,2-dioxazinan-2- or -3- or -5- or -6-yl; 5,6-dihydro-1,4,2-dioxazin-3- or -5- or -6-yl; 1,4,2-dioxazin-3- or -5- or -6-yl; 1,4,2-dioxazepan-2- or -3- or -5- or -6- or -7-yl; 6,7-dihydro-5H-1,4,2-dioxazepin-3- or -5- or -6- or -7-yl; 2,3-dihydro-7H-1,4,2-dioxazepin-2- or -3- or -5- or -6- or -7-yl; 2,3-dihydro-5H-1,4,2-dioxazepin-2- or -3- or -5- or -6- or -7-yl; 5H-1,4,2-dioxazepin-3- or -5- or -6- or -7-yl; 7H-1,4,2-dioxazepin-3- or -5- or -6- or -7-yl. Structural examples of heterocycles which are optionally substituted further are also listed below:

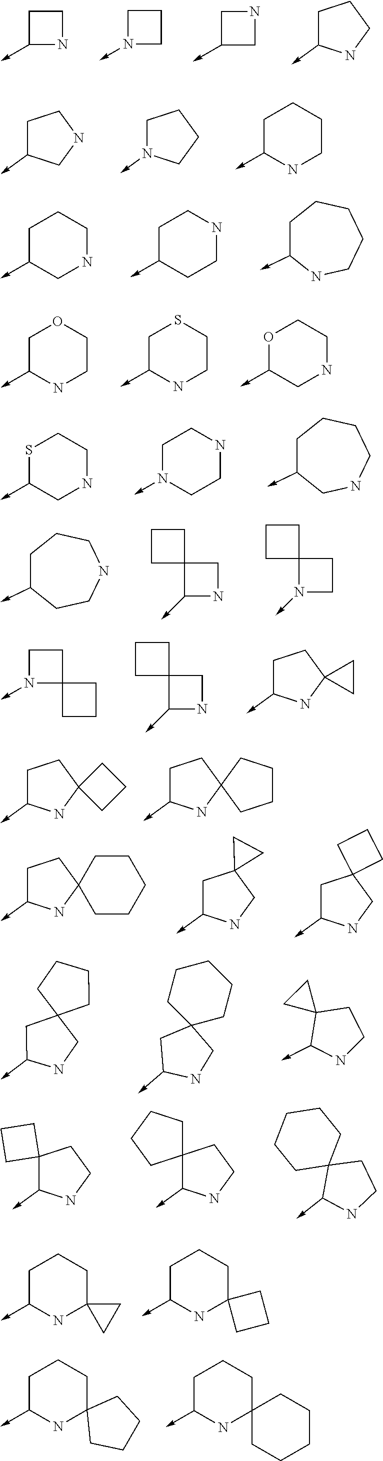

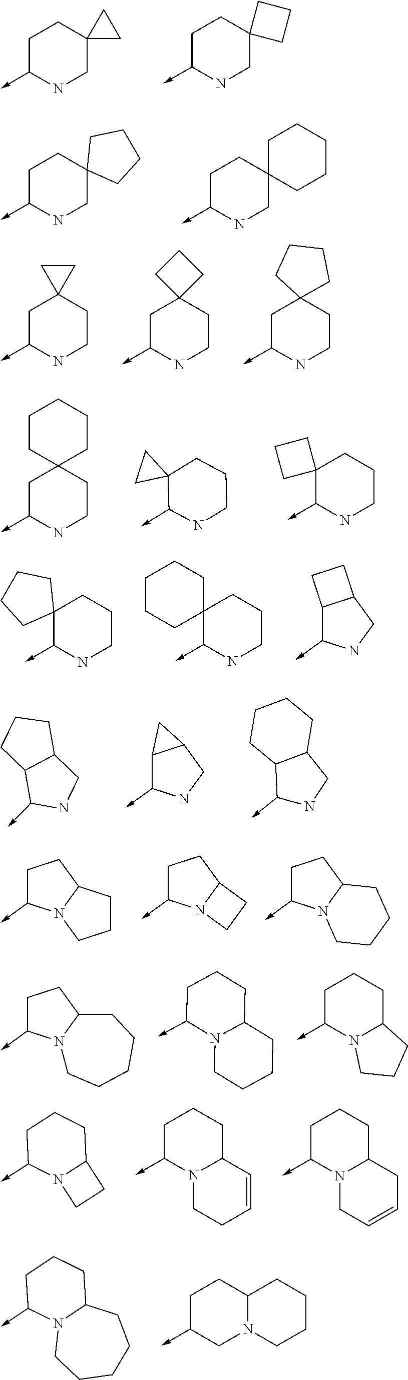

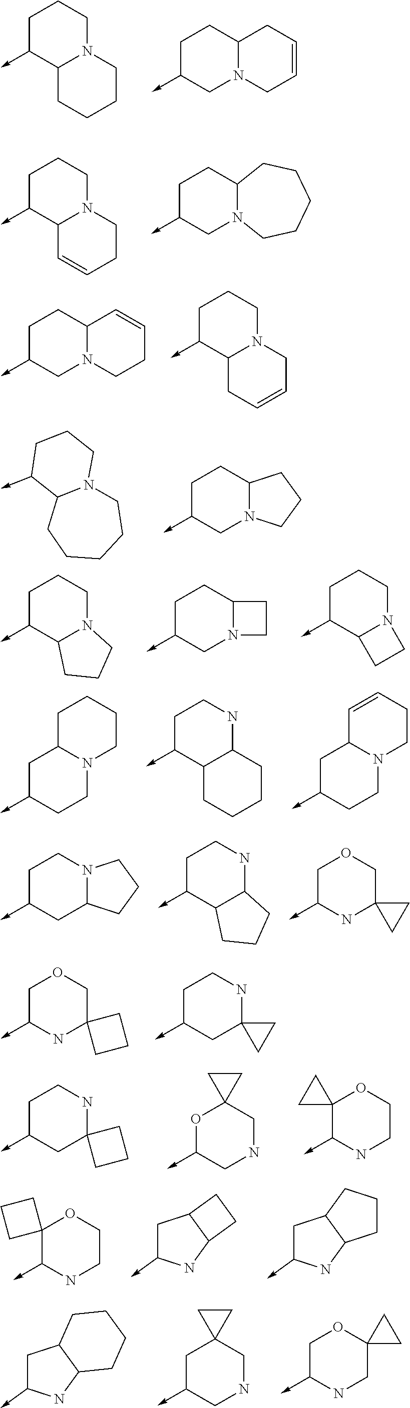

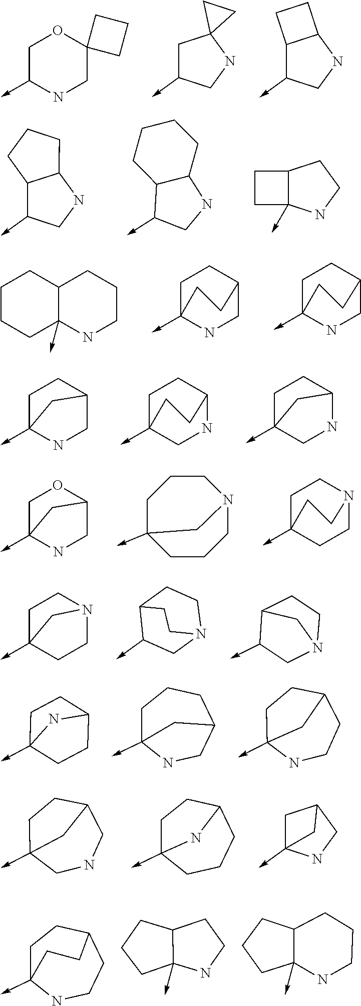

The heterocycles listed above are preferably substituted, for example, by hydrogen, halogen, alkyl, haloalkyl, hydroxyl, alkoxy, cycloalkoxy, aryloxy, alkoxyalkyl, alkoxyalkoxy, cycloalkyl, halocycloalkyl, aryl, arylalkyl, heteroaryl, heterocyclyl, alkenyl, alkylcarbonyl, cycloalkylcarbonyl, arylcarbonyl, heteroarylcarbonyl, alkoxycarbonyl, hydroxycarbonyl, cycloalkoxycarbonyl, cycloalkylalkoxycarbonyl, alkoxycarbonylalkyl, arylalkoxycarbonyl, arylalkoxycarbonylalkyl, alkynyl, alkynylalkyl, alkylalkynyl, trisalkylsilylalkynyl, nitro, amino, cyano, haloalkoxy, haloalkylthio, alkylthio, hydrothio, hydroxyalkyl, oxo, heteroarylalkoxy, arylalkoxy, heterocyclylalkoxy, heterocyclylalkylthio, heterocyclyloxy, heterocyclylthio, heteroaryloxy, bisalkylamino, alkylamino, cycloalkylamino, hydroxycarbonylalkylamino, alkoxycarbonylalkylamino, arylalkoxycarbonylalkylamino, alkoxycarbonylalkyl(alkyl)amino, aminocarbonyl, alkylaminocarbonyl, bisalkylaminocarbonyl, cycloalkylaminocarbonyl, hydroxycarbonylalkylaminocarbonyl, alkoxycarbonylalkylaminocarbonyl, arylalkoxycarbonylalkylaminocarbonyl.

When a base structure is substituted “by one or more radicals” from a list of radicals (=group) or a generically defined group of radicals, this in each case includes simultaneous substitution by a plurality of identical and/or structurally different radicals.

In the case of a partially or fully saturated nitrogen heterocycle, this may be joined to the remainder of the molecule either via carbon or via the nitrogen.

Suitable substituents for a substituted heterocyclic radical are the substituents specified further down, and additionally also oxo and thioxo. The oxo group as a substituent on a ring carbon atom is then, for example, a carbonyl group in the heterocyclic ring. As a result, lactones and lactams are preferably also included. The oxo group may also occur on the ring heteroatoms, which may exist in different oxidation states, for example in the case of N and S, and in that case form, for example, the divalent —N(O)—, —S(O)— (also SO for short) and —S(O)₂— (also SO₂ for short) groups in the heterocyclic ring. In the case of —N(O)— and —S(O)— groups, both enantiomers in each case are included.

According to the invention, the expression “heteroaryl” refers to heteroaromatic compounds, i.e. fully unsaturated aromatic heterocyclic compounds, preferably 5- to 7-membered rings having 1 to 4, preferably 1 or 2, identical or different heteroatoms, preferably 0, S or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepinyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazinyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepinyl, thiepinyl, 1,2,4-triazolonyl and 1,2,4-diazepinyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The heteroaryl groups of the invention may also be substituted by one or more identical or different radicals. If two adjacent carbon atoms are part of a further aromatic ring, the systems are fused heteroaromatic systems, such as benzofused or polyannelated heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, 2H-indazol-7-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

The term “halogen” denotes, for example, fluorine, chlorine, bromine or iodine. If the term is used for a radical, “halogen” denotes, for example, a fluorine, chlorine, bromine or iodine atom.

According to the invention, “alkyl” means a straight-chain or branched open-chain, saturated hydrocarbon radical which is optionally mono- or polysubstituted, and in the latter case is referred to as “substituted alkyl”. Preferred substituents are halogen atoms, alkoxy, haloalkoxy, cyano, alkylthio, haloalkylthio, amino or nitro groups, particular preference being given to methoxy, methyl, fluoroalkyl, cyano, nitro, fluorine, chlorine, bromine or iodine. The prefix “bis” also includes the combination of different alkyl radicals, e.g. methyl(ethyl) or ethyl(methyl).

“Haloalkyl”, “-alkenyl” and “-alkynyl” respectively denote alkyl, alkenyl and alkynyl partially or fully substituted by identical or different halogen atoms, for example monohaloalkyl such as CH₂CH₂C₁, CH₂CH₂Br, CHClCH₃, CH₂C₁, CH₂F; perhaloalkyl such as CCl₃, CClF₂, CFCl₂, CF₂CClF₂, CF₂CClFCF₃; polyhaloalkyl such as CH₂CHFCl, CF₂CClFH, CF₂CBrFH, CH₂CF₃; the term perhaloalkyl also encompasses the term perfluoroalkyl.

“Partially fluorinated alkyl” denotes a straight-chain or branched, saturated hydrocarbon which is mono- or polysubstituted by fluorine, where the fluorine atoms in question may be present as substituents on one or more different carbon atoms of the straight-chain or branched hydrocarbon chain, for example CHFCH₃, CH₂CH₂F, CH₂CH₂CF₃, CHF₂, CH₂F, CHFCF₂CF₃.

“Partially fluorinated haloalkyl” denotes a straight-chain or branched, saturated hydrocarbon which is substituted by different halogen atoms with at least one fluorine atom, where any other halogen atoms optionally present are selected from the group consisting of fluorine, chlorine or bromine, iodine. The corresponding halogen atoms may be present as substituents on one or more different carbon atoms of the straight-chain or branched hydrocarbon chain. Partially fluorinated haloalkyl also includes full substitution of the straight or branched chain by halogen including at least one fluorine atom.

“Haloalkoxy” is, for example, OCF₃, OCHF₂, OCH₂F, OCF₂CF₃, OCH₂CF₃ and OCH₂CH₂Cl; this applies correspondingly to haloalkenyl and other halogen-substituted radicals.

The expression “(C₁-C₄)-alkyl” mentioned here by way of example is a brief notation for straight-chain or branched alkyl having one to 4 carbon atoms according to the range stated for carbon atoms, i.e. encompasses the methyl, ethyl, 1-propyl, 2-propyl, 1-butyl, 2-butyl, 2-methylpropyl or tert-butyl radicals. General alkyl radicals with a larger specified range of carbon atoms, e.g. “(C₁-C₆)-alkyl”, correspondingly also encompass straight-chain or branched alkyl radicals with a greater number of carbon atoms, i.e. according to the example also the alkyl radicals having 5 and 6 carbon atoms.

Unless stated specifically, preference is given to the lower carbon skeletons, for example having from 1 to 6 carbon atoms, or having from 2 to 6 carbon atoms in the case of unsaturated groups, in the case of the hydrocarbon radicals such as alkyl, alkenyl and alkynyl radicals, including in composite radicals. Alkyl radicals, including in composite radicals such as alkoxy, haloalkyl, etc., are, for example, methyl, ethyl, n-propyl or i-propyl, n-, i-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

The term “alkenyl” also includes, in particular, straight-chain or branched open-chain hydrocarbon radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl. Alkenyl denotes, for example, vinyl which may optionally be substituted by further alkyl radicals, for example (but not limited thereto) (C₂-C₆)-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “alkynyl” also includes, in particular, straight-chain or branched open-chain hydrocarbon radicals having more than one triple bond, or else having one or more triple bonds and one or more double bonds, for example 1,3-butatrienyl or 3-penten-1-yn-1-yl. (C₂-C₆)-Alkynyl denotes, for example, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

The term “cycloalkyl” refers to a carbocyclic saturated ring system having preferably 3-8 ring carbon atoms, for example cyclopropyl, cyclobutyl, cyclopentyl or cyclohexyl, which optionally has further substitution, preferably by hydrogen, alkyl, alkoxy, cyano, nitro, alkylthio, haloalkylthio, halogen, alkenyl, alkynyl, haloalkyl, amino, alkylamino, bisalkylamino, alkoxycarbonyl, hydroxycarbonyl, arylalkoxycarbonyl, aminocarbonyl, alkylaminocarbonyl, cycloalkylaminocarbonyl. In the case of optionally substituted cycloalkyl, cyclic systems with substituents are included, also including substituents with a double bond on the cycloalkyl radical, for example an alkylidene group such as methylidene. In the case of optionally substituted cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl and adamantan-2-yl, but also systems such as 1,1′-bi(cyclopropyl)-1-yl, 1,1′-bi(cyclopropyl)-2-yl, for example. The term “(C₃-C₇)-cycloalkyl” is a brief notation for cycloalkyl having three to 7 carbon atoms, corresponding to the range specified for carbon atoms.

In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl.

“Cycloalkenyl” denotes a carbocyclic, nonaromatic, partially unsaturated ring system having preferably 4-8 carbon atoms, e.g. 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

The term “alkylidene”, also, for example, in the form (C₁-C₁₀)-alkylidene, means the radical of a straight-chain or branched open-chain hydrocarbon radical which is bonded via a double bond. Possible bonding sites for alkylidene are naturally only positions on the base structure where two hydrogen atoms can be replaced by the double bond; radicals are, for example, ═CH₂, ═CH—CH₃, ═C(CH₃)—CH₃, ═C(CH₃)—C₂H₅ or ═C(C₂H₅)—C₂H₅ Cycloalkylidene denotes a carbocyclic radical bonded via a double bond.

The term “alkylene”, also, for example, in the form (C₁-C₈)-alkylene, denotes the radical of a straight-chain or branched open-chain hydrocarbon radical which is attached at two positions to further groups.

“Cycloalkylalkyloxy” denotes a cycloalkylalkyl radical bonded via an oxygen atom and “arylalkyloxy” denotes an arylalkyl radical bonded via an oxygen atom.

“Alkoxyalkyl” represents an alkoxy radical bonded via an alkyl group and “alkoxyalkoxy” denotes an alkoxyalkyl radical bonded via an oxygen atom, for example (but not limited to) methoxymethoxy, methoxyethoxy, ethoxyethoxy, methoxy-n-propyloxy.

“Alkylthioalkyl” represents an alkylthio radical bonded via an alkyl group and “alkylthioalkylthio” denotes an alkylthioalkyl radical bonded via an oxygen atom.

“Arylalkoxyalkyl” represents an aryloxy radical bonded via an alkyl group and “heteroaryloxyalkyl” denotes a heteroaryloxy radical bonded via an alkyl group.

“Haloalkoxyalkyl” represents a bonded haloalkoxy radical and “haloalkylthioalkyl” denotes a haloalkylthio radical, bonded via an alkyl group.

“Arylalkyl” represents an aryl radical bonded via an alkyl group, “heteroarylalkyl” denotes a heteroaryl radical bonded via an alkyl group, and “heterocyclylalkyl” denotes a heterocyclyl radical bonded via an alkyl group.

“Cycloalkylalkyl” represents a cycloalkyl radical bonded via an alkyl group, for example (but not limited thereto) cyclopropylmethyl, cyclobutylmethyl, cyclopentylmethyl, cyclohexylmethyl, 1-cyclopropyleth-1-yl, 2-cyclopropyleth-1-yl, 1-cyclopropylprop-1-yl, 3-cyclopropylprop-1-yl.

“Arylalkenyl” represents an aryl radical bonded via an alkenyl group, “heteroarylalkenyl” denotes a heteroaryl radical bonded via an alkenyl group, and “heterocyclylalkenyl” denotes a heterocyclyl radical bonded via an alkenyl group.

“Arylalkynyl” represents an aryl radical bonded via an alkynyl group, “heteroarylalkynyl” denotes a heteroaryl radical bonded via an alkynyl group, and “heterocyclylalkynyl” denotes a heterocyclyl radical bonded via an alkynyl group.

According to the invention, “haloalkylthio”—on its own or as constituent part of a chemical group—represents straight-chain or branched S-haloalkyl, preferably having 1 to 8, or having 1 to 6 carbon atoms, such as (C₁-C₈)-, (C₁-C₈)- or (C₁-C₄)-haloalkylthio, for example (but not limited thereto) trifluoromethylthio, pentafluoroethylthio, difluoromethyl, 2,2-difluoroeth-1-ylthio, 2,2,2-difluoroeth-1-ylthio, 3,3,3-prop-1-ylthio.

“Halocycloalkyl” and “halocycloalkenyl” denote cycloalkyl and cycloalkenyl, respectively, which are partially or fully substituted by identical or different halogen atoms, such as B, F, Cl and Br, or by haloalkyl, such as trifluoromethyl or difluoromethyl, for example 1-fluorocycloprop-1-yl, 2-fluorocycloprop-1-yl, 2,2-difluorocycloprop-1-yl, 1-fluorocyclobut-1-yl, 1-trifluoromethylcycloprop-1-yl, 2-trifluoromethylcycloprop-1-yl, 1-chlorocycloprop-1-yl, 2-chlorocycloprop-1-yl, 2,2-dichlorocycloprop-1-yl, 3,3-difluorocyclobutyl.

According to the invention, “trialkylsilyl”—on its own or as constituent part of a chemical group—represents straight-chain or branched Si-alkyl, preferably having 1 to 8, or having 1 to 6 carbon atoms, such as tri[(C₁-C₈)-, (C₁-C₆)- or (C₁-C₄)-alkyl]silyl, for example (but not limited thereto) trimethylsilyl, triethylsilyl, tri(n-propyl)silyl, tri(isopropyl)silyl, tri(n-butyl)silyl, tri(1-methylprop-1-yl)silyl, tri(2-methylprop-1-yl)silyl, tri(1,1-dimethyleth-1-yl)silyl, tri(2,2-dimethyleth-1-yl)silyl.

“Trialkylsilylalkynyl” represents a trialkylsilyl radical bonded via an alkynyl group.

Synthesis of substituted N-phenyl-N-aminouracils of the general formula (I).

The substituted N-phenyl-N-aminouracils of the general formula (I) according to the invention can be prepared by known methods. The synthesis routes used and examined proceed from commercially available or easily preparable synthesis units. In the schemes which follow, the moieties G, Q, R¹, R², R³, R⁴, R⁵, R⁶, R², R¹⁵, R¹⁶, X and Y of the general formula (I) have the meanings defined above, unless exemplary, but not limiting, definitions are given. The synthesis of the compounds of the general formula (I) proceeds via various key intermediates. Key intermediates (III), where the groups R¹⁵ and R¹⁶ represent, by way of example, but not by way of limitation, hydrogen and X represents, by way of example, but not by way of limitation, sulfur (S), e.g. 3-(4-chloro-2-fluoro-5-mercaptophenyl)-1-amino-6-trifluoromethyl-1H-pyrimidine-2,4-dione (IIIa), are accessible via various synthesis routes (cf. U.S. Pat. No. 5,935,907, WO2004/56785, US2004/186021, WO2002/006244). Here, a suitable phthalimide is used as protective group for the N-amino group. The N-amino group can then be released by cleaving the phthalimide (e.g. with hydrazine). Alternatively, the desired intermediate (III) can also be prepared by direct N-amination as key step. To this end, a suitable substituted aniline, by way of example, but not by way of limitation, 2-fluoro-4-chloroaniline, is converted with a suitable reagent (e.g. triphosgene) in a suitable polar aprotic solvent (e.g. dichloromethane) into the corresponding isocyanate which, in the next step, is converted by reaction with a suitable aminoacrylic ester, e.g. ethyl (2Z)-3-amino-4,4,4-trifluorobut-2-enoate, using a suitable base (e.g. sodium hydride or potassium tert-butoxide) in a suitable polar aprotic solvent (e.g. N,N-dimethylformamide) into the corresponding pyrimidine-2,4-dione, which is optionally substituted further, by way of example, but not by way of limitation, 3-(4-chloro-2-fluorophenyl)-6-trifluoromethyl-1H-pyrimidine-2,4-dione (Scheme 1). The required aminoacrylic ester, e.g. ethyl (2Z)-3-amino-4,4,4-trifluorobut-2-enoate, can be obtained by amination of the corresponding, optionally further-substituted, 3-oxobutanoate, e.g. ethyl 4,4,4-trifluoro-3-oxobutanoate, using a suitable nitrogen source (e.g. ammonium acetate) in a suitable polar-protic solvent (e.g. ethanol) at elevated temperatures. This is followed by N-amination with a suitable amination reagent (e.g. 0-(mesitylsulfonyl)hydroxylamine, 0-(tolylsulfonyl)hydroxylamine, 0-(diphenylphosphoryl)hydroxylamine) and introduction of the SH group by chlorosulfonylation and subsequent reduction with a suitable reducing agent (e.g. tin(II) chloride in an acidic solvent such as acetic acid). A further option of preparing the intermediates (III) is by reacting the corresponding aminoacrylic ester, e.g. ethyl (2Z)-3-amino-4,4,4-trifluorobut-2-enoate, with dimethylcarbamyl chloride in N,N-dimethylformamide (DMF) using a suitable base (e.g. sodium hydride or potassium tert-butoxide) and subsequent reaction with 2-fluoro-4-chloroaniline in a suitable acidic solvent (e.g. acetic acid) at elevated temperature. The resulting 3-(4-chloro-2-fluorophenyl)-6-trifluoromethyl-1H-pyrimidine-2,4-dione can also be converted in reverse order into the desired intermediate (III), by introduction of the SH group, introduction of an S-trityl protective group, N-amination with one of the suitable amination reagents mentioned above and subsequent removal of the S-trityl protective group. In Scheme 1 below, R¹, by way of example, but not by way of limitation, represents hydrogen, R², by way of example, but not by way of limitation, represents fluorine, R³, by way of example, but not by way of limitation, represents fluorine, R⁴, by way of example, but not by way of limitation, represents chlorine, and R¹⁵, R¹⁶, by way of example, but not by way of limitation, represent hydrogen.

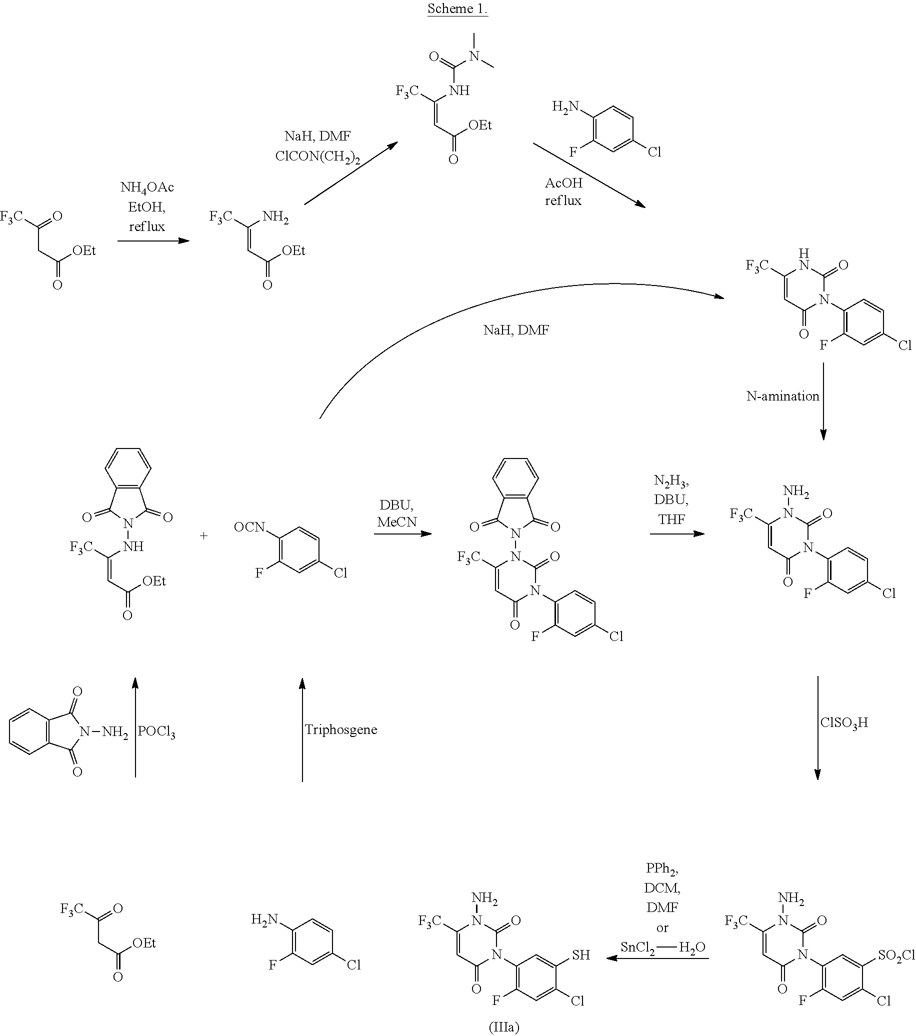

For prolonged storage of the intermediates (III) or the alternative reaction sequence described above, it has in some cases been found to be advantageous to protect the mercapto group with a suitable thio protective group (e.g. trityl). Using suitable reagents (e.g. trifluoroacetic acid, triisopropylsilane), the mercapto group can be deprotected again (cf. Scheme 2). The respective further-substituted N-amino-5-mercaptophenyl-1H-pyrimidine-2,4-dione intermediates (III) can then be converted by various routes into the desired compounds of the general formula (Ia) according to the invention in which X represents sulfur (S) and Y represents oxygen (O) (Scheme 2), after converting the compounds (III) in a first step with the aid of a suitable optionally further-substituted iodopyridone using a suitable base or using a suitable transition metal catalyst (e.g. tris(dibenzylideneacetone)dipalladium(O)) with a suitable ligand (e.g. 4,5-bis(diphenylphosphino)-9,9-dimethylxanthene) and a suitable base (e.g. diispropyl(ethyl)amine) in a suitable polar-aprotic solvent (e.g. dioxane) into intermediates (IV). In Scheme 2 below, Q, R¹, R², R³, R⁴, R¹⁵ and R¹⁶ have the above meanings according to the invention. Furthermore, R⁵, R⁶, R⁷, by way of example, but not by way of limitation, represent hydrogen, X, by way of example, but not by way of limitation, represents sulfur, Y, by way of example, but not by way of limitation, represents oxygen and G, by way of example, but not by way of limitation, represents CH₂.

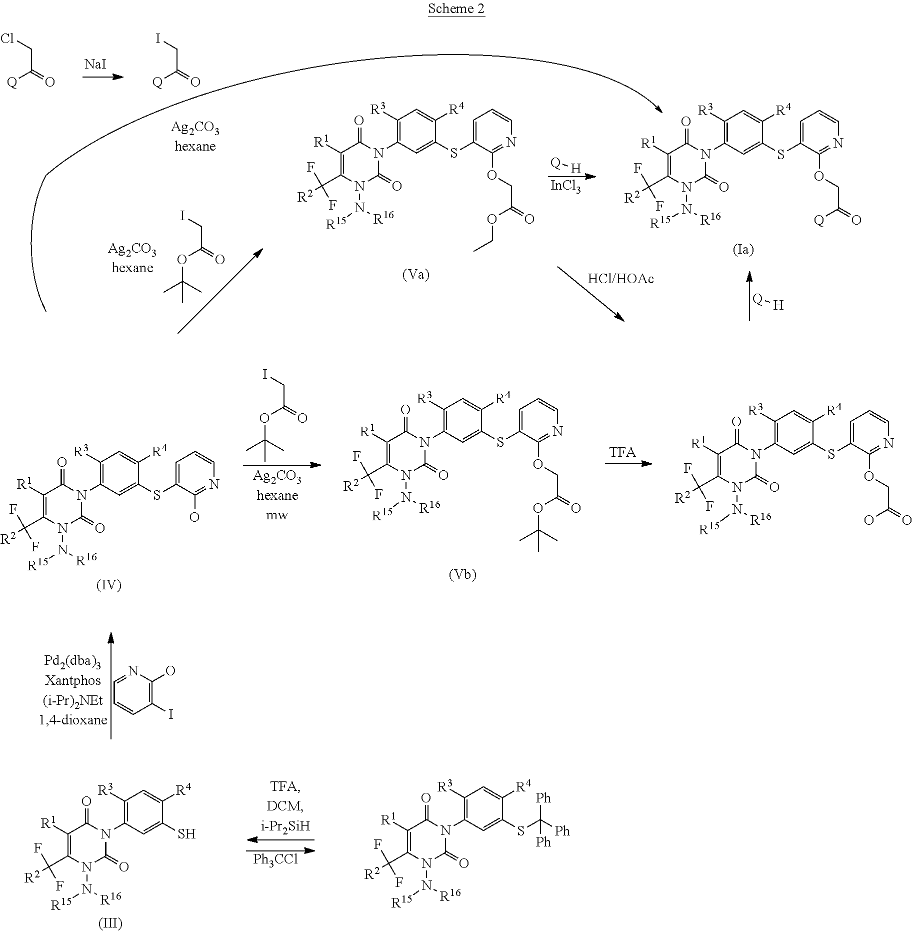

The corresponding intermediate (IV) described by way of example, but not by way of limitation, in Scheme 2 can be converted by reaction with a suitable optionally further-substituted iodoalkanoic ester (in Schema 2 by way of example, but not by way of limitation, an iodoacetic ester) using a suitable base (e.g. silver(I) carbonate) in a suitable polar-aprotic solvent (e.g. n-hexane or cyclohexane) at elevated temperature (e.g. under microwave conditions) into a corresponding optionally further-substituted oxyalkanoic ester (Va, Vb) (cf. Synthesis 2009, 2725). The corresponding iodoalkanoic esters can be prepared by routes known from the literature (cf. Eur. J. Org. Chem., 2006, 71, 8459; WO2012037573; Organometallics, 2009, 28, 132). The ethyl esters (Va) and tert-butyl esters (Vb) described by way of example, but not by way of limitation, can then be converted under suitable reaction conditions [use of a suitable acid such as hydrochloric acid or acetic acid in the case of (Va) or trifluoroacetic acid (TFA) in the case of (Vb)] into the corresponding free acid of the general formula (IIa). By reaction of the corresponding acid described by way of example, but not by way of limitation, in Scheme 2 with a suitable compound Q-H with mediation by suitable coupling reagents (e.g. HOBt=1-hydroxybenzotriazole, EDC=1-ethyl-3-(3-dimethylaminopropyl)carbodiimide, HATU=O-(7-azabenzotriazol-1-yl)-N,N,N′,N′-tetramethyluronium hexafluorophosphate, T3P=2,4,6-tripropyl-1,3,5,2,4,6-trioxatriphosphorinane 2,4,6-trioxide) and suitable bases (e.g. diisopropylethylamine, triethylamine) in a suitable polar-aprotic solvent (e.g. dichloromethane, chloroform), it is possible to prepare the desired substituted N-phenyl-N-aminouracils of the general formula (Ia). Alternatively, the ethyl ester (Va) described by way of example, but not by way of limitation, in Scheme 2 can be converted by coupling with a suitable compound Q-H with mediation by a suitable Lewis acid (e.g. indium(III) chloride) into the corresponding desired substituted N-phenyl-N-aminouracil of the general formula (Ia) (cf. WO2011/1307088).

The preparation of the compounds of the general formula (I) in which the groups R¹⁵ and R¹⁶ represent hydrogen and X and Y, by way of example, but not by way of limitation, represent oxygen (O) proceeds via the synthesis of key intermediates (VI) having a fluorine substituent at position 5, such as 3-(2,5-difluoro-4-nitro)-1-amino-6-trifluoromethyl-1H-pyrimidine-2,4-dione (VIa). Here, a suitable phthalimide can be used as protective group for the amino group, or the desired intermediate (VI) can also be prepared via direct N-amination as key step. If the phthalimide-assisted synthesis route is utilized, the desired N-amino group can be released by cleaving the phthalimide after successful assembly of the substituted pyrimidine-2,4-dione (e.g. with hydrazine). When the key step employed is the N-amination, a suitable substituted aniline, by way of example, but not by way of limitation, 2,5-difluoroaniline, is converted with a suitable reagent (e.g. triphosgene) in a suitable polar aprotic solvent (e.g. dichloromethane) into the corresponding isocyanate which, in the next step, is converted by reaction with a suitable aminoacrylic ester using a suitable base (e.g. sodium hydride or potassium tert-butoxide) in a suitable polar aprotic solvent (e.g. N,N-dimethylformamide) into the corresponding pyrimidine-2,4-dione, which is optionally substituted further, here, by way of example, but not by way of limitation, 3-(2,5-difluorophenyl)-6-trifluoromethyl-1H-pyrimidine-2,4-dione (Scheme 3). By nitration with a suitable nitrating reagent and subsequent N-amination with the aid of a suitable aminating reagent (e.g. 0-(mesitylsulfonyl)hydroxylamine, 0-(tolylsulfonyl)hydroxylamine, 0-(diphenylphosphoryl)hydroxylamine, 0-(2,4-dinitrophenyl)hydroxylamine), the desired 3-(2,5-difluoro-4-nitro)-1-amino-6-trifluoromethyl-1H-pyrimidine-2,4-dione (Via) can be obtained. In Scheme 3 below, R¹, by way of example, but not by way of limitation, represents hydrogen, R², by way of example, but not by way of limitation, represents fluorine, R³, by way of example, but not by way of limitation, represents fluorine, and R⁴, by way of example, but not by way of limitation, represents nitro.

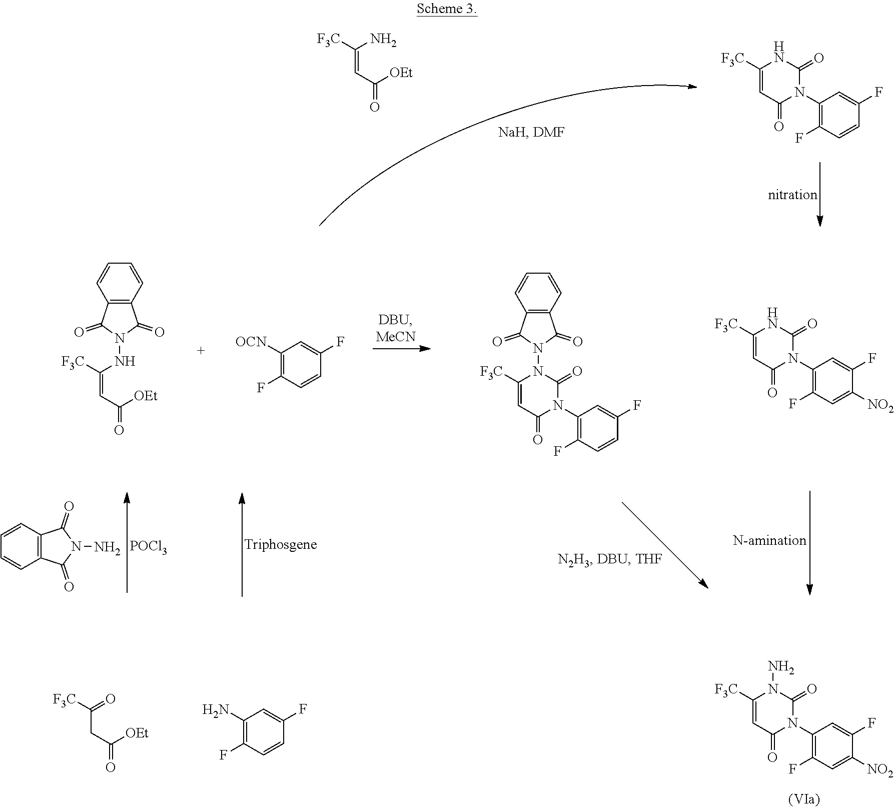

Functionalization of the 3-(2,5-difluoro-4-nitro)-1-amino-6-trifluoromethyl-1H-pyrimidine-2,4-dione (VIa) can be carried out in accordance with processes known to the person skilled in the art by functional group interconversion—examples which may be mentioned are monoalkylation (R¹⁵=H, R¹⁶=alkyl) and bisalkylation (R¹⁵=alkyl, R¹⁶=alkyl)—and is shown in Scheme 3b below. R¹ and R² have the above meanings according to the invention.

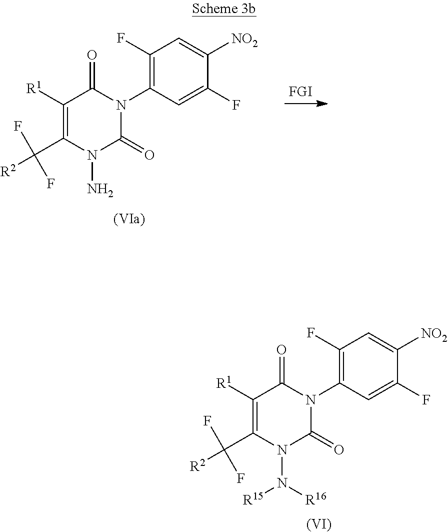

Intermediate (VI), obtained in the manner described above, can then be converted with a suitable substituted 2-carbonylalkyloxy-3-hydroxypyridine (VII) using a suitable base (e.g. potassium carbonate) in a suitable polar-aprotic solvent (e.g. N,N-dimethylformamide (DMF)) into a desired substituted N-phenyl-N-aminouracil (Ib, R⁴=nitro). The intermediate (VII) used for this purpose can be obtained by a multi-step synthesis starting with commercially available 2-chloro-3-nitropyridine, via (i) base-mediated coupling (e.g. with sodium hydride) with a suitable substituted hydroxyalkylcarbonyl reagent in a suitable polar-aprotic solvent (e.g. tetrahydrofuran or dioxane), (ii) reduction of the nitro group with a suitable reducing agent (e.g. hydrogen, palladium on carbon in a suitable polar-protic solvent), (iii) diazotization (with a suitable diazotization reagent, e.g. tert-butyl nitrite (t-BuONO), boron trifluoride etherate (BF₃-OEt₂) in suitable polar-aprotic solvents (e.g. dichloromethane (DCM), dimethoxyethane), (iv) reaction with acetic anhydride and (v) release of the hydroxy group by removal of the acetyl protective group (e.g. base-mediated with potassium carbonate in a polar-protic solvent). The nitro group in compound (Ib) can, if NR¹⁵R¹⁶ does not represent NH₂, then be converted by reduction and subsequent Sandmeyer reaction into a halogen substituent (e.g. chlorine, bromine), such that the desired substituted N-phenyl-N-aminouracil (Ic) can be obtained in this manner. In Scheme 4 below, Q, R¹, R², R¹⁵ and R¹⁶ have the above meanings according to the invention. Furthermore, R³, by way of example, but not by way of limitation, represents fluorine, R⁴, by way of example, but not by way of limitation, represents chlorine or nitro, R⁵, R⁶, R⁷, by way of example, but not by way of limitation, represent hydrogen, X and Y, by way of example, but not by way of limitation, represent oxygen and G, by way of example, but not by way of limitation, represents CH₂.

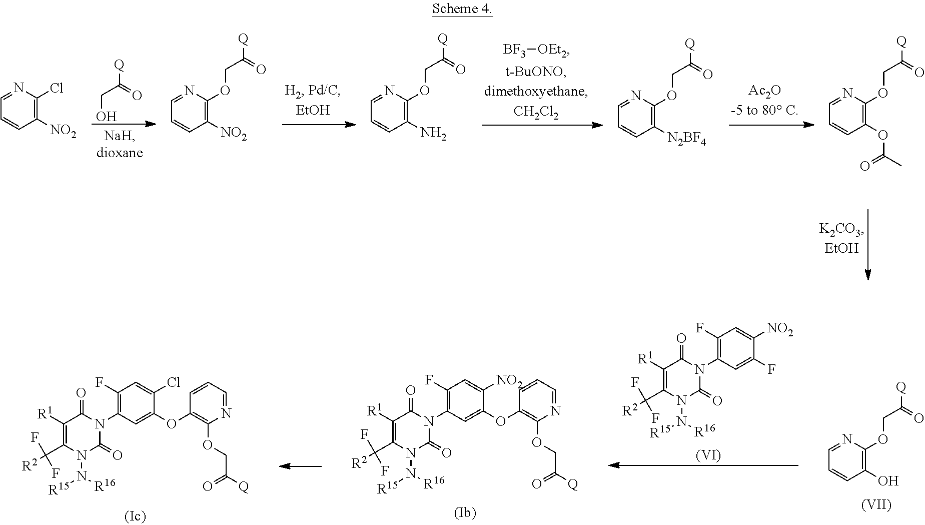

Intermediate (VI), obtained in the manner described above, can then be converted with a suitable substituted 2-carbonylalkylthio-3-hydroxypyridine (VIII) using a suitable base (e.g. potassium carbonate) in a suitable polar-aprotic solvent (e.g. N,N-dimethylformamide (DMF)) into a desired substituted N-phenyl-N-aminouracil (Id, R⁴=nitro) where X=O (oxygen) and Y=S (sulfur). The intermediate (VIII) used for this purpose can be prepared by a multi-step synthesis analogously to the synthesis of intermediate (VII) described in Scheme 4 starting with commercially available 2-chloro-3-nitropyridine. The nitro group in compound (Id) can, if NR¹⁵R¹⁶ does not represent NH₂, then be converted by reduction and subsequent Sandmeyer reaction into a halogen substituent (e.g. chlorine, bromine), such that the desired substituted N-phenyl-N-aminouracil (Ie) can be obtained in this manner. In Scheme 5 below, Q, R¹, R², V and R¹⁶ have the above meanings according to the invention. Furthermore, R³, by way of example, but not by way of limitation, represents fluorine, R⁴, by way of example, but not by way of limitation, represents chlorine or nitro, R⁵, R⁶, R⁷, by way of example, but not by way of limitation, represent hydrogen, X, by way of example, but not by way of limitation, represents oxygen, Y, by way of example, but not by way of limitation, represents sulfur and G, by way of example, but not by way of limitation, represents CH₂.

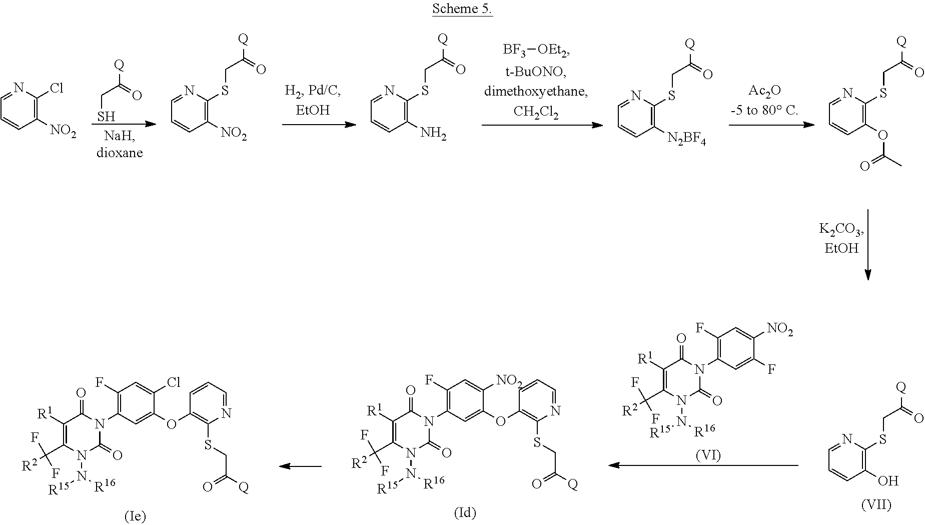

The further-substituted N-amino-5-mercaptophenyl-1H-pyrimidine-2,4-dione intermediates (III) can also be converted into the desired compounds of the general formula (If) according to the invention in which X and Y represent sulfur (S) (Scheme 6), after converting the compounds (III) in a first step with the aid of a suitable optionally further-substituted iodothiopyridine using a suitable base or using a suitable transition metal catalyst (e.g. tris(dibenzylideneacetone)dipalladium(0)) with a suitable ligand (e.g. 4,5-bis(diphenylphosphino)-9,9-dimethylxanthene) and a suitable base (e.g. diispropyl(ethyl)amine) in a suitable polar-aprotic solvent (e.g. dioxane) into intermediates of type (IX). The intermediates (IX) can then be reacted with haloalkanecarboxylic acids having various substitutions using suitable bases, to afford the desired compounds of the general formula (If). In Scheme 6 below, Q, R¹, R², R³, R⁴, R¹⁵ and R¹⁶ have the above meanings according to the invention. Furthermore, R⁵, R⁶, R², by way of example, but not by way of limitation, represent hydrogen, X and Y, by way of example, but not by way of limitation, represent sulfur and G, by way of example, but not by way of limitation, represents CH₂. For clarity, the reaction paths are furthermore described in Scheme 6 below, by way of example, but not by way of limitation, using iodoacetic esters. Also suitable for coupling with intermediate (IX) are comparable haloalkanecarboxylic acids (halogen=bromine or chlorine).

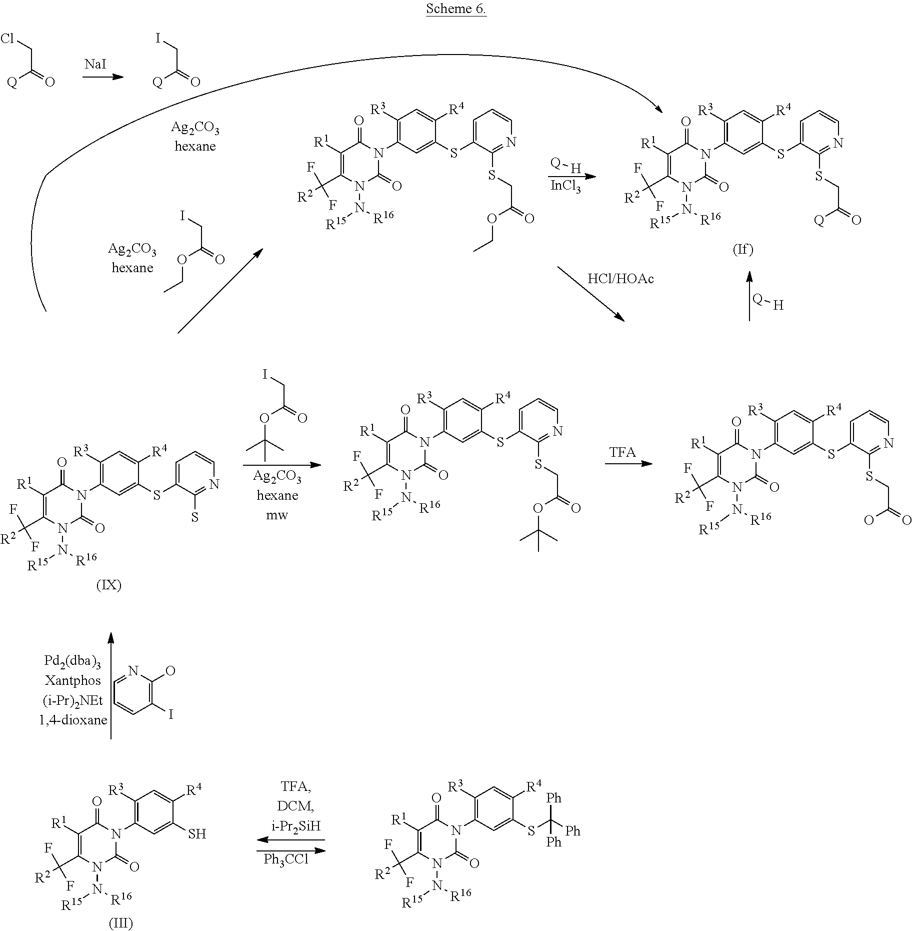

The preparation of the compounds of the general formula (I) in which the groups R¹⁵ and R¹⁶ represent hydrogen and X and Y, by way of example, but not by way of limitation, represent oxygen (O) proceeds via N-amination as key step. To this end, a suitable substituted aniline, by way of example, but not by way of limitation, 2,5-difluoroaniline, is converted with a suitable reagent (e.g. triphosgene) in a suitable polar aprotic solvent (e.g. dichloromethane) into the corresponding isocyanate which, in the next step, is converted by reaction with a suitable aminoacrylic ester using a suitable base (e.g. sodium hydride or potassium tert-butoxide) in a suitable polar aprotic solvent (e.g. N,N-dimethylformamide) into the corresponding pyrimidine-2,4-dione, which is optionally substituted further, here, by way of example, but not by way of limitation, 3-(2,5-difluorophenyl)-6-haloalkyl-1H-pyrimidine-2,4-dione. By nitration with a suitable nitrating reagent, it is possible to obtain the desired intermediate (X), in Scheme 7 by way of example, but not by way of limitation, 3-(2,5-difluoro-4-nitro)-1-amino-6-haloalkyl-1H-pyrimidine-2,4-dione (X). Intermediate (X), obtained in the manner described above, can then be converted with a suitable substituted 2-carbonylalkyloxy-3-hydroxypyridine (VII) using a suitable base (e.g. potassium carbonate) in a suitable polar-aprotic solvent (e.g. N,N-dimethylformamide (DMF)) into the desired substituted N-phenyl-N-1H-uracil XIa (R⁴=nitro). The nitro group in compound (XIa) can then be converted by reduction and subsequent Sandmeyer reaction into a halogen substituent (e.g. chlorine, bromine), such that the desired substituted N-phenyl-1H-uracil (XIb) can be obtained in this manner. This is subsequently converted by N-amination with a suitable aminating reagent (e.g. 0-(mesitylsulfonyl)hydroxylamine, O-(tolylsulfonyl)hydroxylamine, O-(diphenylphosphoryl)hydroxylamine or O-(2,4-dinitrophenyl)hydroxylamine) into the desired substituted N-phenyl-N-aminouracil (Ig).

In Scheme 7 below, Q, R¹ and R² have the above meanings according to the invention. Furthermore, R⁴, by way of example, but not by way of limitation, represents chlorine, R⁵, R⁶, R⁷, by way of example, but not by way of limitation, represent hydrogen, X and Y, by way of example, but not by way of limitation, represent oxygen and G, by way of example, but not by way of limitation, represents CH₂.

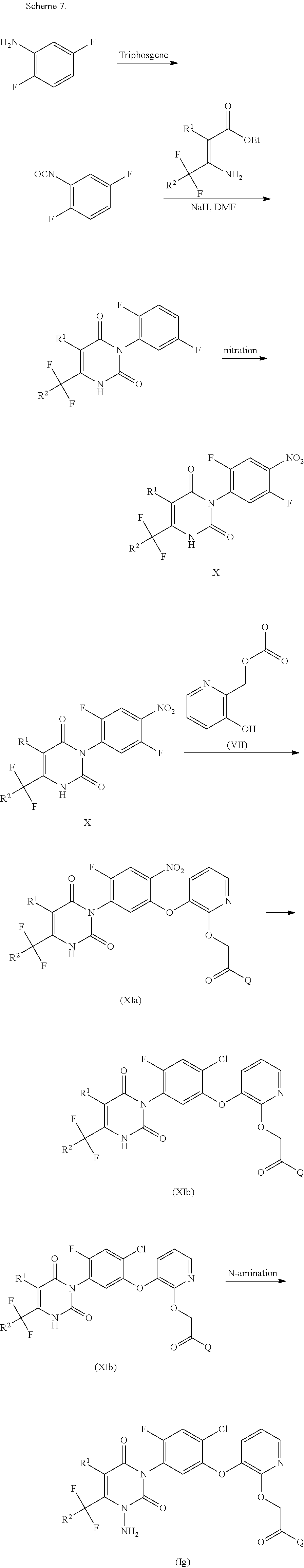

Functionalization of the N-phenyl-N aminouracil (Ig) to give further compounds (Ic) according to the invention can be carried out in accordance with processes known to the person skilled in the art by functional group interconversion—examples which may be mentioned are monoalkylation (R¹⁵=H, R¹⁶=alkyl) and bisalkylation (R¹⁵=alkyl, R¹⁶=alkyl)—and is shown in Scheme 8 below. In the scheme below, Q, R¹, R², R¹⁵ and R¹⁶ have the above meanings according to the invention. Furthermore, R³, by way of example, but not by way of limitation, represents fluorine, R⁴, by way of example, but not by way of limitation, represents chlorine, R⁵, R⁶, R⁷, by way of example, but not by way of limitation, represent hydrogen, X and Y, by way of example, but not by way of limitation, represent oxygen and G, by way of example, but not by way of limitation, represents CH₂.

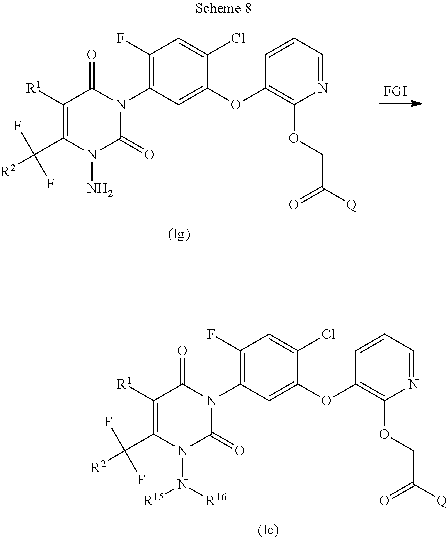

Selected detailed synthesis examples for the compounds of the general formulae (I) according to the invention are given below. The example numbers mentioned correspond to the numbering scheme in Tables I.1 to I.34 below. The ¹H NMR, ¹³C-NMR and ¹⁹F-NMR spectroscopy data reported for the chemical examples described in the sections which follow (400 MHz for ¹H NMR and 150 MHz for ¹³C-NMR and 375 MHz for ¹⁹F-NMR, solvent CDCl₃, CD₃OD or d₆-DMSO, internal standard: tetramethylsilane 6=0.00 ppm) were obtained on a Bruker instrument, and the signals listed have the meanings given below: br=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets. In the case of diastereomer mixtures, either the significant signals for each of the two diastereomers are reported or the characteristic signal of the main diastereomer is reported. The abbreviations used for chemical groups have, for example, the following meanings: Me=CH₃, Et=CH₂CH₃, t-Hex=C(CH₃)₂CH(CH₃)₂, t-Bu=C(CH₃)₃, n-Bu=unbranched butyl, n-Pr=unbranched propyl, i-Pr=branched propyl, c-Pr=cyclopropyl, c-Hex=cyclohexyl.

### SYNTHESIS EXAMPLES

### No. I.1-2: 2-Ethoxyethyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate

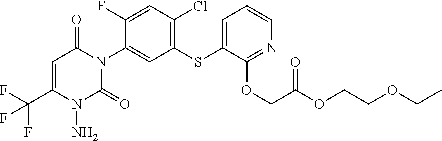 [SMILES: CCOCCOC(=O)COc1ncccc1Sc1cc(-n2c(=O)cc(C(F)(F)F)n(N)c2=O)c(F)cc1Cl]

Successively, 2-fluoro-4-chloroaniline (145 g, 996 mmol) and triethylamine (202 g, 2000 mmol) were added carefully to a solution of triphosgene (119 g, 401 mmol) in abs. dichloromethane (1000 ml) such that the temperature of the resulting reaction mixture remained below 20° C. After the addition had ended, the reaction mixture was stirred at room temperature overnight and then washed with water (3×500 ml) and 1N hydrochloric acid (500 ml), dried over sodium sulfate, filtered and concentrated under reduced pressure. The resulting 2-fluoro-4-chlorophenyl isocyanate was used in the next stage without further purification. Sodium hydride (5.60 g, 140 mmol, 60% dispersion in mineral oil) was suspended in abs. N,N-dimethylformamide, and ethyl (2E)-3-amino-4,4,4-trifluorobut-2-enoate (14.2 g, 77.5 mmol) was added. The reaction mixture was stirred at room temperature for 1 h and then cooled to a temperature of −30° C., and 2-fluoro-4-chlorophenyl isocyanate (12.0 g, 70.0 mmol) was added. After the addition had ended, the resulting reaction mixture was stirred at room temperature for a further 4 h and then added to ice-water. After addition of ethyl acetate and acidification with 1N hydrochloric acid, the aqueous phase was extracted thoroughly with ethyl acetate. The combined organic phases were washed with water, dried over sodium sulfate, filtered and concentrated under reduced pressure. This gave 3-(4-chloro-2-fluorophenyl)-6-(trifluoromethyl)pyrimidine-2,4(1H,3H)-dione (15.2 g, 50.2 mmol, 65%), which was used in the next stage without further purification. It was also possible to prepare 3-(4-chloro-2-fluorophenyl)-6-(trifluoromethyl)pyrimidine-2,4(1H,3H)-dione on a larger scale, either by repeating the synthesis route described above or via the following, slightly modified synthesis route. Ethyl 4,4,4-trifluoro-3-oxobutanoate (200 g, 1087 mmol) was dissolved in ethanol (1.3 l), ammonium acetate (334.7 g, 4347 mmol) was added and the mixture was then stirred under reflux conditions for 16 h. After addition of ethyl acetate and water, the aqueous phase was extracted thoroughly with ethyl acetate. The combined organic phases were washed with water and sat. NaCl solution, dried over sodium sulfate and filtered, and the crude product was then purified carefully by distillation (500 mm/Hg at about 40 to 45° C.). Ethyl (2Z)-3-amino-4,4,4-trifluorobut-2-enoate (180 g, 983 mmol), which was obtained as a slightly yellowish liquid, was then used for the next reaction step without further purification and added to a suspension, cooled to 0° C., of sodium hydride (98.3 g, 2458 mmol) in abs. N,N-dimethylformamide (1.5 l). The resulting reaction mixture was stirred at 0° C. for 1 h and at room temperature for 1 h. After once more cooling to 0° C., dimethylcarbamoyl chloride (210.4 g, 1967 mmol) was added. The reaction mixture was stirred at room temperature for another 2 h. After addition of ethyl acetate and water, the aqueous phase was extracted thoroughly with ethyl acetate. The combined organic phases were washed with water, dried over sodium sulfate, filtered and concentrated under reduced pressure. Purification of the crude product obtained by column chromatography gave ethyl (2Z)-3-[(dimethylcarbamoyl)amino]-4,4,4-trifluorobut-2-enoate (120 g, 38% of theory) in the form of a slightly brownish oil. 2-Chloro-4-fluoroaniline (50 g, 344.8 mmol) and ethyl (2Z)-3-[(dimethylcarbamoyl)amino]-4,4,4-trifluorobut-2-enoate (87.6 g, 344.8 mmol) were dissolved in acetic acid (500 ml) and stirred under reflux conditions for 48 h. After the reaction had ended, the reaction mixture was cooled to room temperature and added to ice-water. The aqueous mixture was filtered and the resulting filtered solid was dissolved in ethyl acetate and washed with sat. NaCl solution. The organic phases were dried over sodium sulfate, filtered and concentrated under reduced pressure. Purification by column chromatography gave 3-(4-chloro-2-fluorophenyl)-6-(trifluoromethyl)pyrimidine-2,4(1H,3H)-dione (40.0 g, 38% of theory) as a colorless solid. 3-(4-Chloro-2-fluorophenyl)-6-(trifluoromethyl)pyrimidine-2,4(1H,3H)-dione (40 g, 130 mmol) was then added a little at a time to chlorosulfonic acid (60 ml) in a round-bottom flask which had been dried by heating. The resulting reaction mixture was then stirred at a temperature of 130° C. under an atmosphere of nitrogen for 5 h and, after cooling to room temperature, added to ice-water and extracted repeatedly with ethyl acetate (3×300 ml). The combined organic phases were dried over sodium sulfate, filtered and concentrated under reduced pressure. This gave 2-chloro-5-[2,6-dioxo-4-(trifluoromethyl)-3,6-dihydropyrimidin-1(2H)-yl]-4-fluorobenzenesulfonyl chloride (30.0 g, 57% of theory), which was used in the next stage without further purification. ¹H-NMR (CDCl₃ δ, ppm) 9.49 (br. s, 1H), 8.15 (d, 1H), 7.57 (d, 1H), 6.29 (s, 1H). 2-Chloro-5-[2,6-dioxo-4-(trifluoromethyl)-3,6-dihydropyrimidin-1(2H)-yl]-4-fluorobenzenesulfonyl chloride (30.0 g, 74 mmol) was initially charged in a round-bottom flask, and hydrochloric acid (105 ml), acetic acid (225 ml) and tin dichloride dihydrate (79 g, 251 mmol) were added in succession. The resulting reaction mixture was stirred at a temperature of 80° C. for 5 h and, after cooling to room temperature, added to ice-water and extracted thoroughly with dichloromethane (3×400 ml). The combined organic phases were dried over sodium sulfate, filtered and concentrated under reduced pressure. Final purification by column chromatography gave 3-(4-chloro-2-fluoro-5-sulfanylphenyl)-6-(trifluoromethyl)pyrimidine-2,4(1H,3H)-dione (25.0 g, 98% of theory) in the form of a colorless solid. ¹H-NMR (CDCl₃ δ, ppm) 8.85 (br. s, 1H), 7.35 (d, 1H), 7.29 (d, 1H), 6.26 (s, 1H), 3.88 (s, 1H). 3-(4-Chloro-2-fluoro-5-sulfanylphenyl)-6-(trifluoromethyl)pyrimidine-2,4(1H,3H)-dione (25.00 g, 73.5 mmol) was dissolved in abs. dichloromethane (500 ml), and trityl chloride (34.15 g, 73.5 mmol) and pyridine (5.81 g, 73.53 mmol) were added. The resulting reaction mixture was stirred at room temperature for 3 h and then water was added and the mixture was extracted thoroughly. The combined organic phases were dried over sodium sulfate, filtered and concentrated under reduced pressure. Final purification by column chromatography gave 3-[4-chloro-2-fluoro-5-(tritylsulfanyl)phenyl]-6-(trifluoromethyl)pyrimidine-2,4(1H,3H)-dione (25 g, 43% of theory) in the form of a colorless solid. ¹H NMR (CDCl₃ δ, ppm) 9.40 (br. s, 1H), 7.39-7.23 (m, 15H), 7.20 (m, 1H), 6.74 (d, 1H), 6.13 (s, 1H). 3-[4-Chloro-2-fluoro-5-(tritylsulfanyl)phenyl]-6-(trifluoromethyl)pyrimidine-2,4(1H,3H)-dione (25 g, 1 equiv.) was dissolved in abs. tetrahydrofuran (625 ml), and sodium carbonate (3 equiv.) was added. O-Mesitylenesulfonylhydroxylamine (3 equiv.) was then added carefully, a little at a time. The resulting reaction mixture was subsequently stirred under nitrogen for 12 h. After complete conversion, water and ethyl acetate were added and the mixture was repeatedly thoroughly extracted. The combined organic phases were dried over sodium sulfate, filtered and carefully concentrated under reduced pressure. Final purification by column chromatography gave 1-amino-3-[4-chloro-2-fluoro-5-(tritylsulfanyl)phenyl]-6-(trifluoromethyl)pyrimidine-2,4(1H,3H)-dione (21.1 g, 82% of theory) in the form of a colorless solid. ¹H-NMR (d₆-DMSO δ, ppm) 7.61 (d, 1H), 7.33-7.23 (m, 15H), 6.88 (d, 1H), 6.34 (s, 1H), 5.54 (br. s, 2H, NH₂). Under argon, 1-amino-3-[4-chloro-2-fluoro-5-(tritylsulfanyl)phenyl]-6-(trifluoromethyl)pyrimidine-2,4(1H,3H)-dione (5.0 g, 8.53 mmol) was dissolved in dichloromethane (50 ml), and the mixture was cooled to 0° C. Trifluoroacetic acid (2.30 ml, 29.86 mmol) and triisopropylsilane (9.16 ml, 44.79 mmol) were then added. The resulting reaction mixture was stirred at room temperature for about 3 h. After complete conversion, water and dichloromethane were added and the mixture was extracted thoroughly. The combined organic phases were dried over sodium sulfate, filtered and carefully concentrated under reduced pressure. Final purification by column chromatography gave 1-amino-3-(4-chloro-2-fluoro-5-sulfanylphenyl)-6-(trifluoromethyl)pyrimidine-2,4(1H,3H)-dione (2.98 g, 98% of theory) in the form of a colorless solid. ¹H-NMR (CDCl₃ δ, ppm) 7.36 (d, 1H), 7.29 (d, 1H), 6.28 (s, 1H), 4.59 (s, 2H), 3.88 (s, 1H). Under argon, 1-amino-3-(4-chloro-2-fluoro-5-sulfanylphenyl)-6-(trifluoromethyl)pyrimidine-2,4(1H,3H)-dione (600 mg, 1.69 mmol) was dissolved in dioxane (18 ml) in a microwave vessel and, after degassing of the solvent, tris(dibenzylideneacetone)dipalladium (39 mg, 0.04 mmol), 4,5-bis(diphenylphosphino)-9,9-dimethylxanthene (49 mg, 0.08 mmol), N,N-diisopropylethylamine (0.59 ml, 3.37 mmol) and 3-bromo-2-hydroxypyridine (333 mg, 1.86 mmol) were added. The resulting reaction mixture was stirred under microwave conditions at a temperature of 160° C. for 2 h. After cooling to room temperature, the reaction mixture was filtered and the filtrate was concentrated. Purification of the resulting crude product by column chromatography gave 1-amino-3-{4-chloro-2-fluoro-5-[(2-hydroxypyridin-3-yl)sulfanyl]phenyl}-6-(trifluoromethyl)pyrimidine-2,4(1H,3H)-dione (720 mg, 86% of theory) in the form of a colorless solid. ¹H-NMR (CDCl₃ δ, ppm) 12.64 (br. s, 1H), 7.45 (d, 1H), 7.40 (d, 1H), 7.35 (d, 1H), 7.24 (d, 1H), 6.30 (m, 1H), 6.23 (s, 1H), 4.58 (s, 2H). In a microwave vessel and under argon, n-hexane (19 ml) was added to 1-amino-3-{4-chloro-2-fluoro-5-[(2-hydroxypyridin-3-yl)sulfanyl]phenyl}-6-(trifluoromethyl)pyrimidine-2,4(1H,3H)-dione (300 mg, 0.69 mmol). Silver(I)carbonate (223 mg, 0.80 mmol) and 2-ethoxyethyl iodoacetate (431 mg, 1.34 mmol) were then added. The reaction mixture was stirred at a temperature of 140° C. under microwave conditions for 40 minutes. After cooling to room temperature, the reaction mixture was filtered and the filtrate was concentrated. Purification of the resulting crude product by column chromatography gave 2-ethoxyethyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate (119 mg, 31% of theory) in the form of a colorless solid. ¹H-NMR (CDCl₃ δ, ppm) 8.12 (d, 1H), 7.77 (m, 1H), 7.32 (d, 1H), 7.24 (d, 1H), 6.95 (m, 1H), 6.19 (s, 1H), 4.99-4.87 (q, 2H), 4.62 (s, 2H), 4.36-4.29 (m, 1H), 4.17-4.11 (m, 1H), 3.64-3.61 (m, 2H), 3.53 (q, 2H), 1.20 (t, 3H).

### No. I.1-23: 2-(2-Methoxyethoxy)ethyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate

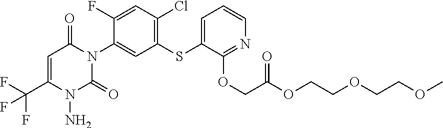 [SMILES: COCCOCCOC(=O)COc1ncccc1Sc1cc(-n2c(=O)cc(C(F)(F)F)n(N)c2=O)c(F)cc1Cl]

In a microwave vessel and under argon, n-hexane (15 ml) was added to 1-amino-3-{4-chloro-2-fluoro-5-[(2-hydroxypyridin-3-yl)sulfanyl]phenyl}-6-(trifluoromethyl)pyrimidine-2,4(1H,3H)-dione (200 mg, 0.45 mmol). Silver(I) carbonate (147 mg, 0.54 mmol) and 2-(2-methoxyethoxy)ethyl iodoacetate (257 mg, 0.89 mmol) were then added. The reaction mixture was stirred at a temperature of 140° C. under microwave conditions for 48 minutes. After cooling to room temperature, the reaction mixture was filtered and the filtrate was concentrated under reduced pressure. Purification of the resulting crude product by column chromatography gave 2-(2-methoxyethoxy)ethyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate (68 mg, 23% of theory) in the form of a colorless solid. ¹H-NMR (CDCl₃ δ, ppm) 8.12 (d, 1H), 7.78 (m, 1H), 7.34 (d, 1H), 7.26 (d, 1H), 6.95 (m, 1H), 6.19 (s, 1H), 4.98-4.87 (q, 2H), 4.66 (s, 2H), 4.35-4.30 (m, 1H), 4.16-4.11 (m, 1H), 3.71-3.66 (m, 2H), 3.62-3.59 (m, 2H), 3.54-3.51 (m, 2H), 3.36 (s, 3H).

### No. I.1-441: Methyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate

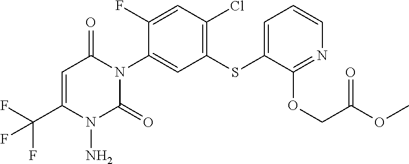 [SMILES: COC(=O)COc1ncccc1Sc1cc(-n2c(=O)cc(C(F)(F)F)n(N)c2=O)c(F)cc1Cl]

In a microwave vessel and under argon, n-hexane (6 ml) was added to 1-amino-3-{4-chloro-2-fluoro-5-[(2-hydroxypyridin-3-yl)sulfanyl]phenyl}-6-(trifluoromethyl)pyrimidine-2,4(1H,3H)-dione (150 mg, 0.33 mmol). Silver(I) carbonate (111 mg, 0.40 mmol) and methyl 2-iodoacetate (0.08 ml, 0.67 mmol) were then added. The reaction mixture was stirred at a temperature of 140° C. under microwave conditions for 30 minutes. After cooling to room temperature, the reaction mixture was filtered and the filtrate was concentrated under reduced pressure. Purification of the resulting crude product by column chromatography gave methyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate (34 mg, 19% of theory) in the form of a colorless solid. ¹H-NMR (CDCl₃ δ, ppm) 8.12 (d, 1H), 7.73 (m, 1H), 7.35 (d, 1H), 7.25 (d, 1H), 6.95 (m, 1H), 6.20 (s, 1H), 4.97-4.84 (q, 2H), 4.52 (s, 2H), 3.70 (s, 3H).

### No. I.1-442: Ethyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate

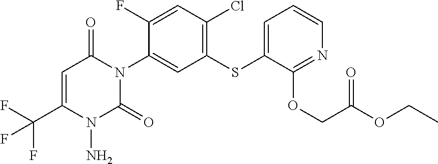 [SMILES: CCOC(=O)COc1ncccc1Sc1cc(-n2c(=O)cc(C(F)(F)F)n(N)c2=O)c(F)cc1Cl]

In a microwave vessel and under argon, n-hexane (17 ml) was added to 1-amino-3-{4-chloro-2-fluoro-5-[(2-hydroxypyridin-3-yl)sulfanyl]phenyl}-6-(trifluoromethyl)pyrimidine-2,4(1H,3H)-dione (300 mg, 0.67 mmol). Silver(I) carbonate (223 mg, 0.80 mmol) and ethyl 2-iodoacetate (0.16 ml, 1.34 mmol) were then added. The reaction mixture was stirred at a temperature of 140° C. under microwave conditions for 30 minutes. After cooling to room temperature, the reaction mixture was filtered and the filtrate was concentrated under reduced pressure. Purification of the resulting crude product by column chromatography gave ethyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate (81 mg, 32% of theory) in the form of a colorless solid. ¹H-NMR (CDCl₃ δ, ppm) 8.11 (d, 1H), 7.72 (m, 1H), 7.35 (d, 1H), 7.28 (d, 1H), 6.94 (m, 1H), 6.19 (s, 1H), 4.95-4.83 (q, 2H), 4.52 (s, 2H), 4.16-4.10 (m, 2H), 1.26 (t, 3H).

### No. I.1-447: n-Butyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate

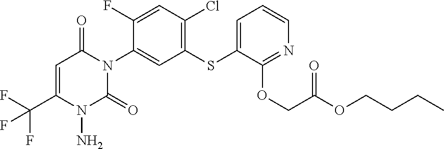 [SMILES: CCCCOC(=O)COc1ncccc1Sc1cc(-n2c(=O)cc(C(F)(F)F)n(N)c2=O)c(F)cc1Cl]

In a microwave vessel and under argon, n-hexane (17 ml) was added to 1-amino-3-{4-chloro-2-fluoro-5-[(2-hydroxypyridin-3-yl)sulfanyl]phenyl}-6-(trifluoromethyl)pyrimidine-2,4(1H,3H)-dione (200 mg, 0.45 mmol). Silver(I) carbonate (149 mg, 0.54 mmol) and n-pentyl 2-iodoacetate (270 mg, 0.89 mmol) were then added. The reaction mixture was stirred at a temperature of 140° C. under microwave conditions for 40 minutes. After cooling to room temperature, the reaction mixture was filtered and the filtrate was concentrated under reduced pressure. Purification of the resulting crude product by column chromatography gave n-butyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate (16 mg, 6% of theory) in the form of a colorless solid. ¹H-NMR (CDCl₃ δ, ppm) 8.11 (d, 1H), 7.72 (m, 1H), 7.33 (d, 1H), 7.28 (d, 1H), 6.94 (m, 1H), 6.19 (s, 1H), 4.95-4.83 (q, 2H), 4.58 (s, 2H), 4.14-4.05 (m, 2H), 1.65-1.55 (m, 4H), 1.41-1.30 (m, 2H), 0.92 (t, 3H).

### No. I.1-448: n-Pentyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate

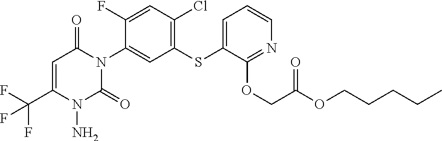 [SMILES: CCCCCOC(=O)COc1ncccc1Sc1cc(-n2c(=O)cc(C(F)(F)F)n(N)c2=O)c(F)cc1Cl]

In a microwave vessel and under argon, n-hexane (17 ml) was added to 1-amino-3-{4-chloro-2-fluoro-5-[(2-hydroxypyridin-3-yl)sulfanyl]phenyl}-6-(trifluoromethyl)pyrimidine-2,4(1H,3H)-dione (180 mg, 0.40 mmol). Silver(I) carbonate (134 mg, 0.48 mmol) and n-pentyl 2-iodoacetate (257 mg, 0.80 mmol) were then added. The reaction mixture was stirred at a temperature of 140° C. under microwave conditions for 40 minutes. After cooling to room temperature, the reaction mixture was filtered and the filtrate was concentrated under reduced pressure. Purification of the resulting crude product by column chromatography gave n-pentyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate (23 mg, 10% of theory) in the form of a colorless solid. ¹H-NMR (CDCl₃ δ, ppm) 8.11 (d, 1H), 7.47-7.43 (m, 2H), 7.13 (d, 1H), 6.98 (m, 1H), 6.25 (s, 1H), 4.68 (s, 2H), 4.58 (s, 2H), 4.16 (t, 2H), 1.68-1.63 (m, 2H), 1.39-1.30 (m, 4H), 0.91 (t, 3H).

### No. I.1-457: Benzyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate

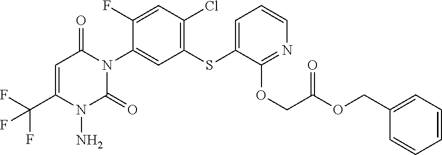 [SMILES: Nn1c(C(F)(F)F)cc(=O)n(-c2cc(Sc3cccnc3OCC(=O)OCc3ccccc3)c(Cl)cc2F)c1=O]

In a microwave vessel and under argon, n-hexane (17 ml) was added to 1-amino-3-{4-chloro-2-fluoro-5-[(2-hydroxypyridin-3-yl)sulfanyl]phenyl}-6-(trifluoromethyl)pyrimidine-2,4(1H,3H)-dione (180 mg, 0.40 mmol). Silver(I) carbonate (134 mg, 0.48 mmol) and benzyl 2-iodoacetate (226 mg, 0.80 mmol) were then added. The reaction mixture was stirred at a temperature of 140° C. under microwave conditions for 40 minutes. After cooling to room temperature, the reaction mixture was filtered and the filtrate was concentrated under reduced pressure. Purification of the resulting crude product by column chromatography gave benzyl {[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}acetate (21 mg, 9% of theory) in the form of a colorless solid. ¹H-NMR (CDCl₃ δ, ppm) 8.07 (d, 1H), 7.72 (m, 1H), 7.38-7.22 (m, 7H), 6.94 (m, 1H), 6.16 (s, 1H), 5.13 (s, 2H), 5.01-4.90 (q, 2H), 4.45 (s, 2H).

### No. I.2-442: Ethyl 2-{[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}propanoate

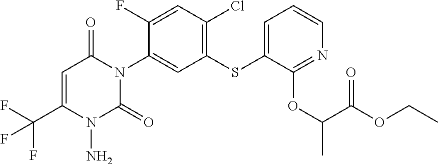 [SMILES: CCOC(=O)C(C)Oc1ncccc1Sc1cc(-n2c(=O)cc(C(F)(F)F)n(N)c2=O)c(F)cc1Cl]

In a microwave vessel and under argon, n-hexane (6 ml) was added to 1-amino-3-{4-chloro-2-fluoro-5-[(2-hydroxypyridin-3-yl)sulfanyl]phenyl}-6-(trifluoromethyl)pyrimidine-2,4(1H,3H)-dione (160 mg, 0.36 mmol). Silver(I) carbonate (118 mg, 0.43 mmol) and ethyl 2-iodopropionate (166 mg, 0.71 mmol) were then added. The reaction mixture was stirred at a temperature of 140° C. under microwave conditions for 2 h. After cooling to room temperature, the reaction mixture was filtered and the filtrate was concentrated under reduced pressure. Purification of the resulting crude product by column chromatography gave ethyl 2-{[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}propanoate (96 mg, 47% of theory) in the form of a colorless solid. ¹H-NMR (CDCl₃ δ, ppm) 8.09 (m, 1H), 7.72 (m, 1H), 7.33 (d, 1H), 7.19 (d, 1H), 6.91 (m, 1H), 6.19 (s, 1H), 5.22 (m, 1H), 4.52 (s, 2H), 4.18-4.09 (m, 2H), 1.49 (d, 3H), 1.23 (t, 3H).

### No. I.5-442: Ethyl 3-{[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}propanoate

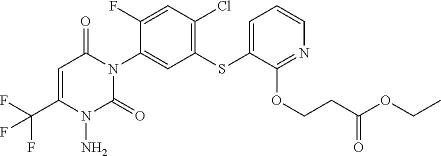 [SMILES: CCOC(=O)CCOc1ncccc1Sc1cc(-n2c(=O)cc(C(F)(F)F)n(N)c2=O)c(F)cc1Cl]

In a microwave vessel and under argon, n-hexane (7 ml) was added to 1-amino-3-{4-chloro-2-fluoro-5-[(2-hydroxypyridin-3-yl)sulfanyl]phenyl}-6-(trifluoromethyl)pyrimidine-2,4(1H,3H)-dione (150 mg, 0.33 mmol). Silver(I) carbonate (111 mg, 0.40 mmol) and ethyl 3-iodopropionate (161 mg, 0.67 mmol) were then added. The reaction mixture was stirred at a temperature of 140° C. under microwave conditions for 30 minutes. After cooling to room temperature, the reaction mixture was filtered and the filtrate was concentrated under reduced pressure. Purification of the resulting crude product by column chromatography gave ethyl 3-{[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}propanoate (65 mg, 34% of theory) in the form of a colorless solid. ¹H-NMR (CDCl₃ δ, ppm) 8.10 (d, 1H), 7.55 (m, 1H), 7.38 (d, 1H), 7.12 (d, 1H), 6.88 (m, 1H), 6.23 (s, 1H), 4.65-4.57 (m, 4H), 4.15 (q, 2H), 2.70 (t, 2H), 1.25 (t, 3H).

[symbol]

### No. I.5-457: Benzyl 3-{[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}propanoate

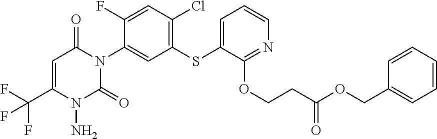 [SMILES: Nn1c(C(F)(F)F)cc(=O)n(-c2cc(Sc3cccnc3OCCC(=O)OCc3ccccc3)c(Cl)cc2F)c1=O]

In a microwave vessel and under argon, n-hexane (5 ml) was added to 1-amino-3-{4-chloro-2-fluoro-5-[(2-hydroxypyridin-3-yl)sulfanyl]phenyl}-6-(trifluoromethyl)pyrimidine-2,4(1H,3H)-dione (140 mg, 0.31 mmol). Silver(I) carbonate (103 mg, 0.37 mmol) and benzyl 3-iodopropionate (201 mg, 0.62 mmol) were then added. The reaction mixture was stirred at a temperature of 140° C. under microwave conditions for 30 minutes. After cooling to room temperature, the reaction mixture was filtered and the filtrate was concentrated under reduced pressure. Purification of the resulting crude product by column chromatography gave benyl 3-{[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}propanoate (70 mg, 35% of theory) in the form of a colorless solid. ¹H-NMR (CDCl₃ δ, ppm) 8.09 (m, 1H), 7.57 (m, 1H), 7.37-7.30 (m, 6H), 7.09 (d, 1H), 6.88 (m, 1H), 6.21 (s, 1H), 5.22 (s, 2H), 4.68-4.62 (m, 2H), 4.50 (s, 2H), 2.77 (t, 2H).

### No. I.6-441: Methyl 4-{[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}butanoate

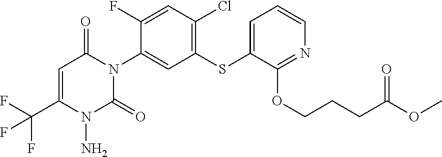 [SMILES: COC(=O)CCCOc1ncccc1Sc1cc(-n2c(=O)cc(C(F)(F)F)n(N)c2=O)c(F)cc1Cl]

In a microwave vessel and under argon, n-hexane (7 ml) was added to 1-amino-3-{4-chloro-2-fluoro-5-[(2-hydroxypyridin-3-yl)sulfanyl]phenyl}-6-(trifluoromethyl)pyrimidine-2,4(1H,3H)-dione (150 mg, 0.33 mmol). Silver(I) carbonate (111 mg, 0.40 mmol) and ethyl 3-iodopropionate (161 mg, 0.67 mmol) were then added. The reaction mixture was stirred at a temperature of 140° C. under microwave conditions for 30 minutes. After cooling to room temperature, the reaction mixture was filtered and the filtrate was concentrated under reduced pressure. Purification of the resulting crude product by column chromatography gave methyl 4-{[3-({5-[3-amino-2,6-dioxo-4-(trifluoromethyl)-3,6-dihydropyrimidin-1(2H)-yl]-2-chloro-4-fluorophenyl}sulfanyl)pyridin-2-yl]oxy}butanoate (104 mg, 54% of theory) in the form of a colorless solid. ¹H-NMR (CDCl₃ δ, ppm) 8.11 (d, 1H), 7.60 (m, 1H), 7.38 (d, 1H), 7.10 (d, 1H), 6.87 (m, 1H), 6.22 (s, 1H), 4.62 (s, 2H), 4.37-4.31 (m, 2H), 3.66 (s, 3H), 2.41-2.36 (m, 2H), 2.08-2.00 (m, 2H).

### No. I.14-442: Ethyl [(3-{5-[3-amino-2,6-dioxo-4-(trifluoromethyl)-3,6-dihydropyrimidin-1(2H)-yl]-4-fluoro-2-nitrophenoxy}pyridin-2-yl)oxy]acetate

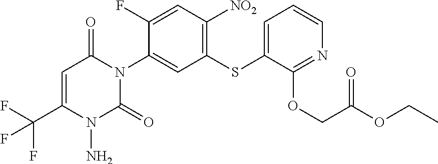 [SMILES: CCOC(=O)COc1ncccc1Sc1cc(-n2c(=O)cc(C(F)(F)F)n(N)c2=O)c(F)cc1[N+](=O)[O-]]

Successively, 2,5-difluoroaniline (1 equiv.) and triethylamine (2 equiv.) were added carefully to a solution of triphosgene (0.4 equiv.) in abs. dichloromethane such that the temperature of the resulting reaction mixture remained below 20° C. After the addition had ended, the reaction mixture was stirred at room temperature overnight and then washed with water and 1N hydrochloric acid, dried over sodium sulfate, filtered and concentrated under reduced pressure. The resulting 2,5-difluorophenyl isocyanate was used in the next stage without further purification. Sodium hydride (2 equiv., 60% dispersion in mineral oil) was suspended in abs. N,N-dimethylformamide, and ethyl (2E)-3-amino-4,4,4-trifluorobut-2-enoate (1.1 equiv.) was added. The reaction mixture was stirred at room temperature for 1 h and then cooled to a temperature of −30° C., and 2,5-difluorophenyl isocyanate (1 equiv) was added. After the addition had ended, the resulting reaction mixture was stirred at room temperature for a further 4 h and then added to ice-water. After addition of ethyl acetate and acidification with 1N hydrochloric acid, the aqueous phase was extracted thoroughly with ethyl acetate. The combined organic phases were washed with water, dried over sodium sulfate, filtered and concentrated under reduced pressure. This gave 3-(2,5-difluorophenyl)-6-(trifluoromethyl)pyrimidine-2,4(1H,3H)-dione, which was used in the next stage without further purification. Sulfuric acid was added to 3-(2,5-difluorophenyl)-6-(trifluoromethyl)pyrimidine-2,4(1H,3H)-dione (1 equiv.), and the resulting reaction mixture was cooled to 0° C. Potassium nitrate (1.1 equiv.) was then added carefully, a little at a time. The resulting reaction mixture was stirred at a temperature of 100° C. for 10 h and, after cooling to room temperature, added to ice-water. Ethyl acetate was then added, and the aqueous phase was repeatedly extracted thoroughly with ethyl acetate. The combined organic phases were dried over sodium sulfate, filtered and carefully concentrated under reduced pressure, which gave 3-(2,5-difluoro-4-nitrophenyl)-6-(trifluoromethyl)pyrimidine-2,4(1H,3H)-dione in the form of a colorless solid. 3-(2,5-Difluoro-4-nitrophenyl)-6-(trifluoromethyl)pyrimidine-2,4(1H,3H)-dione (1 equiv.) was dissolved in abs. N,N-dimethylformamide, and a solution of sodium hydride (1.2 equiv., 60% strength dispersion in mineral oil) in N,N-dimethylformamide was added. 2,4-Dinitrophenylhydroxylamine (1.5 equiv.) was then added carefully, a little at a time. The resulting reaction mixture was subsequently stirred under nitrogen for 12 h. After complete conversion, water and ethyl acetate were added and the mixture was repeatedly thoroughly extracted. The combined organic phases were dried over sodium sulfate, filtered and carefully concentrated under reduced pressure. Final purification by column chromatography gave 1-amino-3-(2,5-difluoro-4-nitrophenyl)-6-(trifluoromethyl)pyrimidine-2,4(1H,3H)-dione in the form of a colorless solid. Under argon, N,N-dimethylformamide (5 ml) was added to 1-amino-3-(2,5-difluoro-4-nitrophenyl)-6-(trifluoromethyl)pyrimidine-2,4(1H,3H)-dione (150 mg, 0.43 mmol) in a round-bottom flask which had been dried by heating. Ethyl [(3-hydroxypyridin-2-yl)oxy]acetate (84 mg, 0.43 mmol) and potassium carbonate (65 mg, 0.47 mmol) were then added. The reaction mixture was stirred at a temperature of 65° C. for 3 h. After cooling to room temperature, water and dichloromethane were added to the reaction mixture and the mixture was extracted thoroughly. The combined organic phases were dried over sodium sulfate, filtered and carefully concentrated under reduced pressure. Final purification by column chromatography gave ethyl [(3-{5-[3-amino-2,6-dioxo-4-(trifluoromethyl)-3,6-dihydropyrimidin-1(2H)-yl]-4-fluoro-2-nitrophenoxy}pyridin-2-yl)oxy]acetate (81 mg, 36% of theory) in the form of a colorless solid. ¹H-NMR (CDCl₃ δ, ppm) 8.01 (m, 1H), 7.89 (d, 1H), 7.72 (m, 1H), 7.53 (m, 1H), 7.18 (d, 1H), 7.00 (m, 1H), 6.19 (s, 1H), 4.99 (d, 1H), 4.85 (d, 2H), 4.51 (s, 2H), 4.15-4.10 (m, 2H), 1.26 (t, 3H).

### No. I.31-23: 2-(2-Methoxyethoxy)ethyl {[3-({5-[3-amino-2,6-dioxo-4-(trifluoromethyl)-3,6-dihydropyrimidin-1(2H)-yl]-2-chloro-4-fluorophenyl}sulfanyl)-5-fluoropyridin-2-yl]oxy}acetate

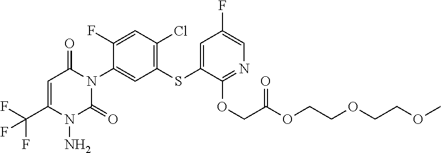 [SMILES: COCCOCCOC(=O)COc1ncc(F)cc1Sc1cc(-n2c(=O)cc(C(F)(F)F)n(N)c2=O)c(F)cc1Cl]

Under argon, 1-amino-3-(4-chloro-2-fluoro-5-sulfanylphenyl)-6-(trifluoromethyl)pyrimidine-2,4(1H,3H)-dione (800 mg, 2.25 mmol) was dissolved in dioxane (18 ml) in a microwave vessel and, after degassing of the solvent, tris(dibenzylideneacetone)dipalladium (51 mg, 0.06 mmol), 4,5-bis(diphenylphosphino)-9,9-dimethylxanthene (65 mg, 0.11 mmol), N,N-diisopropylethylamine (0.78 ml, 4.49 mmol) and 3-bromo-5-fluoro-2-hydroxypyridine (475 mg, 2.47 mmol) were added. The resulting reaction mixture was stirred under microwave conditions at a temperature of 160° C. for 2 h. After cooling to room temperature, the reaction mixture was filtered and the filtrate was concentrated. Purification of the resulting crude product by column chromatography gave 1-amino-3-{4-chloro-2-fluoro-5-[(5-fluoro-2-hydroxypyridin-3-yl)sulfanyl]phenyl}-6-(trifluoromethyl)pyrimidine-2,4(1H,3H)-dione (660 mg, 63% of theory) in the form of a colorless solid. ¹H-NMR (d₆-DMSO δ, ppm) 12.02 (br. s, 1H), 7.98 (d, 1H), 7.70 (d, 1H), 7.59 (m, 1H), 6.97 (m, 1H), 6.41 (s, 1H), 5.59 (s, 2H). In a microwave vessel and under argon, n-hexane (15 ml) was added to 1-amino-3-{4-chloro-2-fluoro-5-[(5-fluoro-2-hydroxypyridin-3-yl)sulfanyl]phenyl}-6-(trifluoromethyl)pyrimidine-2,4(1H,3H)-dione (220 mg, 0.47 mmol). Silver(I) carbonate (156 mg, 0.57 mmol) and 2-(2-methoxyethoxy)ethyl iodoacetate (272 mg, 0.94 mmol) were then added. The reaction mixture was stirred at a temperature of 140° C. under microwave conditions for 48 minutes. After cooling to room temperature, the reaction mixture was filtered and the filtrate was concentrated under reduced pressure. Purification of the resulting crude product by column chromatography gave 2-(2-methoxyethoxy)ethyl {[3-({5-[3-amino-2,6-dioxo-4-(trifluoromethyl)-3,6-dihydropyrimidin-1(2H)-yl]-2-chloro-4-fluorophenyl}sulfanyl)-5-fluoropyridin-2-yl]oxy}acetate (33 mg, 10% of theory) in the form of a colorless solid. ¹H-NMR (CDCl₃ δ, ppm) 7.91 (m, 1H), 7.43 (m, 1H), 7.38 (m, 2H), 6.22 (s, 1H), 4.95 (d, 1H), 4.91 (d, 1H), 4.65 (s, 2H), 4.35-4.30 (m, 1H), 4.20-4.17 (m, 1H), 3.72-3.66 (m, 2H), 3.62-3.59 (m, 2H), 3.58-3.53 (m, 5H), 3.37 (s, 3H).

In analogy to the preparation examples cited above and recited at the appropriate point, and taking account of the general details relating to the preparation of substituted N-heterocyclyl- and N-heteroaryltetrahydropyrimidinones, the compounds cited below are obtained. If in Table 1 a structural element is defined by a structural formula containing a broken line, this broken line means that at this position the group in question is attached to the remainder of the molecule. If in Table 1 a structural element is defined by a structural formula containing an arrow, the arrow represents a bond of the respective group Q to the carbonyl group in the general formula (I).

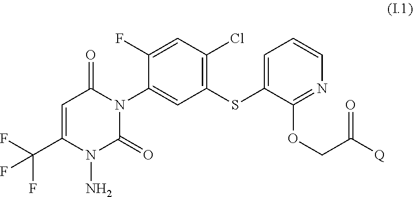

Table I.1: Preferred compounds of the formula (I.1) are the compounds I.1-1 to I.1-486 in which Q has the meanings of Table 1 indicated in the respective row. Thus, the compounds I.1-1 to I.1-486 of Table I.1 are defined by the meaning of the respective entries Nos. 1 to 486 for Q of Table 1.

TABLE 1

| No. | Q |
|---|---|
| 1 | Q-1 |
| 2 | Q-2 |
| 3 | Q-3 |
| 4 | Q-4 |
| 5 | Q-5 |
| 6 | Q-6 |
| 7 | Q-7 |
| 8 | Q-8 |
| 9 | Q-9 |
| 10 | Q-10 |
| 11 | Q-11 |
| 12 | Q-12 |
| 13 | Q-13 |
| 14 | Q-14 |
| 15 | Q-15 |
| 16 | Q-16 |
| 17 | Q-17 |
| 18 | Q-18 |
| 19 | Q-19 |
| 20 | Q-20 |
| 21 | Q-21 |
| 22 | Q-22 |
| 23 | Q-23 |
| 24 | Q-24 |
| 25 | Q-25 |
| 26 | Q-26 |
| 27 | Q-27 |
| 28 | Q-28 |
| 29 | Q-29 |
| 30 | Q-30 |
| 31 | Q-31 |
| 32 | Q-32 |
| 33 | Q-33 |
| 34 | Q-34 |
| 35 | Q-35 |
| 36 | Q-36 |
| 37 | Q-37 |
| 38 | Q-38 |
| 39 | Q-39 |
| 40 | Q-40 |
| 41 | Q-41 |
| 42 | Q-42 |
| 43 | Q-43 |
| 44 | Q-44 |
| 45 | Q-45 |
| 46 | Q-46 |
| 47 | Q-47 |
| 48 | Q-48 |
| 49 | Q-49 |
| 50 | Q-50 |
| 51 | Q-51 |
| 52 | Q-52 |
| 53 | Q-53 |
| 54 | Q-54 |
| 55 | Q-55 |
| 56 | Q-56 |
| 57 | Q-57 |
| 58 | Q-58 |
| 59 | Q-59 |
| 60 | Q-60 |
| 61 | Q-61 |
| 62 | Q-62 |
| 63 | Q-63 |
| 64 | Q-64 |
| 65 | Q-65 |
| 66 | Q-66 |
| 67 | Q-67 |
| 68 | Q-68 |
| 69 | Q-69 |
| 70 | Q-70 |
| 71 | Q-71 |
| 72 | Q-72 |
| 73 | Q-73 |
| 74 | Q-74 |
| 75 | Q-75 |
| 76 | Q-76 |
| 77 | Q-77 |
| 78 | Q-78 |
| 79 | Q-79 |
| 80 | Q-80 |
| 81 | Q-81 |
| 82 | Q-82 |
| 83 | Q-83 |
| 84 | Q-84 |
| 85 | Q-85 |
| 86 | Q-86 |
| 87 | Q-87 |
| 88 | Q-88 |
| 89 | Q-89 |
| 90 | Q-90 |
| 91 | Q-91 |
| 92 | Q-92 |
| 93 | Q-93 |
| 94 | Q-94 |
| 95 | Q-95 |
| 96 | Q-96 |
| 97 | Q-97 |
| 98 | Q-98 |
| 99 | Q-99 |
| 100 | Q-100 |
| 101 | Q-101 |
| 102 | Q-102 |
| 103 | Q-103 |
| 104 | Q-104 |
| 105 | Q-105 |
| 106 | Q-106 |
| 107 | Q-107 |
| 108 | Q-108 |
| 109 | Q-109 |
| 110 | Q-110 |
| 111 | Q-111 |
| 112 | Q-112 |
| 113 | Q-113 |
| 114 | Q-114 |
| 115 | Q-115 |
| 116 | Q-116 |
| 117 | Q-117 |
| 118 | Q-118 |
| 119 | Q-119 |
| 120 | Q-120 |
| 121 | Q-121 |
| 122 | Q-122 |
| 123 | Q-123 |
| 124 | Q-124 |
| 125 | Q-125 |
| 126 | Q-126 |
| 127 | Q-127 |
| 128 | Q-128 |
| 129 | Q-129 |
| 130 | Q-130 |
| 131 | Q-131 |
| 132 | Q-132 |
| 133 | Q-133 |
| 134 | Q-134 |
| 135 | Q-135 |
| 136 | Q-136 |
| 137 | Q-137 |
| 138 | Q-138 |
| 139 | Q-139 |
| 140 | Q-140 |
| 141 | Q-141 |
| 142 | Q-142 |
| 143 | Q-143 |
| 144 | Q-144 |
| 145 | Q-145 |
| 146 | Q-146 |
| 147 | Q-147 |
| 148 | Q-148 |
| 149 | Q-149 |
| 150 | Q-150 |
| 151 | Q-151 |
| 152 | Q-152 |
| 153 | Q-153 |
| 154 | Q-154 |
| 155 | Q-155 |
| 156 | Q-156 |
| 157 | Q-157 |
| 158 | Q-158 |
| 159 | Q-159 |
| 160 | Q-160 |
| 161 | Q-161 |
| 162 | Q-162 |
| 163 | Q-163 |
| 164 | Q-164 |
| 165 | Q-165 |
| 166 | Q-166 |
| 167 | Q-167 |
| 168 | Q-168 |
| 169 | Q-169 |
| 170 | Q-170 |
| 171 | Q-171 |
| 172 | Q-172 |
| 173 | Q-173 |
| 174 | Q-174 |
| 175 | Q-175 |
| 176 | Q-176 |
| 177 | Q-177 |
| 178 | Q-178 |
| 179 | Q-179 |
| 180 | Q-180 |
| 181 | Q-181 |
| 182 | Q-182 |
| 183 | Q-183 |
| 184 | Q-184 |
| 185 | Q-185 |
| 186 | Q-186 |
| 187 | Q-187 |
| 188 | Q-188 |
| 189 | Q-189 |
| 190 | Q-190 |
| 191 | Q-191 |
| 192 | Q-192 |
| 193 | Q-193 |
| 194 | Q-194 |
| 195 | Q-195 |
| 196 | Q-196 |
| 197 | Q-197 |
| 198 | Q-198 |
| 199 | Q-199 |
| 200 | Q-200 |
| 201 | Q-201 |
| 202 | Q-202 |
| 203 | Q-203 |
| 204 | Q-204 |
| 205 | Q-205 |
| 206 | Q-206 |
| 207 | Q-207 |
| 208 | Q-208 |
| 209 | Q-209 |
| 210 | Q-210 |
| 211 | Q-211 |
| 212 | Q-212 |
| 213 | Q-213 |
| 214 | Q-214 |
| 215 | Q-215 |
| 216 | Q-216 |
| 217 | Q-217 |
| 218 | Q-218 |
| 219 | Q-219 |
| 220 | Q-220 |
| 221 | Q-221 |
| 222 | Q-222 |
| 223 | Q-223 |
| 224 | Q-224 |
| 225 | Q-225 |
| 226 | Q-226 |
| 227 | Q-227 |
| 228 | Q-228 |
| 229 | Q-229 |
| 230 | Q-230 |
| 231 | Q-231 |
| 232 | Q-232 |
| 233 | Q-233 |
| 234 | Q-234 |
| 235 | Q-235 |
| 236 | Q-236 |
| 237 | Q-237 |
| 238 | Q-238 |
| 239 | Q-239 |
| 240 | Q-240 |
| 241 | Q-241 |
| 242 | Q-242 |
| 243 | Q-243 |
| 244 | Q-244 |
| 245 | Q-245 |
| 246 | Q-246 |
| 247 | Q-247 |
| 248 | Q-248 |
| 249 | Q-249 |
| 250 | Q-250 |
| 251 | Q-251 |
| 252 | Q-252 |
| 253 | Q-253 |
| 254 | Q-254 |
| 255 | Q-255 |
| 256 | Q-256 |
| 257 | Q-257 |
| 258 | Q-258 |
| 259 | Q-259 |
| 260 | Q-260 |
| 261 | Q-261 |
| 262 | Q-262 |
| 263 | Q-263 |
| 264 | Q-264 |
| 265 | Q-265 |
| 266 | Q-266 |
| 267 | Q-267 |
| 268 | Q-268 |
| 269 | Q-269 |
| 270 | Q-270 |
| 271 | Q-271 |
| 272 | Q-272 |
| 273 | Q-273 |
| 274 | Q-274 |
| 275 | Q-275 |
| 276 | Q-276 |
| 277 | Q-277 |
| 278 | Q-278 |
| 279 | Q-279 |
| 280 | Q-280 |
| 281 | Q-281 |
| 282 | Q-282 |
| 283 | Q-283 |
| 284 | Q-284 |
| 285 | Q-285 |
| 286 | Q-286 |
| 287 | Q-287 |
| 288 | Q-288 |
| 289 | Q-289 |
| 290 | Q-290 |
| 291 | Q-291 |
| 292 | Q-292 |
| 293 | Q-293 |
| 294 | Q-294 |
| 295 | Q-295 |
| 296 | Q-296 |
| 297 | Q-297 |
| 298 | Q-298 |
| 299 | Q-299 |
| 300 | Q-300 |
| 301 | Q-301 |
| 302 | Q-302 |
| 303 | Q-303 |
| 304 | Q-304 |
| 305 | Q-305 |
| 306 | Q-306 |
| 307 | Q-307 |
| 308 | Q-308 |
| 309 | Q-309 |
| 310 | Q-310 |
| 311 | Q-311 |
| 312 | Q-312 |
| 313 | Q-313 |
| 314 | Q-314 |
| 315 | Q-315 |
| 316 | Q-316 |
| 317 | Q-317 |
| 318 | Q-318 |
| 319 | Q-319 |
| 320 | Q-320 |
| 321 | Q-321 |
| 322 | Q-322 |
| 323 | Q-323 |
| 324 | Q-324 |
| 325 | Q-325 |
| 326 | Q-326 |
| 327 | Q-327 |
| 328 | Q-328 |
| 329 | Q-329 |
| 330 | Q-330 |
| 331 | Q-331 |
| 332 | Q-332 |
| 333 | Q-333 |
| 334 | Q-334 |
| 335 | Q-335 |
| 336 | Q-336 |
| 337 | Q-337 |
| 338 | Q-338 |
| 339 | Q-339 |
| 340 | Q-340 |
| 341 | Q-341 |
| 342 | Q-342 |
| 343 | Q-343 |
| 344 | Q-344 |
| 345 | Q-345 |
| 346 | Q-346 |
| 347 | Q-347 |
| 348 | Q-348 |
| 349 | Q-349 |
| 350 | Q-350 |
| 351 | Q-351 |
| 352 | Q-352 |
| 353 | Q-353 |
| 354 | Q-354 |
| 355 | Q-355 |
| 356 | Q-356 |
| 357 | Q-357 |
| 358 | Q-358 |
| 359 | Q-359 |
| 360 | Q-360 |
| 361 | Q-361 |
| 362 | Q-362 |
| 363 | Q-363 |
| 364 | Q-364 |
| 365 | Q-365 |
| 366 | Q-366 |
| 367 | Q-367 |
| 368 | Q-368 |
| 369 | Q-369 |
| 370 | Q-370 |
| 371 | Q-371 |
| 372 | Q-372 |
| 373 | Q-373 |
| 374 | Q-374 |
| 375 | Q-375 |
| 376 | Q-376 |
| 377 | Q-377 |
| 378 | Q-378 |
| 379 | Q-379 |
| 380 | Q-380 |
| 381 | Q-381 |
| 382 | Q-382 |
| 383 | Q-383 |
| 384 | Q-384 |
| 385 | Q-385 |
| 386 | Q-386 |
| 387 | Q-387 |
| 388 | Q-388 |
| 389 | Q-389 |
| 390 | Q-390 |
| 391 | Q-391 |
| 392 | Q-392 |
| 393 | Q-393 |
| 394 | Q-394 |
| 395 | Q-395 |
| 396 | Q-396 |
| 397 | Q-397 |
| 398 | Q-398 |
| 399 | Q-399 |
| 400 | Q-400 |
| 401 | Q-401 |
| 402 | Q-402 |
| 403 | Q-403 |
| 404 | Q-404 |
| 405 | Q-405 |
| 406 | Q-406 |
| 407 | Q-407 |
| 408 | Q-408 |
| 409 | Q-409 |
| 410 | Q-410 |
| 411 | Q-411 |
| 412 | Q-412 |
| 413 | Q-413 |
| 414 | Q-414 |
| 415 | Q-415 |
| 416 | Q-416 |
| 417 | Q-417 |
| 418 | Q-418 |
| 419 | Q-419 |
| 420 | Q-420 |
| 421 | Q-421 |
| 422 | Q-422 |
| 423 | Q-423 |
| 424 | Q-424 |
| 425 | Q-425 |
| 426 | Q-426 |
| 427 | Q-427 |
| 428 | Q-428 |
| 429 | Q-429 |
| 430 | Q-430 |
| 431 | Q-431 |
| 432 | Q-432 |
| 433 | Q-433 |
| 434 | Q-434 |
| 435 | Q-435 |
| 436 | Q-436 |
| 437 | Q-437 |
| 438 | Q-438 |
| 439 | Q-439 |
| 440 | Q-440 |
| 441 | Q-441 |
| 442 | Q-442 |
| 443 | Q-443 |
| 444 | Q-444 |
| 445 | Q-445 |
| 446 | Q-446 |
| 447 | Q-447 |
| 448 | Q-448 |
| 449 | Q-449 |
| 450 | Q-450 |
| 451 | Q-451 |
| 452 | Q-452 |
| 453 | Q-453 |
| 454 | Q-454 |
| 455 | Q-455 |
| 456 | Q-456 |
| 457 | Q-457 |
| 458 | Q-458 |
| 459 | Q-459 |
| 460 | Q-460 |
| 461 | Q-461 |
| 462 | Q-462 |
| 463 | Q-463 |
| 464 | Q-464 |
| 465 | Q-465 |
| 466 | Q-466 |
| 467 | Q-467 |
| 468 | Q-468 |
| 469 | Q-469 |
| 470 | Q-470 |
| 471 | Q-471 |
| 472 | Q-472 |
| 473 | Q-473 |
| 474 | Q-474 |
| 475 | Q-475 |
| 476 | Q-476 |
| 477 | Q-477 |
| 478 | Q-478 |
| 479 | Q-479 |
| 480 | Q-480 |
| 481 | Q-481 |
| 482 | Q-482 |
| 483 | Q-483 |
| 484 | Q-484 |
| 485 | Q-485 |
| 486 | Q-486 |

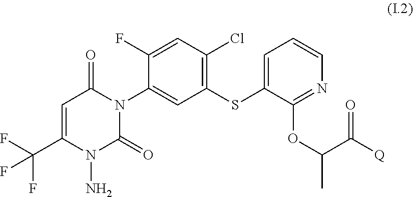

Table I.2: Preferred compounds of the formula (I.2) are the compounds I.2-1 to I.2-486 in which Q has the meanings of Table 1 indicated in the respective row. Thus, the compounds I.2-1 to I.2-486 of Table 1.2 are defined by the meaning of the respective entries Nos. 1 to 486 for Q of Table 1.

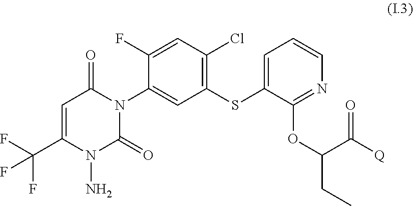

Table I.3: Preferred compounds of the formula (I.3) are the compounds I.3-1 to I.3-486 in which Q has the meanings of Table 1 indicated in the respective row. Thus, the compounds I.3-1 to I.3-486 of Table 1.3 are defined by the meaning of the respective entries Nos. 1 to 486 for Q of Table 1.

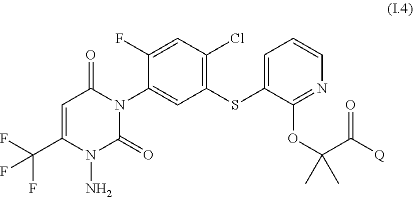

Table I.4: Preferred compounds of the formula (I.4) are the compounds I.4-1 to I.4-486 in which Q has the meanings of Table 1 indicated in the respective row. Thus, the compounds I.4-1 to I.4-486 of Table 1.4 are defined by the meaning of the respective entries Nos. 1 to 486 for Q of Table 1.

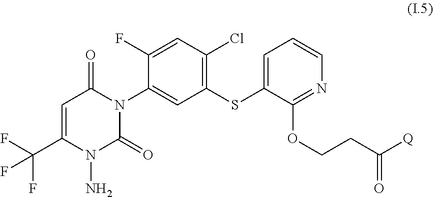

Table I.5: Preferred compounds of the formula (I.5) are the compounds I.5-1 to I.5-486 in which Q has the meanings of Table 1 indicated in the respective row. Thus, the compounds I.5-1 to I.5-486 of Table 1.5 are defined by the meaning of the respective entries Nos. 1 to 486 for Q of Table 1.

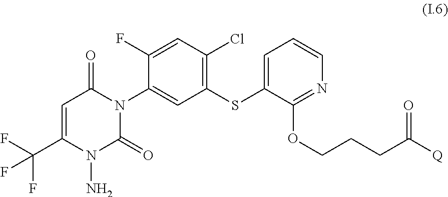

Table I.6: Preferred compounds of the formula (I.6) are the compounds I.6-1 to I.6-486 in which Q has the meanings of Table 1 indicated in the respective row. Thus, the compounds I.6-1 to I.6-486 of Table 1.6 are defined by the meaning of the respective entries Nos. 1 to 486 for Q of Table 1.

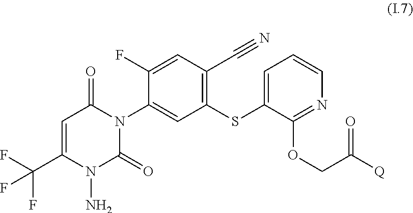

Table I.7: Preferred compounds of the formula (I.7) are the compounds I.7-1 to I.7-486 in which Q has the meanings of Table 1 indicated in the respective row. Thus, the compounds I.7-1 to I.7-486 of Table 7.1 are defined by the meaning of the respective entries Nos. 1 to 486 for Q of Table 1.

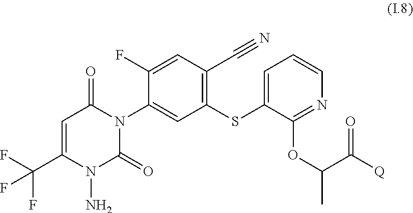

Table I.8: Preferred compounds of the formula (I.8) are the compounds I.8-1 to I.8-486 in which Q has the meanings of Table 1 indicated in the respective row. Thus, the compounds I.8-1 to I.8-486 of Table 8.1 are defined by the meaning of the respective entries Nos. 1 to 486 for Q of Table 1.

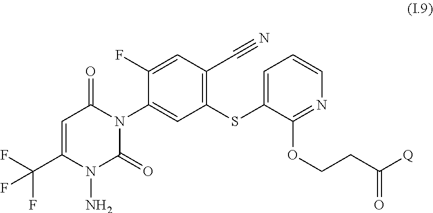

Table I.9: Preferred compounds of the formula (I.9) are the compounds I.9-1 to I.9-486 in which Q has the meanings of Table 1 indicated in the respective row. Thus, the compounds I.9-1 to I.9-486 of Table 1.9 are defined by the meaning of the respective entries Nos. 1 to 486 for Q of Table 1.

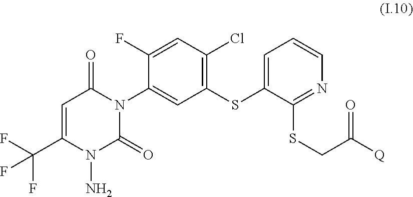

Table I.10: Preferred compounds of the formula (I.10) are the compounds I.10-1 to I.10-486 in which Q has the meanings of Table 1 indicated in the respective row. Thus, the compounds I.10-1 to I.10-486 of Table I.1 are defined by the meaning of the respective entries Nos. 1 to 486 for Q of Table 1.

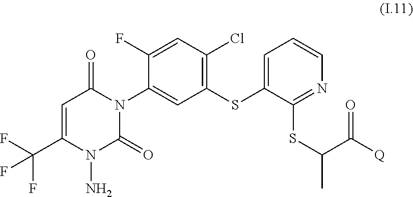

Table I.11: Preferred compounds of the formula (I.11) are the compounds I.11-1 to I.11-486 in which Q has the meanings of Table 1 indicated in the respective row. Thus, the compounds I.11-1 to I.11-486 of Table I.11 are defined by the meaning of the respective entries Nos. 1 to 486 for Q of Table 1.

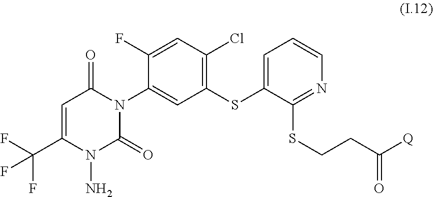

Table I.12: Preferred compounds of the formula (I.12) are the compounds I.12-1 to I.12-486 in which Q has the meanings of Table 1 indicated in the respective row. Thus, the compounds I.12-1 to I.12-486 of Table I.12 are defined by the meaning of the respective entries Nos. 1 to 486 for Q of Table 1.

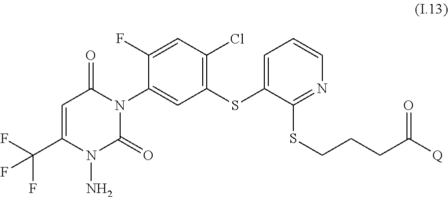

Table I.13: Preferred compounds of the formula (I.13) are the compounds I.13-1 to I.13-486 in which Q has the meanings of Table 1 indicated in the respective row. Thus, the compounds I.13-1 to I.13-486 of Table I.13 are defined by the meaning of the respective entries Nos. 1 to 486 for Q of Table 1.

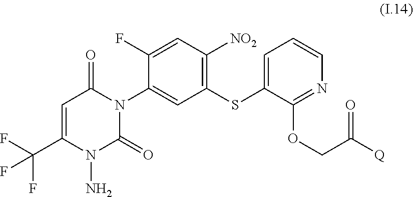

Table I.14: Preferred compounds of the formula (I.14) are the compounds I.14-1 to I.14-486 in which Q has the meanings of Table 1 indicated in the respective row. Thus, the compounds I.14-1 to I.14-486 of Table I.14 are defined by the meaning of the respective entries Nos. 1 to 486 for Q of Table 1.

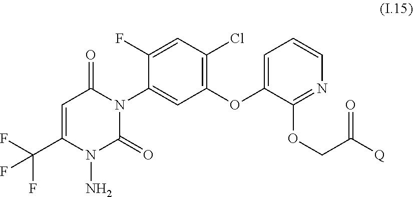

Table I.15: Preferred compounds of the formula (I.15) are the compounds I.15-1 to I.15-486 in which Q has the meanings of Table 1 indicated in the respective row. Thus, the compounds I.15-1 to I.15-486 of Table I.15 are defined by the meaning of the respective entries Nos. 1 to 486 for Q of Table 1.

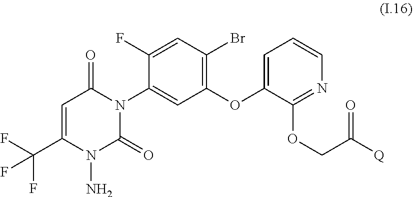

Table I.16: Preferred compounds of the formula (I.16) are the compounds I.16-1 to I.16-486 in which Q has the meanings of Table 1 indicated in the respective row. Thus, the compounds I.16-1 to I.16-486 of Table I.16 are defined by the meaning of the respective entries Nos. 1 to 486 for Q of Table 1.

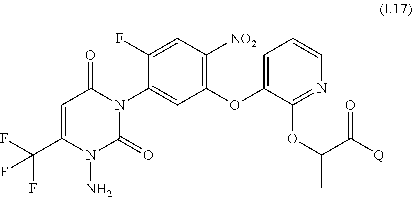

Table I.17: Preferred compounds of the formula (I.17) are the compounds I.17-1 to I.17-486 in which Q has the meanings of Table 1 indicated in the respective row. Thus, the compounds I.17-1 to I.17-486 of Table I.17 are defined by the meaning of the respective entries Nos. 1 to 486 for Q of Table 1.

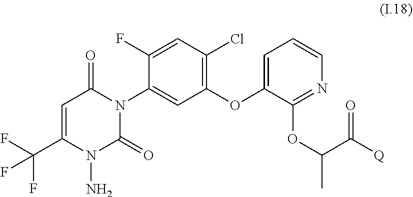

Table I.18: Preferred compounds of the formula (I.18) are the compounds I.18-1 to I.18-486 in which Q has the meanings of Table 1 indicated in the respective row. Thus, the compounds I.18-1 to I.18-486 of Table I.18 are defined by the meaning of the respective entries Nos. 1 to 486 for Q of Table 1.

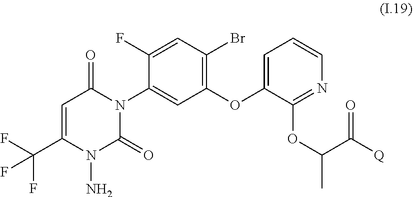

Table I.19: Preferred compounds of the formula (I.19) are the compounds I.19-1 to I.19-486 in which Q has the meanings of Table 1 indicated in the respective row. Thus, the compounds I.19-1 to I.19-486 of Table I.19 are defined by the meaning of the respective entries Nos. 1 to 486 for Q of Table 1.

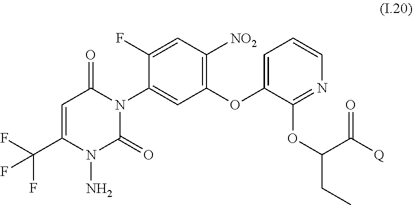

Table I.20: Preferred compounds of the formula (I.20) are the compounds I.20-1 to I.20-486 in which Q has the meanings of Table 1 indicated in the respective row. Thus, the compounds I.20-1 to I.20-486 of Table I.20 are defined by the meaning of the respective entries Nos. 1 to 486 for Q of Table 1.

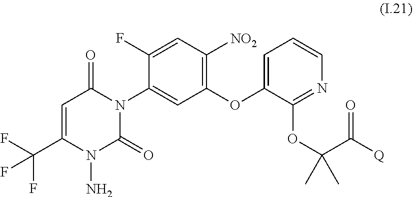

Table I.21: Preferred compounds of the formula (I.21) are the compounds I.21-1 to I.21-486 in which Q has the meanings of Table 1 indicated in the respective row. Thus, the compounds I.21-1 to I.21-486 of Table I.21 are defined by the meaning of the respective entries Nos. 1 to 486 for Q of Table 1.

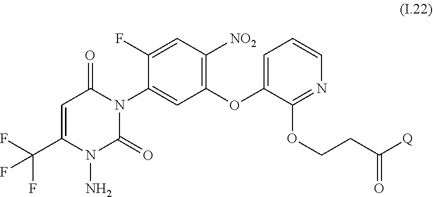

Table I.22: Preferred compounds of the formula (I.22) are the compounds I.22-1 to I.22-486 in which Q has the meanings of Table 1 indicated in the respective row. Thus, the compounds I.22-1 to I.22-486 of Table I.22 are defined by the meaning of the respective entries Nos. 1 to 486 for Q of Table 1.

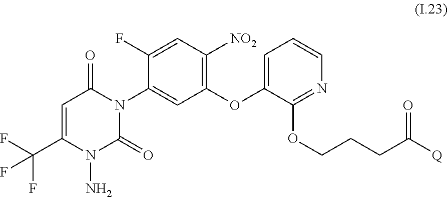

Table I.23: Preferred compounds of the formula (I.23) are the compounds I.23-1 to I.23-486 in which Q has the meanings of Table 1 indicated in the respective row. Thus, the compounds I.23-1 to I.23-486 of Table I.23 are defined by the meaning of the respective entries Nos. 1 to 486 for Q of Table 1.

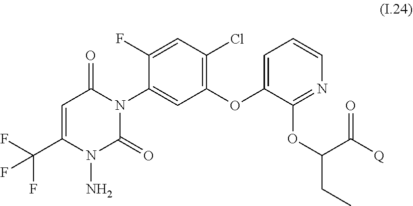

Table I.24: Preferred compounds of the formula (I.24) are the compounds I.24-1 to I.24-486 in which Q has the meanings of Table 1 indicated in the respective row. Thus, the compounds I.24-1 to I.24-486 of Table I.24 are defined by the meaning of the respective entries Nos. 1 to 486 for Q of Table 1.

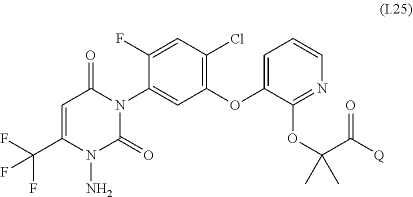

Table I.25: Preferred compounds of the formula (I.25) are the compounds I.25-1 to I.25-486 in which Q has the meanings of Table 1 indicated in the respective row. Thus, the compounds I.25-1 to I.25-486 of Table I.25 are defined by the meaning of the respective entries Nos. 1 to 486 for Q of Table 1.

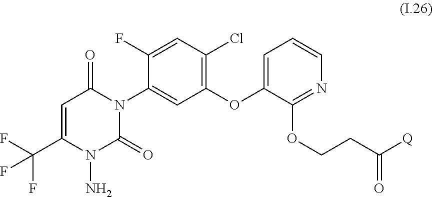

Table I.26: Preferred compounds of the formula (I.26) are the compounds I.26-1 to I.26-486 in which Q has the meanings of Table 1 indicated in the respective row. Thus, the compounds I.26-1 to I.26-486 of Table I.26 are defined by the meaning of the respective entries Nos. 1 to 486 for Q of Table 1.

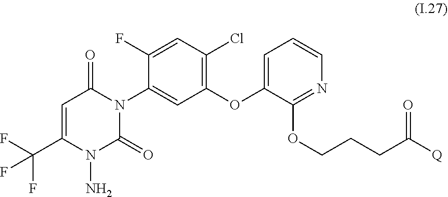

Table I.27: Preferred compounds of the formula (I.27) are the compounds I.27-1 to I.27-486 in which Q has the meanings of Table 1 indicated in the respective row. Thus, the compounds I.27-1 to I.27-486 of Table I.27 are defined by the meaning of the respective entries Nos. 1 to 486 for Q of Table 1.

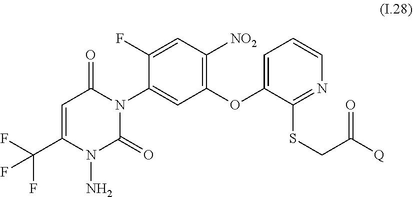

Table I.28: Preferred compounds of the formula (I.28) are the compounds I.28-1 to I.28-486 in which Q has the meanings of Table 1 indicated in the respective row. Thus, the compounds I.28-1 to I.28-486 of Table I.28 are defined by the meaning of the respective entries Nos. 1 to 486 for Q of Table 1.

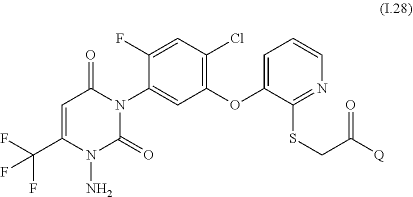

Table I.29: Preferred compounds of the formula (I.29) are the compounds I.29-1 to I.29-486 in which Q has the meanings of Table 1 indicated in the respective row. Thus, the compounds I.29-1 to I.29-486 of Table I.29 are defined by the meaning of the respective entries Nos. 1 to 486 for Q of Table 1.

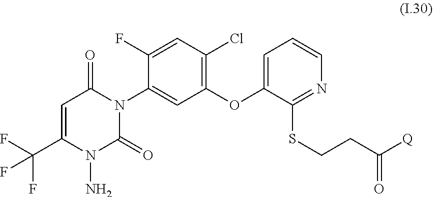

Table I.30: Preferred compounds of the formula (I.30) are the compounds I.30-1 to I.30-486 in which Q has the meanings of Table 1 indicated in the respective row. Thus, the compounds I.30-1 to I.30-486 of Table I.30 are defined by the meaning of the respective entries Nos. 1 to 486 for Q of Table 1.

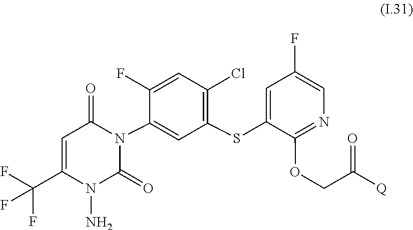

Table I.31: Preferred compounds of the formula (I.31) are the compounds I.31-1 to I.31-486 in which Q has the meanings of Table 1 indicated in the respective row. Thus, the compounds I.31-1 to I.31-486 of Table I.31 are defined by the meaning of the respective entries Nos. 1 to 486 for Q of Table 1.

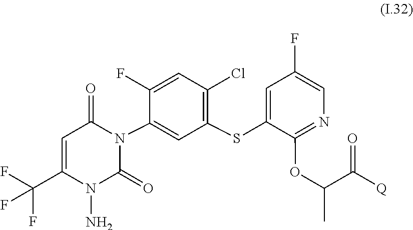

Table I.32: Preferred compounds of the formula (I.32) are the compounds I.32-1 to I.32-486 in which Q has the meanings of Table 1 indicated in the respective row. Thus, the compounds I.32-1 to I.32-486 of

Table I.32 are defined by the meaning of the respective entries Nos. 1 to 486 for Q of Table 1.

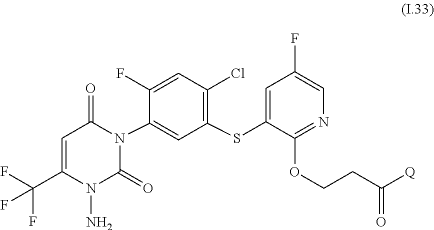

Table I.33: Preferred compounds of the formula (I.33) are the compounds I.33-1 to I.33-486 in which Q has the meanings of Table 1 indicated in the respective row. Thus, the compounds I.33-1 to I.33-486 of Table I.33 are defined by the meaning of the respective entries Nos. 1 to 486 for Q of Table 1.

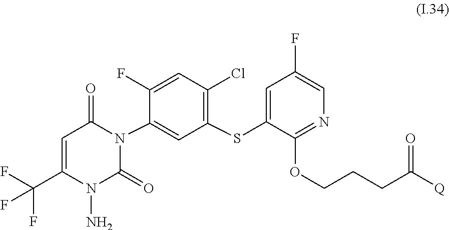

Table I.34: Preferred compounds of the formula (I.34) are the compounds I.34-1 to I.34-486 in which Q has the meanings of Table 1 indicated in the respective row. Thus, the compounds I.34-1 to I.34-486 of Table I.34 are defined by the meaning of the respective entries Nos. 1 to 486 for Q of Table 1.

NMR data of selected examples: The ¹H NMR data of selected examples of compounds of the general formula (I) are stated in two different ways, namely (a) conventional NMR evaluation and interpretation or (b) in the form of ¹H NMR peak lists according to the method described below.

### a) Conventional NMR Interpretation

### Example No. I.1-1

¹H-NMR (CDCl₃ δ, ppm) 8.12 (d, 1H), 7.77 (m, 1H), 7.34 (d, 1H), 7.26 (d, 1H), 6.96 (m, 1H), 6.19 (s, 1H), 4.98-4.88 (q, 2H), 4.61 (s, 2H), 4.32-4.27 (m, 1H), 4.17-4.11 (m, 1H), 3.58-3.56 (m, 2H), 3.36 (s, 3H).

### Example No. I.1-115

¹H-NMR (CDCl₃, δ, ppm) 8.08 (m, 1H), 7.67 (m, 1H), 7.36 (d, 1H), 7.17 (d, 1H), 6.95 (m, 1H), 6.21 (s, 1H), 5.42 (quin, 1H), 5.00-4.87 (dd, 2H), 4.86-4.83 (m, 2H), 4.63-4.59 (m, 2H), 4.54 (s, 2H).

### Example No. I.1-176

¹H-NMR (CDCl₃, δ, ppm) 8.10 (m, 1H), 7.75 (m, 1H), 7.36 (d, 1H), 7.14 (d, 1H), 6.99 (m, 1H), 6.22 (s, 1H), 4.96 (s, 2H), 4.72 (s, 1H), 4.58 (s, 2H).

### Example No. I.1-286

¹H-NMR (CDCl₃, δ, ppm) 8.54 (m, 1H), 8.10 (m, 1H), 7.76 (m, 1H), 7.72-7.68 (m, 1H), 7.33 (d, 1H), 7.33-7.30 (m, 1H), 7.28-7.22 (m, 1H), 7.16 (d, 1H), 6.13 (s, 1H), 5.26 (d, 1H), 5.21 (d, 1H), 5.08 (d, 1H), 4.95 (d, 1H), 4.76 (s, 2H).

### Example No. I.1-471

¹H-NMR (CDCl₃ δ, ppm) 8.11 (d, 1H), 7.73 (m, 1H), 7.35 (d, 1H), 7.26 (d, 1H), 6.97-6.93 (m, 1H), 6.19 (s, 1H), 5.93-5.85 (m, 1H), 5.33-5.23 (m, 2H), 4.99 (d, 1H), 4.92 (d, 1H), 4.59 (m, 2H), 4.51 (s, 2H).

### b) NMR Peak List Method

The NMR data of selected examples may also be stated in the form of NMR peak lists. For each signal peak, first the δ value in ppm and then the signal intensity in round brackets are listed. The pairs of δ value-signal intensity numbers for different signal peaks are listed with separation from one another by semicolons. The peak list for one example therefore takes the form of:

δ₁ (intensity₁); δ₂ (intensity₂); . . . ; δᵢ (intensityᵢ); . . . ; δₙ (intensity.)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum. For calibration of the chemical shift of NMR spectra, we use tetramethylsilane and/or the chemical shift of the solvent, particularly in the case of spectra which are measured in DMSO. Therefore, the tetramethylsilane peak may but need not occur in NMR peak lists. The lists of the NMR peaks are similar to the conventional NMR printouts and thus usually contain all peaks listed in a conventional NMR interpretation. In addition, like conventional NMR printouts, they may show solvent signals, signals of stereoisomers of the target compounds which are likewise provided by the invention, and/or peaks of impurities. In the reporting of compound signals within the delta range of solvents and/or water, our lists of NMR peaks show the standard solvent peaks, for example peaks of DMSO in DMSO-D₆ and the peak of water, which usually have a high intensity on average. The peaks of stereoisomers of the target compounds and/or peaks of impurities usually have a lower intensity on average than the peaks of the target compounds (for example with a purity of >90%). Such stereoisomers and/or impurities may be typical of the particular preparation process. Their peaks can thus help in identifying reproduction of our preparation process with reference to “by-product fingerprints”. An expert calculating the peaks of the target compounds by known methods (MestreC, ACD simulation, but also with empirically evaluated expected values) can, if required, isolate the peaks of the target compounds, optionally using additional intensity filters. This isolation would be similar to the peak picking in question in conventional NMR interpretation.

Further details of NMR peak lists can be found in the Research Disclosure Database Number 564025.

I.31-1: ¹H-NMR (599.6 MHz, CDCl₃):

δ=7.9013 (8.2); 7.8966 (8.2); 7.4147 (4.0); 7.4099 (3.8); 7.4023 (4.2); 7.3976 (3.9); 7.3923 (6.3); 7.3861 (6.8); 7.3801 (6.4); 7.3711 (6.5); 7.2609 (13.8); 6.9275 (0.3); 6.2150 (13.9); 4.9445 (2.3); 4.9179 (11.7); 4.9070 (11.5); 4.8803 (2.2); 4.6156 (18.3); 4.3155 (1.5); 4.3070 (2.7); 4.2987 (1.8); 4.2956 (2.3); 4.2882 (2.5); 4.2860 (2.6); 4.2788 (2.0); 4.1948 (2.0); 4.1882 (3.0); 4.1804 (2.1); 4.1748 (1.5); 4.1677 (2.9); 4.1605 (1.4); 3.7651 (0.4); 3.7361 (0.6); 3.5981 (0.4); 3.5864 (7.1); 3.5793 (11.1); 3.5708 (5.8); 3.5581 (0.3); 3.4019 (1.1); 3.3989 (1.1); 3.3882 (0.5); 3.3651 (50.0); 2.0435 (1.3); 1.5688 (28.2); 1.2704 (0.6); 1.2651 (0.6); 1.2585 (1.1); 1.2466 (0.5); 0.8935 (0.6); 0.8820 (1.4); 0.8700 (0.7); 0.0052 (0.8); -0.0001 (18.3); -0.0056 (0.6)

I.31-442: ¹H-NMR (599.6 MHz, CDCl₃):

δ=7.8899 (11.6); 7.8852 (11.5); 7.4322 (0.4); 7.4192 (8.8); 7.4069 (9.2); 7.4005 (9.2); 7.3856 (9.0); 7.3547 (5.7); 7.3499 (5.4); 7.3422 (5.8); 7.3375 (5.1); 7.2604 (27.9); 6.2204 (19.3); 4.9135 (6.2); 4.8870 (13.8); 4.8487 (13.8); 4.8222 (6.2); 4.5478 (25.9); 4.2296 (0.3); 4.1862 (0.5); 4.1742 (1.4); 4.1683 (2.5); 4.1624 (3.8); 4.1564 (7.1); 4.1506 (7.5); 4.1444 (7.4); 4.1388 (7.2); 4.1326 (3.4); 4.1270 (3.2); 4.1209 (1.1); 4.1153 (1.0); 4.1090 (0.3); 4.1034 (0.3); 3.6847 (0.4); 2.0438 (4.4); 1.5527 (50.0); 1.3017 (0.8); 1.2919 (1.3); 1.2838 (1.4); 1.2798 (2.0); 1.2713 (18.7); 1.2594 (36.4); 1.2475 (17.6); 0.8936 (1.5); 0.8821 (3.5); 0.8701 (1.7); 0.0052 (2.2); -0.0001 (36.7); -0.0056 (1.3)

I.1-481: ¹H-NMR (599.6 MHz, CDCl₃):

δ=8.1359 (2.5); 8.1331 (2.6); 8.1277 (2.6); 8.1248 (2.5); 7.7639 (2.5); 7.7610 (2.6); 7.7515 (2.7); 7.7486 (2.6); 7.3577 (3.9); 7.3427 (3.9); 7.2602 (39.9); 7.0566 (3.6); 7.0444 (3.6); 6.9885 (2.5); 6.9802 (2.5); 6.9761 (2.5); 6.9678 (2.4); 6.2255 (8.5); 5.2992 (6.9); 4.9646 (2.4); 4.9378 (5.0); 4.8955 (4.9); 4.8686 (2.3); 4.6087 (7.2); 2.1723 (0.5); 2.0906 (0.3); 1.3128 (0.4); 1.3016 (0.8); 1.2900 (1.0); 1.2801 (1.0); 1.2653 (2.2); 0.8937 (2.1); 0.8822 (4.9); 0.8702 (2.4); 0.0052 (2.1); −0.0001 (50.0); −0.0054 (2.0)

The present invention furthermore provides the use of one or more compounds of the general formula (I) according to the invention and/or salts thereof, as defined above, preferably in one of the embodiments identified as preferred or particularly preferred, in particular one or more compounds of the formulae (I.1) to (I.34) and/or salts thereof, in each case as defined above, as herbicide and/or plant growth regulator, preferably in crops of useful plants and/or ornamental plants.

The present invention further provides a method for controlling harmful plants and/or for regulating the growth of plants, characterized in that an effective amount
of one or more compounds of the general formula (I) according to the invention and/or salts thereof, as defined above, preferably in one of the embodiments identified as preferred or particularly preferred, in particular one or more compounds of the formulae (I.1) to (I.34) and/or salts thereof, in each case as defined above, or
of a composition according to the invention, as defined below,
   is applied to the (harmful) plants, seeds of (harmful) plants, the soil in which or on which the (harmful) plants grow or the area under cultivation.

The present invention also provides a method for controlling unwanted plants, preferably in crops of useful plants, characterized in that an effective amount
of one or more compounds of the general formula (I) and/or salts thereof, as defined above, preferably in one of the embodiments identified as preferred or particularly preferred, in particular one or more compounds of the formulae (I.1) to (I.34) and/or salts thereof, in each case as defined above, or
of a composition according to the invention, as defined below,
   is applied to unwanted plants (for example harmful plants such as mono- or dicotyledonous weeds or unwanted crop plants), the seed of the unwanted plants (i.e. plant seeds, for example grains, seeds or vegetative propagation organs such as tubers or shoot parts with buds), the soil in which or on which the unwanted plants grow (for example the soil of crop land or non-crop land) or the area under cultivation (i.e. the area on which the unwanted plants will grow).

The present invention also further provides methods for controlling for regulating the growth of plants, preferably of useful plants, characterized in that an effective amount
of one or more compounds of the general formula (I) and/or salts thereof, as defined above, preferably in one of the embodiments identified as preferred or particularly preferred, in particular one or more compounds of the formulae (I.1) to (I.34) and/or salts thereof, in each case as defined above, or
of a composition according to the invention, as defined below,
   is applied to the plant, the seed of the plant (i.e. plant seed, for example grains, seeds or vegetative propagation organs such as tubers or shoot parts with buds), the soil in which or on which the plants grow (for example the soil of crop land or non-crop land) or the area under cultivation (i.e. the area on which the plants will grow).

In this context, the compounds according to the invention or the compositions according to the invention can be applied for example by pre-sowing (if appropriate also by incorporation into the soil), pre-emergence and/or post-emergence processes. Specific examples of some representatives of the monocotyledonous and dicotyledonous weed flora which can be controlled by the compounds according to the invention are as follows, though there is no intention to restrict the enumeration to particular species.

In a method according to the invention for controlling harmful plants or for regulating the growth of plants, one or more compounds of the general formula (I) and/or salts thereof are preferably employed for controlling harmful plants or for regulating growth in crops of useful plants or ornamental plants, where in a preferred embodiment the useful plants or ornamental plants are transgenic plants.

The compounds of the general formula (I) according to the invention and/or their salts are suitable for controlling the following genera of monocotyledonous and dicotyledonous harmful plants: Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous harmful plants of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the compounds according to the invention are applied to the soil surface before germination of the harmful plants (weed grasses and/or broad-leaved weeds) (pre-emergence method), either the seedlings of the weed grasses or broad-leaved weeds are prevented completely from emerging or they grow until they have reached the cotyledon stage, but then stop growing and eventually, after three to four weeks have elapsed, die completely.

If the active compounds are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

Although the compounds according to the invention display an outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Miscanthus, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, are damaged only to an insignificant extent, or not at all, depending on the structure of the respective compound according to the invention and its application rate. For these reasons, the present compounds are very suitable for selective control of unwanted plant growth in plant crops such as agriculturally useful plants or ornamental plants.

In addition, the compounds of the general formula (I) according to the invention (depending on their particular structure and the application rate deployed) have outstanding growth-regulating properties in crop plants. They intervene in the plants' own metabolism with regulatory effect, and can thus be used for the controlled influencing of plant constituents and to facilitate harvesting, for example by triggering desiccation and stunted growth. Furthermore, they are also suitable for the general control and inhibition of unwanted vegetative growth without killing the plants in the process. Inhibition of vegetative growth plays a major role for many mono- and dicotyledonous crops since, for example, this can reduce or completely prevent lodging.

By virtue of their herbicidal and plant growth regulatory properties, the compounds of the general formula (I) according to the invention can also be used to control harmful plants in crops of genetically modified plants or plants modified by conventional mutagenesis. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material.

It is preferred with a view to transgenic crops to use the compounds according to the invention and/or their salts in economically important transgenic crops of useful plants and ornamentals, for example of cereals such as wheat, barley, rye, oats, millet, rice and corn or else crops of sugar beet, cotton, soybean, oilseed rape, potato, tomato, peas and other vegetables.

It is preferred to employ the compounds according to the invention as herbicides in crops of useful plants which are resistant, or have been made resistant by recombinant means, to the phytotoxic effects of the herbicides.

By virtue of their herbicidal and plant growth regulatory properties, the active compounds can also be used to control harmful plants in crops of genetically modified plants which are known or are yet to be developed. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further special properties may be tolerance or resistance to abiotic stressors, for example heat, cold, drought, salinity and ultraviolet radiation.

Preference is given to the use of the compounds of the general formula (I) according to the invention or salts thereof in economically important transgenic crops of useful plants and ornamentals, for example of cereals such as wheat, barley, rye, oats, triticale, millet, rice, cassava and corn, or else crops of sugar beet, cotton, soybean, oilseed rape, potatoes, tomatoes, peas and other vegetables.

It is preferable to employ the compounds of the general formula (I) as herbicides in crops of useful plants which are resistant, or have been made resistant by recombinant means, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods.

A large number of molecular-biological techniques by means of which novel transgenic plants with modified properties can be generated are known to the person skilled in the art. For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. To connect the DNA fragments to each other, adapters or linkers may be added to the fragments.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product.

To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Thus, transgenic plants can be obtained whose properties are altered by overexpression, suppression or inhibition of homologous (=natural) genes or gene sequences or expression of heterologous (=foreign) genes or gene sequences.

It is preferable to employ the compounds of the general formula (I) according to the invention in transgenic crops which are resistant to growth regulators such as, for example, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, glyphosates, glufosinates or benzoylisoxazoles and analogous active compounds.

When the active compounds of the invention are employed in transgenic crops, not only do the effects towards harmful plants to be observed in other crops occur, but frequently also effects which are specific to the application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the compounds of the general formula (I) according to the invention and/or their salts as herbicides for controlling harmful plants in crops of useful plants or ornamentals, optionally in transgenic crop plants.

Preference is given to the use in cereals, here preferably corn, wheat, barley, rye, oats, millet or rice, by the pre- or post-emergence method.

Preference is also given to the use in soybeans by the pre- or post-emergence method.

The use according to the invention for the control of harmful plants or for growth regulation of plants also includes the case in which a compound of the general formula (I) or its salt is not formed from a precursor substance (“prodrug”) until after application on the plant, in the plant or in the soil. The invention also provides the use of one or more compounds of the general formula (I) or salts thereof or of a composition according to the invention (as defined below) (in a method) for controlling harmful plants or for regulating the growth of plants which comprises applying an effective amount of one or more compounds of the general formula (I) or salts thereof onto the plants (harmful plants, if appropriate together with the useful plants), plant seeds, the soil in which or on which the plants grow or the area under cultivation.

The invention also provides a herbicidal and/or plant growth-regulating composition, characterized in that the composition comprises

(a) one or more compounds of the general formula (I) and/or salts thereof, as defined above, preferably in one of the embodiments identified as preferred or particularly preferred, in particular one or more compounds of the formulae (I.1) to (I.34) and/or salts thereof, in each case as defined above, and

(b) one or more further substances selected from groups (i) and/or (ii):
(i) one or more further agrochemically active substances, preferably selected from the group consisting of insecticides, acaricides, nematicides, further herbicides (i.e. those not conforming to the general formula (I) defined above), fungicides, safeners, fertilizers and/or further growth regulators,
(ii) one or more formulation auxiliaries customary in crop protection.

Here, the further agrochemically active substances of component (i) of a composition according to the invention are preferably selected from the group of substances mentioned in “The Pesticide Manual”, 16th edition, The British Crop Protection Council and the Royal Soc. of Chemistry, 2012.

A herbicidal or plant growth-regulating composition according to the invention comprises preferably one, two, three or more formulation auxiliaries (ii) customary in crop protection selected from the group consisting of surfactants, emulsifiers, dispersants, film-formers, thickeners, inorganic salts, dusting agents, carriers solid at 25° C. and 1013 mbar, preferably adsorptive granulated inert materials, wetting agents, antioxidants, stabilizers, buffer substances, antifoam agents, water, organic solvents, preferably organic solvents miscible with water in any ratio at 25° C. and 1013 mbar.

The compounds of the general formula (I) according to the invention can be used in the form of wettable powders, emulsifiable concentrates, sprayable solutions, dusting products or granules in the customary formulations. The invention therefore also provides herbicidal and plant growth-regulating compositions which comprise compounds of the general formula (I) and/or salts thereof.

The compounds of the general formula (I) and/or salts thereof can be formulated in various ways according to which biological and/or physicochemical parameters are required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes.

These individual formulation types and the formulation assistants, such as inert materials, surfactants, solvents and further additives, are known to the person skilled in the art and are described, for example, in: Watkins, “Handbook of Insecticide Dust Diluents and Carriers”, 2nd Ed., Darland Books, Caldwell N.J.; H.v. Olphen, “Introduction to Clay Colloid Chemistry”, 2nd ed., J. Wiley & Sons, N.Y.; C. Marsden, “Solvents Guide”, 2nd ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Interface-active Ethylene Oxide Adducts], Wiss. Verlagsgesellschaft, Stuttgart 1976; Winnacker-Küchler, “Chemische Technologie” [Chemical Technology], volume 7, C. Hanser Verlag Munich, 4th Ed. 1986.

Wettable powders are preparations which can be dispersed uniformly in water and, in addition to the active compound, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the herbicidal active compounds are finely ground, for example in customary apparatuses such as hammer mills, blower mills and air-jet mills, and simultaneously or subsequently mixed with the formulation auxiliaries.

Emulsifiable concentrates are produced by dissolving the active compound in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonate salts, for example calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active compound with finely distributed solids, for example talc, natural clays, such as kaolin, bentonite and pyrophyllite, or diatomaceous earth.

Suspension concentrates may be water- or oil-based. They can be produced, for example, by wet grinding by means of standard commercial bead mills and optionally the addition of surfactants, as have already been listed e.g. above for the other types of formulation.

Emulsions, e.g. oil-in-water emulsions (EW), can be prepared, for example, by means of stirrers, colloid mills and/or static mixers using aqueous organic solvents and optionally surfactants, as have already been listed e.g. above for the other formulation types.

Granules can be prepared either by spraying the active compound onto granular inert material capable of adsorption or by applying active compound concentrates to the surface of carrier substances, such as sand, kaolinites or granular inert material, by means of adhesives, for example polyvinyl alcohol, sodium polyacrylate or else mineral oils. Suitable active compounds can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan granules, fluidized bed granules, extruder granules and spray granules, see, for example, processes in “Spray-Drying Handbook” 3rd ed. 1979, G. Goodwin Ltd., London; J. E. Browning, “Agglomeration”, Chemical and Engineering 1967, pages 147 ff; “Perry's Chemical Engineer's Handbook”, 5th Ed., McGraw Hill, New York 1973, p. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th ed., Blackwell Scientific Publications, Oxford, 1968, pages 101-103.

The agrochemical preparations, preferably herbicidal or plant growth-regulating compositions, of the present invention preferably comprise a total amount of from 0.1 to 99% by weight, preferably 0.5 to 95% by weight, particularly preferably 1 to 90% by weight, especially preferably 2 to 80% by weight, of active compounds of the general formula (I) and their salts.

In wettable powders, the active compound concentration is, for example, about 10% to 90% by weight, the remainder to 100% by weight consisting of customary formulation constituents. In emulsifiable concentrates, the active compound concentration may be about 1% to 90% and preferably 5% to 80% by weight. Formulations in the form of dusts comprise 1% to 30% by weight of active compound, preferably usually 5% to 20% by weight of active compound; sprayable solutions contain about 0.05% to 80% by weight, preferably 2% to 50% by weight of active compound. In the case of water-dispersible granules, the active compound content depends partially on whether the active compound is in liquid or solid form and on which granulation auxiliaries, fillers, etc., are used. In the water-dispersible granules, the content of active compound is, for example, between 1 and 95% by weight, preferably between 10 and 80% by weight.

In addition, the active compound formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity. Examples of formulation auxiliaries are described, inter alia, in “Chemistry and Technology of Agrochemical Formulations”, ed. D. A. Knowles, Kluwer Academic Publishers (1998).

The compounds of the general formula (I) or salts thereof can be used as such or in the form of their preparations (formulations) in a combination with other pesticidally active substances, for example insecticides, acaricides, nematicides, herbicides, fungicides, safeners, fertilizers and/or growth regulators, for example in the form of a finished formulation or of a tank mix. The combination formulations can be prepared on the basis of the abovementioned formulations, while taking account of the physical properties and stabilities of the active compounds to be combined.

Active compounds which can be employed in combination with the compounds of the general formula (I) according to the invention in mixture formulations or in a tank mix are, for example, known active compounds based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II, protoporphyrinogen oxidase, as described, for example, in Weed Research 26 (1986) 441-445 or “The Pesticide Manual”, 16th edition, The British Crop Protection Council and the Royal Soc. of Chemistry, 2012 and literature cited therein.

Of particular interest is the selective control of harmful plants in crops of useful plants and ornamentals. Although the compounds of the general formula (I) according to the invention have already demonstrated very good to adequate selectivity in a large number of crops, in principle, in some crops and in particular also in the case of mixtures with other, less selective herbicides, phytotoxicities on the crop plants may occur. In this connection, combinations of compounds of the general formula (I) according to the invention are of particular interest which comprise the compounds (I) or their combinations with other herbicides or pesticides and safeners. The safeners, which are used in an antidotically effective amount, reduce the phytotoxic side effects of the herbicides/pesticides employed, for example in economically important crops, such as cereals (wheat, barley, rye, corn, rice, millet), sugarbeet, sugarcane, oilseed rape, cotton and soybeans, preferably cereals.

The weight ratios of herbicide (mixture) to safener depend generally on the herbicide application rate and the efficacy of the safener in question and may vary within wide limits, for example in the range from 200:1 to 1:200, preferably 100:1 to 1:100, in particular 20:1 to 1:20. Analogously to the compounds of the general formula (I) or mixtures thereof, the safeners can be formulated with further herbicides/pesticides and be provided and employed as a finished formulation or tank mix with the herbicides.

For application, the herbicide or herbicide/safener formulations present in commercial form are, if appropriate, diluted in a customary manner, for example in the case of wettable powders, emulsifiable concentrates, dispersions and water-dispersible granules with water. Dust-type preparations, granules for soil application or granules for scattering and sprayable solutions are not normally diluted further with other inert substances prior to application.

The application rate of the compounds of the general formula (I) and/or their salts is affected to a certain extent by external conditions such as temperature, humidity, etc. Here, the application rate may vary within wide limits. For the application as a herbicide for controlling harmful plants, the total amount of compounds of the general formula (I) and their salts is preferably in the range from 0.001 to 10.0 kg/ha, with preference in the range from 0.005 to 5 kg/ha, more preferably in the range from 0.01 to 1.5 kg/ha, particularly preferably in the range from 0.05 to 1 kg/ha. This applies both to the pre-emergence and the post-emergence application.

When compounds of the general formula (I) and/or their salts are used as plant growth regulator, for example as culm stabilizer for crop plants like those mentioned above, preferably cereal plants, such as wheat, barley, rye, triticale, millet, rice or corn, the total application rate is preferably in the range of from 0.001 to 2 kg/ha, preferably in the range of from 0.005 to 1 kg/ha, in particular in the range of from 10 to 500 g/ha, very particularly preferably in the range from 20 to 250 g/ha. This applies both to the pre-emergence and the post-emergence application.

The application as culm stabilizer may take place at various stages of the growth of the plants. Preferred is, for example, the application after the tittering phase, at the beginning of the longitudinal growth.

As an alternative, application as plant growth regulator is also possible by treating the seed, which includes various techniques for dressing and coating seed. Here, the application rate depends on the particular techniques and can be determined in preliminary tests.

Active compounds which can be employed in combination with the compounds of the general formula (I) according to the invention in compositions according to the invention (for example in mixed formulations or in the tank mix) are, for example, known active compounds which are based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase, as are described in, for example, Weed Research 26 (1986) 441-445 or “The Pesticide Manual”, 16th edition, The British Crop Protection Council and the Royal Soc. of Chemistry, 2012 and the literature cited therein. Known herbicides or plant growth regulators which can be combined with the compounds of the invention are, for example, the following, where said active compounds are designated either with their “common name” in accordance with the International Organization for Standardization (ISO) or with the chemical name or with the code number. They always encompass all the use forms, for example acids, salts, esters and also all isomeric forms such as stereoisomers and optical isomers, even if they are not mentioned explicitly.

Examples of such herbicidal mixing partners are:

acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammonium sulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamine, -ethyl, 2-ethylhexyl, -isobutyl, -isooctyl, -isopropylammonium, -potassium,-triisopropanolammonium and -trolamine, 2,4-DB, 2,4-DB-butyl, -dimethylammonium, isooctyl, -potassium and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-[2-chloro-4-fluoro-5-[4-(3-fluoropropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium,-sodium and -trimesium, H-9201, i.e. 0-(2,4-dimethyl-6-nitrophenyl) O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium,-potassium and -sodium, MCPB, MCPB-methyl, -ethyl and -sodium, mecoprop, mecoprop-sodium, and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinate, monolinuron, monosulfuron, monosulfuron-ester, MT-5950, i.e. N-[3-chloro-4-(1-methylethyl)phenyl]-2-methylpentanamide, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorophenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trifluoroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, and the following compounds:

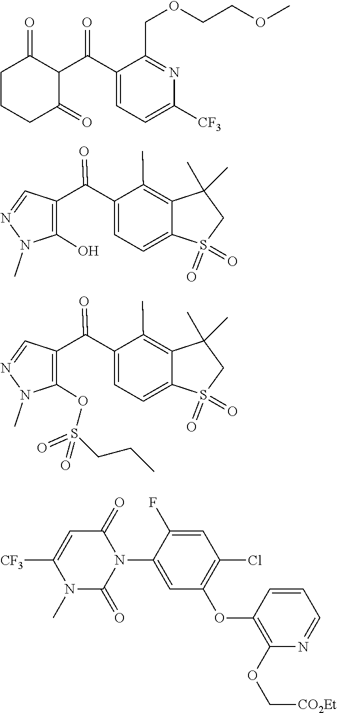

Examples of plant growth regulators as possible mixing partners are:

acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenolate mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

Useful combination partners for the compounds of the general formula (I) according to the invention also include, for example, the following safeners:
S1) Compounds from the group of heterocyclic carboxylic acid derivatives:
S1^{a}) Compounds of the dichlorophenylpyrazoline-3-carboxylic acid type (S1^{a}), preferably compounds such as
   1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
S1^{b}) Derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333131 and EP-A-269806;

S1^{c}) Derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described, for example, in EP-A-268554;

S1^{d}) Compounds of the triazolecarboxylic acid type (S1^{d}), preferably compounds such as fenchlorazole(-ethyl ester), i.e. ethyl 1-(2,4-dichlorophenyl)-5-trichloromethyl-1H-1,2,4-triazole-3-carboxylate (51-7), and related compounds, as described in EP-A-174562 and EP-A-346620;

S1^{e}) Compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (51-9) and related compounds as described in WO-A-91/08202, or 5,5-diphenyl-2-isoxazolinecarboxylic acid (51-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (51-12) or ethyl 5-(4-fluorophenyl)-5-phenyl-2-isoxazoline-3-carboxylate (51-13) as described in patent application WO-A-95/07897.

S2) Compounds from the group of the 8-quinolinoxy derivatives (S2):

S2^{a}) Compounds of the 8-quinolinoxyacetic acid type (S2^{a}), preferably 1-methylhexyl (5-chloro-8-quinolinoxy)acetate (“cloquintocet-mexyl”) (S2-1), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5),
methyl (5-chloro-8-quinolinoxy)acetate (S2-6),
allyl (5-chloro-8-quinolinoxy)acetate (52-7), 2-(2-propylideneiminoxy)-1-ethyl (5-chloro-8-quinolinoxy)acetate (52-8), 2-oxoprop-1-yl (5-chloro-8-quinolinoxy)acetate (52-9) and related compounds, as described in EP-A-86750, EP-A-94349 and EP-A-191736 or EP-A-0 492 366, and also (5-chloro-8-quinolinoxy)acetic acid (52-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;
S2^{b}) Compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Active compounds of the dichloroacetamide type (S3), which are frequently used as pre-emergence safeners (soil-acting safeners), for example
“dichlormid” (N,N-diallyl-2,2-dichloroacetamide) (53-1),
“R-29148” (3-dichloroacetyl-2,2,5-trimethyl-1,3-oxazolidine) from Stauffer (S3-2),
“R-28725” (3-dichloroacetyl-2,2-dimethyl-1,3-oxazolidine) from Stauffer (53-3),
“benoxacor” (4-dichloroacetyl-3,4-dihydro-3-methyl-2H-1,4-benzoxazine) (53-4),
“PPG-1292” (N-allyl-N-[(1,3-dioxolan-2-yl)methyl]dichloroacetamide) from PPG Industries (53-5),
“DKA-24” (N-allyl-N-[(allylaminocarbonyl)methyl]dichloroacetamide) from Sagro-Chem (53-6),
“AD-67” or “MON 4660” (3-dichloroacetyl-1-oxa-3-azaspiro[4.5]decane) from Nitrokemia or Monsanto (S3-7),
“TI-35” (1-dichloroacetylazepane) from TRI-Chemical RT (S3-8),
“diclonon” (dicyclonon) or “BAS145138” or “LAB145138” (S3-9)
((RS)-1-dichloroacetyl-3,3,8a-trimethylperhydropyrrolo[1,2-a]pyrimidin-6-one) from BASF,
“furilazole” or “MON 13900” ((RS)-3-dichloroacetyl-5-(2-furyl)-2,2-dimethyloxazolidine) (53-10), and the (R) isomer thereof (S3-11).
S4) Compounds from the class of the acylsulfonamides (S4):
S4^{a}) N-Acylsulfonamides of the formula (S4^{a}) and salts thereof, as described in WO-A-97/45016,

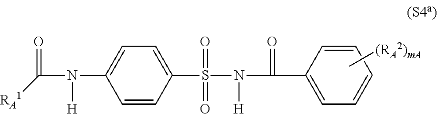

in which
R_{A}¹ represents (C₁-C₆)-alkyl, (C₃-C₆)-cycloalkyl, where the 2 latter radicals are substituted by VA substituents from the group of halogen, (C₁-C₄)-alkoxy, (C₁-C₆)-haloalkoxy and (C₁-C₄)-alkylthio and, in the case of cyclic radicals, also by (C₁-C₄)-alkyl and (C₁-C₄)-haloalkyl;
R_{A}² represents halogen, (C₁-C₄)-alkyl, (C₁-C₄)-alkoxy, CF₃;
m_{A} represents 1 or 2;
v_{A} represents 0, 1, 2 or 3;
S4^{b}) Compounds of the 4-(benzoylsulfamoyl)benzamide type of the formula (S4^{b}) and salts thereof, as described in WO-A-99/16744,

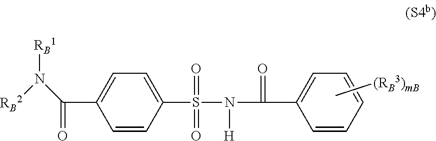

in which
R_{B}¹, R_{B}² independently of one another represent hydrogen, (C₁-C₆)-alkyl, (C₃-C₆)-cycloalkyl, (C₃-C₆)-alkenyl, (C₃-C₆)-alkynyl,
R_{B}³ represents halogen, (C₁-C₄)-alkyl, (C₁-C₄)-haloalkyl or (C₁-C₄)-alkoxy and
m_{B} represents 1 or 2,
e.g. those in which
R_{B}′=cyclopropyl, R_{B}²=hydrogen and (R_{B}³)=2-OMe (“cyprosulfamide”, S4-1),
R_{B}¹=cyclopropyl, R_{B}²=hydrogen and (R_{B}³)=5-Cl-2-OMe (S4-2),
R_{B}¹=ethyl, R_{B}²=hydrogen and (R_{B}³)=2-OMe (S4-3),
R_{B}¹=isopropyl, R_{B}²=hydrogen and (R_{B}³)=5-Cl-2-OMe (S4-4) and
R_{B}¹=isopropyl, R_{B}²=hydrogen and (R_{B}³)=2-OMe (S4-5);
S4^{c}) Compounds from the class of the benzoylsulfamoylphenylureas of the formula (S4^{c}), as described in EP-A-365484,

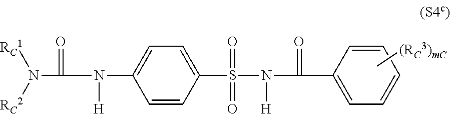

in which
R_{C}¹, R_{C}² independently of one another represent hydrogen, (C₁-C₈)-alkyl, (C₃-C₈-cycloalkyl, (C₃-C₆)-alkenyl, (C₃-C₆)-alkynyl,
R_{C}³ represents halogen, (C₁-C₄)-alkyl, (C₁-C₄)-alkoxy, CF₃ and
m_{C} represents 1 or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea;
S4^{d}) Compounds of the N-phenylsulfonylterephthalamide type of the formula (S4^{d}) and salts thereof, which are known, for example, from CN 101838227,

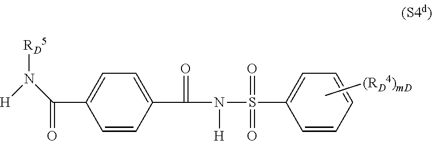

in which
R_{D}⁴ is halogen, (C₁-C₄)-alkyl, (C₁-C₄)-alkoxy, CF₃;
m_{D} is 1 or 2;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.
S5) Active compounds from the class of the hydroxyaromatics and the aromatic-aliphatic carboxylic acid derivatives (S5), for example
ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
S6) Active compounds from the class of the 1,2-dihydroquinoxalin-2-ones (S6), for example
1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds from the class of the diphenylmethoxyacetic acid derivatives (S7), e.g. methyl diphenylmethoxyacetate (CAS Reg. No. 41858-19-9) (S7-1), ethyl diphenylmethoxyacetate or diphenylmethoxyacetic acid, as described in WO-A-98/38856.
S8) Compounds of the formula (S8), as described in WO-A-98/27049,

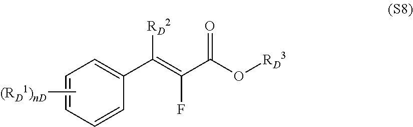

in which the symbols and indices are defined as follows:
R_{D}¹ represents halogen, (C₁-C₄)-alkyl, (C₁-C₄)-haloalkyl, (C₁-C₄)-alkoxy, (C₁-C₄)-haloalkoxy,
R_{D}² represents hydrogen or (C₁-C₄)-alkyl,
R_{D}³ represents hydrogen, (C₁-C₈)-alkyl, (C₂-C₄)-alkenyl, (C₂-C₄)-alkynyl or aryl, where each of the aforementioned carbon-containing radicals is unsubstituted or substituted by one or more, preferably up to three, identical or different radicals from the group consisting of halogen and alkoxy; or salts thereof,
n_{D} represents an integer from 0 to 2.
S9) Active compounds from the class of the 3-(5-tetrazolylcarbonyl)-2-quinolones (S9), for example
1,2-dihydro-4-hydroxy-1-ethyl-3-(5-tetrazolylcarbonyl)-2-quinolone (CAS Reg. No.: 219479-18-2), 1,2-dihydro-4-hydroxy-1-methyl-3-(5-tetrazolylcarbonyl)-2-quinolone (CAS Reg. No. 95855-00-8), as described in WO-A-1999/000020.
S10) Compounds of the formulae (S10^{a}) or (S10^{b})
   as described in WO-A-2007/023719 and WO-A-2007/023764,

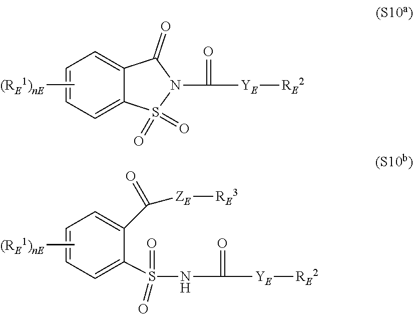

in which
R_{E}¹ represents halogen, (C₁-C₄)-alkyl, methoxy, nitro, cyano, CF₃, OCF₃,
Y_{E}, Z_{E} independently of one another represent O or S,
n_{E} represents an integer from 0 to 4,
R_{E}² represents (C₁-C₁₆)-alkyl, (C₂-C₆)-alkenyl, (C₃-C₆)-cycloalkyl, aryl; benzyl, halobenzyl,
R_{E}³ represents hydrogen or (C₁-C₆)-alkyl.
S11) Active compounds of the oxyimino compounds type (S11), which are known as seed-dressing agents, for example
“oxabetrinil” ((Z)-1,3-dioxolan-2-ylmethoxyimino(phenyl)acetonitrile) (S11-1), which is known as a seed-dressing safener for millet/sorghum against metolachlor damage,
   “fluxofenim” (1-(4-chlorophenyl)-2,2,2-trifluoro-1-ethanone O-(1,3-dioxolan-2-ylmethyfloxime) (S11-2), which is known as a seed-dressing safener for millet/sorghum against metolachlor damage, and
   “cyometrinil” or “CGA-43089” ((Z)-cyanomethoxyimino(phenyl)acetonitrile) (S11-3), which is known as a seed-dressing safener for millet/sorghum against metolachlor damage.
S12) Active compounds from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.
S13) One or more compounds from group (S13):
   “naphthalic anhydride” (1,8-naphthalenedicarboxylic anhydride) (S13-1), which is known as a seed-dressing safener for corn against thiocarbamate herbicide damage,
   “fenclorim” (4,6-dichloro-2-phenylpyrimidine) (S13-2), which is known as a safener for pretilachlor in sown rice,
   “flurazole” (benzyl 2-chloro-4-trifluoromethyl-1,3-thiazole-5-carboxylate) (S13-3), which is known as a seed-dressing safener for millet/sorghum against alachlor and metolachlor damage,
   “CL 304415” (CAS Reg. No. 31541-57-8)
   (4-carboxy-3,4-dihydro-2H-1-benzopyran-4-acetic acid) (S13-4) from American Cyanamid, which is known as a safener for corn against damage by imidazolinones,
   “MG 191” (CAS Reg. No. 96420-72-3) (2-dichloromethyl-2-methyl-1,3-dioxolane) (S13-5) from Nitrokemia, which is known as a safener for corn,
   “MG 838” (CAS Reg. No. 133993-74-5) (2-propenyl 1-oxa-4-azaspiro[4.5]decane-4-carbodithioate) (S13-6) from Nitrokemia
   “disulfoton” (0,0-diethyl S-2-ethylthioethyl phosphorodithioate) (S13-7),
   “dietholate” (0,0-diethyl 0-phenyl phosphorothioate) (513-8),
   “mephenate” (4-chlorophenyl methylcarbamate) (S13-9).
S14) Active compounds which, in addition to herbicidal action against harmful plants, also have safener action on crop plants such as rice, for example
“dimepiperate” or “MY-93” (S-1-methyl 1-phenylethylpiperidine-1-carbothioate), which is known as a safener for rice against damage by the herbicide molinate,
   “daimuron” or “SK 23” (1-(1-methyl-1-phenylethyl)-3-p-tolylurea), which is known as a safener for rice against damage by the herbicide imazosulfuron,
   “cumyluron”=“JC-940” (3-(2-chlorophenylmethyl)-1-(1-methyl-1-phenylethyl)urea, see JP-A-60087254), which is known as a safener for rice against damage by some herbicides,
   “methoxyphenone” or “NK 049” (3,3′-dimethyl-4-methoxybenzophenone), which is known as a safener for rice against damage by some herbicides,
   “CSB” (1-bromo-4-(chloromethylsulfonyl)benzene) from Kumiai, (CAS Reg. No. 54091-06-4), which is known as a safener against damage by some herbicides in rice.
S15) Compounds of the formula (S15) or tautomers thereof

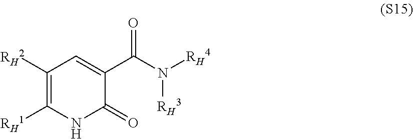

as described in WO-A-2008/131861 and WO-A-2008/131860
in which
R_{H}¹ is a (C₁-C₆)-haloalkyl radical and
R_{H}² is hydrogen or halogen and
R_{H}³, R_{H}⁴ independently of one another represent hydrogen, (C₁-C₁₆)-alkyl, (C₂-C₁₆)-alkenyl or
   (C₂-C₁₆)-alkynyl,
      where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
      where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or
R_{H}³ is (C₁-C₄)-alkoxy, (C₂-C₄)-alkenyloxy, (C₂-C₆)-alkynyloxy or (C₂-C₄)-haloalkoxy and
R_{H}⁴ is hydrogen or (C₁-C₄)-alkyl or
R_{H}³ and R_{H}⁴ together with the directly attached nitrogen atom represent a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.
S16) Active compounds which are used primarily as herbicides but also have safener action on crop plants, for example
   (2,4-dichlorophenoxy)acetic acid (2,4-D),
   (4-chlorophenoxy)acetic acid,
   (R,S)-2-(4-chloro-o-tolyloxy)propionic acid (mecoprop),
   4-(2,4-dichlorophenoxy)butyric acid (2,4-DB),
   (4-chloro-o-tolyloxy)acetic acid (MCPA),
   4-(4-chloro-o-tolyloxy)butyric acid,
   4-(4-chlorophenoxy)butyric acid,
   3,6-dichloro-2-methoxybenzoic acid (dicamba),
   1-(ethoxycarbonyl)ethyl 3,6-dichloro-2-methoxybenzoate (lactidichlor-ethyl).

Preferred safeners in combination with the compounds of the general formula (I) according to the invention and/or salts thereof, in particular with the compounds of the formulae (I.1) to (I.34) and/or salts thereof, are: cloquintocet-mexyl, cyprosulfamide, fenchlorazole ethyl ester, isoxadifen-ethyl, mefenpyr-diethyl, fenclorim, cumyluron, S4-1 and S4-5, and particularly preferred safeners are: cloquintocet-mexyl, cyprosulfamide, isoxadifen-ethyl and mefenpyr-diethyl.

### Biological Examples

### A. Post-Emergence Herbicidal Action and Crop Plant Compatibility

Seeds of monocotyledonous and dicotyledonous weeds and crop plants were placed in sandy loam in plastic or wood-fiber pots, covered with soil and cultivated in a greenhouse under controlled growth conditions. 2 to 3 weeks after sowing, the test plants were treated at the one-leaf stage. The compounds of the invention, formulated in the form of wettable powders (WP) or as emulsion concentrates (EC), were then sprayed onto the green parts of the plants as aqueous suspension or emulsion with addition of 0.5% additive at a water application rate of 6001/ha (converted). After the test plants had been kept in the greenhouse under optimum growth conditions for about 3 weeks, the activity of the preparations was rated visually in comparison to untreated controls. For example, 100% activity=the plants have died, 0% activity=like control plants.

Tables A1 to A14 below show the effects of selected compounds of the general formula (I) according to Tables I.1 to I.34 on various harmful plants and at an application rate corresponding to 20 g/ha and less, which were obtained by the experimental procedure mentioned above.

TABLE A1

| Compound | Alopecurus myosuroides | Application rate |
|---|---|---|
| Example No. | (efficacy in %) | [g/ha] |
| I.1-1 | 90 | 20 |
| I.1-2 | 100 | 20 |
| I.1-23 | 90 | 20 |
| I.1-115 | 100 | 20 |
| I.1-115 | 80 | 5 |
| I.1-176 | 100 | 20 |
| I.1-176 | 90 | 5 |
| I.1-286 | 100 | 20 |
| I.1-286 | 100 | 5 |
| I.1-447 | 90 | 20 |
| I.1-447 | 80 | 5 |
| I.1-457 | 80 | 20 |
| I.1-457 | 80 | 5 |
| I.1-471 | 100 | 20 |
| I.1-471 | 80 | 5 |
| I.1-481 | 100 | 20 |
| I.1-481 | 100 | 5 |
| I.6-441 | 80 | 20 |
| I.14-442 | 90 | 20 |
| I.31-1 | 80 | 20 |
| I.31-23 | 100 | 20 |
| I.31-442 | 90 | 20 |
| I.31-442 | 80 | 5 |

TABLE A2

| Compound | Digitaria sanguinalis | Application rate |
|---|---|---|
| Example No. | (efficacy in %) | [g/ha] |
| I.1-1 | 100 | 20 |
| I.1-1 | 100 | 5 |
| I.1-2 | 100 | 20 |
| I.1-2 | 100 | 5 |
| I.1-23 | 100 | 20 |
| I.1-23 | 100 | 5 |
| I.1-441 | 100 | 20 |
| I.1-441 | 90 | 5 |
| I.1-442 | 100 | 20 |
| I.1-447 | 100 | 20 |
| I.1-447 | 100 | 5 |
| I.1-448 | 100 | 20 |
| I.1-457 | 100 | 20 |
| I.1-457 | 100 | 5 |
| I.2-442 | 100 | 20 |
| I.6-441 | 100 | 20 |
| I.6-441 | 80 | 5 |
| I.14-442 | 100 | 20 |

TABLE A3

| Compound | Echinochloa crus-galli | Application rate |
|---|---|---|
| Example No. | (efficacy in %) | [g/ha] |
| I.1-1 | 100 | 20 |
| I.1-1 | 90 | 5 |
| I.1-2 | 100 | 20 |
| I.1-2 | 90 | 5 |
| I.1-23 | 100 | 20 |
| I.1-23 | 80 | 5 |
| I.1-115 | 100 | 20 |
| I.1-115 | 90 | 5 |
| I.1-176 | 100 | 20 |
| I.1-176 | 90 | 5 |
| I.1-286 | 100 | 20 |
| I.1-286 | 90 | 20 |
| I.1-441 | 100 | 20 |
| I.1-441 | 80 | 5 |
| I.1-442 | 100 | 20 |
| I.1-442 | 100 | 5 |
| I.1-447 | 100 | 20 |
| I.1-447 | 100 | 5 |
| I.1-457 | 100 | 20 |
| I.1-457 | 80 | 5 |
| I.1-471 | 100 | 20 |
| I.1-471 | 90 | 5 |
| I.1-481 | 100 | 20 |
| I.1-481 | 90 | 5 |
| I.1-441 | 100 | 20 |
| I.1-442 | 100 | 20 |
| I.2-442 | 100 | 20 |
| I.5-457 | 80 | 20 |
| I.6-441 | 90 | 20 |
| I.6-441 | 80 | 5 |
| I.14-442 | 100 | 20 |
| I.31-1 | 90 | 20 |
| I.31-23 | 100 | 20 |
| I.31-23 | 80 | 5 |
| I.31-442 | 90 | 20 |
| I.31-442 | 90 | 5 |

TABLE A4

| Compound | Lolium rigidum | Application rate |
|---|---|---|
| Example No. | (efficacy in %) | [g/ha] |
| I.1-2 | 80 | 20 |
| I.1-23 | 80 | 20 |
| I.1-441 | 80 | 20 |
| I.1-471 | 80 | 20 |
| I.1-481 | 80 | 20 |

TABLE A5

| Compound | Setaria viridis | Application rate |
|---|---|---|
| Example No. | (efficacy in %) | [g/ha] |
| I.1-1 | 100 | 20 |
| I.1-1 | 100 | 5 |
| I.1-2 | 100 | 20 |
| I.1-2 | 100 | 5 |
| I.1-23 | 100 | 20 |
| I.1-23 | 100 | 5 |
| I.1-441 | 100 | 20 |
| I.1-441 | 100 | 5 |
| I.1-442 | 100 | 20 |
| I.1-442 | 100 | 5 |
| I.1-447 | 100 | 20 |
| I.1-447 | 100 | 5 |
| I.1-448 | 100 | 20 |
| I.1-448 | 80 | 5 |
| I.1-457 | 100 | 20 |
| I.1-457 | 100 | 5 |
| I.2-442 | 100 | 20 |
| I.2-442 | 100 | 5 |
| I.5-442 | 90 | 5 |
| I.5-457 | 100 | 20 |
| I.5-457 | 100 | 5 |
| I.6-441 | 100 | 20 |
| I.6-441 | 90 | 5 |
| I.14-442 | 100 | 20 |
| I.14-442 | 100 | 5 |

TABLE A6

| Compound | Abutilon theophrasti | Application rate |
|---|---|---|
| Example No. | (efficacy in %) | [g/ha] |
| I.1-1 | 100 | 20 |
| I.1-1 | 100 | 5 |
| I.1-2 | 100 | 20 |
| I.1-2 | 100 | 5 |
| I.1-23 | 100 | 20 |
| I.1-23 | 100 | 5 |
| I.1-115 | 100 | 20 |
| I.1-115 | 90 | 5 |
| I.1-176 | 100 | 20 |
| I.1-176 | 100 | 5 |
| I.1-286 | 100 | 20 |
| I.1-286 | 100 | 5 |
| I.1-441 | 100 | 20 |
| I.1-441 | 100 | 5 |
| I.1-442 | 100 | 20 |
| I.1-442 | 100 | 5 |
| I.1-447 | 100 | 20 |
| I.1-447 | 100 | 5 |
| I.1-448 | 80 | 20 |
| I.1-448 | 80 | 5 |
| I.1-457 | 100 | 20 |
| I.1-457 | 100 | 5 |
| I.1-471 | 100 | 20 |
| I.1-471 | 100 | 5 |
| I.1-481 | 100 | 20 |
| I.1-481 | 100 | 5 |
| I.2-442 | 100 | 20 |
| I.2-442 | 100 | 5 |
| I.5-442 | 100 | 5 |
| I.5-457 | 80 | 20 |
| I.6-441 | 100 | 20 |
| I.6-441 | 100 | 5 |
| I.31-1 | 100 | 20 |
| I.31-1 | 100 | 5 |
| I.14-442 | 100 | 20 |
| I.14-442 | 100 | 5 |
| I.31-23 | 100 | 20 |
| I.31-23 | 100 | 5 |
| I.31-442 | 100 | 20 |
| I.31-442 | 100 | 5 |

TABLE A7

| Compound | Amaranthus retroflexus | Application rate |
|---|---|---|
| Example No. | (efficacy in %) | [g/ha] |
| I.1-1 | 100 | 20 |
| I.1-1 | 100 | 5 |
| I.1-2 | 100 | 20 |
| I.1-2 | 100 | 5 |
| I.1-23 | 100 | 20 |
| I.1-23 | 100 | 5 |
| I.1-115 | 100 | 20 |
| I.1-115 | 100 | 5 |
| I.1-176 | 100 | 20 |
| I.1-176 | 100 | 5 |
| I.1-286 | 100 | 20 |
| I.1-286 | 100 | 5 |
| I.1-441 | 100 | 20 |
| I.1-441 | 100 | 5 |
| I.1-442 | 100 | 20 |
| I.1-442 | 100 | 5 |
| I.1-447 | 100 | 20 |
| I.1-447 | 100 | 5 |
| I.1-448 | 100 | 20 |
| I.1-448 | 100 | 5 |
| I.1-457 | 100 | 20 |
| I.1-457 | 100 | 5 |
| I.1-471 | 100 | 20 |
| I.1-471 | 100 | 5 |
| I.1-481 | 100 | 20 |
| I.1-481 | 100 | 5 |
| I.2-442 | 100 | 20 |
| I.2-442 | 100 | 5 |
| I.5-442 | 100 | 5 |
| I.5-457 | 100 | 20 |
| I.5-457 | 100 | 5 |
| I.6-441 | 100 | 20 |
| I.6-441 | 100 | 5 |
| I.14-442 | 100 | 20 |
| I.14-442 | 100 | 5 |
| I.31-1 | 100 | 20 |
| I.31-1 | 100 | 5 |
| I.31-23 | 100 | 20 |
| I.31-23 | 100 | 5 |
| I.31-442 | 100 | 20 |
| I.31-442 | 100 | 5 |

TABLE A8

| Compound | Matricaria inodora | Application rate |
|---|---|---|
| Example No. | (efficacy in %) | [g/ha] |
| I.1-1 | 100 | 20 |
| I.1-1 | 90 | 5 |
| I.1-2 | 100 | 20 |
| I.1-2 | 100 | 5 |
| I.1-23 | 100 | 20 |
| I.1-23 | 80 | 5 |
| I.1-115 | 100 | 20 |
| I.1-115 | 100 | 5 |
| I.1-176 | 100 | 20 |
| I.1-176 | 100 | 5 |
| I.1-286 | 100 | 20 |
| I.1-286 | 100 | 5 |
| I.1-441 | 100 | 20 |
| I.1-441 | 100 | 5 |
| I.1-442 | 100 | 20 |
| I.1-442 | 90 | 5 |
| I.1-447 | 100 | 20 |
| I.1-447 | 100 | 5 |
| I.1-457 | 90 | 20 |
| I.1-471 | 100 | 20 |
| I.1-471 | 100 | 5 |
| I.1-481 | 100 | 20 |
| I.1-481 | 90 | 5 |
| I.2-442 | 90 | 20 |
| I.5-442 | 80 | 5 |
| I.6-441 | 80 | 20 |
| I.14-442 | 100 | 20 |
| I.14-442 | 100 | 5 |
| I.31-1 | 100 | 20 |
| I.31-1 | 80 | 5 |
| I.31-23 | 100 | 20 |
| I.31-23 | 100 | 5 |
| I.31-442 | 100 | 20 |

TABLE A9

| Compound | Pharbitis purpurea | Application rate |
|---|---|---|
| Example No. | (efficacy in %) | [g/ha] |
| I.1-1 | 100 | 20 |
| I.1-1 | 100 | 5 |
| I.1-2 | 100 | 20 |
| I.1-2 | 100 | 5 |
| I.1-23 | 100 | 20 |
| I.1-23 | 80 | 5 |
| I.1-115 | 100 | 20 |
| I.1-115 | 100 | 5 |
| I.1-176 | 100 | 20 |
| I.1-176 | 100 | 5 |
| I.1-286 | 100 | 5 |
| I.1-441 | 100 | 20 |
| I.1-441 | 100 | 5 |
| I.1-442 | 100 | 5 |
| I.1-448 | 100 | 20 |
| I.5-442 | 100 | 5 |
| I.1-447 | 100 | 20 |
| I.1-447 | 100 | 5 |
| I.1-457 | 100 | 20 |
| I.1-457 | 100 | 5 |
| I.1-471 | 100 | 20 |
| I.1-471 | 100 | 5 |
| I.1-481 | 100 | 20 |
| I.1-481 | 80 | 5 |
| I.5-457 | 90 | 20 |
| I.5-457 | 90 | 5 |
| I.6-441 | 90 | 20 |
| I.6-441 | 80 | 5 |
| I.14-442 | 100 | 20 |
| I.14-442 | 90 | 5 |
| I.31-1 | 100 | 20 |
| I.31-1 | 100 | 5 |
| I.31-23 | 100 | 20 |
| I.31-23 | 100 | 5 |
| I.31-442 | 100 | 20 |
| I.31-442 | 80 | 5 |

TABLE A10

| Compound | Polygonum convolvulus | Application rate |
|---|---|---|
| Example No. | (efficacy in %) | [g/ha] |
| I.1-1 | 100 | 20 |
| I.1-1 | 80 | 5 |
| I.1-2 | 100 | 20 |
| I.1-2 | 100 | 5 |
| I.1-23 | 100 | 20 |
| I.1-23 | 100 | 5 |
| I.1-441 | 100 | 20 |
| I.1-441 | 80 | 5 |
| I.1-442 | 100 | 20 |
| I.1-448 | 100 | 20 |
| I.1-447 | 100 | 20 |
| I.1-447 | 100 | 5 |
| I.1-457 | 100 | 20 |
| I.1-457 | 100 | 5 |
| I.2-442 | 100 | 20 |
| I.2-442 | 100 | 5 |
| I.5-442 | 100 | 5 |
| I.5-457 | 100 | 20 |
| I.5-457 | 90 | 5 |
| I.6-441 | 100 | 20 |
| I.6-441 | 100 | 5 |
| I.14-442 | 100 | 20 |

TABLE A11

| Compound | Stellaria media | Application rate |
|---|---|---|
| Example No. | (efficacy in %) | [g/ha] |
| I.1-441 | 100 | 5 |
| I.1-442 | 100 | 20 |
| I.2-442 | 100 | 20 |
| I.2-442 | 80 | 5 |
| I.5-457 | 100 | 20 |
| I.5-457 | 80 | 5 |
| I.6-441 | 100 | 20 |
| I.6-441 | 100 | 5 |

TABLE A12

| Compound | Viola tricolor | Application rate |
|---|---|---|
| Example No. | (efficacy in %) | [g/ha] |
| I.1-1 | 100 | 20 |
| I.1-1 | 100 | 5 |
| I.1-2 | 100 | 20 |
| I.1-2 | 100 | 5 |
| I.1-23 | 100 | 20 |
| I.1-23 | 100 | 5 |
| I.1-115 | 100 | 20 |
| I.1-115 | 100 | 5 |
| I.1-176 | 100 | 20 |
| I.1-176 | 100 | 5 |
| I.1-286 | 100 | 20 |
| I.1-286 | 100 | 5 |
| I.1-441 | 100 | 20 |
| I.1-441 | 100 | 5 |
| I.1-442 | 100 | 20 |
| I.1-442 | 100 | 5 |
| I.1-447 | 100 | 20 |
| I.1-447 | 100 | 5 |
| I.1-448 | 100 | 20 |
| I.1-448 | 100 | 5 |
| I.1-457 | 100 | 20 |
| I.1-457 | 100 | 5 |
| I.1-471 | 100 | 20 |
| I.1-471 | 100 | 5 |
| I.1-481 | 100 | 20 |
| I.1-481 | 100 | 5 |
| I.2-442 | 100 | 20 |
| I.2-442 | 100 | 5 |
| I.5-442 | 100 | 5 |
| I.5-457 | 100 | 20 |
| I.5-457 | 100 | 5 |
| I.6-441 | 100 | 20 |
| I.6-441 | 100 | 5 |
| I.14-442 | 100 | 20 |
| I.14-442 | 100 | 5 |
| I.31-1 | 100 | 20 |
| I.31-1 | 100 | 5 |
| I.31-23 | 100 | 20 |
| I.31-23 | 100 | 5 |
| I.31-442 | 100 | 20 |
| I.31-442 | 100 | 5 |

TABLE A13

| Compound | Veronica persica | Application rate |
|---|---|---|
| Example No. | (efficacy in %) | [g/ha] |
| I.1-1 | 100 | 20 |
| I.1-1 | 100 | 5 |
| I.1-2 | 100 | 20 |
| I.1-2 | 100 | 5 |
| I.1-23 | 100 | 20 |
| I.1-23 | 100 | 5 |
| I.1-115 | 100 | 20 |
| I.1-115 | 100 | 5 |
| I.1-176 | 100 | 20 |
| I.1-176 | 100 | 5 |
| I.1-286 | 100 | 20 |
| I.1-286 | 100 | 5 |
| I.1-441 | 100 | 20 |
| I.1-441 | 100 | 5 |
| I.1-442 | 100 | 20 |
| I.1-442 | 100 | 5 |
| I.1-447 | 100 | 20 |
| I.1-447 | 100 | 5 |
| I.1-448 | 80 | 20 |
| I.1-448 | 80 | 5 |
| I.1-457 | 100 | 20 |
| I.1-457 | 100 | 5 |
| I.1-471 | 100 | 20 |
| I.1-471 | 100 | 5 |
| I.1-481 | 100 | 20 |
| I.1-481 | 100 | 5 |
| I.2-442 | 100 | 20 |
| I.2-442 | 100 | 5 |
| I.5-442 | 100 | 5 |
| I.5-457 | 100 | 20 |
| I.5-457 | 100 | 5 |
| I.6-441 | 100 | 20 |
| I.6-441 | 100 | 5 |
| I.14-442 | 100 | 20 |
| I.14-442 | 100 | 5 |
| I.31-1 | 100 | 20 |
| I.31-1 | 100 | 5 |
| I.31-23 | 100 | 20 |
| I.31-23 | 100 | 5 |
| I.31-442 | 100 | 20 |
| I.31-442 | 100 | 5 |

TABLE A14

| Compound | Hordeum murinum | Application rate |
|---|---|---|
| Example No. | (efficacy in %) | [g/ha] |
| I.1-441 | 100 | 20 |
| I.1-441 | 80 | 5 |

TABLE A15

| Compound | Avena fatua | Application rate |
|---|---|---|
| Example No. | (efficacy in %) | [g/ha] |
| I.1-1 | 80 | 20 |
| I.1-2 | 80 | 20 |
| I.1-23 | 100 | 20 |
| I.1-176 | 80 | 20 |
| I.1-286 | 80 | 20 |
| I.1-457 | 80 | 20 |
| I.1-471 | 80 | 20 |
| I.14-442 | 80 | 20 |
| I.31-23 | 80 | 20 |

Tables A16 to A20 below show the crop plant compatibilities of selected compounds of the general formula (I) according to Tables I.1 to I.34 at an application rate corresponding to 5 g/ha and less, which were observed in trials by the experimental procedure mentioned above. Here, the observed effects on selected crop plants are stated in comparison to the untreated controls (values in %).

TABLE A16

| Compound | Oryza sativa | Application rate |
|---|---|---|
| Example No. | (efficacy in %) | [g/ha] |
| I.1-1 | 10 | 1.25 |
| I.1-2 | 10 | 5 |
| I.1-2 | 0 | 1.25 |
| I.1-23 | 20 | 1.25 |
| I.1-286 | 0 | 1.25 |
| I.1-447 | 10 | 5 |
| I.1-447 | 0 | 1.25 |
| I.1-448 | 0 | 5 |
| I.1-457 | 0 | 1.25 |
| I.1-481 | 20 | 5 |
| I.1-481 | 10 | 1.25 |
| I.2-442 | 0 | 5 |
| I.31-1 | 10 | 5 |
| I.31-1 | 0 | 1.25 |
| I.31-23 | 20 | 5 |
| I.1-441 | 20 | 5 |
| I.1-441 | 10 | 1.25 |
| I.1-442 | 20 | 5 |
| I.1-442 | 10 | 1.25 |
| I.1-471 | 0 | 1.25 |
| I.2-442 | 0 | 5 |
| I.5-442 | 20 | 5 |
| I.5-457 | 10 | 5 |
| I.5-457 | 0 | 1.25 |
| I.6-441 | 20 | 5 |
| I.14-442 | 20 | 5 |
| I.14-442 | 10 | 1.25 |
| I.31-442 | 10 | 5 |
| I.31-442 | 0 | 1.25 |

TABLE A17

| Compound | Zea mays | Application rate |
|---|---|---|
| Example No. | (efficacy in %) | [g/ha] |
| I.1-23 | 20 | 1.25 |
| I.1-441 | 20 | 1.25 |
| I.1-442 | 20 | 1.25 |
| I.1-448 | 20 | 5 |
| I.1-457 | 20 | 1.25 |
| I.1-471 | 20 | 5 |
| I.2-442 | 20 | 5 |
| I.2-442 | 10 | 1.25 |
| I.5-457 | 20 | 5 |
| I.6-441 | 20 | 5 |
| I.6-441 | 10 | 1.25 |
| I.14-442 | 20 | 1.25 |

TABLE A18

| Compound | Brassica napus | Application rate |
|---|---|---|
| Example No. | (efficacy in %) | [g/ha] |
| I.1-442 | 10 | 1.25 |
| I.5-442 | 20 | 5 |
| I.5-442 | 0 | 1.55 |
| I.5-457 | 10 | 5 |
| I.5-457 | 0 | 1.25 |
| I.6-441 | 20 | 1.25 |

TABLE A19

| Compound | Glycine max | Application rate |
|---|---|---|
| Example No. | (efficacy in %) | [g/ha] |
| I.1-441 | 20 | 5 |
| I.1-442 | 20 | 5 |
| I.1-442 | 10 | 1.25 |
| I.2-442 | 10 | 1.25 |
| I.5-457 | 10 | 5 |
| I.5-457 | 0 | 1.25 |
| I.14-442 | 20 | 1.25 |

TABLE A20

| Compound | Triticum aestivum | Application rate |
|---|---|---|
| Example No. | (efficacy in %) | [g/ha] |
| I.1-1 | 0 | 1.25 |
| I.1-2 | 0 | 1.25 |
| I.1-23 | 20 | 1.25 |
| I.1-115 | 20 | 1.25 |
| I.1-176 | 20 | 1.25 |
| I.1-286 | 20 | 1.25 |
| I.1-441 | 20 | 1.25 |
| I.1-448 | 10 | 5 |
| I.1-448 | 0 | 1.25 |
| I.1-457 | 20 | 1.25 |
| I.2-442 | 20 | 5 |
| I.31-1 | 10 | 5 |
| I.31-23 | 20 | 5 |
| I.31-23 | 10 | 5 |
| I.1-447 | 10 | 1.25 |
| I.1-471 | 20 | 5 |
| I.1-471 | 10 | 1.25 |
| I.1-481 | 20 | 1.25 |
| I.2-442 | 20 | 5 |
| I.5-442 | 20 | 5 |
| I.5-442 | 10 | 1.25 |
| I.5-457 | 20 | 5 |
| I.6-441 | 20 | 5 |
| I.14-442 | 20 | 1.25 |
| I.31-442 | 20 | 5 |
| I.31-442 | 10 | 1.25 |

As the results show, compounds of the general formula (I) according to the invention, in post-emergence treatment, have good herbicidal activity against harmful plants such as Abutilon theophrasti, Alopecurus myosuroides, Amaranthus retroflexus, Avena fatua, Digitaria sanguinalis, Echinochloa crus-galli, Hordeum murinum, Lolium rigidum, Matricaria inodora, Pharbitis purpurea, Polygonum convolvulus, Setaria viridis, Stellaria media, Veronica persica and Viola tricolor at an application rate of between 0.005 and 0.02 kg of active substance or less per hectare, and good crop plant compatibility with organisms such as Oryza sativa, Zea mays, Brassica napus, Glycine max and Triticum aestivum at an application rate of 0.005 kg and less per hectare.

### B. Pre-Emergence Herbicidal Action and Crop Plant Compatibility

Seeds of monocotyledonous and dicotyledonous weed plants and crop plants were placed in plastic or organic planting pots and covered with soil. The compounds of the invention, formulated in the form of wettable powders (WP) or as emulsion concentrates (EC), were then applied to the surface of the covering soil as aqueous suspension or emulsion with addition of 0.5% additive at a water application rate equivalent to 6001/ha (converted). After the treatment, the pots were placed in a greenhouse and kept under good growth conditions for the test plants. After about 3 weeks, the effect of the preparations was scored visually in comparison with untreated controls as percentages. For example, 100% activity=the plants have died, 0% activity=like control plants.

Tables B1 to B11 below show the effects of selected compounds of the general formula (I) according to Tables I.1 to I.34 on various harmful plants and at an application rate corresponding to 80 g/ha and less, which were obtained by the experimental procedure mentioned above.

TABLE B1

| Compound | Alopecurus myosuroides | Application rate |
|---|---|---|
| Example No. | (efficacy in %) | [g/ha] |
| I.1-442 | 90 | 80 |
| I.5-442 | 80 | 80 |

TABLE B2

| Compound | Digitaria sanguinalis | Application rate |
|---|---|---|
| Example No. | (efficacy in %) | [g/ha] |
| I.1-442 | 100 | 80 |
| I.1-442 | 100 | 20 |
| I.5-442 | 80 | 80 |

TABLE B3

| Compound | Echinochloa crus-galli | Application rate |
|---|---|---|
| Example No. | (efficacy in %) | [g/ha] |
| I.1-442 | 100 | 80 |
| I.5-442 | 80 | 80 |

TABLE B4

| Compound | Setaria viridis | Application rate |
|---|---|---|
| Example No. | (efficacy in %) | [g/ha] |
| I.1-442 | 100 | 80 |
| I.1-442 | 90 | 20 |
| I.5-442 | 100 | 80 |

TABLE B5

| Compound | Abutilon theophrasti | Application rate |
|---|---|---|
| Example No. | (efficacy in %) | [g/ha] |
| I.1-442 | 100 | 80 |
| I.5-442 | 100 | 80 |

TABLE B6

| Compound | Amaranthus retroflexus | Application rate |
|---|---|---|
| Example No. | (efficacy in %) | [g/ha] |
| I.1-442 | 100 | 80 |
| I.1-442 | 100 | 20 |
| I.5-442 | 100 | 80 |
| I.5-442 | 100 | 20 |

TABLE B7

| Compound | Matricaria inodora | Application rate |
|---|---|---|
| Example No. | (efficacy in %) | [g/ha] |
| I.1-442 | 100 | 80 |
| I.1-442 | 100 | 20 |
| I.5-442 | 90 | 80 |

TABLE B8

| Compound | Pharbitis purpurea | Application rate |
|---|---|---|
| Example No. | (efficacy in %) | [g/ha] |
| I.1-442 | 100 | 80 |
| I.5-442 | 100 | 80 |

TABLE B9

| Compound | Polygonum convolvulus | Application rate |
|---|---|---|
| Example No. | (efficacy in %) | [g/ha] |
| I.1-442 | 100 | 80 |
| I.1-442 | 90 | 20 |
| I.5-442 | 100 | 80 |
| I.5-442 | 100 | 20 |

TABLE B10

| Compound | Viola tricolor | Application rate |
|---|---|---|
| Example No. | (efficacy in %) | [g/ha] |
| I.1-442 | 100 | 80 |
| I.1-442 | 100 | 20 |
| I.5-442 | 100 | 20 |
| I.5-442 | 100 | 20 |

TABLE B11

| Compound | Veronica persica | Application rate |
|---|---|---|
| Example No. | (efficacy in %) | [g/ha] |
| I.1-442 | 100 | 80 |
| I.1-442 | 80 | 20 |
| I.5-442 | 100 | 80 |
| I.5-442 | 90 | 20 |

TABLE B12

| Compound | Lolium rigidum | Application rate |
|---|---|---|
| Example No. | (efficacy in %) | [g/ha] |
| I.1-442 | 100 | 80 |

Tables B13 to B16 below show the crop plant compatibilities of selected compounds of the general formula (I) according to Tables I.1 to I.34 at an application rate corresponding to 80 g/ha or less, which were observed in trials by the experimental procedure mentioned above. Here, the observed effects on selected crop plants are stated in comparison to the untreated controls (values in %).

TABLE B13

| Compound | Zea mays | Application rate |
|---|---|---|
| Example No. | (efficacy in %) | [g/ha] |
| I.1-442 | 0 | 20 |
| I.5-442 | 20 | 80 |
| I.5-442 | 0 | 20 |

TABLE B14

| Compound | Brassica napus | Application rate |
|---|---|---|
| Example No. | (efficacy in %) | [g/ha] |
| I.1-442 | 0 | 20 |
| I.5-442 | 0 | 20 |

TABLE B15

| Compound | Glycine max | Application rate |
|---|---|---|
| Example No. | (efficacy in %) | [g/ha] |
| I.5-442 | 0 | 80 |

TABLE B16

| Compound | Triticum aestivum | Application rate |
|---|---|---|
| Example No. | (efficacy in %) | [g/ha] |
| I.1-442 | 10 | 20 |
| I.5-442 | 20 | 20 |

As the results show, compounds of the general formula (I) according to the invention, in pre-emergence treatment, have good herbicidal activity against harmful plants, for example against harmful plants such as Abutilon theophrasti, Alopecurus myosuroides, Amaranthus retroflexus, Digitaria sanguinalis, Echinochloa crus-galli, Lolium rigidum, Matricaria inodora, Pharbitis purpurea, Polygonum convolvulus, Setaria viridis, Veronica persica and Viola tricolor at an application rate of 0.08 kg of active substance or less per hectare, and good crop plant compatibility with organisms such as Zea mays, Brassica napus, Glycine max and Triticum aestivum at an application rate of 0.08 kg or less per hectare.

C. Comparative Herbicidal Action and Crop Plant Compatibility of a Compound According to the Invention (I.14-442) with a Structurally Related Compound Known from the Literature (WO2019/101551, No. 16) by the Post-Emergence Method

Seeds of mono- or dicotyledonous weed plants were sown in pots (diameter 7 cm) with sandy loam soil, germinated under optimium growth conditions and cultivated for 21 days.

The compounds, formulated in the form of wettable powders (WP) or as emulsion concentrates (EC), were sprayed onto the green parts of the plants as aqueous suspension or emulsion with addition of 1.5 l/ha Mero and 2 kg/ha AMS at a water application rate of 300 l/ha (converted).

After the test plants had been kept in the greenhouse under optimum growth conditions for 21 days, the activity of the preparations was rated visually in comparison to untreated control plants.

For example, 100% action=the plants have died and

0% action=like control plants.

Tables C₁-C₃ below show the effects of a compound according to the invention (I.14-442) and a structurally related compound from WO2019/101551 (No. 16) on various harmful plants and at an application rate corresponding to 18 g/ha and less, which were obtained by the experimental procedure mentioned above.

TABLE C1

| | Polygonum | Application |
|---|---|---|
| | convolvulus | rate |
| Compound | (efficacy in %) | [g/ha] |
| I.14-442 (according to the invention) | 100 | 18 |
| 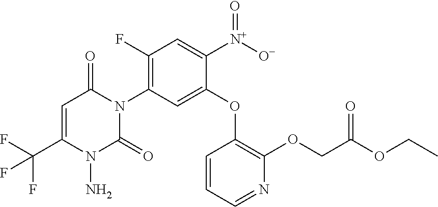 [SMILES: CCOC(=O)COc1ncccc1Oc1cc(-n2c(=O)cc(C(F)(F)F)n(N)c2=O)c(F)cc1[N+](=O)[O-]] | | |
| No. 16 (from WO2019/101551) | 75 | 18 |
| 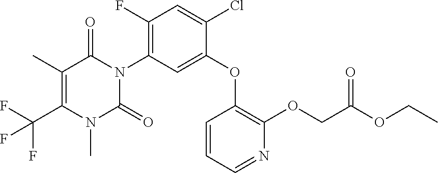 [SMILES: CCOC(=O)COc1ncccc1Oc1cc(-n2c(=O)c(C)c(C(F)(F)F)n(C)c2=O)c(F)cc1Cl] | | |
| I.14-442 (according to the invention) | 99 | 6 |
| No. 16 (from WO2019/101551) | 65 | 6 |

TABLE C2

| | | Application |
|---|---|---|
| | Solanum nigrum | rate |
| Compound | (efficacy in %) | [g/ha] |
| I.14-442 (according to the invention) | 100 | 18 |
| No. 16 (from WO2019/101551) | 80 | 18 |
| I.14-442 (according to the invention) | 100 | 6 |
| No. 16 (from WO2019/101551) | 80 | 6 |

TABLE C3

| | Xanthium | Application |
|---|---|---|
| | strumarium | rate |
| Compound | (efficacy in %) | [g/ha] |
| I.14-442 (according to the invention) | 100 | 18 |
| No. 16 (from WO2019/101551) | 85 | 18 |
| I.14-442 (according to the invention) | 96 | 6 |
| No. 16 (from WO2019/101551) | 50 | 6 |

As shown by the results displayed in Tables C1 to C3, compound 114-442 according to the invention, in comparison to the compound known from the literature (WO2019/101551, No. 16), has a considerably improved herbicidal action against harmful plants such as Polygonum convolvulus, Solanum nigrum and Xanthium strumarium at an application rate of 18 g or less per hectare.

Tables C4 and C5 below show the effects of a compound according to the invention (114-442) and a compound from WO2019/101551 (No. 16) on various crop plants and at an application rate corresponding to 6 g/ha and less, which were obtained by the experimental procedure mentioned above.

TABLE C4

| | | Application |
|---|---|---|
| Compound | Zea mays | rate |
| Example No. | (efficacy in %) | [g/ha] |
| I.14-442 (according to the invention) | 20 | 6 |
| No. 16 (from WO2019/101551) | 50 | 6 |
| I.14-442 (according to the invention) | 20 | 2 |
| No. 16 (from WO2019/101551) | 40 | 2 |

TABLE C5

| | | Application |
|---|---|---|
| Compound | Glycine max | rate |
| Example No. | (efficacy in %) | [g/ha] |
| I.14-442 (according to the invention) | 20 | 6 |
| No. 16 (from WO2019/101551) | 50 | 6 |
| I.14-442 (according to the invention) | 10 | 2 |
| No. 16 (from WO2019/101551) | 40 | 2 |

As shown by the results displayed in Tables C4 and C5, compound 114-442 according to the invention, in comparison to the compound known from the literature (WO2019/101551, No. 16), has a considerably improved crop plant compatibility with organisms such as Zea mays and Glycine max at an application rate of 6 g and less per hectare.

1. A substituted N-phenyl-N-aminouracil of formula (I) or salt thereof, 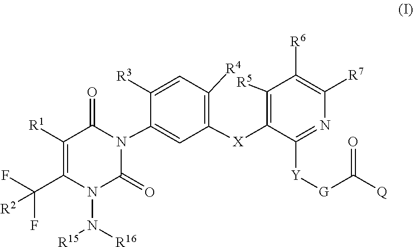
in which
R¹ represents hydrogen, (C₁-C₈)-haloalkyl,
R² represents hydrogen, fluorine, chlorine, bromine, trifluoromethyl, (C₁-C₈)-alkoxy,
R³ represents hydrogen, halogen, (C₁-C₈)-alkoxy,
R⁴ represents halogen, cyano, NO₂, C(O)NH₂, C(S)NH₂, (C₁-C₈)-haloalkyl, (C₂-C₈)-alkynyl,
R⁵, R⁶ and R⁷ independently of one another represent hydrogen, halogen, cyano, (C₁-C₈)-alkyl, (C₁-C₈)-haloalkyl, (C₁-C₈)-alkoxy, (C₁-C₈)-haloalkoxy,
G represents straight-chain or branched (C₁-C₈)-alkylene,
Q represents hydroxy or a radical of formula below 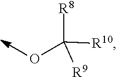
R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, (C₁-C₈)-alkoxy-(C₁-C₈)-alkyl,
R⁹ represents hydrogen or (C₁-C₈)-alkyl,
R¹⁰ represents hydrogen, halogen, cyano, NO₂, (C₁-C₈)-alkyl, (C₁-C₈)-haloalkyl, (C₃-C₈)-cycloalkyl, (C₃-C₈)-cycloalkyl-(C₁-C₈)-alkyl, (C₃-C₈)-halocycloalkyl, (C₃-C₈)-halocycloalkyl-(C₁-C₈)-alkyl, (C₂-C₈)-alkenyl, (C₂-C₈)-alkynyl, aryl, aryl-(C₁-C₈)-alkyl, heteroaryl, heteroaryl-(C₁-C₈)-alkyl, heterocyclyl, heterocyclyl-(C₁-C₈)-alkyl, R¹¹R¹²N—(C₁-C₈)-alkyl, R¹³O—(C₁-C₈)-alkyl, cyano-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, OR¹³, NR¹¹R¹², SR¹⁴, S(O)R¹⁴, SO₂R¹⁴, R¹⁴S—(C₁-C₈)-alkyl, R¹⁴(O)S—(C₁-C₈)-alkyl, R¹⁴O₂S—(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, tris-[(C₁-C₈)-alkyl]silyl, bis-hydroxyboryl-(C₁-C₈)-alkyl, bis-[(C₁-C₈)-alkoxy]boryl-(C₁-C₈)-alkyl, tetramethyl-1,3,2-dioxaborolan-2-yl, tetramethyl-1,3,2-dioxaborolan-2-yl-(C₁-C₈)-alkyl, nitro-(C₁-C₈)-alkyl, C(O)OR¹³, C(O)R¹³, C(O)NR¹¹R¹², R¹³O(O)C—(C₁-C₈)-alkyl, R¹¹R¹²N(O)C—(C₁-C₈)-alkyl, bis-(C₁-C₈)-alkoxy-(C₁-C₈)-alkyl, or
R⁸ and R¹⁰ together with the carbon atom to which they are attached form a fully saturated or partially saturated 3- to 10-membered monocyclic or bicyclic ring optionally interrupted by heteroatoms and optionally having further substitution,
R¹¹ and R¹² are identical or different and independently of one another represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, COR¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, heterocyclyl-(C₁-C₈)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution,
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl-amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₁₀-alkyl, aryloxy-(C₁-C₈)-alkyl, heteroaryloxy-(C₁-C₈)-alkyl, (C₁-C₈)-alkoxycarbonyl,
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkyl-amino, aryl-(C₁-C₈)-amino, aryl-(C₁-C₆)-alkyl-amino, aryl-[(C₁-C₈)-alkyl]amino, (C₃-C₈)-cycloalkyl-amino, (C₃-C₈)-cycloalkyl-[(C₁-C₈)-alkyl]amino; N-azetidinyl, N-pyrrolidinyl, N-piperidinyl, N-morpholinyl,
R¹⁵ and R¹⁶ independently of one another represent hydrogen, (C₁-C₈)-alkyl, (C₂-C₈)-alkenyl, C(O)R¹³, C(O)OR¹³, C(O)NR¹¹R¹², SO₂R¹⁴, or
R¹⁵ and R¹⁶ together with the nitrogen atom to which they are attached form an imino group which is optionally substituted further by hydrogen, (C₁-C₈)-alkyl, aryl-(C₁-C₈)-alkyl, (C₃-C₈)-cycloalkyl, aryl, heteroaryl, heterocyclyl, (C₁-C₈)-alkoxycarbonyl-(C₁-C₈)-alkyl, aryl-(C₁-C₈)-alkoxycarbonyl-(C₁-C₈)-alkyl and
X and Y independently of one another represent O (oxygen) or S (sulfur).

2. The compound of formula (I) as claimed in claim 1 and/or salt thereof, wherein
R¹ represents hydrogen,
R² represents hydrogen, fluorine, chlorine, bromine, trifluoromethyl, (C₁-C₆)-alkoxy,
R³ represents hydrogen, halogen, (C₁-C₆)-alkoxy,
R⁴ represents halogen, cyano, NO₂, C(O)NH₂, C(S)NH₂, (C₁-C₆)-haloalkyl, (C₂-C₆)-alkynyl,
R⁵, R⁶ and R⁷ independently of one another represent hydrogen, halogen, cyano, (C₁-C₆)-alkyl, (C₁-C₆)-haloalkyl, (C₁-C₆)-alkoxy, (C₁-C₆)-haloalkoxy,
G represents straight-chain or branched (C₁-C₆)-alkylene,
Q represents hydroxy or a radical of formula below 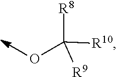
R⁸ represents hydrogen, (C₁-C₆)-alkyl, (C₁-C₆)-haloalkyl, aryl, aryl-(C₁-C₆)-alkyl, heteroaryl, (C₂-C₆)-alkynyl, (C₂-C₆)-alkenyl, C(O)R¹³, C(O)OR¹³, (C₁-C₆)-alkoxy-(C₁-C₆)-alkyl,
R⁹ represents hydrogen or (C₁-C₄)-alkyl,
R¹⁰ represents hydrogen, halogen, cyano, NO₂, (C₁-C₆)-alkyl, (C₁-C₆)-haloalkyl, (C₃-C₆)-cycloalkyl, (C₃-C₆)-cycloalkyl-(C₁-C₆)-alkyl, (C₃-C₆)-halocycloalkyl, (C₃-C₆)-halocycloalkyl-(C₁-C₆)-alkyl, (C₂-C₆)-alkenyl, (C₂-C₆)-alkynyl, aryl, aryl-(C₁-C₆)-alkyl, heteroaryl, heteroaryl-(C₁-C₆)-alkyl, heterocyclyl, heterocyclyl-(C₁-C₆)-alkyl, R¹¹R¹²N—(C₁-C₆)-alkyl, R¹³O—(C₁-C₆)-alkyl, cyano-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, OR¹³, NR¹¹R¹², SR¹⁴, S(O)R¹⁴, SO₂R¹⁴, R¹⁴S—(C₁-C₆)-alkyl, R¹⁴(O)S—(C₁-C₆)-alkyl, R¹⁴O₂S—(C₁-C₆)-alkyl, tris-[(C₁-C₆)-alkyl]silyl-(C₁-C₆)-alkyl, bis-[(C₁-C₆)-alkyl](aryl)silyl(C₁-C₆)-alkyl, [(C₁-C₆)-alkyl]-bis-(aryl)silyl-(C₁-C₆)-alkyl, tris-[(C₁-C₆)-alkyl]silyl, bis-hydroxyboryl-(C₁-C₆)-alkyl, bis-[(C₁-C₆)-alkoxy]boryl-(C₁-C₆)-alkyl, tetramethyl-1,3,2-dioxaborolan-2-yl, tetramethyl-1,3,2-dioxaborolan-2-yl-(C₁-C₆)-alkyl, nitro-(C₁-C₆)-alkyl, C(O)OR¹³, C(O)R¹³, C(O)NR¹¹R¹², R¹³O(O)C—(C₁-C₆)-alkyl, R¹¹R¹²N(O)C—(C₁-C₆)-alkyl, bis-(C₁-C₆)-alkoxy-(C₁-C₆)-alkyl, or
R⁸ and R¹⁰ together with the carbon atom to which they are attached form a fully saturated or partially saturated 3- to 10-membered monocyclic or bicyclic ring optionally interrupted by heteroatoms and optionally having further substitution,
R¹¹ and R¹² are identical or different and independently of one another represent hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, COR¹³, SO₂R¹⁴, heterocyclyl, (C₁-C₆)-alkoxycarbonyl, bis-[(C₁-C₆)-alkyl]aminocarbonyl-(C₁-C₆)-alkyl, (C₁-C₆)-alkyl-aminocarbonyl-(C₁-C₆)-alkyl, aryl-(C₁-C₆)-alkyl-aminocarbonyl-(C₁-C₆)-alkyl, aryl-(C₁-C₆)-alkoxycarbonyl, heteroaryl-(C₁-C₆)-alkoxycarbonyl, (C₂-C₆)-alkenyloxycarbonyl, (C₂-C₆)-alkynyloxycarbonyl, heterocyclyl-(C₁-C₆)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution,
R¹³ represents hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkoxy-(C₁-C₆)-haloalkyl, (C₁-C₆)-alkoxy-(C₁-C₆)-alkoxy-(C₁-C₆)-alkyl, (C₁-C₆)-alkoxy-(C₁-C₆)-alkoxy-(C₁-C₆)-alkoxy-(C₁-C₆)-alkyl, (C₁-C₆)-alkoxy-(C₁-C₆)-alkoxy-(C₁-C₆)-alkoxy-(C₁-C₆)-alkoxy-(C₁-C₆)-alkyl, aryl, aryl-(C₁-C₆)-alkyl, aryl-(C₁-C₆)-alkoxy-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, bis-[(C₁-C₆)-alkyl]aminocarbonyl-(C₁-C₆)-alkyl, (C₁-C₆)-alkyl-aminocarbonyl-(C₁-C₆)-alkyl, aryl-(C₁-C₆)-alkyl-aminocarbonyl-(C₁-C₆)-alkyl, bis-[(C₁-C₆)-alkyl]amino-(C₂-C₄)-alkyl, (C₁-C₆)-alkyl-amino-(C₂-C₄)-alkyl, aryl-(C₁-C₆)-alkyl-amino-(C₂-C₄)-alkyl, R¹⁴S—(C₁-C₆)-alkyl, R¹⁴(O)S—(C₁-C₆)-alkyl, R¹⁴O₂S—(C₁-C₆)-alkyl, hydroxycarbonyl-(C₁-C₆)-alkyl, heterocyclyl, heterocyclyl-(C₁-C₆)-alkyl, tris-[(C₁-C₆)-alkyl]silyl-(C₁-C₆)-alkyl, bis-[(C₁-C₆)-alkyl](aryl)silyl(C₁-C₆)-alkyl, [(C₁-C₆)-alkyl]-bis-(aryl)silyl-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, aryloxy-(C₁-C₆)-alkyl, heteroaryloxy-(C₁-C₆)-alkyl, (C₁-C₆)-alkoxycarbonyl,
R¹⁴ represents hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, heterocyclyl-(C₁-C₆)-alkyl, (C₃-C₆)-cycloalkyl-(C₁-C₆)-alkyl, (C₄-C₁₀)-cycloalkenyl-(C₁-C₆)-alkyl, bis-[(C₁-C₆)-alkyl]amino, (C₁-C₆)-alkyl-amino, aryl-(C₁-C₆)-amino, aryl-(C₁-C₂)-alkyl-amino, aryl-[(C₁-C₆)-alkyl]amino, (C₃-C₆)-cycloalkyl-amino, (C₃-C₆)-cycloalkyl-[(C₁-C₆)-alkyl]amino; N-azetidinyl, N-pyrrolidinyl, N-piperidinyl, N-morpholinyl,
R¹⁵ and R¹⁶ independently of one another represent hydrogen, (C₁-C₆)-alkyl, (C₂-C₆)-alkenyl, C(O)R¹³, C(O)OR¹³, C(O)NR¹¹R¹², SO₂R¹⁴, or
R¹⁵ and R¹⁶ together with the nitrogen atom to which they are attached form an imino group which is optionally substituted further by hydrogen, (C₁-C₆)-alkyl, aryl-(C₁-C₆)-alkyl, (C₃-C₆)-cycloalkyl, aryl, heteroaryl, heterocyclyl, (C₁-C₆)-alkoxycarbonyl-(C₁-C₆)-alkyl, aryl-(C₁-C₆)-alkoxycarbonyl-(C₁-C₆)-alkyl and
X and Y independently of one another represent O (oxygen) or S (sulfur).

3. The compound of formula (I) as claimed in claim 1 and/or salt thereof, wherein
R¹ represents hydrogen,
R² represents hydrogen, fluorine, chlorine, bromine, trifluoromethyl, methoxy, ethoxy, prop-1-yloxy, but-1-yloxy,
R³ represents hydrogen, fluorine, chlorine, bromine, methoxy, ethoxy, prop-1-yloxy, prop-2-yloxy, but-1-yloxy, but-2-yloxy, 2-methylprop-1-yloxy, 1,1-dimethyleth-1-yloxy,
R⁴ represents fluorine, chlorine, bromine, cyano, NO₂, C(O)NH₂, C(S)NH₂, trifluoromethyl, difluoromethyl, pentafluoroethyl, ethynyl, propyn-1-yl, 1-butyn-1-yl, pentyn-1-yl, hexyn-1-yl,
R⁵, R⁶ and R⁷ independently of one another represent hydrogen, fluorine, chlorine, bromine, iodine, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, difluoromethyl, pentafluoroethyl, 2,2-difluoroethyl, 2,2,2-trifluoroethyl, methoxy, ethoxy, prop-1-yloxy, prop-2-yloxy, but-1-yloxy, but-2-yloxy, 2-methylprop-1-yloxy, 1,1-dimethyleth-1-yloxy, difluoromethoxy, trifluoromethoxy, pentafluoroethoxy, 2,2-difluoroethoxy, 2,2,2-trifluoroethoxy,
G represents methylene, (methyl)methylene, (ethyl)methylene, (prop-1-yl)methylene, (prop-2-yl)methylene, (but-1-yl)methylene, (but-2-yl)methylene, (pent-1-yl)methylene, (pent-2-yl)methylene, (pent-3-yl)methylene, (dimethyl)methylene, (diethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, 1,1,2-trimethylethyl-1-ene, 1,2,2-trimethylethyl-1-ene, 1,1,2,2-tetramethylethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene, 1-methylpentyl-1-ene, 2-methylpentyl-1-ene, 3-methylpentyl-1-ene, 4-methylpentyl-1-ene, 1,1-dimethylbutyl-1-ene, 1,2-dimethylbutyl-1-ene, 1,3-dimethylbutyl-1-ene, 2,2-dimethylbutyl-1-ene, 2,3-dimethylbutyl-1-ene, 3,3-dimethylbutyl-1-ene, 1-ethylbutyl-1-ene, 2-ethylbutyl-1-ene, 1,1,2-trimethylpropyl-1-ene, 1,2,2-trimethylpropyl-1-ene, 1-ethyl-1-methylpropyl-1-ene, 1-ethyl-2-methylpropyl-1-ene,
R¹⁵ and R¹⁶ independently of one another represent hydrogen, methyl, ethyl, methylcarbonyl, ethylcarbonyl, methoxycarbonyl, ethoxycarbonyl, tert-butyloxycarbonyl,
X and Y independently of one another represent O (oxygen) or S (sulfur)
and Q represents one of the moieties Q-1 to Q-486 specifically mentioned below:

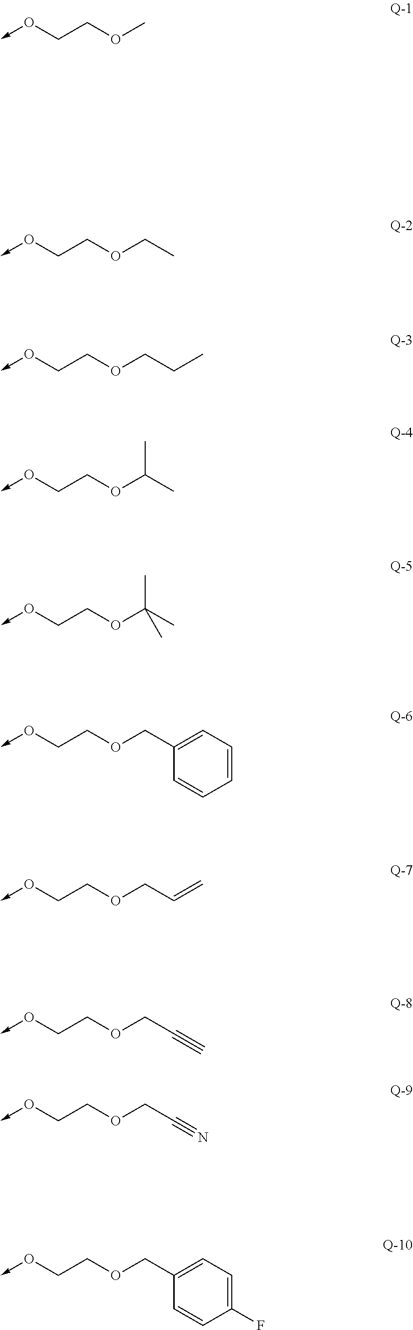

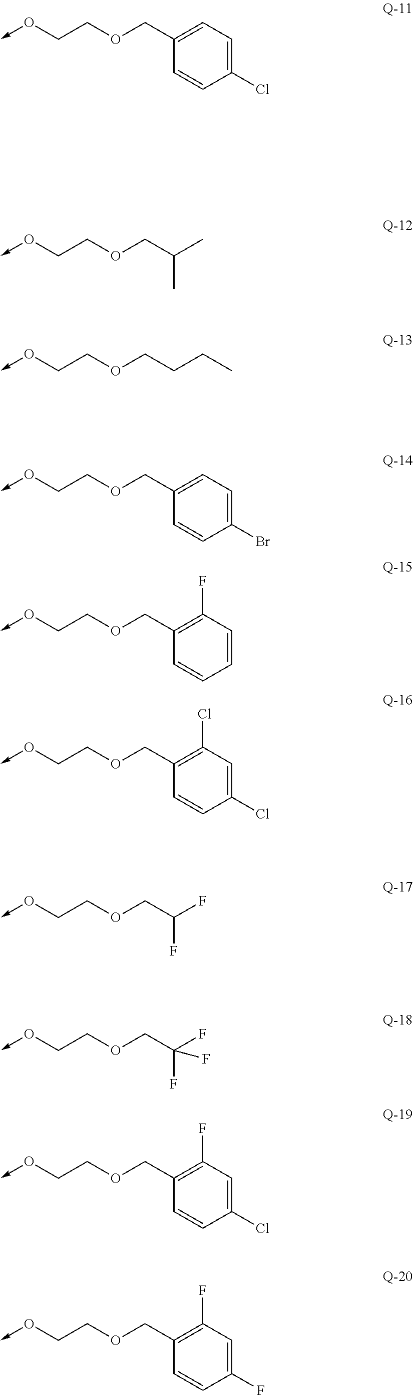

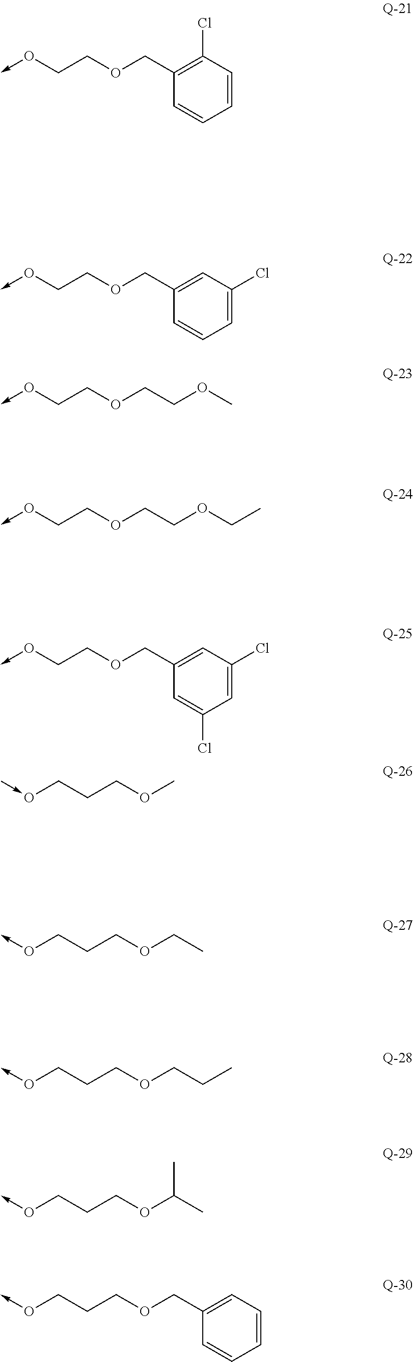

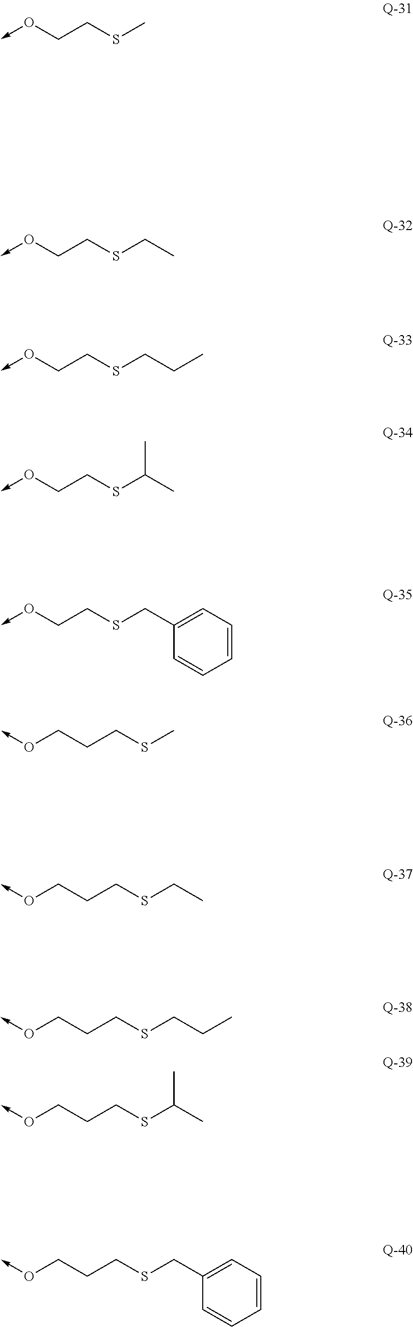

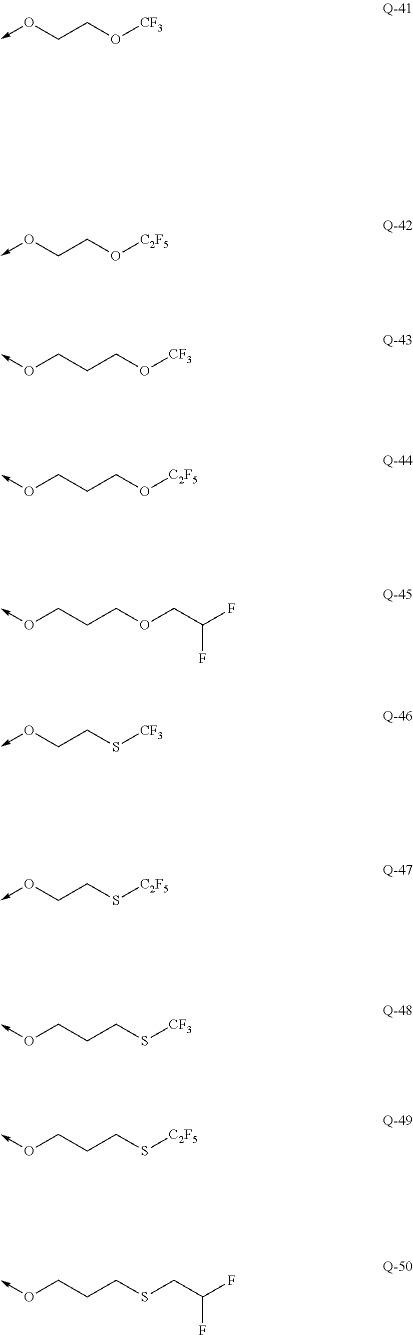

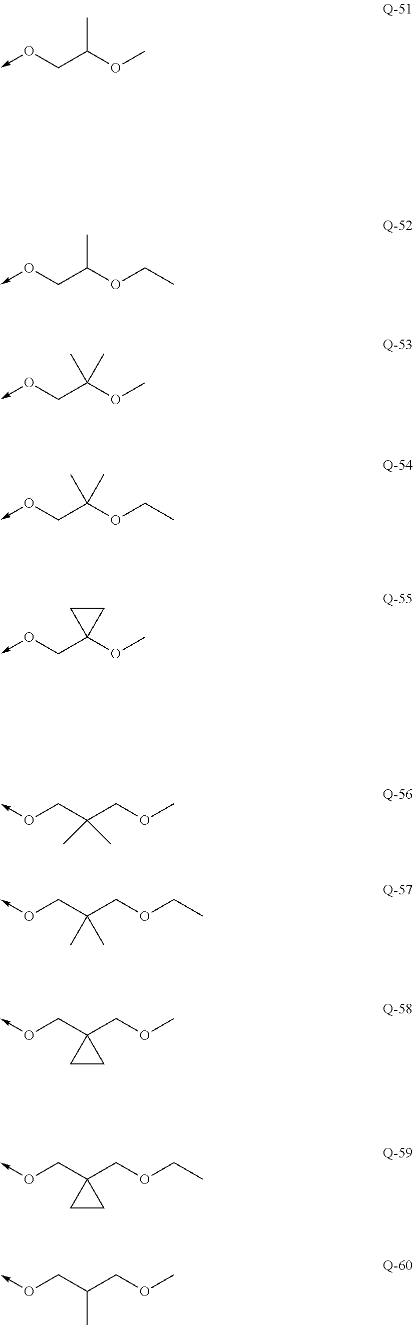

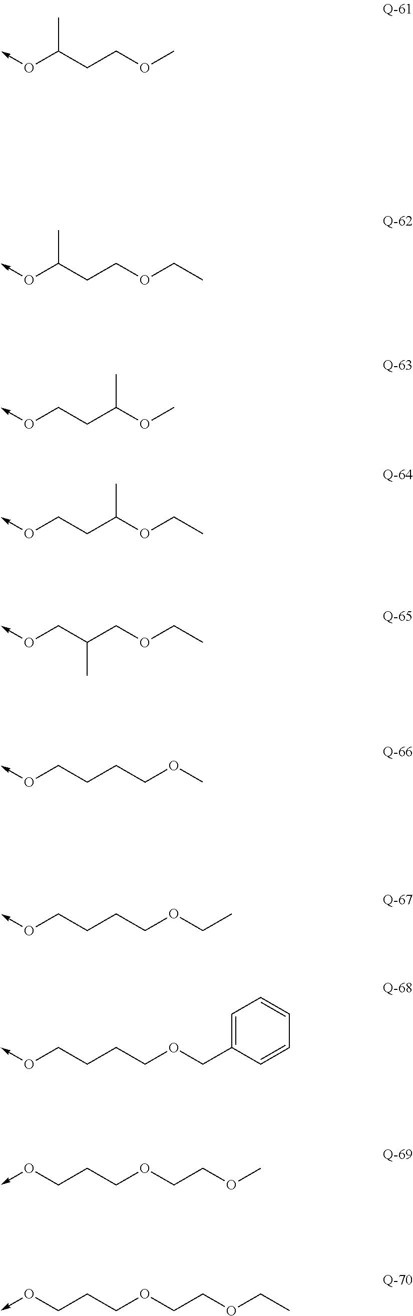

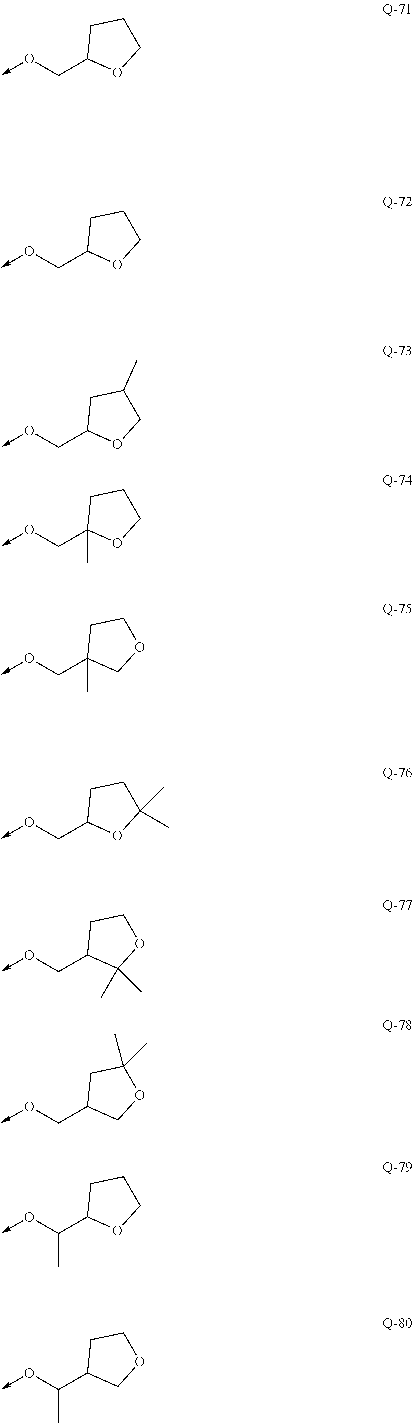

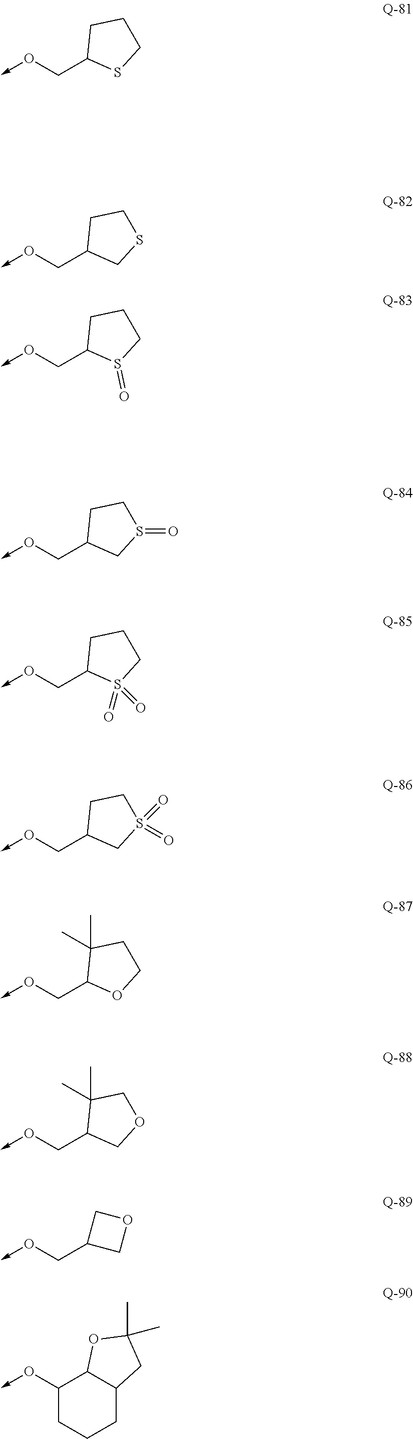

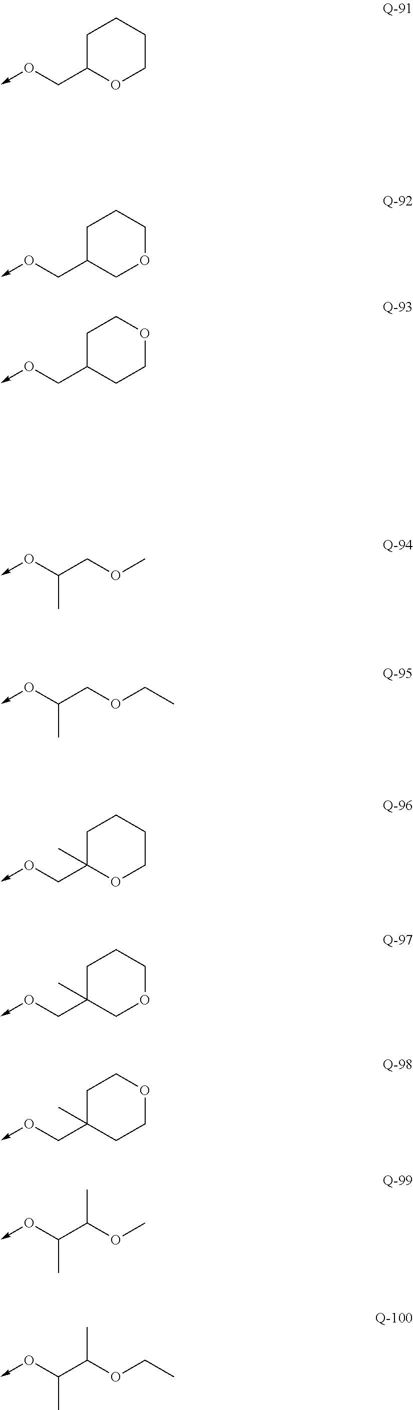

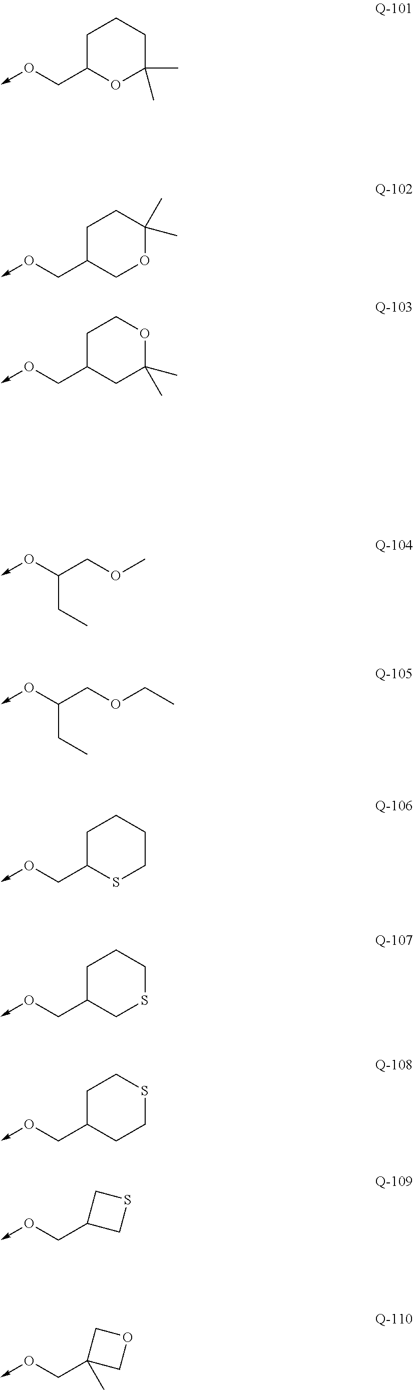

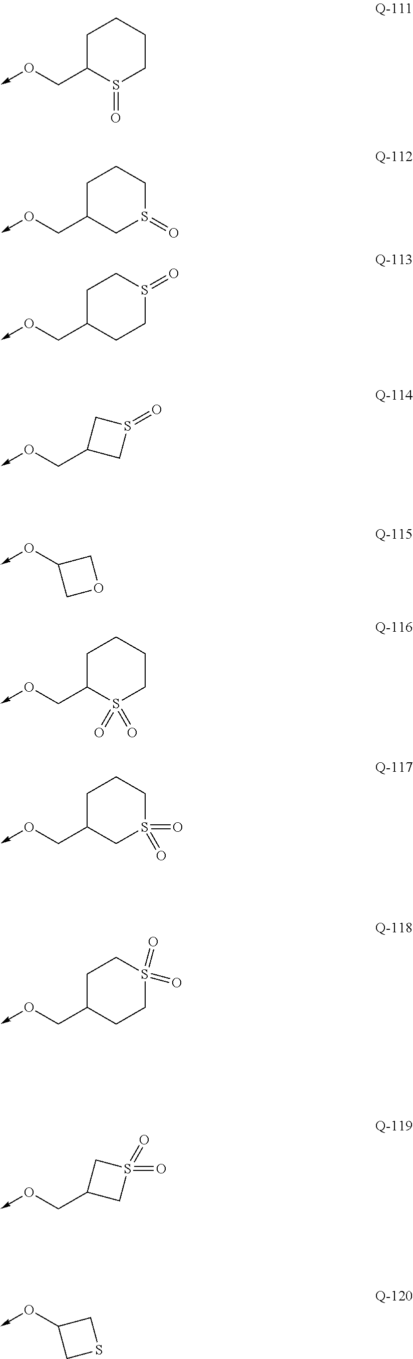

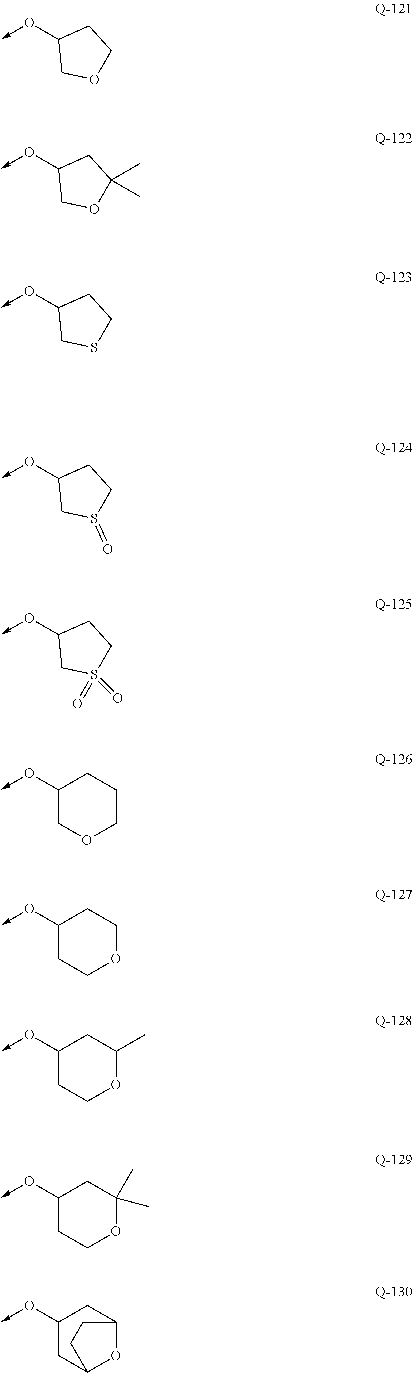

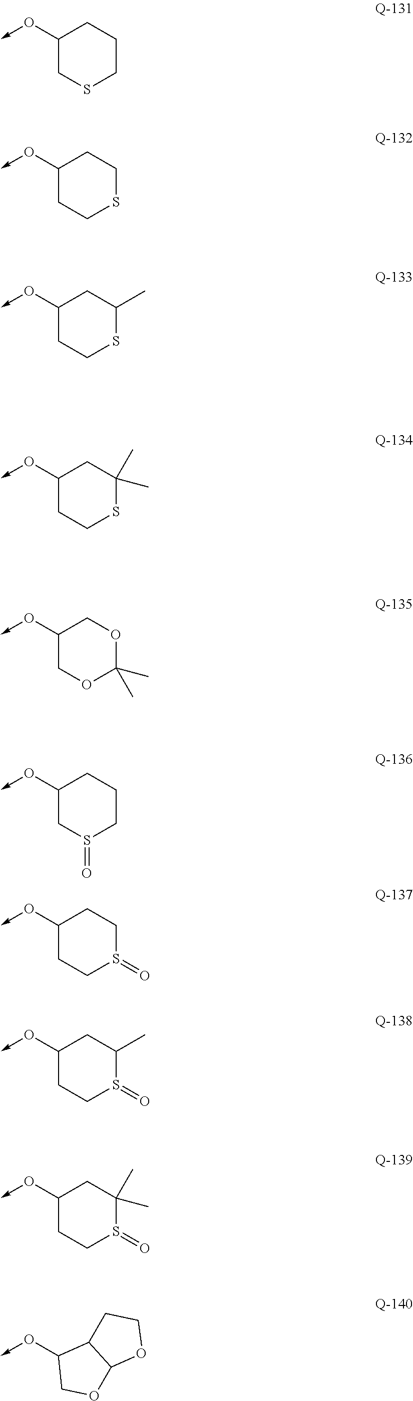

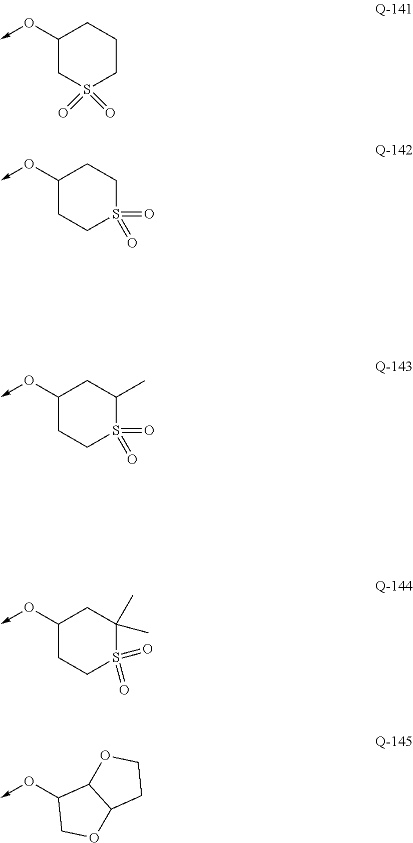

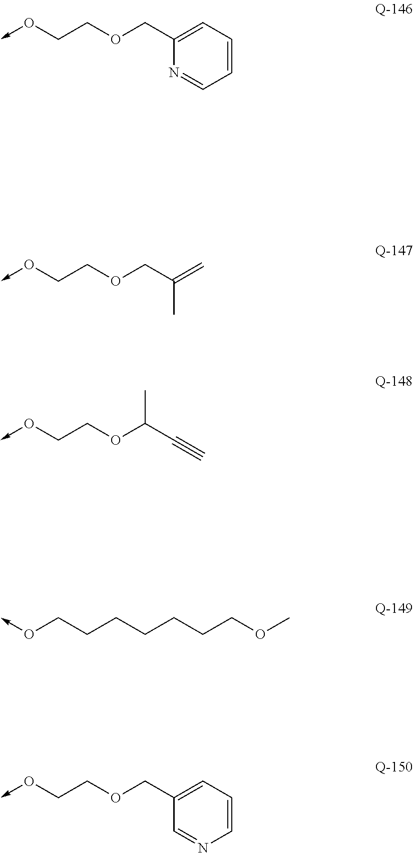

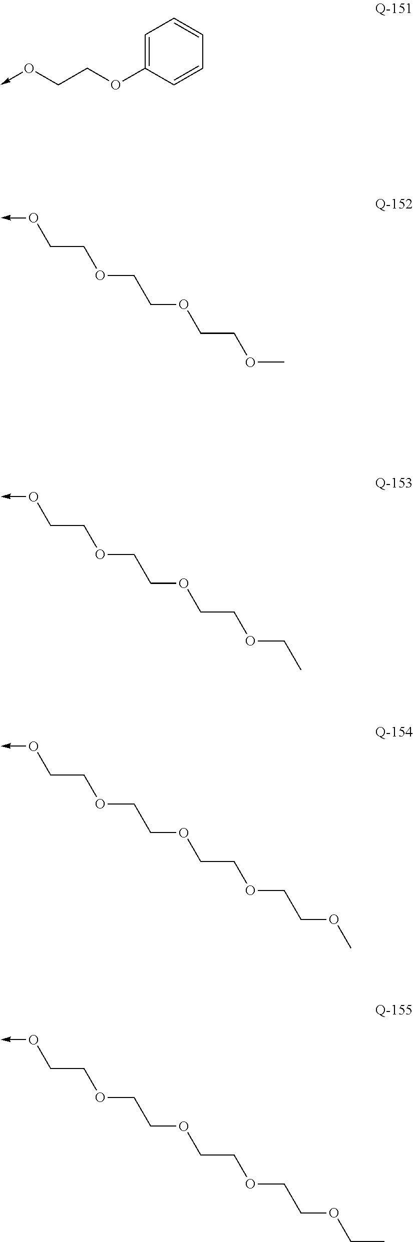

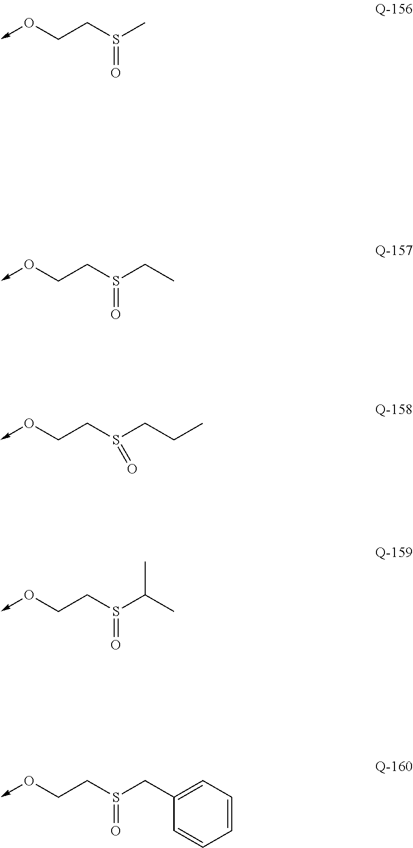

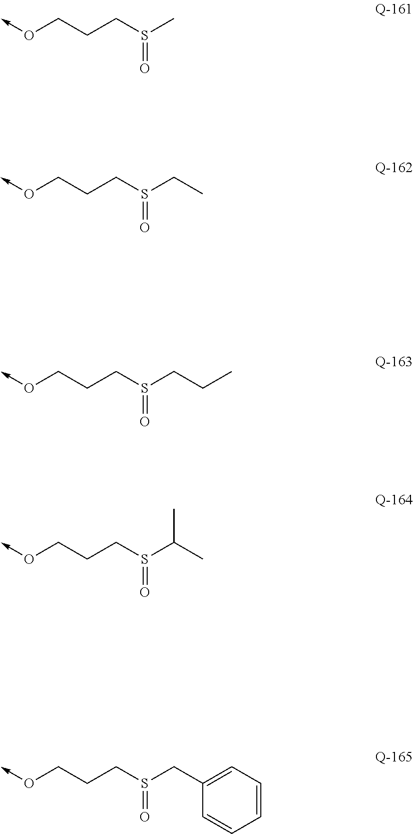

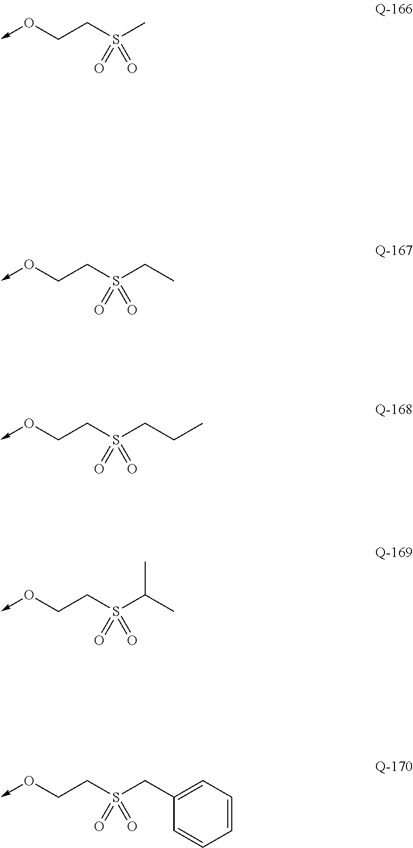

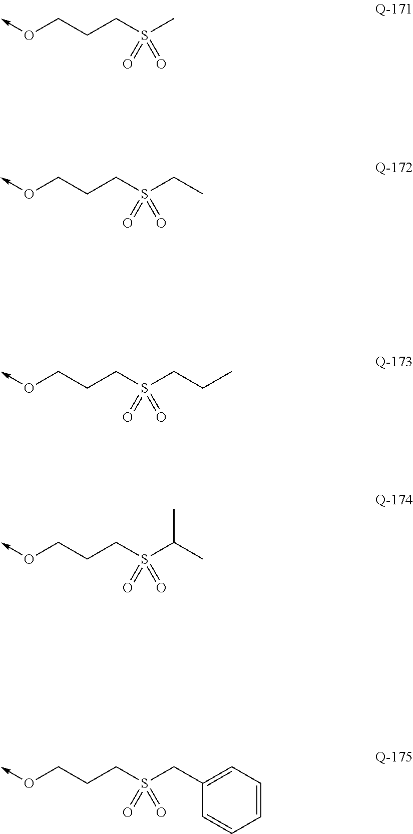

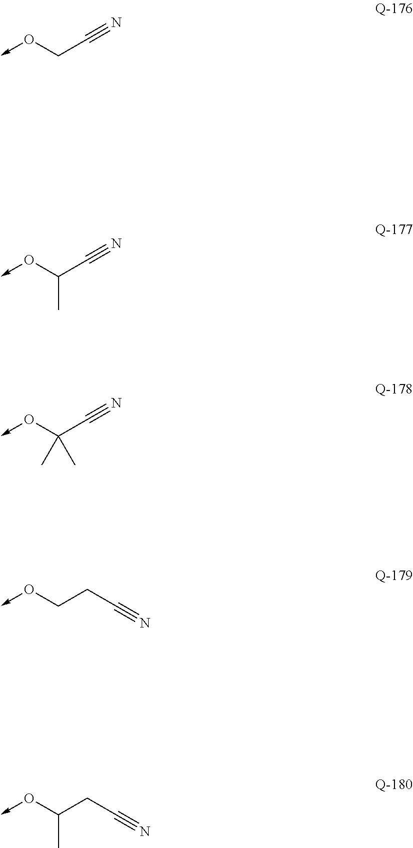

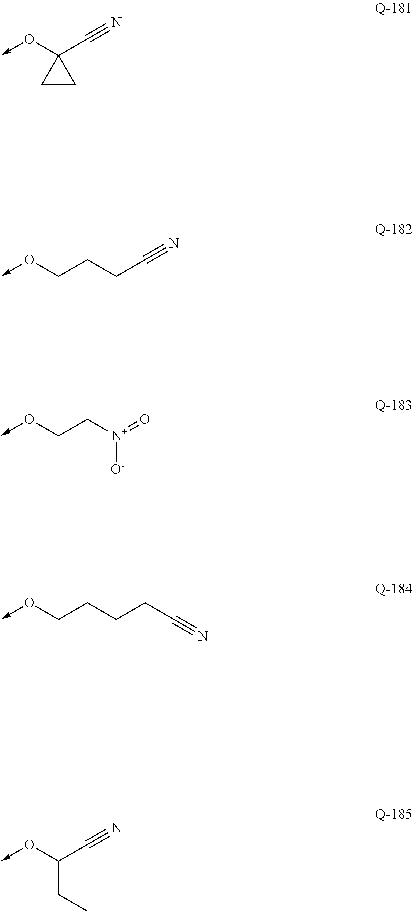

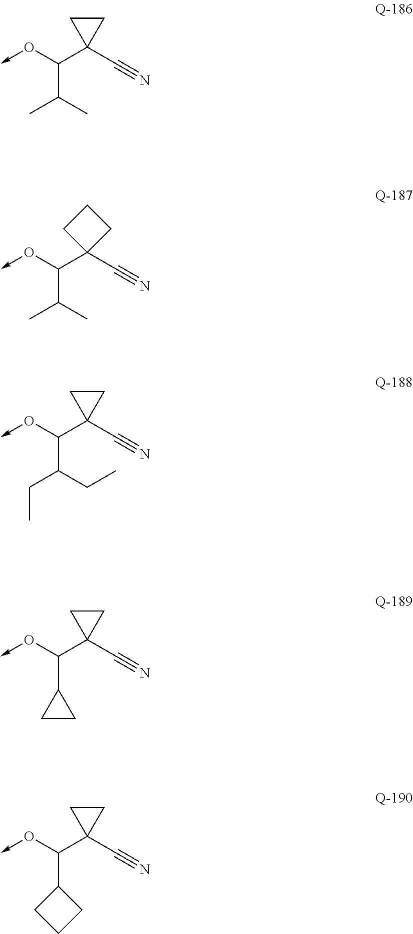

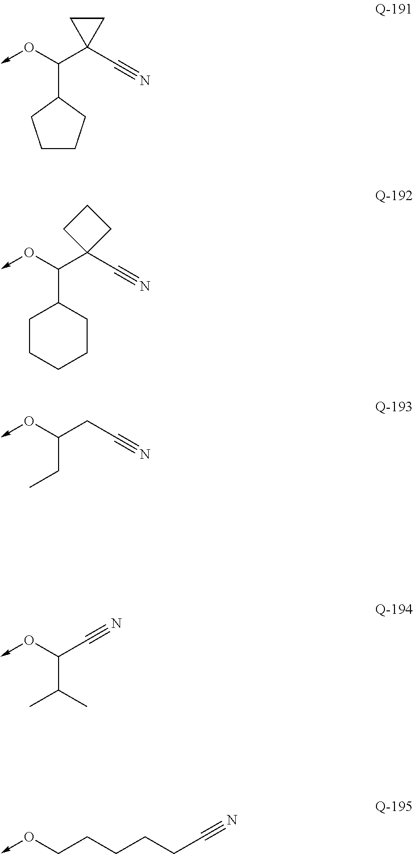

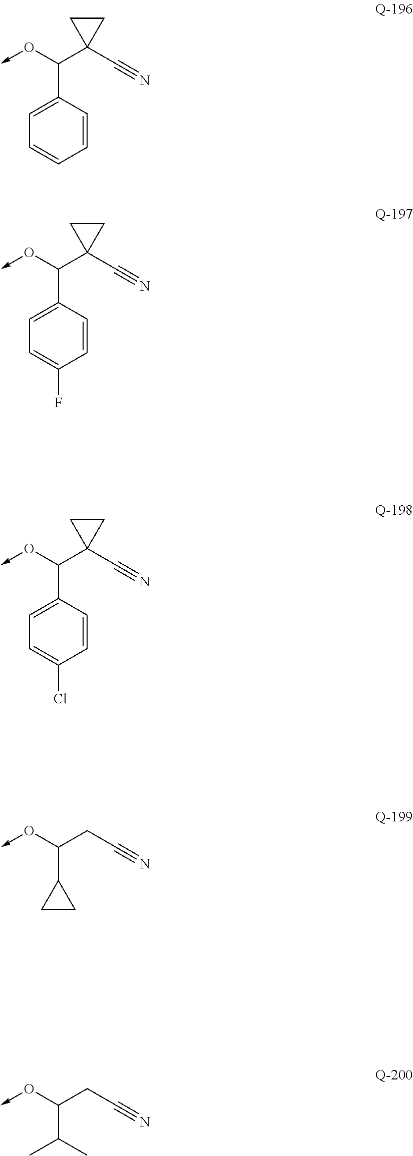

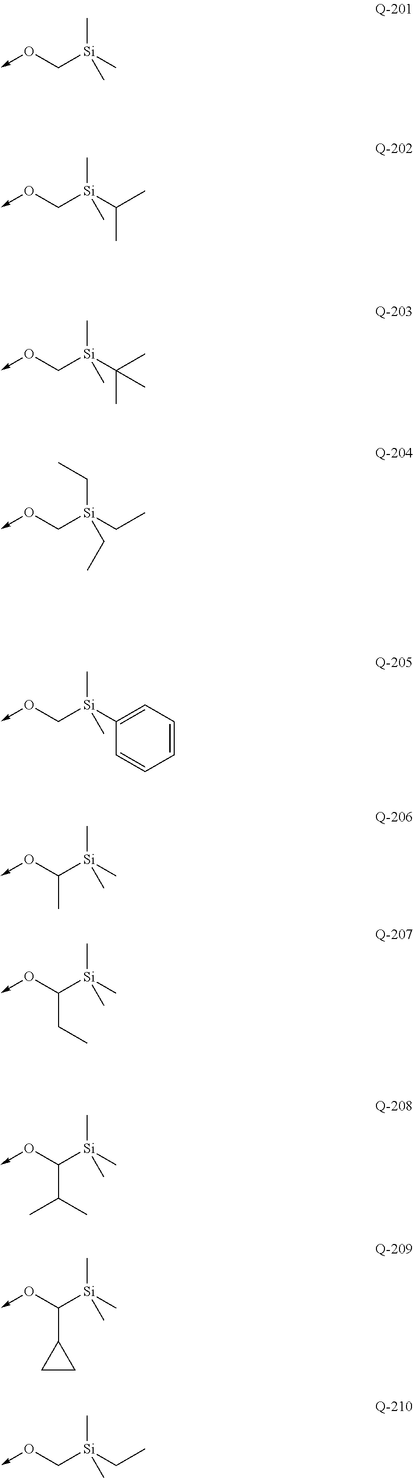

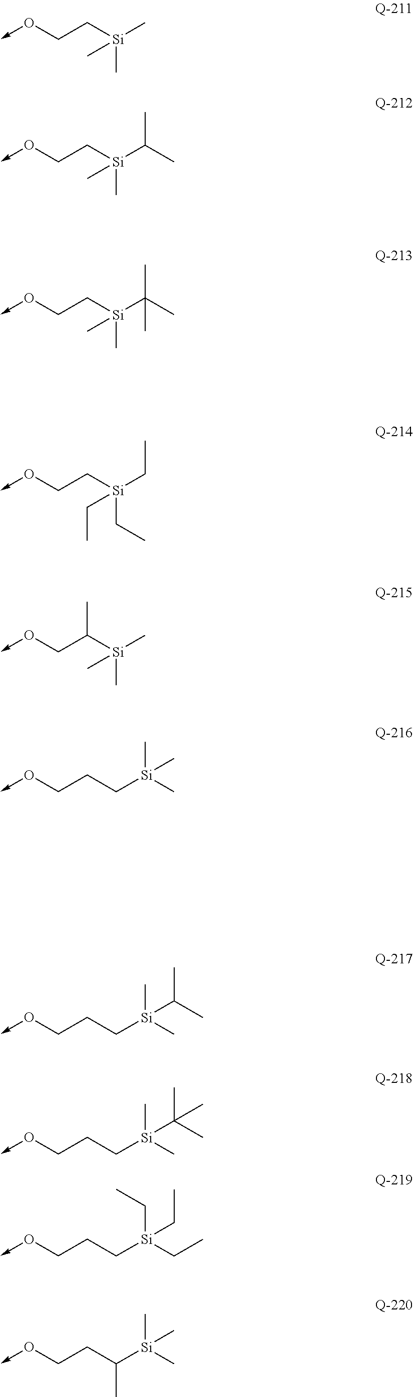

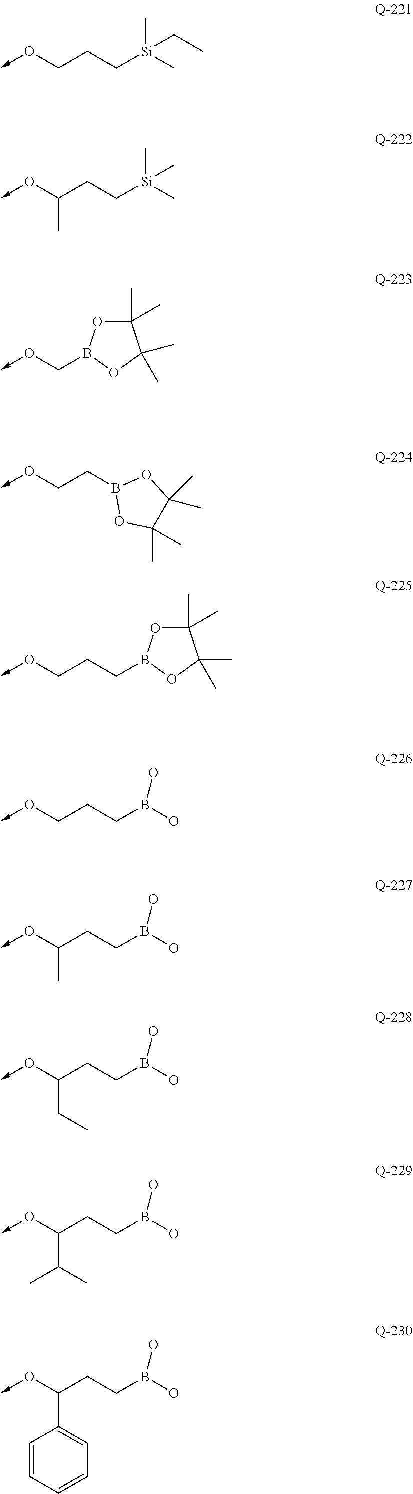

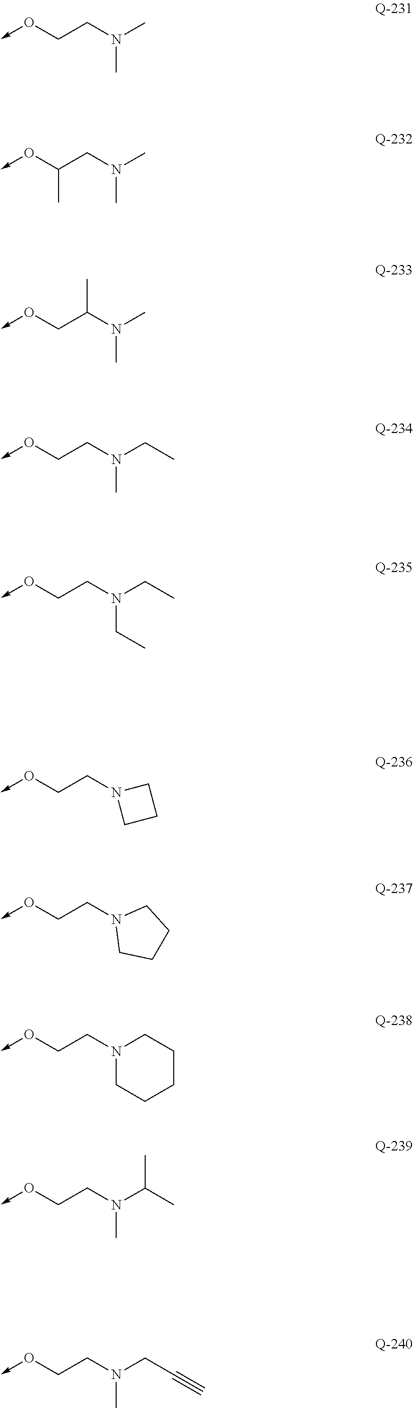

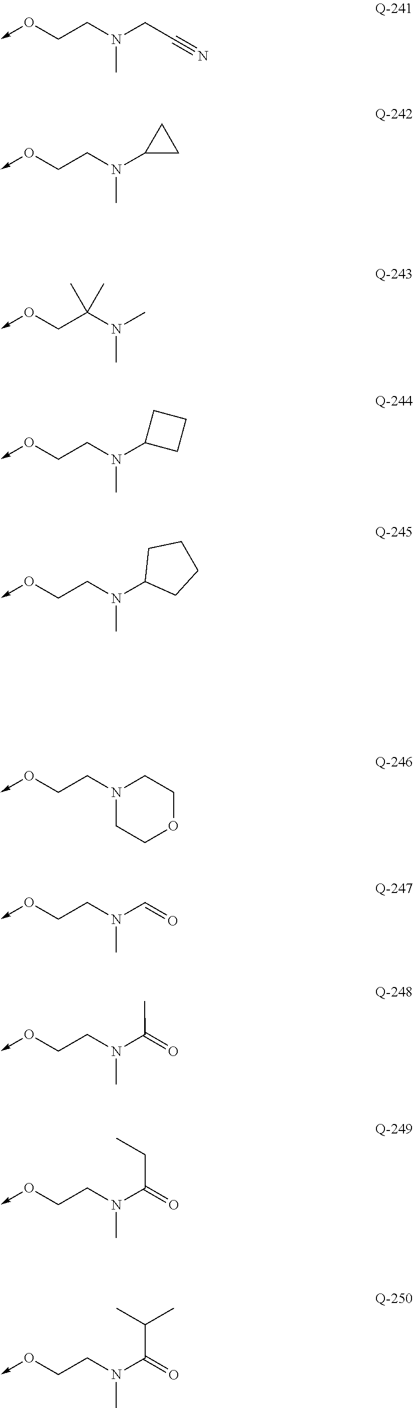

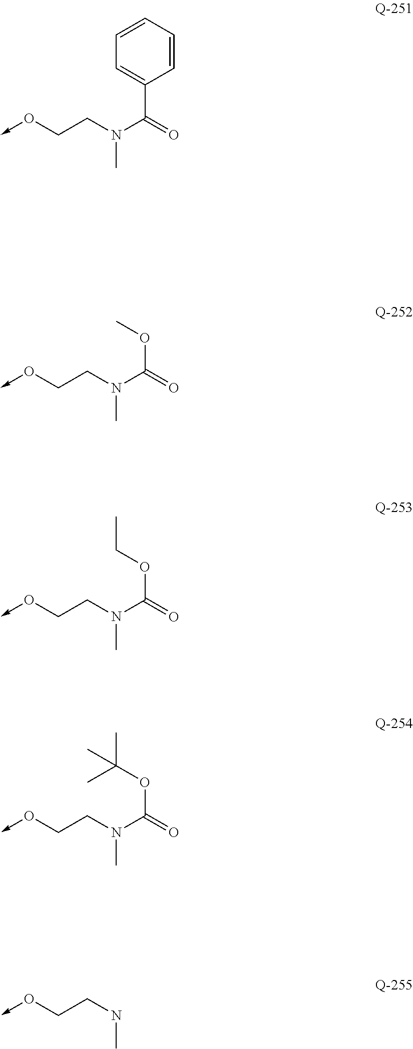

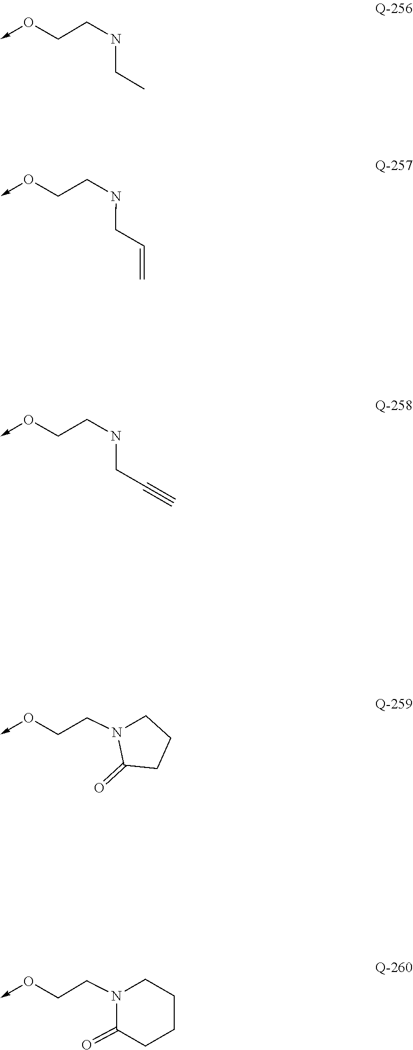

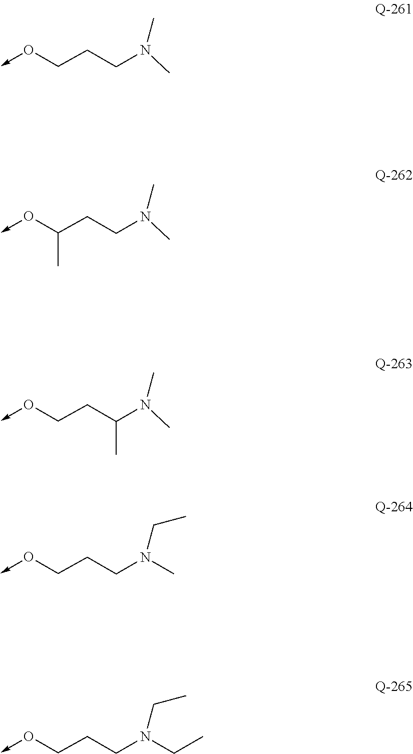

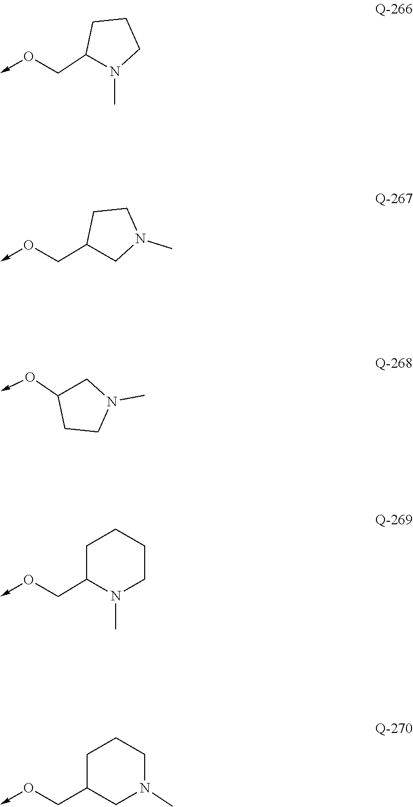

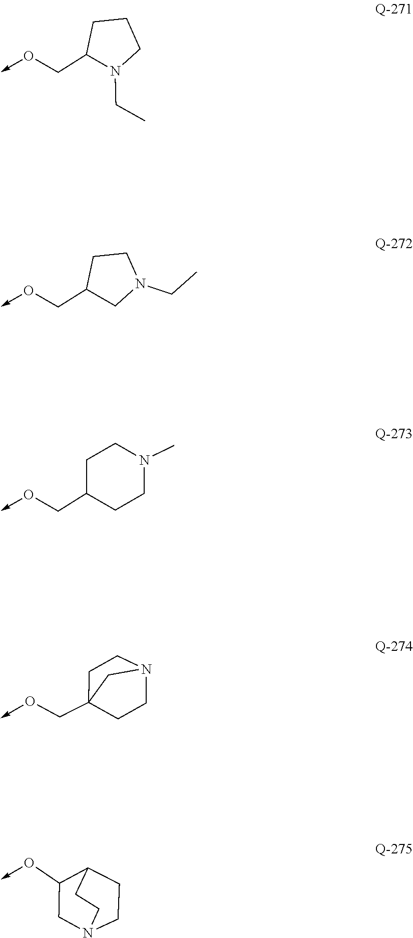

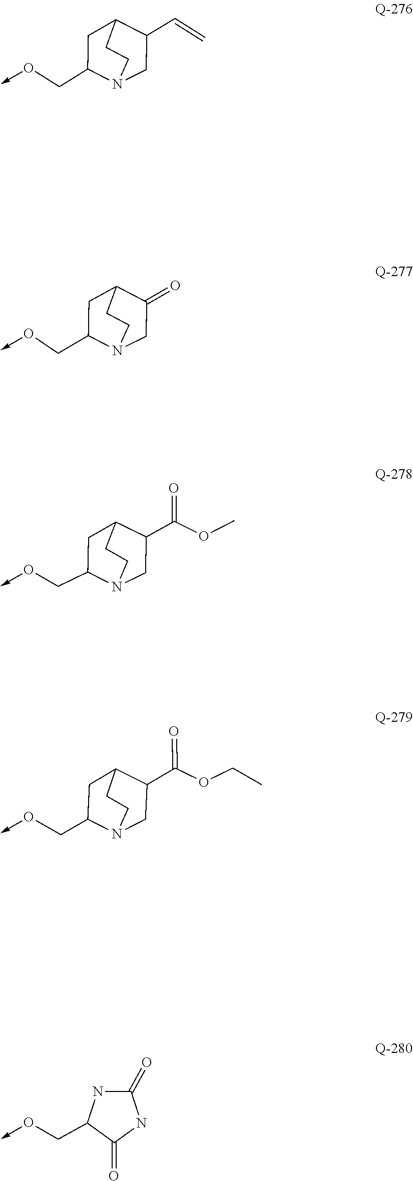

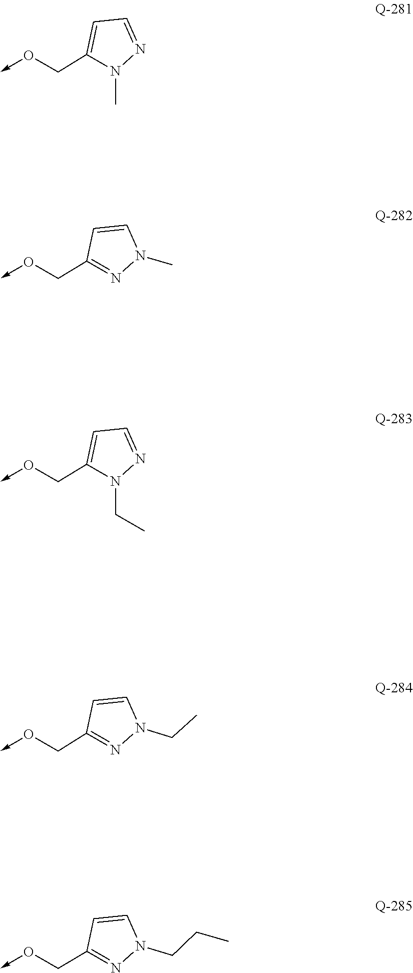

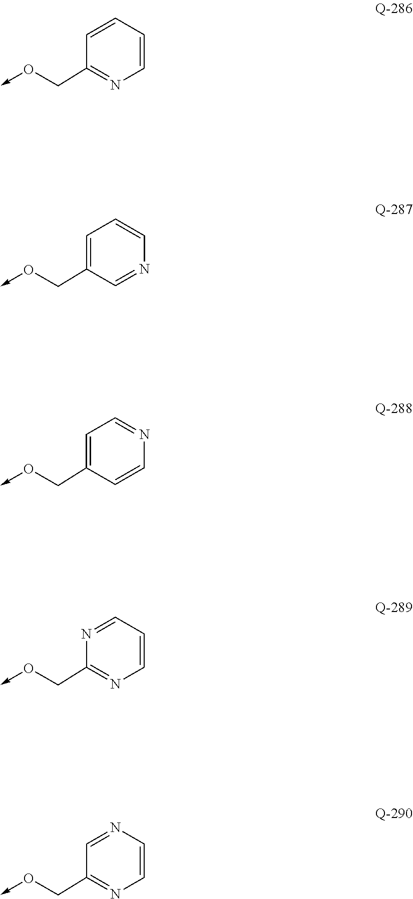

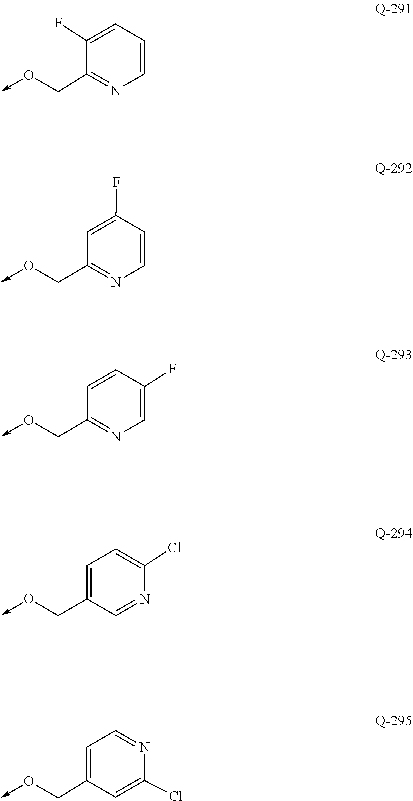

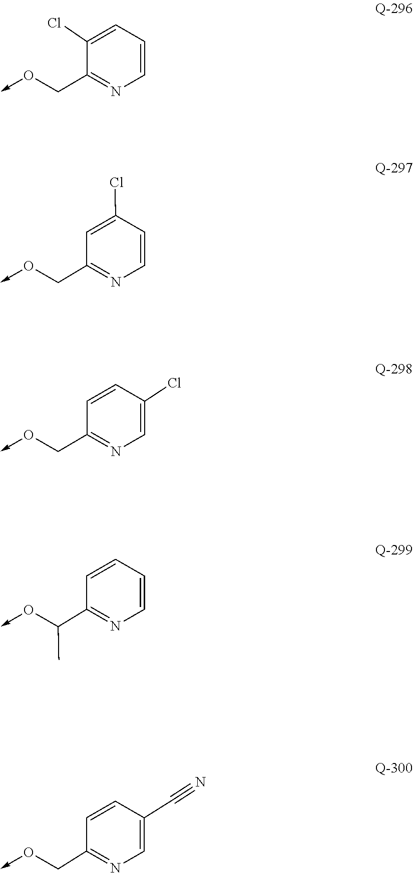

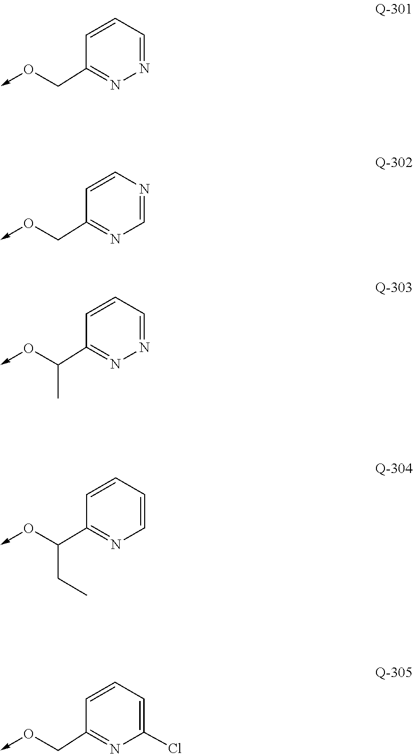

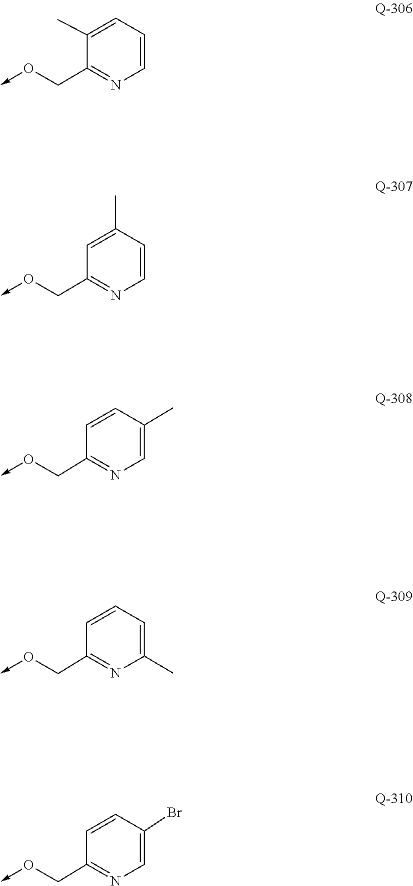

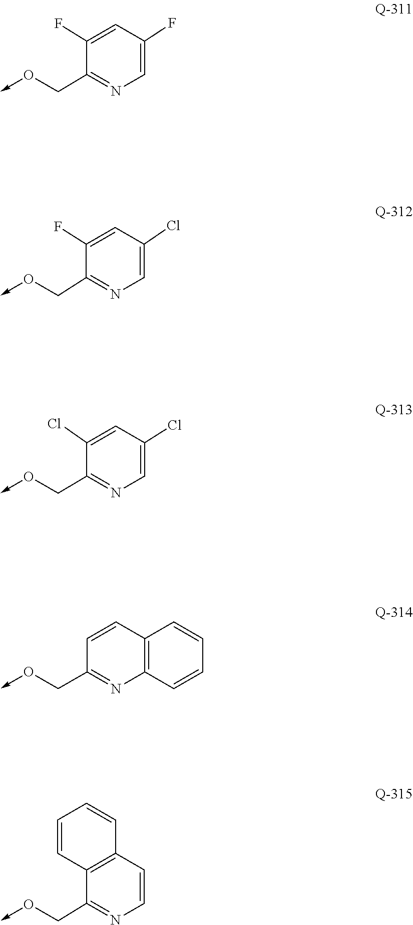

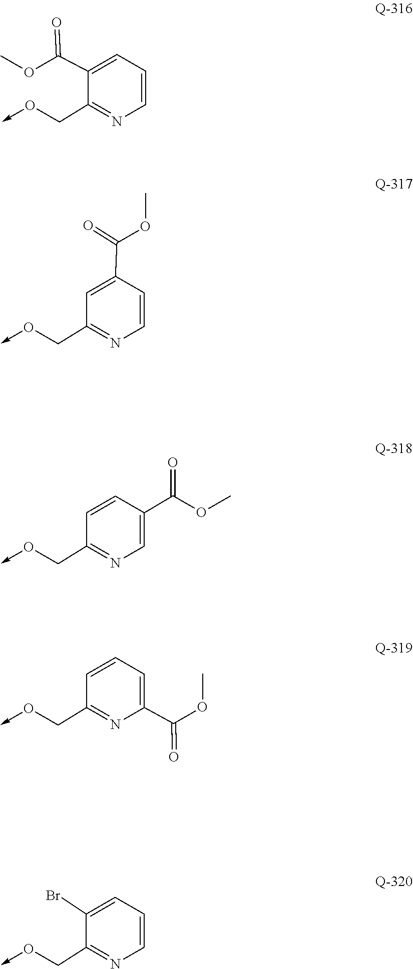

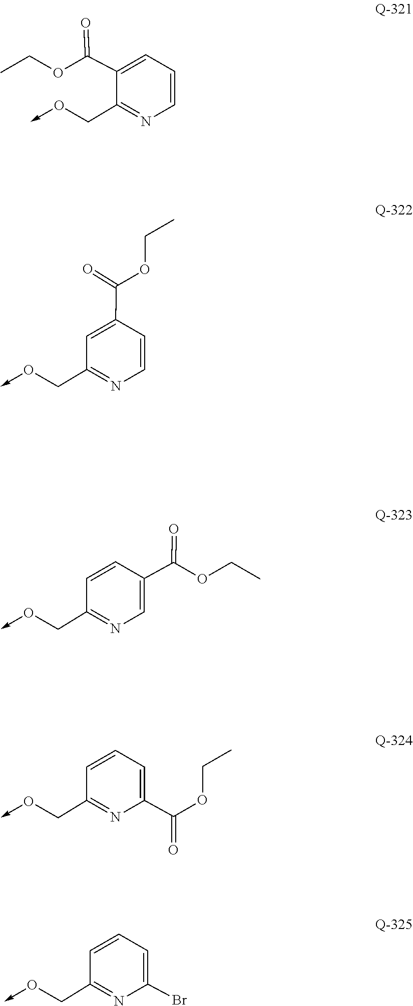

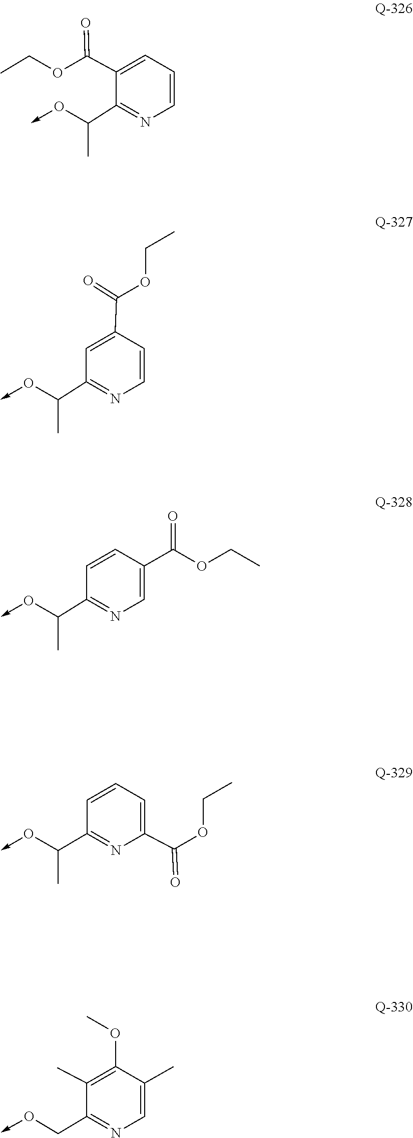

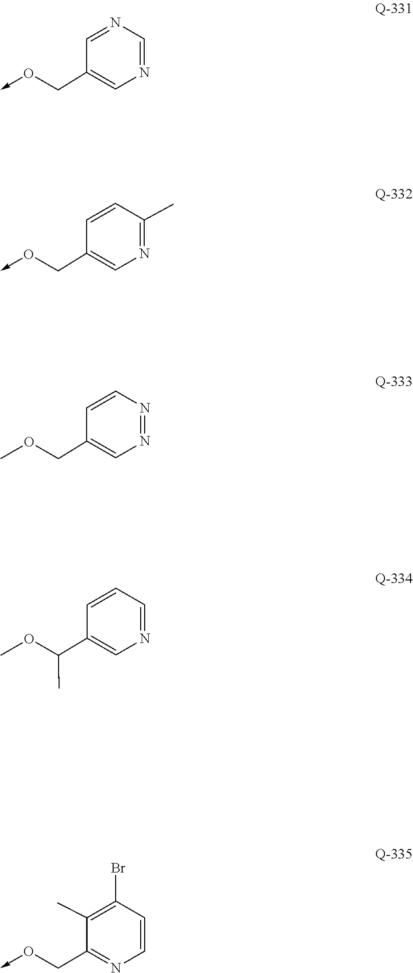

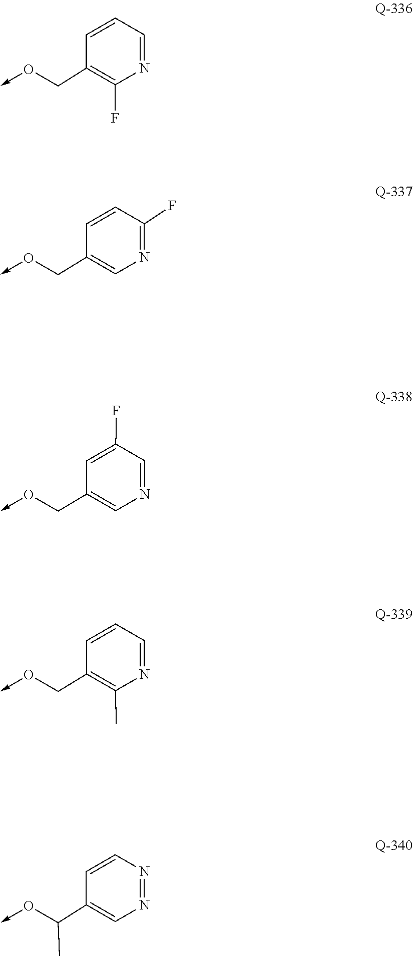

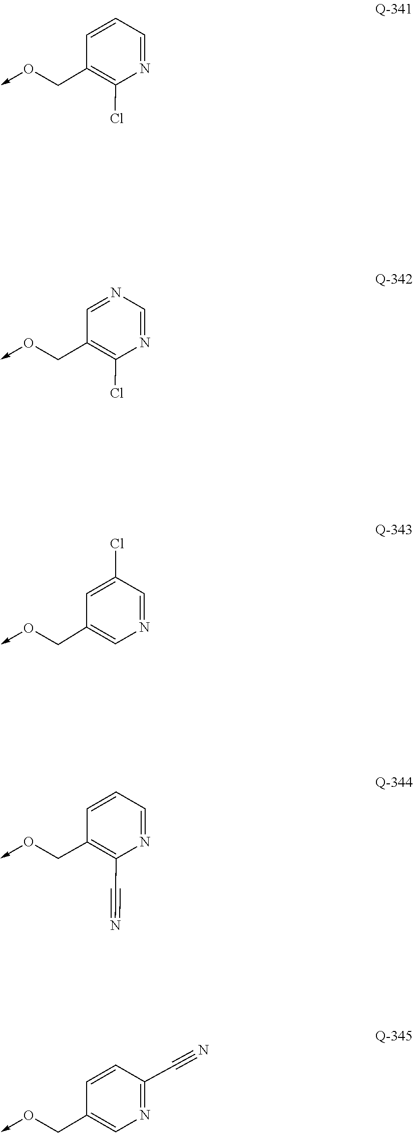

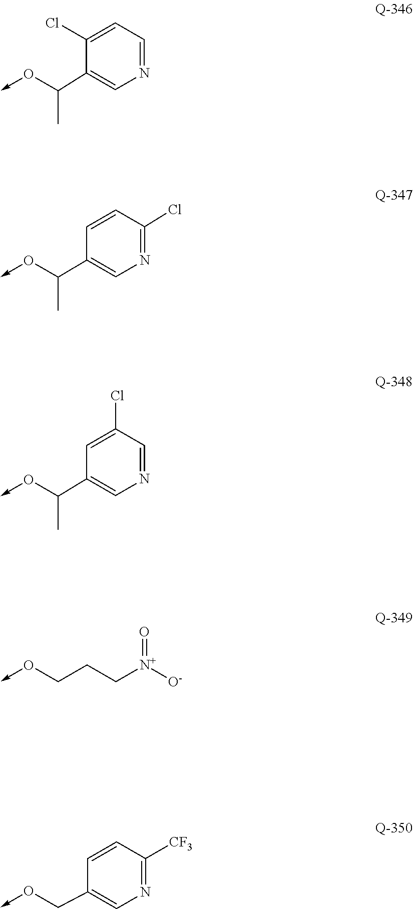

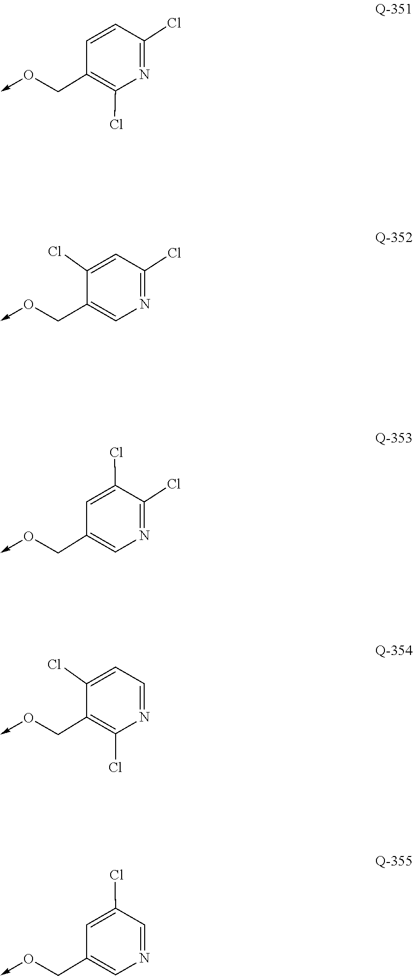

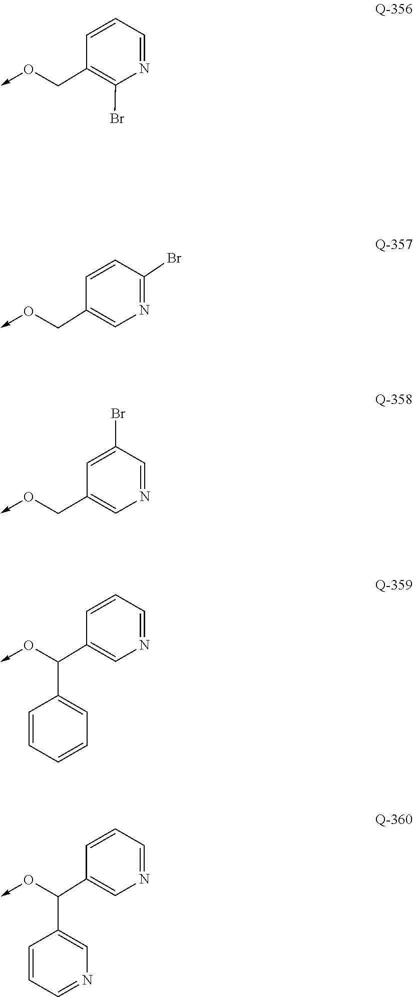

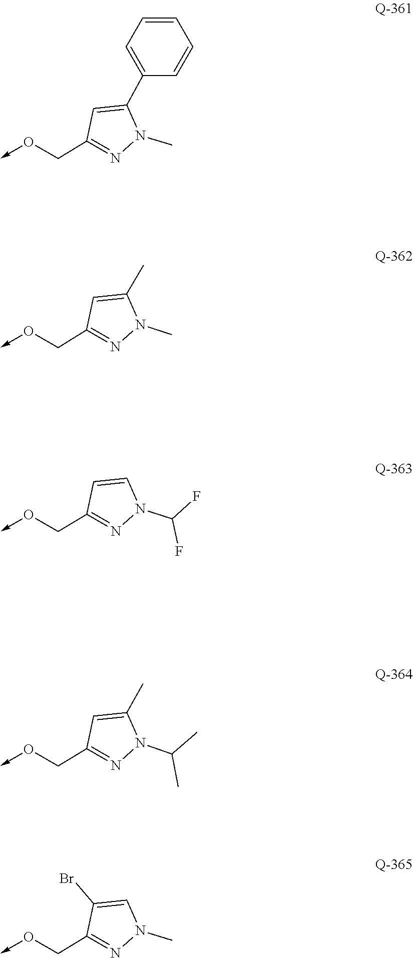

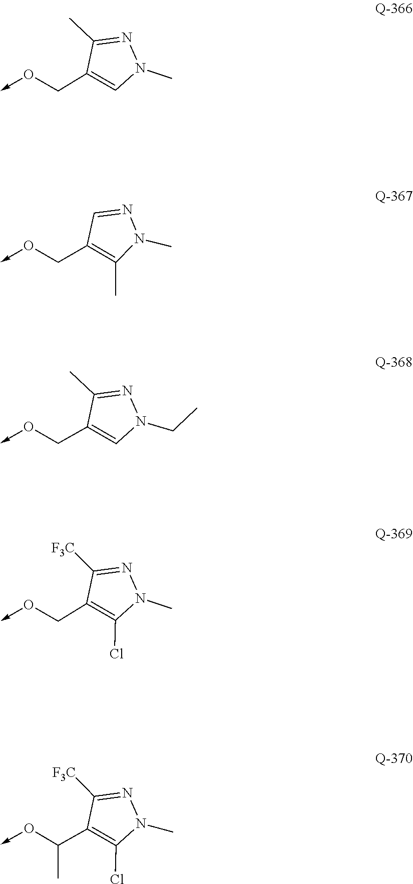

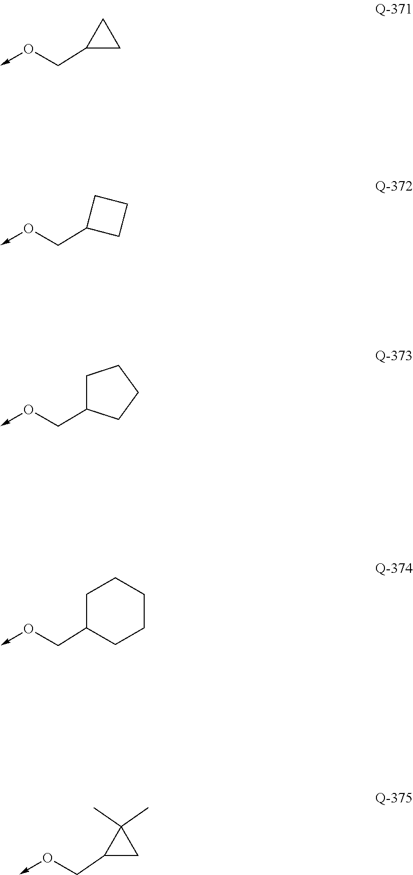

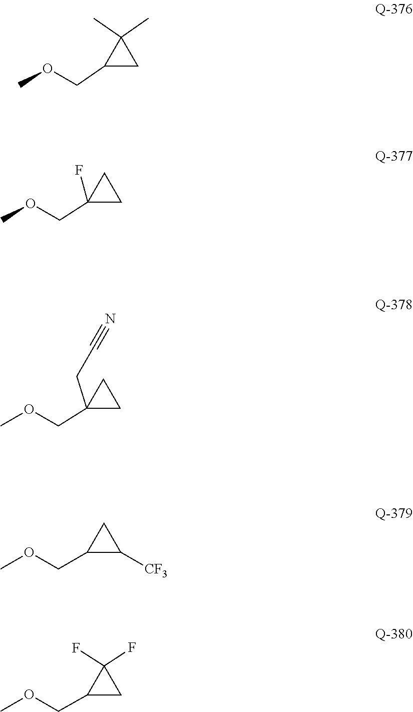

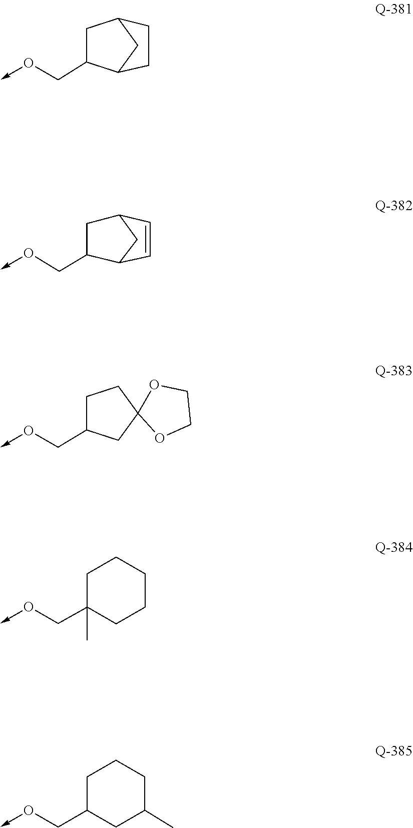

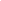

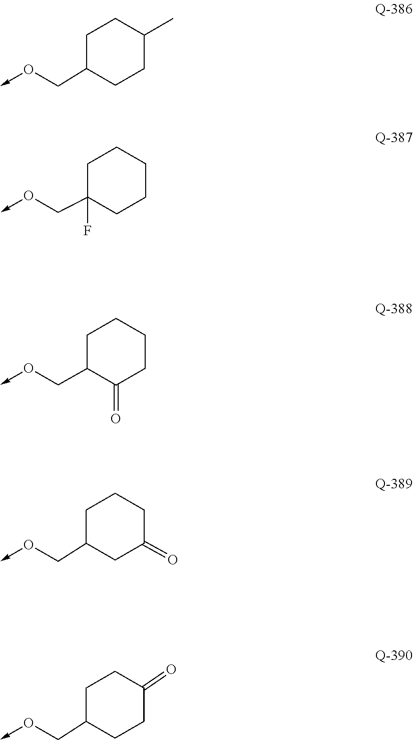

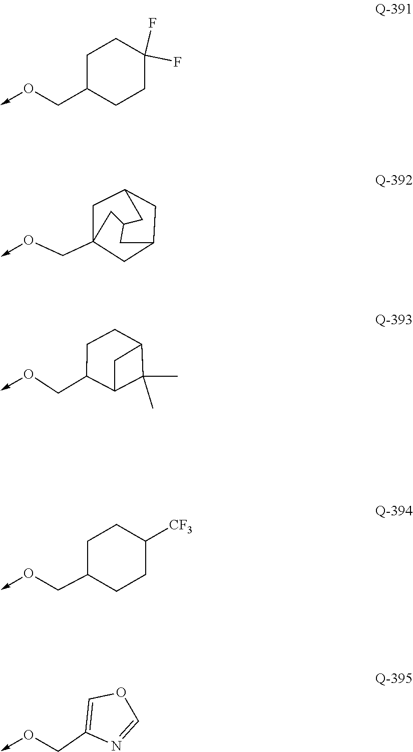

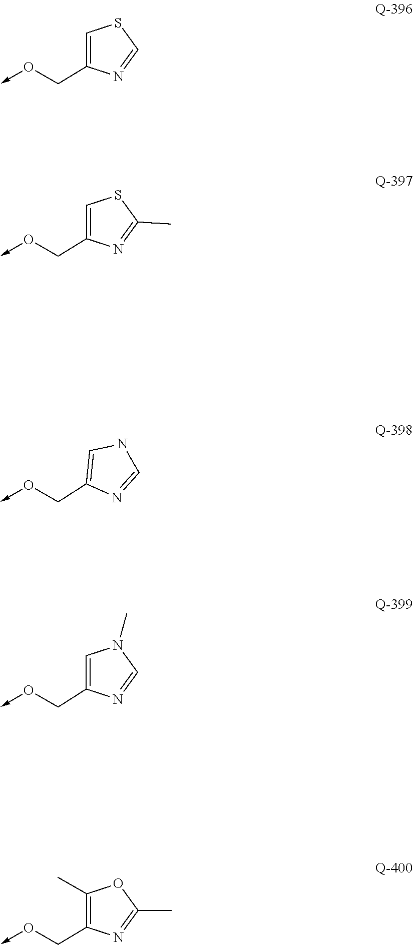

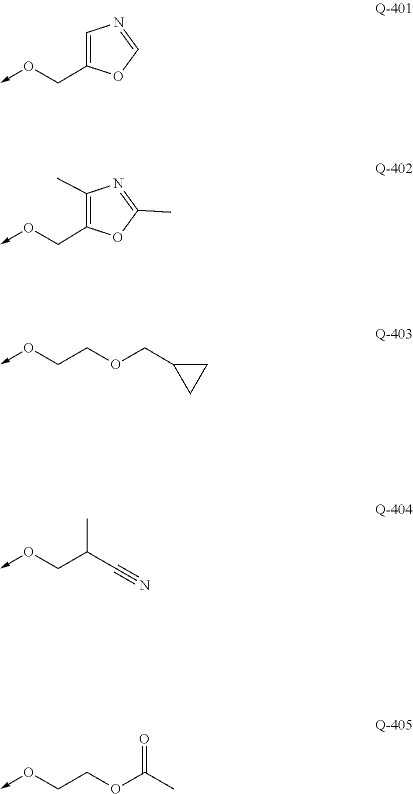

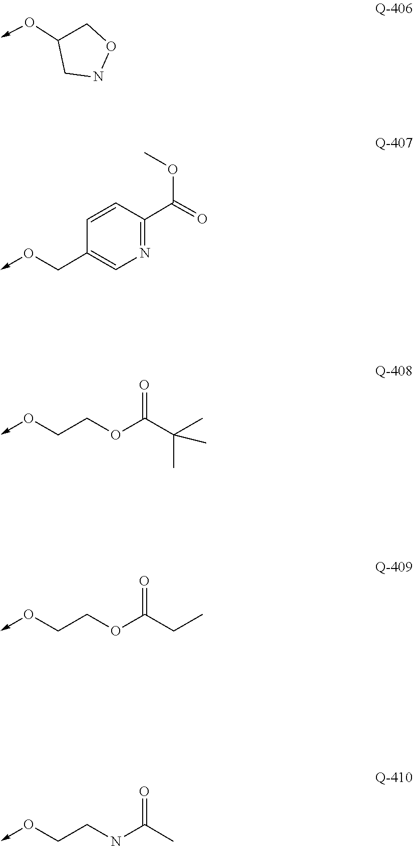

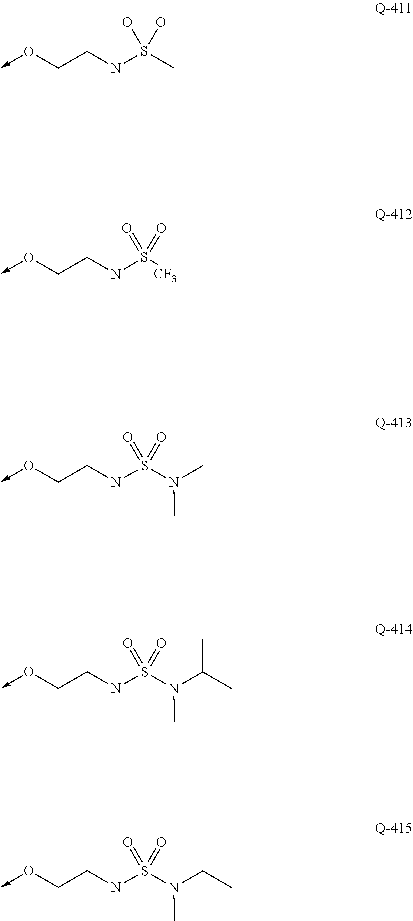

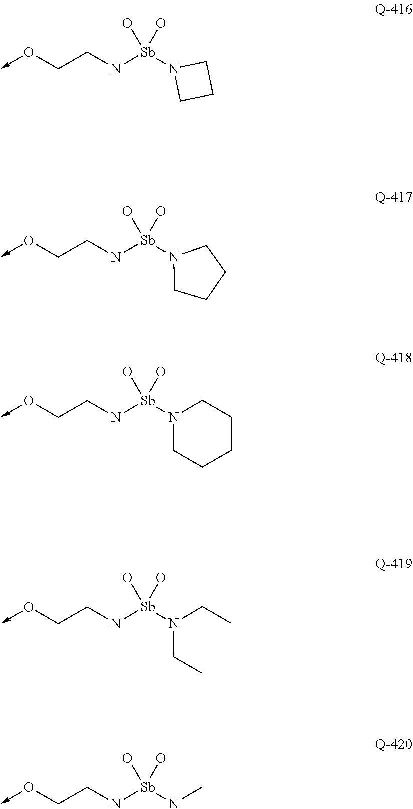

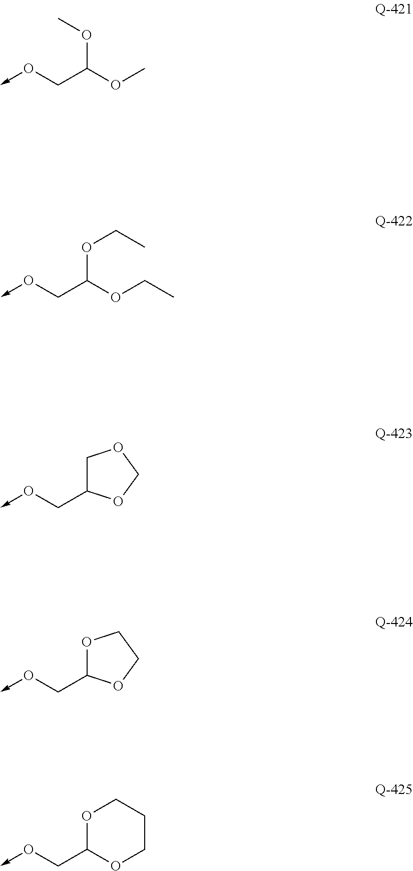

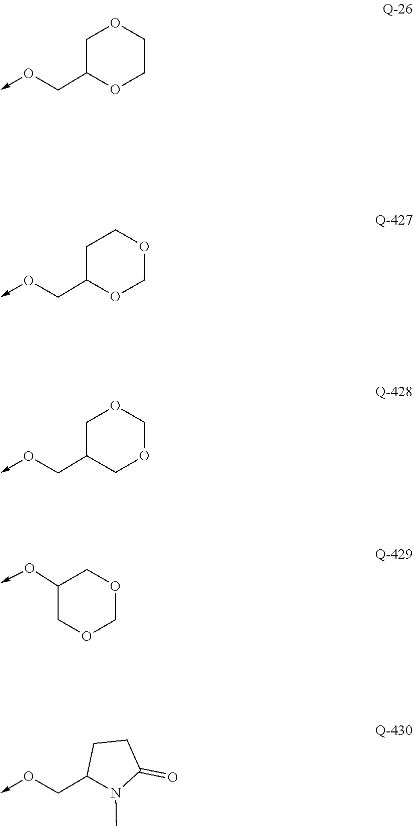

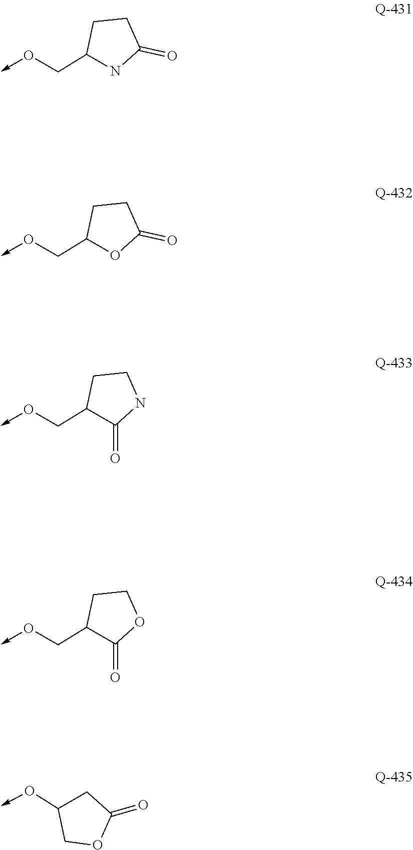

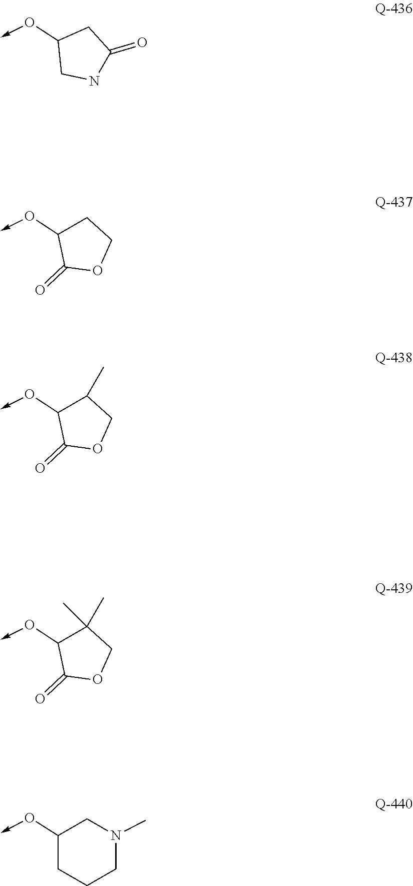

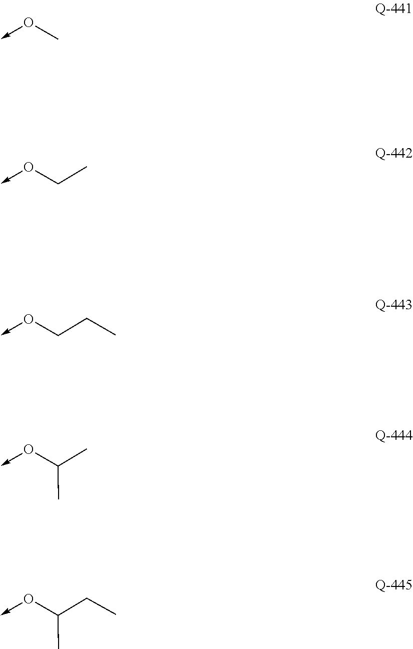

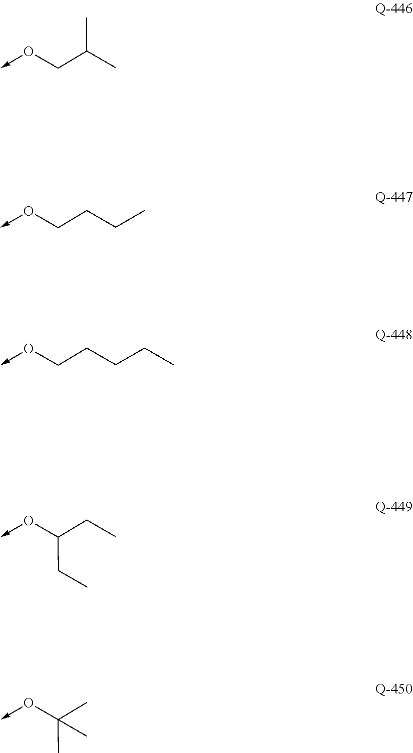

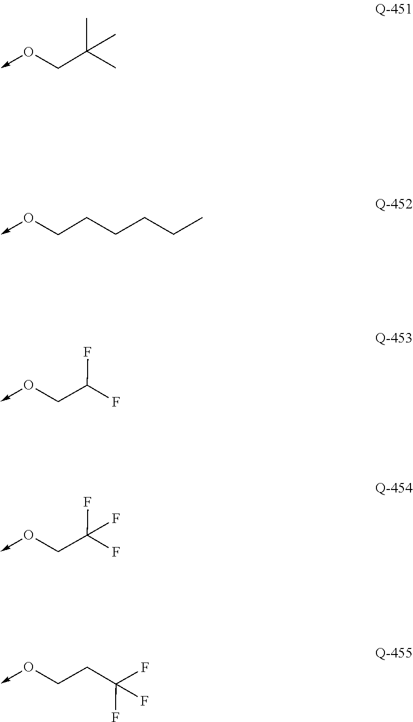

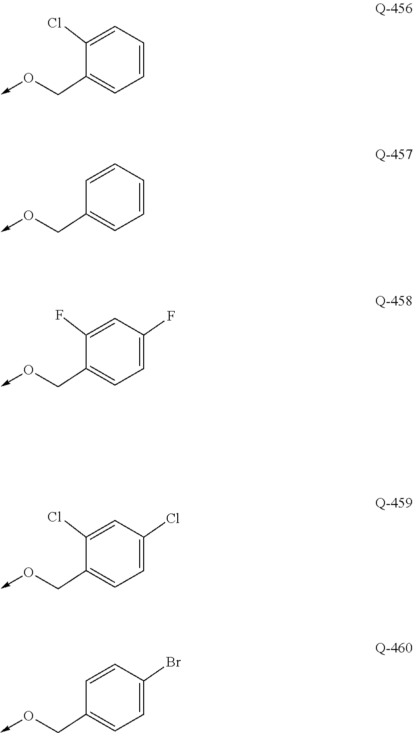

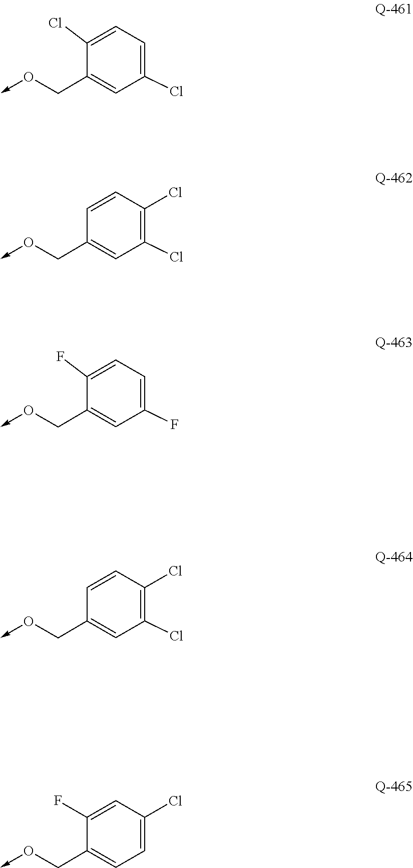

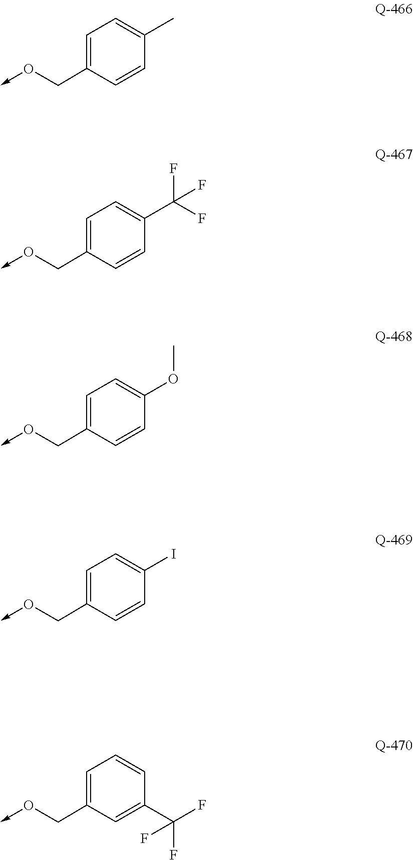

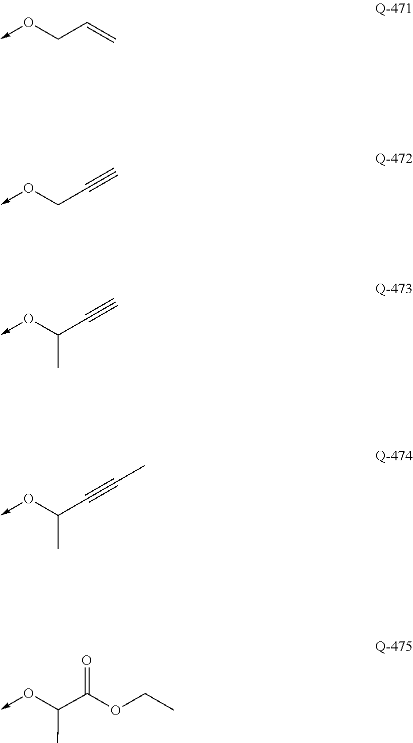

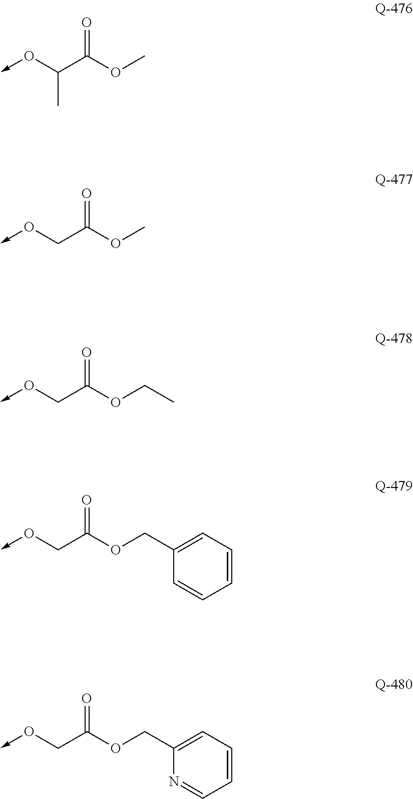

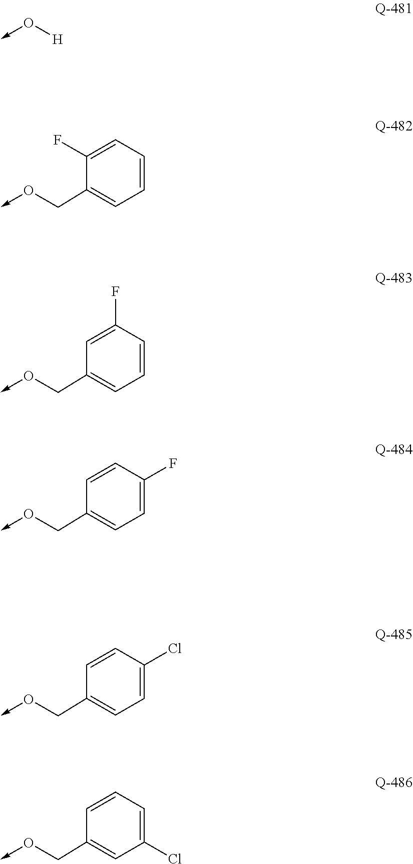

4. The compound of formula (I) as claimed in claim 3 and/or salt thereof, wherein
R¹ represents hydrogen,
R² represents fluorine,
R³ represents fluorine,
R⁴ represents chlorine, bromine, cyano, NO₂, C(O)NH₂, C(S)NH₂,
R⁵, R⁶ and R⁷ independently of one another represent hydrogen, fluorine, chlorine, bromine, cyano, methyl, trifluoromethyl, methoxy, trifluoromethoxy,
G represents methylene, (methyl)methylene, (ethyl)methylene, (dimethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, n-pentylene, n-hexylene,
R¹⁵ and R¹⁶ represent hydrogen,
X and Y independently of one another represent O (oxygen) or S (sulfur) and
Q represents one of the moieties Q-1 to Q-486.

5. The compound of formula (I) as claimed in claim 3 and/or salt thereof, wherein
R¹ represents hydrogen,
R² represents fluorine,
R³ represents fluorine,
R⁴ represents chlorine, bromine, cyano, NO₂, C(O)NH₂, C(S)NH₂,
R⁵, R⁶ and R⁷ independently of one another represent hydrogen, fluorine, chlorine, bromine, cyano, methyl, trifluoromethyl, methoxy, trifluoromethoxy,
G represents methylene, (methyl)methylene, (ethyl)methylene, (dimethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, n-pentylene, n-hexylene,
R¹⁵ and R¹⁶ represent hydrogen,
X and Y independently of one another represent O (oxygen) or S (sulfur) and
Q represents one of the moieties Q-1 to Q-481.

6. The compound of formula (I) as claimed in claim 3 and/or salt thereof, wherein
R¹ represents hydrogen,
R² represents fluorine,
R³ represents fluorine,
R⁴ represents chlorine, bromine, cyano, NO₂,
R⁵, R⁶ and R⁷ independently of one another represent hydrogen, fluorine, chlorine, bromine, methyl,
G represents methylene, (methyl)methylene, (ethyl)methylene, (dimethyl)methylene, ethylene, n-propylene, n-butylene, n-pentylene, n-hexylene,
R¹⁵ and R¹⁶ represent hydrogen,
X and Y independently of one another represent O (oxygen) or S (sulfur) and
Q represents one of the moieties Q-1 to Q-486.

7. The compound of formula (I) as claimed in claim 3 and/or salt thereof, wherein
R¹ represents hydrogen,
R² represents fluorine,
R³ represents fluorine,
R⁴ represents chlorine, NO₂,
R⁵ represents hydrogen,
R⁶ represents hydrogen, fluorine,
R⁷ represents hydrogen,
G represents methylene, (methyl)methylene, ethylene, n-propylene,
R¹⁵ and R¹⁶ represent hydrogen,
X represents O (oxygen) or S (sulfur),
Y represents O (oxygen) and
Q represents one of the moieties Q-1, Q-2, Q-23, Q-115, Q-176, Q-286, Q-441, Q-442, Q-447, Q-448, Q-457, Q-471, Q-481.

8. A product comprising one or more compounds of formula (I) as defined in claim 1 and/or salts thereof, as herbicide and/or plant growth regulator, optionally in crops of useful plants and/or ornamentals.

9. A herbicidal and/or plant growth-regulating composition, wherein the composition comprises one or more compounds of formula (I) as defined in claim 1 and/or salts thereof, and one or more further substances selected from groups (i) and/or (ii),
(i) one or more further agrochemically active substances, optionally selected from the group consisting of insecticides, acaricides, nematicides, further herbicides, fungicides, safeners, fertilizers and/or further growth regulators,
(ii) one or more formulation auxiliaries customary in crop protection.

10. A method for controlling one or more harmful plants or for regulating the growth of one or more plants, comprising applying an effective amount
of one or more compounds of formula (I), as defined in claim 1 and/or salts thereof, or
a composition thereof,
to the plants, seed of plants, soil in which or on which plants grow or an area under cultivation.
